(19) United States
(12) Patent Application Publication
(10) Pub. No.: US 2024/0415980 A1
(43) Pub. Date: Dec. 19, 2024

(54) CRISPR/CAS-RELATED METHODS AND COMPOSITIONS FOR KNOCKING OUT C5

(71) Applicant: Regeneron Pharmaceuticals, Inc., Tarrytown, NY (US)

(72) Inventors: Kishor Devalaraja-Narashimha, Yorktown Heights, NY (US); Lori Morton, Chappaqua, NY (US); Evangelos Pefanis, Somers, NY (US); Suzanne Hartford, Putnam Valley, NY (US); Arti Mahendra Prakash Kanjolia, Malden, MA (US); Sarah Hesse, Arlington, MA (US)

(21) Appl. No.: 18/704,681

(22) PCT Filed: Oct. 28, 2022
(86) PCT No.: PCT/US2022/078855

§ 371 Date: Apr. 25, 2024

Related U.S. Application Data
(60) Provisional application No. 63/272,863, filed on Oct. 28, 2021.

(51) Int. Cl. A61K 48/00; A61K 9/00; A61K 9/127; A61P 7/06; C07K 16/18; C12N 9/22; C12N 15/11; C12N 15/88

(52) U.S. Cl. CPC A61K 48/005; A61K 9/0019; A61K 9/1272; A61K 48/0075; A61P 7/06; C07K 16/18; C12N 9/22; C12N 15/111; C12N 15/88; C07K 2317/565; C12N 2310/20; C12N 2310/316; C12N 2310/321

(57) ABSTRACT

Guide RNAs and CRISPR/Cas systems targeting a C5 locus or gene, lipid nanoparticles or viral vectors comprising such guide RNAs or CRISPR/Cas systems, and cells or animals comprising such guide RNAs or systems are provided. Methods of modifying or knocking down or knocking out a C5 locus or gene using the CRISPR/Cas systems are also provided, as well as use of the CRISPR/Cas systems in prophylactic and therapeutic applications for treatment and/or prevention of a disease, disorder, or condition associated with C5 and/or for ameliorating at least one symptom associated with such disease, disorder, or condition.

### CROSS-REFERENCE TO RELATED APPLICATIONS

This application claims the benefit of U.S. Application No. 63/272,863, filed Oct. 28, 2021, which is herein incorporated by reference in its entirety for all purposes.

### REFERENCE TO A SEQUENCE LISTING SUBMITTED AS A TEXT FILE VIA EFS WEB

The Sequence Listing written in file 057766-586086.xml is 914 kilobytes, was created on Oct. 28, 2022, and is hereby incorporated by reference.

### BACKGROUND

The complement system is a group of plasma proteins that when activated lead to target cell lysis and facilitate phagocytosis through opsonization. Complement is activated through a series of proteolytic steps by three major pathways: the classical pathway, which is typically activated by immune complexes; the alternative pathway that can be induced by unprotected cell surfaces; and the mannose binding lectin pathway. All three pathways of complement cascade converge on proteolytic cleavage of complement component 5 (C5) protein. Cleavage of complement component 5 (C5) results in the production of fragments C5a and C5b, a process that is critical during the activation of the complement cascade. C5a can generate pleiotropic physiological responses through binding to its receptors. C5a is a potent proinflammatory mediator that induces chemotactic migration, enhances cell adhesion, stimulates the oxidative burst, and induces the release of various inflammatory mediators such as histamine or cytokines. C5b mediates the formation of the membrane-attack complex (MAC, or C5b-9) leading to cell lysis in the late phases of the complement dependent cytotoxicity (CDC). Further, in nucleated cells that are resistant to cytolysis by C5b-9, sublytic quantities of C5b-9 can cause cellular activation which results in cell proliferation, generation of proinflammatory mediators and production of extracellular matrix.

There remains a need for therapeutics that target a C5 gene for the prevention and treatment of C5-associated diseases.

### SUMMARY

Provided are compositions comprising a guide RNA or a DNA encoding the guide RNA, cells comprising the compositions, methods of modifying a C5 gene in a cell, methods of modifying a C5 gene or reducing expression of a C5 gene or reducing activity of complement C5 protein in a subject, and methods of preventing, treating, or ameliorating at least one symptom or indication of a disease or disorder associated with C5.

In one aspect, provided is a composition comprising a guide RNA or a DNA encoding the guide RNA, wherein the guide RNA comprises a DNA-targeting segment that targets a guide RNA target sequence in a C5 gene, and wherein the guide RNA binds to a Cas protein and targets the Cas protein to the guide RNA target sequence in the C5 gene.

In some such compositions, the guide RNA target sequence is in coding exon 27, 22, 21, 15, 12, or 1 of the C5 gene, or wherein the guide RNA target sequence is in coding exon 15 or 12 of the C5 gene. In some such compositions, the C5 gene is a human C5 gene.

In some such compositions, the DNA-targeting segment comprises at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97. In some such compositions, the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273. In some such compositions, the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

In some such compositions, the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97. In some such compositions, the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

In some such compositions, the composition comprises the guide RNA in the form of RNA. In some such compositions, the composition comprises the DNA encoding the guide RNA. In some such compositions, the guide RNA comprises at least one modification. In some such compositions, the at least one modification comprises a 2′-O-methyl-modified nucleotide. In some such compositions, the at least one modification comprise a phosphorothioate bond between nucleotides. In some such compositions, the at least one modification comprise a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the guide RNA comprises the modified nucleotides of SEQ ID NO: 29.

In some such compositions, the guide RNA is a single guide RNA (sgRNA). In some such compositions, the guide RNA comprises, consists essentially of, or consists of the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

In some such compositions, the guide RNA is a dual guide RNA (dgRNA) comprising two separate RNA molecules comprising a CRISPR RNA (crRNA) and a trans-activating crRNA (tracrRNA). In some such compositions, the crRNA comprises the sequence set forth in any one of SEQ ID NOS: 16-17. In some such compositions, the tracrRNA comprises the sequence set forth in any one of SEQ ID NOS: 18-20.

In some such compositions, the composition is associated with a lipid nanoparticle. In some such compositions, the lipid nanoparticle comprises a cationic lipid, a neutral lipid, a helper lipid, and a stealth lipid. In some such compositions, the cationic lipid is Lipid A. In some such compositions, the neutral lipid is DSPC. In some such compositions, the helper lipid is cholesterol. In some such compositions, the stealth lipid is PEG2k-DMG. In some such compositions, the cationic lipid is Lipid A, the neutral lipid is DSPC, the helper lipid is cholesterol, and the stealth lipid is PEG2k-DMG.

In some such compositions, the composition is a pharmaceutical composition comprising a pharmaceutically acceptable carrier.

In some such compositions, the composition further comprises the Cas protein or a nucleic acid encoding the Cas protein. In some such compositions, the Cas protein is a Cas9 protein. In some such compositions, the Cas protein is derived from a Streptococcus pyogenes Cas9 protein. In some such compositions, the composition comprises the Cas protein in the form of a protein. In some such compositions, the composition comprises the nucleic acid encoding the Cas protein, wherein the nucleic acid comprises a DNA encoding the Cas protein, optionally wherein the composition comprises the DNA encoding the guide RNA. In some such compositions, the composition comprises the nucleic acid encoding the Cas protein, wherein the nucleic acid comprises an mRNA encoding the Cas protein, optionally wherein the composition comprises the guide RNA in the form of RNA. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is pseudouridine. In some such compositions, the modified uridine is N1-methyl-pseudouridine. In some such compositions, the mRNA encoding the Cas protein is fully substituted with pseudouridine. In some such compositions, the mRNA encoding the Cas protein is fully substituted with N1-methyl-pseudouridine. In some such compositions, the mRNA encoding the Cas protein comprises a 5′ cap. In some such compositions, the mRNA encoding the Cas protein comprises a poly(A) tail. In some such compositions, the mRNA encoding the Cas protein comprises the sequence set forth in SEQ ID NO: 339, 338, or 12. In some such compositions, the nucleic acid encoding the Cas protein is codon-optimized for expression in a mammalian cell or a human cell. In some such compositions, the Cas protein comprises the sequence set forth in SEQ ID NO: 11 or 8.

In some such compositions, the compositions further comprises a second guide RNA or a DNA encoding the second guide RNA, wherein the second guide RNA comprises a DNA-targeting segment that targets a second guide RNA target sequence in the C5 gene, and wherein the second guide RNA binds to the Cas protein and targets the Cas protein to the second guide RNA target sequence in the C5 gene.

In some such compositions, the composition is in association with an antigen-binding protein that binds specifically to C5. In some such compositions, the antigen-binding protein that binds specifically to C5 is an antibody or an antigen-binding fragment thereof. In some such compositions, the antigen-binding protein that binds specifically to C5 comprises: (1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof, (2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof, (3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof, (4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof, (5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof, (6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof, (7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof, (8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof, (9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof, (10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof, (11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof, (12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof, (13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof, (14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof, (15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof, (16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof, (17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof, (18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof, (19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof, (20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof, (21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof, (22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof, (23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof, (24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof, (25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof, (26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof, (27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof, (28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or (29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or competes for binding to C5 with an antigen-binding protein selected from the group consisting of (1)-(29); or binds to the same epitope on C5 as an antigen-binding protein selected from the group consisting of (1)-(29). In some such compositions, the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof. In some such compositions, the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698. In some such compositions, the antigen-binding protein that binds specifically to C5 is pozelimab.

In another aspect, provided is a cell comprising any of the above compositions.

In another aspect, provided are methods of modifying a C5 gene in a cell. Some such methods comprise introducing any of the above compositions into the cell, wherein the guide RNA forms a complex with the Cas protein and targets the guide RNA target sequence in the C5 gene, and the Cas protein cleaves the guide RNA target sequence to generate a targeted genetic modification in the C5 gene.

In some such methods, cleavage by the Cas protein creates a double-strand break in the C5 gene. In some such methods, cleavage by the Cas protein creates a single-strand break in the C5 gene. In some such methods, the targeted genetic modification is generated by repair of the cleaved guide RNA target sequence by non-homologous end-joining. In some such methods, the method results in reduced expression or activity of the C5 gene in the cell or wherein the method results in loss of function or inactivation of the C5 gene in the cell.

In some such methods, the cell is a hepatocyte. In some such methods, the cell is a mammalian cell, and the C5 gene is a mammalian C5 gene. In some such methods, the cell is a human cell, and the C5 gene is a human C5 gene. In some such methods, the cell is in vitro or ex vivo. In some such methods, the cell is in an animal in vivo.

In some such methods, the method results in at least 50%, at least 55%, at least 60%, at least 65%, at least 70%, at least 75%, at least 80%, at least 85%, at least 90%, or at least 95% percent editing of the C5 gene in a target population of cells in the animal. In some such methods, the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the animal.

In some such methods, the method results in reduced serum levels of complement C5 protein in the animal, optionally wherein serum levels of complement C5 protein are reduced by at least 50%, at least 55%, at least 60%, at least 65%, at least 70%, at least 75%, at least 80%, at least 85%, at least 90%, or at least 95%. In some such methods, the method results in reduced complement C5 protein activity in the animal, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells.

In some such methods, C5 complement activity is reduced by about 95-100% as measured by CH50 assay of complement-mediated sheep red blood cell lysis. In some such methods, the method further comprises administering to the animal a further therapeutic agent. In some such methods, the further therapeutic agent is an antigen-binding protein that binds specifically to C5, acetaminophen, an albumin infusion, ancrod, an angiotensin-converting enzyme inhibitor, an antibiotic, an anti-CD20 agent, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a meningococcal vaccine, methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin, and/or warfarin.

In some such methods, the therapeutic agent is the antigen-binding protein that binds specifically to C5. In some such methods, the antigen-binding protein is administered to the animal intravenously or subcutaneously. In some such methods, a first dose of the antigen-binding protein is administered to the animal intravenously, and one or more additional doses of the antigen-binding protein are administered subcutaneously. In some such methods, the antigen-binding protein that binds specifically to C5 is an antibody or an antigen-binding fragment thereof. In some such methods, the antigen-binding protein that binds specifically to C5 comprises: (1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof, (2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof, (3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof, (4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof, (5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof, (6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof, (7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof, (8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof, (9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof, (10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof, (11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof, (12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof, (13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof, (14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof, (15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof, (16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof, (17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof, (18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof, (19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof, (20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof, (21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof, (22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof, (23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof, (24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof, (25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof, (26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof, (27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof, (28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or (29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or competes for binding to C5 with an antigen-binding protein selected from the group consisting of (1)-(29); or binds to the same epitope on C5 as an antigen-binding protein selected from the group consisting of (1)-(29). In some such methods, the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof. In some such methods, the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698. In some such methods, the antigen-binding protein that binds specifically to C5 is pozelimab.

In another aspect, provided are methods of modifying a C5 gene in a cell. Some such methods comprise contacting the genome of the cell with: (a) a Cas protein; and (b) a guide RNA that forms a complex with the Cas protein and targets a guide RNA target sequence in the C5 gene, wherein the Cas protein cleaves the guide RNA target sequence to generate a targeted genetic modification in the C5 gene.

In some such methods, the guide RNA target sequence is in coding exon 27, 22, 21, 15, 12, or 1 of the C5 gene, or wherein the guide RNA target sequence is in coding exon 15 or 12 of the C5 gene. In some such methods, the guide RNA comprises a DNA-targeting segment that targets the guide RNA target sequence, wherein the DNA-targeting segment comprises, consists essentially of, or consists of at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97. In some such methods, the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273. In some such methods, the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

In some such methods, the guide RNA comprises a DNA-targeting segment that targets the guide RNA target sequence, wherein the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97. In some such methods, the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

In some such methods, the method comprises introducing into the cell: (a) the Cas protein or a nucleic acid encoding the Cas protein; and (b) the guide RNA or a DNA encoding the guide RNA. In some such methods, the Cas protein or the nucleic acid encoding the Cas protein and/or the guide RNA or the DNA encoding the guide RNA are introduced into the cell via lipid-nanoparticle-mediated delivery. In some such methods, the guide RNA in the form of RNA and the nucleic acid encoding the Cas protein are introduced into the cell via lipid-nanoparticle-mediated delivery, wherein the nucleic acid encoding the Cas protein is an mRNA. In some such methods, the lipid nanoparticle comprises a cationic lipid, a neutral lipid, a helper lipid, and a stealth lipid. In some such methods, the cationic lipid is Lipid A. In some such methods, the neutral lipid is DSPC. In some such methods, the helper lipid is cholesterol. In some such methods, the stealth lipid is PEG2k-DMG. In some such methods, the cationic lipid is Lipid A, the neutral lipid is DSPC, the helper lipid is cholesterol, and the stealth lipid is PEG2k-DMG.

In some such methods, the Cas protein or the nucleic acid encoding the Cas protein and/or the guide RNA or the DNA encoding the guide RNA are introduced into the cell via adeno-associated virus.

In some such methods, the method comprises introducing into the cell the nucleic acid encoding the Cas protein. In some such methods, the nucleic acid encoding the Cas protein is codon-optimized for expression in a mammalian cell or a human cell. In some such methods, the nucleic acid encoding the Cas protein comprises DNA, optionally wherein the method comprises introducing into the cell the DNA encoding the guide RNA. In some such methods, the nucleic acid encoding the Cas protein comprises RNA, optionally wherein the method comprises introducing into the cell the guide RNA in the form of RNA. In some such methods, the RNA encoding the Cas protein comprises at least one modification. In some such methods, the RNA encoding the Cas protein is modified to comprise a modified uridine at one or more or all uridine positions. In some such methods, the modified uridine is pseudouridine. In some such methods, the modified uridine is N1-methyl-pseudouridine. In some such methods, the RNA encoding the Cas protein is fully substituted with pseudouridine. In some such methods, the RNA encoding the Cas protein is fully substituted with N1-methyl-pseudouridine. In some such methods, the RNA encoding the Cas protein comprises a 5′ cap. In some such methods, the RNA encoding the Cas protein comprises a poly(A) tail. In some such methods, the RNA encoding the Cas protein comprises the sequence set forth in SEQ ID NO: 339, 338, or 12.

In some such methods, the method comprises introducing into the cell the guide RNA in the form of RNA. In some such methods, the method comprises introducing into the cell the DNA encoding the guide RNA. In some such methods, the guide RNA comprises at least one modification. In some such methods, the at least one modification comprises a 2′-O-methyl-modified nucleotide. In some such methods, the at least one modification comprise a phosphorothioate bond between nucleotides. In some such methods, the at least one modification comprise a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA comprises the modified nucleotides of SEQ ID NO: 29.

In some such methods, the guide RNA is a single guide RNA (sgRNA). In some such methods, the guide RNA comprises the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

In some such methods, the guide RNA is a dual guide RNA (dgRNA) comprising two separate RNA molecules comprising a CRISPR RNA (crRNA) and a trans-activating crRNA (tracrRNA). In some such methods, the crRNA comprises the sequence set forth in any one of SEQ ID NOS: 16-17. In some such methods, the tracrRNA comprises the sequence set forth in any one of SEQ ID NOS: 18-20.

In some such methods, the Cas protein is a Cas9 protein. In some such methods, the Cas protein is derived from a Streptococcus pyogenes Cas9 protein. In some such methods, the Cas protein comprises the sequence set forth in SEQ ID NO: 11 or 8.

In some such methods, the method further comprises introducing into the cell a second guide RNA or a DNA encoding the second guide RNA, wherein the second guide RNA forms a complex with the Cas protein and targets the Cas protein to a second guide RNA target sequence in the C5 gene, and wherein the Cas protein cleaves the second guide RNA target sequence to generate a targeted genetic modification in the C5 gene.

In some such methods, cleavage by the Cas protein creates a double-strand break in the C5 gene. In some such methods, cleavage by the Cas protein creates a single-strand break in the C5 gene. In some such methods, the targeted genetic modification is generated by repair of the cleaved guide RNA target sequence by non-homologous end-joining. In some such methods, the method results in reduced expression or activity of the C5 gene in the cell or wherein the method results in loss of function or inactivation of the C5 gene in the cell.

In some such methods, the cell is a hepatocyte. In some such methods, the cell is a mammalian cell, and the C5 gene is a mammalian C5 gene. In some such methods, the cell is a human cell, and the C5 gene is a human C5 gene. In some such methods, the cell is in vitro or ex vivo. In some such methods, the cell is in an animal in vivo.

In some such methods, the method results in at least 50%, at least 55%, at least 60%, at least 65%, at least 70%, at least 75%, at least 80%, at least 85%, at least 90%, or at least 95% percent editing of the C5 gene in a target population of cells in the animal. In some such methods, the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the animal.

In some such methods, the method results in reduced serum levels of complement C5 protein in the animal, optionally wherein serum levels of complement C5 protein are reduced by at least 50%, at least 55%, at least 60%, at least 65%, at least 70%, at least 75%, at least 80%, at least 85%, at least 90%, or at least 95%. In some such methods, the method results in reduced complement C5 protein activity in the animal, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells.

In another aspect, provided are methods of modifying a C5 gene or reducing expression of a C5 gene or reducing activity of complement C5 protein in a subject. Some such methods comprise administering to a subject: (a) a Cas protein or a nucleic acid encoding the Cas protein; and (b) a guide RNA or a DNA encoding the guide RNA, wherein the guide RNA forms a complex with the Cas protein and targets a guide RNA target sequence in a C5 gene, wherein the Cas protein cleaves the guide RNA target sequence to generate a targeted genetic modification in the C5 gene. In another aspect, provided are methods of preventing, treating, or ameliorating at least one symptom or indication of a disease or disorder associated with C5. Some such methods comprise administering to a subject in need thereof a pharmaceutical composition comprising a therapeutically effective amount of: (a) a Cas protein or a nucleic acid encoding the Cas protein; and (b) a guide RNA or a DNA encoding the guide RNA, wherein the guide RNA forms a complex with the Cas protein and targets a guide RNA target sequence in a C5 gene, wherein the Cas protein cleaves the guide RNA target sequence to generate a targeted genetic modification in the C5 gene.

In some such methods, the disease or disorder is adult respiratory distress syndrome; age-related macular degeneration (AMD); allergy; Alport's syndrome; Alzheimer's disease; amyotrophic lateral sclerosis (ALS); antiphospholipid syndrome (APS); asthma; atherosclerosis; atypical hemolytic uremic syndrome (aHUS); an autoimmune disease; autoimmune hemolytic anemia (AIHA); balloon angioplasty; bronchoconstriction; bullous pemphigoid; burns; C3 glomerulopathy; capillary leak syndrome; a cardiovascular disorder; catastrophic antiphospholipid syndrome (CAPS); a cerebrovascular disorder; CHAPLE disease (CD55 deficiency with hyperactivation of complement, angiopathic thrombosis, and protein-losing enteropathy); a chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; eclampsia; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain-Barre Syndrome; Hashimoto's thyroiditis; hemodialysis complications; hemolysis-elevated liver enzymes- and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; an immune complex disorder; immune complex vasculitis; immune complex-associated inflammation; an infectious disease; inflammation caused by an autoimmune disease; an inflammatory disorder; inherited CD59 deficiency; injury due to inert dusts and/or minerals; interleukin-2 induced toxicity during IL-2 therapy; ischemia-reperfusion injury; Kawasaki's disease; a lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); pauci-immune vasculitis; pemphigus; percutaneous transluminal coronary angioplasty (PTCA); peripheral vascular disorder; pneumonia; post-ischemic reperfusion condition; post-pump syndrome in cardiopulmonary bypass; post-pump syndrome in renal bypass; pre-eclampsia; progressive kidney failure; proliferative nephritis; proteinuric kidney disease; psoriasis; pulmonary embolism; pulmonary fibrosis; pulmonary infarction; pulmonary vasculitis; recurrent fetal loss; a renal disorder; renal ischemia; renal ischemia-reperfusion injury; a renovascular disorder; restenosis following stent placement; rheumatoid arthritis (RA); rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome (tHUS); uveitis; vasculitis; vasculitis associated with rheumatoid arthritis; venous gas embolus (VGE); and/or xenograft rejection.

In some such methods, the disease or disorder is selected from the group consisting of atypical hemolytic uremic syndrome (aHUS), paroxysmal nocturnal hemoglobinuria (PNH), age-related macular degeneration, geographic atrophy, uveitis, neuromyelitis optica, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during TL-2 therapy, inflammatory disorders, inflammation of autoimmune diseases, Crohn's disease, adult respiratory distress syndrome, thermal injury including burns or frostbite, post-ischemic reperfusion conditions, myocardial infarction, capillary leak syndrome, obesity, diabetes, Alzheimer's disease, schizophrenia, stroke, epilepsy, atherosclerosis, vasculitis, bullous pemphigoid, C3 glomerulopathy, membranoproliferative glomerulonephritis, diabetic nephropathy, Alport's syndrome, progressive kidney failure, proteinuric kidney diseases, renal ischemia-reperfusion injury, lupus nephritis, balloon angioplasty, post-pump syndrome in cardiopulmonary bypass or renal bypass, hemodialysis, renal ischemia, mesenteric artery reperfusion after aortic reconstruction, infectious disease or sepsis, immune complex disorders and autoimmune diseases, renal disorders, rheumatoid arthritis, systemic lupus erythematosus (SLE), SLE nephritis, proliferative nephritis, hemolytic anemia, asthma, chronic obstructive pulmonary disease (COPD), emphysema, pulmonary embolisms and infarcts, pneumonia, and myasthenia gravis. In some such methods, the disease or disorder is atypical hemolytic uremic syndrome (aHUS), paroxysmal nocturnal hemoglobinuria (PNH), refractory myasthenia gravis (rMG), neuromyelitis optica (NMO), IgA nephropathy, membranous nephropathy, lupus nephritis, C3 glomerulopathy, and ANCA-vasculitis.

In some such methods, the disease or disorder is aHUS or PNH. In some such methods, the disease or disorder is aHUS. In some such methods, the disease or disorder is PNH. In some such methods, the disease or disorder is PNH, wherein the method is for reducing serum lactate dehydrogenase (LDH) levels, intravascular hemolysis, and/or the need for transfusions of red blood cells in the subject. In some such methods, the disease or disorder is CD55-deficient protein-losing enteropathy (CHAPLE disease). In some such methods, the disease or disorder is CD55-deficient protein-losing enteropathy (CHAPLE disease), wherein the method is for (i) normalizing and/or increasing serum albumin or decreasing loss thereof through the gastrointestinal tract; increasing total serum protein level, or decreasing loss thereof through the gastrointestinal tract; increasing serum vitamin B12 or gastrointestinal absorption thereof, decreasing platelet counts or decreasing coagulation cascade activation or decreasing the incidence of thrombotic events; decreasing the loss of alpha-1-antitrypsin through the gastrointestinal tract; treating or preventing facial and/or peripheral edema; decreasing the frequency of bowel movements; treating or preventing diarrhea; treating or preventing abdominal pain; decreasing the use of corticosteroids; and/or decreasing the incidence of hospitalization in the subject; or (ii) reducing therapeutic interventions in the subject, wherein the therapeutic intervention is one or more selected from the group consisting of: administration of a corticosteroid; administration of an immunoglobulin; administration of albumin; administration of an anti-tumor necrosis factor alpha therapeutic agent; administration of an immunomodulator; administration of a micronutrient; administration of enteral or parenteral supplementation; administration of an anti-coagulant; administration of an antibiotic; and administration of an anti-platelet agent. Optionally, the method is for increasing serum albumin by at least 1 g/dL and/or for normalizing serum albumin to about 3.5 to about 5.5 g/dL.

In some such methods, the pharmaceutical composition is administered prophylactically or therapeutically to the subject in need thereof. In some such methods, the pharmaceutical composition is administered intravenously.

In some such methods, the guide RNA target sequence is in coding exon 27, 22, 21, 15, 12, or 1 of the C5 gene, or wherein the guide RNA target sequence is in coding exon 15 or 12 of the C5 gene. In some such methods, the guide RNA comprises a DNA-targeting segment that targets the guide RNA target sequence, wherein the DNA-targeting segment comprises, consists essentially of, or consists of at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97. In some such methods, the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273. In some such methods, the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

In some such methods, the guide RNA comprises a DNA-targeting segment that targets the guide RNA target sequence, wherein the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97. In some such methods, the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

In some such methods, the Cas protein or the nucleic acid encoding the Cas protein and/or the guide RNA or the DNA encoding the guide RNA are administered to the subject via lipid-nanoparticle-mediated delivery. In some such methods, the guide RNA in the form of RNA and the nucleic acid encoding the Cas protein are administered to the subject via lipid-nanoparticle-mediated delivery, wherein the nucleic acid encoding the Cas protein is an mRNA. In some such methods, the lipid nanoparticle comprises a cationic lipid, a neutral lipid, a helper lipid, and a stealth lipid. In some such methods, the cationic lipid is Lipid A. In some such methods, the neutral lipid is DSPC. In some such methods, the helper lipid is cholesterol. In some such methods, the stealth lipid is PEG2k-DMG. In some such methods, the cationic lipid is Lipid A, the neutral lipid is DSPC, the helper lipid is cholesterol, and the stealth lipid is PEG2k-DMG.

In some such methods, the Cas protein or the nucleic acid encoding the Cas protein and/or the guide RNA or the DNA encoding the guide RNA are administered to the subject via adeno-associated virus.

In some such methods, the method comprises administering the nucleic acid encoding the Cas protein. In some such methods, the nucleic acid encoding the Cas protein is codon-optimized for expression in a mammalian cell or a human cell. In some such methods, the nucleic acid encoding the Cas protein comprises DNA, optionally wherein the method comprises administering the DNA encoding the guide RNA. In some such methods, the nucleic acid encoding the Cas protein comprises RNA, optionally wherein the method comprises administering the guide RNA in the form of RNA. In some such methods, the RNA encoding the Cas protein comprises at least one modification. In some such methods, the RNA encoding the Cas protein is modified to comprise a modified uridine at one or more or all uridine positions. In some such methods, the modified uridine is pseudouridine. In some such methods, the modified uridine is N1-methyl-pseudouridine. In some such methods, the RNA encoding the Cas protein is fully substituted with pseudouridine. In some such methods, the RNA encoding the Cas protein is fully substituted with N1-methyl-pseudouridine. In some such methods, the RNA encoding the Cas protein comprises a 5′ cap. In some such methods, the RNA encoding the Cas protein comprises a poly(A) tail. In some such methods, the RNA encoding the Cas protein comprises the sequence set forth in SEQ ID NO: 339, 338, or 12.

In some such methods, the method comprises administering the guide RNA in the form of RNA. In some such methods, the method comprises administering the DNA encoding the guide RNA. In some such methods, the guide RNA comprises at least one modification. In some such methods, the at least one modification comprises a 2′-O-methyl-modified nucleotide. In some such methods, the at least one modification comprise a phosphorothioate bond between nucleotides. In some such methods, the at least one modification comprise a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA comprises the modified nucleotides of SEQ ID NO: 29.

In some such methods, the guide RNA is a single guide RNA (sgRNA). In some such methods, the guide RNA comprises the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

In some such methods, the guide RNA is a dual guide RNA (dgRNA) comprising two separate RNA molecules comprising a CRISPR RNA (crRNA) and a trans-activating crRNA (tracrRNA). In some such methods, the crRNA comprises the sequence set forth in any one of SEQ ID NOS: 16-17. In some such methods, the tracrRNA comprises the sequence set forth in any one of SEQ ID NOS: 18-20.

In some such methods, the Cas protein is a Cas9 protein. In some such methods, the Cas protein is derived from a Streptococcus pyogenes Cas9 protein. In some such methods, the Cas protein comprises the sequence set forth in SEQ ID NO: 11 or 8.

In some such methods, the method further comprises administering to the subject a second guide RNA or a DNA encoding the second guide RNA, wherein the second guide RNA forms a complex with the Cas protein and targets the Cas protein to a second guide RNA target sequence in the C5 gene, wherein the Cas protein cleaves the second guide RNA target sequence to generate a targeted genetic modification in the C5 gene.

In some such methods, cleavage by the Cas protein creates a double-strand break in the C5 gene. In some such methods, cleavage by the Cas protein creates a single-strand break in the C5 gene. In some such methods, the targeted genetic modification is generated by repair of the cleaved guide RNA target sequence by non-homologous end-joining. In some such methods, the method results in reduced expression or activity of the C5 gene in the cell. In some such methods, the method results in loss of function or inactivation of the C5 gene in the cell.

In some such methods, the subject is a mammal, and the C5 gene is a mammalian C5 gene. In some such methods, the subject is a human, and the C5 gene is a human C5 gene.

In some such methods, the method results in at least 50%, at least 55%, at least 60%, at least 65%, at least 70%, at least 75%, at least 80%, at least 85%, at least 90%, or at least 95% percent editing of the C5 gene in a target population of cells in the subject. In some such methods, the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the subject.

In some such methods, the method results in reduced serum levels of complement C5 protein in the subject, optionally wherein serum levels of complement C5 protein are reduced by at least 50%, at least 55%, at least 60%, at least 65%, at least 70%, at least 75%, at least 80%, at least 85%, at least 90%, or at least 95%. In some such methods, the method results in reduced complement C5 protein activity in the subject, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells. In some such methods, C5 complement activity is reduced by about 95-100% as measured by CH50 assay of complement-mediated sheep red blood cell lysis.

In some such methods, the composition is administered in association with a further therapeutic agent. In some such methods, the further therapeutic agent is an antigen-binding protein that binds specifically to C5, acetaminophen, an albumin infusion, ancrod, an angiotensin-converting enzyme inhibitor, an antibiotic, an anti-CD20 agent, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a meningococcal vaccine, methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin, and/or warfarin.

In some such methods, the further therapeutic agent is the antigen-binding protein that binds specifically to C5. In some such methods, the antigen-binding protein is administered to the subject intravenously or subcutaneously. In some such methods, a first dose of the antigen-binding protein is administered to the subject intravenously, and one or more additional doses of the antigen-binding protein are administered subcutaneously. In some such methods, the antigen-binding protein that binds specifically to C5 is an antibody or an antigen-binding fragment thereof. In some such methods, the antigen-binding protein that binds specifically to C5 comprises: (1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof, (2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof, (3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof, (4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof, (5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof, (6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof, (7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof, (8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof, (9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof, (10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof, (11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof, (12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof, (13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof, (14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof, (15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof, (16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof, (17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof, (18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof, (19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof, (20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof, (21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof, (22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof, (23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof, (24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof, (25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof, (26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof, (27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof, (28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or (29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or competes for binding to C5 with an antigen-binding protein selected from the group consisting of (1)-(29); or binds to the same epitope on C5 as an antigen-binding protein selected from the group consisting of (1)-(29). In some such methods, the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof. In some such methods, wherein the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698. In some such methods, the antigen-binding protein that binds specifically to C5 is pozelimab.

### BRIEF DESCRIPTION OF THE FIGURES

FIG. 1A shows human C5 insertion/deletion (indel) frequency at Day 5 following administration of 10 nM Cas9 ribonucleoprotein (RNP) complex with different C5 targeting sgRNAs to primary human hepatocytes. Positive control sgRNAs PCSK9_1264 and TTR_G000489 target regions in PCSK9 and TTR genes, respectively. Negative control sgRNA msHcl is a human non-targeting sgRNA.

FIG. 1B shows human complement C5 protein expression in culture media at Day 5 following administration of 10 nM Cas9 ribonucleoprotein (RNP) complex with different C5 targeting sgRNAs to primary human hepatocytes.

FIG. 2 shows human C5 insertion/deletion (indel) frequency at Day 3 following administration of 0.5 μg Cas9 mRNA with 25 nM of different C5 targeting sgRNAs to primary human hepatocytes.

FIG. 3A shows plasma levels of human complement C5 at 3 weeks post-injection of LNPs formulated with Cas9 mRNA and different C5 targeting sgRNAs at a dose of 1 mg/kg to humanized C5 mice.

FIG. 3B shows plasma levels of human complement C5 at 3 weeks post-injection of LNPs formulated with Cas9 mRNA and different C5 targeting sgRNAs at a dose of 2 mg/kg to humanized C5 mice.

FIGS. 4A and 4B show classical pathway hemolysis and classical pathway hemolysis as a percent reduction over control, respectively, as measured ex vivo using sensitized sheep red blood cells at 3 weeks post-injection of LNPs formulated with Cas9 mRNA and different C5 targeting sgRNAs at a dose of 2 mg/kg to humanized C5 mice.

FIGS. 5A and 5B show plasma levels of human complement C5 and classical pathway hemolysis as measured ex vivo using sensitized sheep red blood cells at 3 weeks post-injection of LNP formulated with new Cas9 mRNA and two different C5 targeting sgRNAs at a dose of 0.1, 0.3, or 1 mg/kg to humanized C5 mice.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), lipid droplets, proteins, and nucleic acids includes cells, tissues (e.g., liver samples), lipid droplets, proteins, and nucleic acids that are relatively purified with respect to other bacterial, viral, cellular, or other components that may normally be present in situ, up to and including a substantially pure preparation of the cells, tissues (e.g., liver samples), lipid droplets, proteins, and nucleic acids. The term “isolated” also includes cells, tissues (e.g., liver samples), lipid droplets, proteins, and nucleic acids that have no naturally occurring counterpart, have been chemically synthesized and are thus substantially uncontaminated by other cells, tissues (e.g., liver samples), lipid droplets, proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a rat cell or rat. For example, an endogenous C5 sequence of a mouse refers to a native C5 sequence that naturally occurs at the C5 locus in the mouse.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a complement C5 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “C5 locus” may refer to the specific location of a C5 gene, C5 DNA sequence, complement-C5-encoding sequence, or C5 position on a chromosome of the genome of an organism that has been identified as to where such a sequence resides. A “C5 locus” may comprise a regulatory element of a C5 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Prc | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values 5% of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Disclosed herein are guide RNAs and CRISPR/Cas systems targeting a C5 locus or gene, lipid nanoparticles or viral vectors comprising such guide RNAs or CRISPR/Cas systems, and cells or animals comprising such guide RNAs or systems. Such CRISPR/Cas systems can be alone or in combination with or in association with C5 antigen-binding proteins or antibodies such as, but not limited to, those disclosed herein or those disclosed in WO 2021/034639 A1, US 2021-0046182, WO 2021/081277 A1, US 2021-0139573, WO 2017/218515 A1, US 2020-0262901, US 2017-0355757, or US 2020-0262900, each of which is herein incorporated by reference in its entirety for all purposes. Also disclosed herein are methods of modifying or knocking down or knocking out a C5 locus or gene using the CRISPR/Cas systems described herein, as well as use of the CRISPR/Cas systems in prophylactic and therapeutic applications for treatment and/or prevention of a disease, disorder, or condition associated with C5 and/or for ameliorating at least one symptom associated with such disease, disorder, or condition. Such methods can comprise using CRISPR/Cas systems alone or in combination with C5 antigen-binding proteins or antibodies such as, but not limited to, those disclosed herein or those disclosed in WO 2021/034639 A1, US 2021-0046182, WO 2021/081277 A1, US 2021-0139573, WO 2017/218515 A1, US 2020-0262901, US 2017-0355757, or US 2020-0262900, each of which is herein incorporated by reference in its entirety for all purposes.

### II. CRISPR/Cas Systems Targeting a C5 Locus or Gene

The methods and compositions disclosed herein utilize Clustered Regularly Interspersed Short Palindromic Repeats (CRISPR)/CRISPR-associated (Cas) systems or components of such systems to modify a C5 gene or locus (e.g., C5 genomic locus) within a cell. CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting a C5 gene or C5 locus comprises a Cas protein (or a nucleic acid encoding the Cas protein) and one or more guide RNAs (or DNAs encoding the one or more guide RNAs), with each of the one or more guide RNAs targeting a different guide RNA target sequence in the C5 gene or C5 locus. Optionally, such CRISPR/Cas systems targeting a C5 gene or C5 locus can further comprise one or more exogenous donor sequences (e.g., targeting vectors) that target the C5 gene or genomic locus.

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### A. C5

The compositions and methods described herein are for targeting the C5 gene (also known as CPAMD4), which encodes complement C5 (also known as complement component C5, complement C5 isoform 1 preproprotein, or C3 and PZP-like alpha-2-macroglobulin domain-containing protein 4). Complement C5 is a component of the complement system, a part of the innate immune system that plays an important role in inflammation, host homeostasis, and host defense against pathogens. The encoded preproprotein is proteolytically processed to generate multiple protein products, including the C5 alpha chain, C5 beta chain, C5a anaphylatoxin, and C5b. The C5 protein is comprised of the C5 alpha and beta chains, which are linked by a disulfide bridge. Cleavage of the alpha chain by a convertase enzyme results in the formation of the C5a anaphylatoxin, which possesses potent spasmogenic and chemotactic activity, and the C5b macromolecular cleavage product, a subunit of the membrane attack complex (MAC). Mutations in this gene cause complement component 5 deficiency, a disease characterized by recurrent bacterial infections. Alternative splicing results in multiple transcript variants.

Activation of C5 by a C5 convertase initiates the spontaneous assembly of the late complement components, C5-C9, into the membrane attack complex. C5b has a transient binding site for C6. The C5b-C6 complex is the foundation upon which the lytic complex is assembled. Derived from proteolytic degradation of complement C5, C5 anaphylatoxin is a mediator of local inflammatory process. Binding to the receptor C5AR1 induces a variety of responses including intracellular calcium release, contraction of smooth muscle, increased vascular permeability, and histamine release from mast cells and basophilic leukocytes. C5a is also a potent chemokine which stimulates the locomotion of polymorphonuclear leukocytes and directs their migration toward sites of inflammation. Complement C5 is a terminal effector molecule of all three-complement activation pathways and is a central meditator in several complement-driven diseases.

Human C5 maps to 9q33.2 on chromosome 9 (NCBI RefSeq Gene ID 727; Assembly GRCh38.p13 (GCF_000001405.39); location NC_000009.12 (120952335..121074865, complement)). The gene has been reported to have 41 coding exons. The human complement C5 protein has been assigned UniProt Accession No. P01031. The sequence for the canonical isoform, NCBI Accession No. NP_0017626.2 and UniProt Accession No. P01031-1, is set forth in SEQ ID NO: 1. An mRNA (cDNA) encoding the canonical isoform is assigned NCBI Accession No. NM_001735.3 and is set forth in SEQ ID NO: 2. An exemplary coding sequence (CDS) encoding the canonical isoform is set forth in SEQ ID NO: 3 (CCDS ID CCDS6826.1). Another isoform of human complement C5 protein is assigned NCBI Accession No. NP_001304092.1 and is set forth in SEQ ID NO: 4. An mRNA (cDNA) encoding this other isoform is assigned NCBI Accession No. NM_001317163.2 and is set forth in SEQ ID NO: 5.

The full-length human complement C5 protein set forth in SEQ ID NO: 1 has 1676 amino acids. Reference to human complement C5 includes the canonical (wild type) forms as well as all allelic forms and isoforms. Any other forms of human complement C5 have amino acids numbered for maximal alignment with the wild type form, aligned amino acids being designated the same number.

Mouse C5 (also known as He or hemolytic complement) maps to 2 B; 2 23.22 cM on chromosome 2 (NCBI RefSeq Gene ID 15139; Assembly GRCm38.p6 (GCF_000001635.26); location NC_000068.7 (34983329..35068506, complement)). The gene has been reported to have 41 coding exons. The mouse component C5 protein has been assigned UniProt Accession No. P06684 and NCBI Accession No. NP_034536.3. An exemplary mRNA (cDNA) encoding the canonical isoform is assigned NCBI Accession No. NM_010406.2. An exemplary coding sequence (CDS) encoding the canonical isoform is assigned CCDS ID CCDS15957.1.

### B. Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 8 (encoded by the DNA sequence set forth in SEQ ID NO: 9). An exemplary SpCas9 mRNA (cDNA) sequence is set forth in SEQ ID NO: 10. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence is set forth in SEQ ID NO: 11. An exemplary SpCas9 mRNA sequence encoding that SpCas9 protein sequence comprises the sequence set forth in SEQ ID NO: 339, 338, or 12. Other exemplary SpCas9 open reading frame sequences are set forth in SEQ ID NOS: 335-337 (e.g., SEQ ID NO: 336).

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) and a substitution at position D10 (e.g., D10A substitution) to generate a nuclease-inactive Cas protein. See, e.g., WO 2016/106236, herein incorporated by reference in its entirety for all purposes. Examples of inactivating mutations in the catalytic domains of Nme2Cas9 are also known (e.g., combination of D16A and H588A). Examples of inactivating mutations in the catalytic domains of St1Cas9 are also known (e.g., combination of D9A, D598A, H599A, and N622A). Examples of inactivating mutations in the catalytic domains of St3Cas9 are also known (e.g., combination of D10A and N870A). Examples of inactivating mutations in the catalytic domains of CjCas9 are also known (e.g., combination of D8A and H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain, an epigenetic modification domain, or a transcriptional repressor domain. See WO 2014/089290, herein incorporated by reference in its entirety for all purposes. Examples of transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, and MeCP2. Other examples include transcriptional repressor domains from A/B, KOX, TGF-beta-inducible early gene (TIEG), v-erbA, SID, SID4X, MBD2, MBD3, DNMT1, DNMG3A, DNMT3B, Rb, ROM2, See, e.g., EP3045537 and WO 2011/146121, each of which is incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 13) or PKKKRRV (SEQ ID NO: 14). The NLS may be a bipartite sequence, such as the NLS of nucleoplasmin, KRPAATKKAGQAKKKK (SEQ ID NO: 15). In a specific example, a single PKKKRKV (SEQ ID NO: 13) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AUl, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an endogenous C5 locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### C. Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a C5 gRNA is a S. pyogenes Cas9 gRNA or an equivalent thereof. In some of the methods and compositions disclosed herein, a C5 gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 16) or GUUUUAGAGCUAUGCUGUUUUG (SEQ ID NO: 17). Any of the DNA-targeting segments disclosed herein can be joined to the 5′ end of SEQ ID NO: 16 or 17 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 18), AAACAGCAUAGCAAGUUAAAAUAAGGCUAGUCCGUUAUCAACUUGAAAAAGUGG CACCGAGUCGGUGCUUUU (SEQ ID NO: 19), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 20).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting a C5 gene can comprise a DNA-targeting segment (i.e., guide sequence) comprising, consisting essentially of, or consisting of the sequence (DNA-targeting segment) set forth in any one of SEQ ID NOS: 33-120. Alternatively, a guide RNA targeting a C5 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 33-120. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 33-120. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 33-120. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 33-120. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 33-120. Alternatively, a guide RNA targeting a C5 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 33-120. Alternatively, a guide RNA targeting a C5 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 33-120.

As one example, a guide RNA targeting a C5 gene can comprise a DNA-targeting segment (i.e., guide sequence) comprising, consisting essentially of, or consisting of the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 60, 65, 67, 82, 85, 87, 97, and 119. Alternatively, a guide RNA targeting a C5 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 60, 65, 67, 82, 85, 87, 97, and 119.

As one example, a guide RNA targeting a C5 gene can comprise a DNA-targeting segment (i.e., guide sequence) comprising, consisting essentially of, or consisting of the sequence (DNA-targeting segment) set forth in any one of SEQ ID NOS: 85 and 97. Alternatively, a guide RNA targeting a C5 gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 85 and 97. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 85 and 97. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 85 and 97. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 85 and 97. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 85 and 97. Alternatively, a guide RNA targeting a C5 gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 85 and 97. Alternatively, a guide RNA targeting a C5 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 85 and 97.

As one example, a guide RNA targeting a C5 gene can comprise a DNA-targeting segment (i.e., guide sequence) comprising, consisting essentially of, or consisting of the sequence (DNA-targeting segment) set forth in SEQ ID NO: 85. Alternatively, a guide RNA targeting a C5 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 85. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 85. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 85. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 85. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 85. Alternatively, a guide RNA targeting a C5 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 85. Alternatively, a guide RNA targeting a C5 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 85.

As one example, a guide RNA targeting a C5 gene can comprise a DNA-targeting segment (i.e., guide sequence) comprising, consisting essentially of, or consisting of the sequence (DNA-targeting segment) set forth in SEQ ID NO: 97. Alternatively, a guide RNA targeting a C5 gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 97. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 97. Alternatively, a guide RNA targeting a C5 gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 97. Alternatively, a guide RNA targeting a C5 gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 97. Alternatively, a guide RNA targeting a C5 gene can comprise a DNA-targeting segment that is at least 90%, at least 91%, at least 92%, at least 93% at least 94% at least 95% at least 96%, at least 97%, at least 98%, or at least 99% identical to at least 17, at least 18, at least 19, or at least 20 contiguous nucleotides of the sequence (DNA-targeting segment) set forth in SEQ TD NO: 97. Alternatively, a guide RNA targeting a C5 gene can comprise a DNA-targeting segment comprising, consisting essentially of, or consisting of a sequence that differs by no more than 3, no more than 2, or no more than 1 nucleotide from the sequence (DNA-targeting segment) set forth in SEQ TD NO: 97. Alternatively, a guide RNA targeting a C5 gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ TD NO: 97.

TABLE 2
C5 Guide Sequences and Chromosomal Coordinates.

| | | | SEQ | | SEQ | |
|---|---|---|---|---|---|---|
| ID | Exon | Guide | ID | crRNA | ID | Location |
| CR005628 | 41 | CCUUGACCUG | 33 | CCUUGACCUGGAUU | 121 | chr9: 120952828- |
| | | GAUUGAAUAC | | GAAUACGUUUUAGA | | 120952848 |
| | | | | GCUAUGCUGUUUUG | | |
| CR005629 | 41 | ACCCUUUAGA | 34 | ACCCUUUAGAUUCC | 122 | chr9: 120952840- |
| | | UUCCUUGACC | | UUGACCGUUUUAGA | | 120952860 |
| | | | | GCUAUGCUGUUUUG | | |
| CR005630 | 40 | GGAAGACAGU | 35 | GGAAGACAGUACUU | 123 | chr9: 120953771- |
| | | ACUUAAUUAU | | AAUUAUGUUUUAGA | | 120953791 |
| | | | | GCUAUGCUGUUUUG | | |
| CR005631 | 40 | AGGAAGACAG | 36 | AGGAAGACAGUACU | 124 | chr9: 120953772- |
| | | UACUUAAUUA | | UAAUUAGUUUUAGA | | 120953792 |
| | | | | GCUAUGCUGUUUUG | | |
| CR005632 | 39 | AGUUUUGUAG | 37 | AGUUUUGUAGAUAU | 125 | chr9: 120957285- |
| | | AUAUCCAGAA | | CCAGAAGUUUUAGA | | 120957305 |
| | | | | GCUAUGCUGUUUUG | | |
| CR005633 | 39 | CAGUUUUGUA | 38 | CAGUUUUGUAGAUA | 126 | chr9: 120957284- |
| | | GAUAUCCAGA | | UCCAGAGUUUUAGA | | 120957304 |
| | | | | GCUAUGCUGUUUUG | | |
| CR005634 | 38 | AACUCACCAU | 39 | AACUCACCAUAUGC | 127 | chr9: 120960240- |
| | | AUGCAAUCUC | | AAUCUCGUUUUAGA | | 120960260 |
| | | | | GCUAUGCUGUUUUG | | |
| CR005635 | 37 | GACUUUCUGA | 40 | GACUUUCUGAAUUU | 128 | chr9: 120961512- |
| | | AUUUUGAUAU | | UGAUAUGUUUUAGA | | 120961532 |
| | | | | GCUAUGCUGUUUUG | | |
| CR005636 | 36 | UACACAAAGG | 41 | UACACAAAGGAAAU | 129 | chr9: 120962749- |
| | | AAAUCACUGG | | CACUGGGUUUUAGA | | 120962769 |
| | | | | GCUAUGCUGUUUUG | | |
| CR005637 | 36 | GGAGUUGAUU | 42 | GGAGUUGAUUACCU | 130 | chr9: 120962658- |
| | | ACCUGGUCUG | | GGUCUGGUUUUAGA | | 120962678 |
| | | | | GCUAUGCUGUUUUG | | |
| CR005638 | 36 | UUAGCAUGGA | 43 | UUAGCAUGGAGUUG | 131 | chr9: 120962651- |
| | | GUUGAUUACC | | AUUACCGUUUUAGA | | 120962671 |
| | | | | GCUAUGCUGUUUUG | | |
| CR005639 | 35 | CUCUUUUCCC | 44 | CUCUUUUCCCCCUU | 132 | chr9: 120962963- |
| | | CCUUAGCUUG | | AGCUUGGUUUUAGA | | 120962983 |
| | | | | GCUAUGCUGUUUUG | | |
| CR005640 | 35 | UUCCCCCUUA | 45 | UUCCCCCUUAGCUU | 133 | chr9: 120962958- |
| | | GCUUGUGGAA | | GUGGAAGUUUUAGA | | 120962978 |
| | | | | GCUAUGCUGUUUUG | | |
| CR005641 | 34 | AUGGACAUCU | 46 | AUGGACAUCUCCUU | 134 | chr9: 120963669- |
| | | CCUUGCCUAC | | GCCUACGUUUUAGA | | 120963689 |
| | | | | GCUAUGCUGUUUUG | | |
| CR005642 | 33 | GCAUCCCACU | 47 | GCAUCCCACUACAG | 135 | chr9: 120969099- |
| | | ACAGAGGCUA | | AGGCUAGUUUUAGA | | 120969119 |
| | | | | GCUAUGCUGUUUUG | | |
| CR005643 | 32 | UCACAAAACC | 48 | UCACAAAACCAGUA | 136 | chr9: 120970217- |
| | | AGUACCUCUG | | CCUCUGGUUUUAGA | | 120970237 |
| | | | | GCUAUGCUGUUUUG | | |
| CR005644 | 32 | AAAGCUGCAA | 49 | AAAGCUGCAAACUU | 137 | chr9: 120970200- |
| | | ACUUCCUCAG | | CCUCAGGUUUUAGA | | 120970220 |
| | | | | GCUAUGCUGUUUUG | | |
| CR005645 | 31 | UACAGGAUUU | 50 | UACAGGAUUUGGCA | 138 | chr9: 120971943- |
| | | GGCAGUGGCU | | GUGGCUGUUUUAGA | | 120971963 |
| | | | | GCUAUGCUGUUUUG | | |
| CR005646 | 31 | GUCAGUACAG | 51 | GUCAGUACAGGAUU | 139 | chr9: 120971948- |
| | | GAUUUGGCAG | | UGGCAGGUUUUAGA | | 120971968 |
| | | | | GCUAUGCUGUUUUG | | |
| CR005647 | 30 | CAGACAAGAA | 52 | CAGACAAGAAUUUC | 140 | chr9: 120974790- |
| | | UUUCCUUGGG | | CUUGGGGUUUUAGA | | 120974810 |
| | | | | GCUAUGCUGUUUUG | | |
| CR005648 | 30 | AGUUCUUCAU | 53 | AGUUCUUCAUUUAC | 141 | chr9: 120974764- |
| | | UUACCUCUAC | | CUCUACGUUUUAGA | | 120974784 |
| | | | | GCUAUGCUGUUUUG | | |
| CR005649 | 29 | CUAUCAGAAG | 54 | CUAUCAGAAGAGCA | 142 | chr9: 120976724- |
| | | AGCAGAGGUA | | GAGGUAGUUUUAGA | | 120976744 |
| | | | | GCUAUGCUGUUUUG | | |
| CR005650 | 29 | GACAGCUCUG | 55 | GACAGCUCUGUACC | 143 | chr9: 120976838- |
| | | UACCUAACAC | | UAACACGUUUUAGA | | 120976858 |
| | | | | GCUAUGCUGUUUUG | | |
| CR005651 | 29 | AUCCACCCAU | 56 | AUCCACCCAUUUAU | 144 | chr9: 120976882- |
| | | UUAUCGUUUU | | CGUUUUGUUUUAGA | | 120976902 |
| | | | | GCUAUGCUGUUUUG | | |
| CR005652 | 28 | GAAACAAUUG | 57 | GAAACAAUUGAACG | 145 | chr9: 120980111- |
| | | AACGAAACUG | | AAACUGGUUUUAGA | | 120980131 |
| | | | | GCUAUGCUGUUUUG | | |
| CR005653 | 28 | AGCCCAGAGC | 58 | AGCCCAGAGCACCU | 146 | chr9: 120980178- |
| | | ACCUUUACAU | | UUACAUGUUUUAGA | | 120980198 |
| | | | | GCUAUGCUGUUUUG | | |
| CR005654 | 27 | CUGUUCUCUC | 59 | CUGUUCUCUCGGGC | 147 | chr9: 120981907- |
| | | GGGCUUCAAC | | UUCAACGUUUUAGA | | 120981927 |
| | | | | GCUAUGCUGUUUUG | | |
| CR005655 | 27 | CUCUCGGGCU | 60 | CUCUCGGGCUUCAA | 148 | chr9: 120981912- |
| | | UCAACAGGCA | | CAGGCAGUUUUAGA | | 120981932 |
| | | | | GCUAUGCUGUUUUG | | |
| CR005656 | 24 | UGCACUCCCU | 61 | UGCACUCCCUUUGG | 149 | chr9: 120989697- |
| | | UUGGGGAGGU | | GGAGGUGUUUUAGA | | 120989717 |
| | | | | GCUAUGCUGUUUUG | | |
| CR005657 | 24 | AUUCUAUGUU | 62 | AUUCUAUGUUUUUC | 150 | chr9: 120989648- |
| | | UUUCACUACC | | ACUACCGUUUUAGA | | 120989668 |
| | | | | GCUAUGCUGUUUUG | | |
| CR005658 | 23 | GACGAAAGGA | 63 | GACGAAAGGAGUUC | 151 | chr9: 120991248- |
| | | GUUCCCAUAC | | CCAUACGUUUUAGA | | 120991268 |
| | | | | GCUAUGCUGUUUUG | | |
| CR005659 | 23 | AGGUACCAUU | 64 | AGGUACCAUUAGCA | 152 | chr9: 120991262- |
| | | AGCAGACGAA | | GACGAAGUUUUAGA | | 120991282 |
| | | | | GCUAUGCUGUUUUG | | |
| CR005660 | 22 | UGCCUACCAU | 65 | UGCCUACCAUAAAU | 153 | chr9: 120996232- |
| | | AAAUACCCCU | | ACCCCUGUUUUAGA | | 120996252 |
| | | | | GCUAUGCUGUUUUG | | |
| CR005661 | 22 | UUAGCCAGAA | 66 | UUAGCCAGAAGGUG | 154 | chr9: 120996284- |
| | | GGUGUCAAAA | | UCAAAAGUUUUAGA | | 120996304 |
| | | | | GCUAUGCUGUUUUG | | |
| CR005662 | 21 | UGCCCUGAUG | 67 | UGCCCUGAUGAUCA | 155 | chr9: 120997704- |
| | | AUCAAUGACU | | AUGACUGUUUUAGA | | 120997724 |
| | | | | GCUAUGCUGUUUUG | | |
| CR005663 | 21 | GUGCCCUGAU | 68 | GUGCCCUGAUGAUC | 156 | chr9: 120997703- |
| | | GAUCAAUGAC | | AAUGACGUUUUAGA | | 120997723 |
| | | | | GCUAUGCUGUUUUG | | |
| CR005664 | 21 | AGCCCAGUCA | 69 | AGCCCAGUCAUUGA | 157 | chr9: 120997709- |
| | | UUGAUCAUCA | | UCAUCAGUUUUAGA | | 120997729 |
| | | | | GCUAUGCUGUUUUG | | |
| CR005665 | 21 | AAGCCCAGUC | 70 | AAGCCCAGUCAUUG | 158 | chr9: 120997710- |
| | | AUUGAUCAUC | | AUCAUCGUUUUAGA | | 120997730 |
| | | | | GCUAUGCUGUUUUG | | |
| CR005666 | 20 | UUUACAACUA | 71 | UUUACAACUAUAGG | 159 | chr9: 121005927- |
| | | UAGGACUUCU | | ACUUCUGUUUUAGA | | 121005947 |
| | | | | GCUAUGCUGUUUUG | | |
| CR005667 | 20 | UCUCCUCGUA | 72 | UCUCCUCGUACAAC | 160 | chr9: 121005970- |
| | | CAACAGAAUA | | AGAAUAGUUUUAGA | | 121005990 |
| | | | | GCUAUGCUGUUUUG | | |
| CR005668 | 20 | AAGGAACUGU | 73 | AAGGAACUGUUUAC | 161 | chr9: 121005936- |
| | | UUACAACUAU | | AACUAUGUUUUAGA | | 121005956 |
| | | | | GCUAUGCUGUUUUG | | |
| CR005669 | 20 | GUUUACAACU | 74 | GUUUACAACUAUAG | 162 | chr9: 121005928- |
| | | AUAGGACUUC | | GACUUCGUUUUAGA | | 121005948 |
| | | | | GCUAUGCUGUUUUG | | |
| CR005670 | 20 | AUACCAUAUU | 75 | AUACCAUAUUCUGU | 163 | chr9: 121005976- |
| | | CUGUUGUACG | | UGUACGGUUUUAGA | | 121005996 |
| | | | | GCUAUGCUGUUUUG | | |
| CR005671 | 18 | CGAAUUUCUG | 76 | CGAAUUUCUGGCUU | 164 | chr9: 121008458- |
| | | GCUUGCUUAC | | GCUUACGUUUUAGA | | 121008478 |
| | | | | GCUAUGCUGUUUUG | | |
| CR005672 | 18 | CAGUAAGCAA | 77 | CAGUAAGCAAGCCA | 165 | chr9: 121008460- |
| | | GCCAGAAAUU | | GAAAUUGUUUUAGA | | 121008480 |
| | | | | GCUAUGCUGUUUUG | | |
| CR005673 | 18 | UGGCUUGCUU | 78 | UGGCUUGCUUACUG | 166 | chr9: 121008466- |
| | | ACUGGUAACA | | GUAACAGUUUUAGA | | 121008486 |
| | | | | GCUAUGCUGUUUUG | | |
| CR005674 | 18 | CUGGCUUGCU | 79 | CUGGCUUGCUUACU | 167 | chr9: 121008465- |
| | | UACUGGUAAC | | GGUAACGUUUUAGA | | 121008485 |
| | | | | GCUAUGCUGUUUUG | | |
| CR005675 | 17 | CCGUGCAGCU | 80 | CCGUGCAGCUCGCU | 168 | chr9: 121013978- |
| | | CGCUGCUCAC | | GCUCACGUUUUAGA | | 121013998 |
| | | | | GCUAUGCUGUUUUG | | |
| CR005676 | 17 | GUGAAGAAAU | 81 | GUGAAGAAAUGUUG | 169 | chr9: 121014027- |
| | | GUUGUUACGA | | UUACGAGUUUUAGA | | 121014047 |
| | | | | GCUAUGCUGUUUUG | | |
| CR005677 | 15 | UUCUUAGAGA | 82 | UUCUUAGAGAAGAG | 170 | chr9: 121016354- |
| | | AGAGUGAUCU | | UGAUCUGUUUUAGA | | 121016374 |
| | | | | GCUAUGCUGUUUUG | | |
| CR005678 | 15 | AUUCUUAGAG | 83 | AUUCUUAGAGAAGA | 171 | chr9: 121016355- |
| | | AAGAGUGAUC | | GUGAUCGUUUUAGA | | 121016375 |
| | | | | GCUAUGCUGUUUUG | | |
| CR005679 | 15 | GCCAAUGUGU | 84 | GCCAAUGUGUUCCA | 172 | chr9: 121016303- |
| | | UCCACCUAGC | | CCUAGCGUUUUAGA | | 121016323 |
| | | | | GCUAUGCUGUUUUG | | |
| CR005680 | 15 | UGAGGAAGGU | 85 | UGAGGAAGGUAAGU | 173 | chr9: 121016286- |
| | | AAGUCCAGCU | | CCAGCUGUUUUAGA | | 121016306 |
| | | | | GCUAUGCUGUUUUG | | |
| CR005681 | 15 | GGAAGGUAAG | 86 | GGAAGGUAAGUCCA | 174 | chr9: 121016289- |
| | | UCCAGCUAGG | | GCUAGGGUUUUAGA | | 121016309 |
| | | | | GCUAUGCUGUUUUG | | |
| CR005682 | 15 | UCCAGCUAGG | 87 | UCCAGCUAGGUGGA | 175 | chr9: 121016299- |
| | | UGGAACACAU | | ACACAUGUUUUAGA | | 121016319 |
| | | | | GCUAUGCUGUUUUG | | |
| CR005683 | 14 | AACUCUUUCC | 88 | AACUCUUUCCAAGG | 176 | chr9: 121017358- |
| | | AAGGGCUUUU | | GCUUUUGUUUUAGA | | 121017378 |
| | | | | GCUAUGCUGUUUUG | | |
| CR005684 | 14 | CAUUACUUAA | 89 | CAUUACUUAACUCU | 177 | chr9: 121017350- |
| | | CUCUUUCCAA | | UUCCAAGUUUUAGA | | 121017370 |
| | | | | GCUAUGCUGUUUUG | | |
| CR005685 | 14 | CCAUAUUAAG | 90 | CCAUAUUAAGAGAC | 178 | chr9: 121017450- |
| | | AGACACAGUU | | ACAGUUGUUUUAGA | | 121017470 |
| | | | | GCUAUGCUGUUUUG | | |
| CR005686 | 13 | AUUUGUGGCU | 91 | AUUUGUGGCUUACC | 179 | chr9: 121017629- |
| | | UACCUGGAGC | | UGGAGCGUUUUAGA | | 121017649 |
| | | | | GCUAUGCUGUUUUG | | |
| CR005687 | 13 | AAGGGCAAAA | 92 | AAGGGCAAAAUUAU | 180 | chr9: 121017823- |
| | | UUAUCCACUU | | CCACUUGUUUUAGA | | 121017843 |
| | | | | GCUAUGCUGUUUUG | | |
| CR005688 | 13 | AAUUAGUGUC | 93 | AAUUAGUGUCUGAU | 181 | chr9: 121017681- |
| | | UGAUUCAGUC | | UCAGUCGUUUUAGA | | 121017701 |
| | | | | GCUAUGCUGUUUUG | | |
| CR005689 | 12 | UUUGUCAAUA | 94 | UUUGUCAAUAUAUG | 182 | chr9: 121019996- |
| | | UAUGGGCUUU | | GGCUUUGUUUUAGA | | 121020016 |
| | | | | GCUAUGCUGUUUUG | | |
| CR005690 | 12 | GAGUUAUUUU | 95 | GAGUUAUUUUGUCA | 183 | chr9: 121019989- |
| | | GUCAAUAUAU | | AUAUAUGUUUUAGA | | 121020009 |
| | | | | GCUAUGCUGUUUUG | | |
| CR005691 | 12 | UUGUCAAUAU | 96 | UUGUCAAUAUAUGG | 184 | chr9: 121019997- |
| | | AUGGGCUUUU | | GCUUUUGUUUUAGA | | 121020017 |
| | | | | GCUAUGCUGUUUUG | | |
| CR005692 | 12 | GAGAUGAGUA | 97 | GAGAUGAGUAUGCU | 185 | chr9: 121020103- |
| | | UGCUAUUGCU | | AUUGCUGUUUUAGA | | 121020123 |
| | | | | GCUAUGCUGUUUUG | | |
| CR005693 | 11 | UUUGUGCAUU | 98 | UUUGUGCAUUCAGU | 186 | chr9: 121021630- |
| | | CAGUGUUACU | | GUUACUGUUUUAGA | | 121021650 |
| | | | | GCUAUGCUGUUUUG | | |
| CR005694 | 11 | GUUUGUGCAU | 99 | GUUUGUGCAUUCAG | 187 | chr9: 121021629- |
| | | UCAGUGUUAC | | UGUUACGUUUUAGA | | 121021649 |
| | | | | GCUAUGCUGUUUUG | | |
| CR005695 | 11 | AGUGUAACAC | 100 | AGUGUAACACGUGU | 188 | chr9: 121021566- |
| | | GUGUUGAUGA | | UGAUGAGUUUUAGA | | 121021586 |
| | | | | GCUAUGCUGUUUUG | | |
| CR005696 | 11 | CCAGCACCGU | 101 | CCAGCACCGUCACU | 189 | chr9: 121021516- |
| | | CACUCCAGAU | | CCAGAUGUUUUAGA | | 121021536 |
| | | | | GCUAUGCUGUUUUG | | |
| CR005697 | 11 | UUGUUUUGAC | 102 | UUGUUUUGACAAAC | 190 | chr9: 121021690- |
| | | AAACAGGUGC | | AGGUGCGUUUUAGA | | 121021710 |
| | | | | GCUAUGCUGUUUUG | | |
| CR005698 | 11 | CCCAUCUGGA | 103 | CCCAUCUGGAGUGA | 191 | chr9: 121021519- |
| | | GUGACGGUGC | | CGGUGCGUUUUAGA | | 121021539 |
| | | | | GCUAUGCUGUUUUG | | |
| CR005699 | 11 | UCCAGCACCG | 104 | UCCAGCACCGUCAC | 192 | chr9: 121021515- |
| | | UCACUCCAGA | | UCCAGAGUUUUAGA | | 121021535 |
| | | | | GCUAUGCUGUUUUG | | |
| CR005700 | 10 | CUUAGGUGGA | 105 | CUUAGGUGGAUUUU | 193 | chr9: 121023504- |
| | | UUUUCUGAAG | | CUGAAGGUUUUAGA | | 121023524 |
| | | | | GCUAUGCUGUUUUG | | |
| CR005701 | 10 | AUGCUUGCCA | 106 | AUGCUUGCCAUUUC | 194 | chr9: 121023518- |
| | | UUUCCUUAGG | | CUUAGGGUUUUAGA | | 121023538 |
| | | | | GCUAUGCUGUUUUG | | |
| CR005702 | 10 | CUCUCCCUAC | 107 | CUCUCCCUACAAAC | 195 | chr9: 121023453- |
| | | AAACUGAAUU | | UGAAUUGUUUUAGA | | 121023473 |
| | | | | GCUAUGCUGUUUUG | | |
| CR005703 | 10 | GAGAGGACAU | 108 | GAGAGGACAUAUUU | 196 | chr9: 121023470- |
| | | AUUUGAUGCC | | GAUGCCGUUUUAGA | | 121023490 |
| | | | | GCUAUGCUGUUUUG | | |
| CR005704 | 10 | GCAACCAAAU | 109 | GCAACCAAAUUCAG | 197 | chr9: 121023446- |
| | | UCAGUUUGUA | | UUUGUAGUUUUAGA | | 121023466 |
| | | | | GCUAUGCUGUUUUG | | |
| CR005705 | 6 | GCUUCCACAG | 110 | GCUUCCACAGAUAU | 198 | chr9: 121032185- |
| | | AUAUGGUAUG | | GGUAUGGUUUUAGA | | 121032205 |
| | | | | GCUAUGCUGUUUUG | | |
| CR005706 | 5 | UUAGACGGAA | 111 | UUAGACGGAAUCUU | 199 | chr9: 121034808- |
| | | UCUUGAAGUC | | GAAGUCGUUUUAGA | | 121034828 |
| | | | | GCUAUGCUGUUUUG | | |
| CR005707 | 4 | AAGACAGUUU | 112 | AAGACAGUUUCUCU | 200 | chr9: 121037890- |
| | | CUCUUUUGGC | | UUUGGCGUUUUAGA | | 121037910 |
| | | | | GCUAUGCUGUUUUG | | |
| CR005708 | 3 | AAUCCAUUGU | 113 | AAUCCAUUGUCAUA | 201 | chr9: 121043050- |
| | | CAUAGGUUAU | | GGUUAUGUUUUAGA | | 121043070 |
| | | | | GCUAUGCUGUUUUG | | |
| CR005709 | 2 | UCUCUGAGGA | 114 | UCUCUGAGGAUAAA | 202 | chr9: 121046219- |
| | | UAAAUGAACA | | UGAACAGUUUUAGA | | 121046239 |
| | | | | GCUAUGCUGUUUUG | | |
| CR005710 | 2 | GGAUAAAUGA | 115 | GGAUAAAUGAACAU | 203 | chr9: 121046226- |
| | | ACAUGGCCUG | | GGCCUGGUUUUAGA | | 121046246 |
| | | | | GCUAUGCUGUUUUG | | |
| CR005711 | 2 | GCACCAAAAA | 116 | GCACCAAAAAUAUU | 204 | chr9: 121046350- |
| | | UAUUCCGUGU | | CCGUGUGUUUUAGA | | 121046370 |
| | | | | GCUAUGCUGUUUUG | | |
| CR005712 | 2 | GCUCCAACAC | 117 | GCUCCAACACGGAA | 205 | chr9: 121046344- |
| | | GGAAUAUUUU | | UAUUUUGUUUUAGA | | 121046364 |
| | | | | GCUAUGCUGUUUUG | | |
| CR005713 | 1 | UUUCCUGCUA | 118 | UUUCCUGCUACCUC | 206 | chr9: 121050245- |
| | | CCUCCAACCA | | CAACCAGUUUUAGA | | 121050265 |
| | | | | GCUAUGCUGUUUUG | | |
| CR005714 | 1 | CCUCCAACCA | 119 | CCUCCAACCAUGGG | 207 | chr9: 121050235- |
| | | UGGGCCUUUU | | CCUUUUGUUUUAGA | | 121050255 |
| | | | | GCUAUGCUGUUUUG | | |
| CR005715 | 1 | ACCUCCAACC | 120 | ACCUCCAACCAUGG | 208 | chr9: 121050236- |
| | | AUGGGCCUUU | | GCCUUUGUUUUAGA | | 121050256 |
| | | | | GCUAUGCUGUUUUG | | |

TABLE 3
C5 sgRNA Sequences.

| | | | SEQ | | SEQ |
|---|---|---|---|---|---|
| gRNA | crRNA | sgRNA | ID | Modified sgRNA | ID |
| G012393 | CR005660 | UGCCUACCAUAAA | 297 | mU*mG*mC*CUACCAUAAAUACCC | 316 |
| | | UACCCCUGUUUUA | | CUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012394 | CR005655 | CUCUCGGGCUUCA | 298 | mC*mU*mC*UCGGGCUUCAACAGG | 317 |
| | | ACAGGCAGUUUUA | | CAGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012395 | CR005692 | GAGAUGAGUAUGC | 299 | mG*mA*mG*AUGAGUAUGCUAUUG | 318 |
| | | UAUUGCUGUUUUA | | CUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012396 | CR005682 | UCCAGCUAGGUGG | 300 | mU*mC*mC*AGCUAGGUGGAACAC | 319 |
| | | AACACAUGUUUUA | | AUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012397 | CR005680 | UGAGGAAGGUAAG | 301 | mU*mG*mA*GGAAGGUAAGUCCAG | 320 |
| | | UCCAGCUGUUUUA | | CUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012398 | CR005662 | UGCCCUGAUGAUC | 302 | mU*mG*mC*CCUGAUGAUCAAUGA | 321 |
| | | AAUGACUGUUUUA | | CUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012399 | CR005714 | CCUCCAACCAUGG | 303 | mC*mC*mU*CCAACCAUGGGCCUU | 322 |
| | | GCCUUUUGUUUUA | | UUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G012400 | CR005677 | UUCUUAGAGAAGA | 304 | mU*mU*mC*UUAGAGAAGAGUGAU | 323 |
| | | GUGAUCUGUUUUA | | CUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013040 | CR005635 | GACUUUCUGAAUU | 305 | mG*mA*mC*UUUCUGAAUUUUGAU | 324 |
| | | UUGAUAUGUUUUA | | AUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013041 | CR005650 | GACAGCUCUGUAC | 306 | mG*mA*mC*AGCUCUGUACCUAAC | 325 |
| | | CUAACACGUUUUA | | ACGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013042 | CR005656 | UGCACUCCCUUUG | 307 | mU*mG*mC*ACUCCCUUUGGGGAG | 326 |
| | | GGGAGGUGUUUUA | | GUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013043 | CR005672 | CAGUAAGCAAGCC | 308 | mC*mA*mG*UAAGCAAGCCAGAAA | 327 |
| | | AGAAAUUGUUUUA | | UUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013044 | CR005679 | GCCAAUGUGUUCC | 309 | mG*mC*mC*AAUGUGUUCCACCUA | 328 |
| | | ACCUAGCGUUUUA | | GCGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013045 | CR005689 | UUUGUCAAUAUAU | 310 | mU*mU*mU*GUCAAUAUAUGGGCU | 329 |
| | | GGGCUUUGUUUUA | | UUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013046 | CR005701 | AUGCUUGCCAUUU | 311 | mA*mU*mG*CUUGCCAUUUCCUUA | 330 |
| | | CCUUAGGGUUUUA | | GGGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |
| G013047 | CR005651 | AUCCACCCAUUUA | 312 | mA*mU*mC*CACCCAUUUAUCGUU | 331 |
| | | UCGUUUUGUUUUA | | UUGUUUUAGAmGmCmUmAmGmA | |
| | | GAGCUAGAAAUAG | | mAmAmUmAmGmCAAGUUAAAAU | |
| | | CAAGUUAAAAUAA | | AAGGCUAGUCCGUUAUCAmAmCm | |
| | | GGCUAGUCCGUUA | | UmUmGmAmAmAmAmAmGmUmGm | |
| | | UCAACUUGAAAAA | | GmCmAmCmCmGmAmGmUmCmGm | |
| | | GUGGCACCGAGUC | | GmUmGmCmU*mU*mU*mU | |
| | | GGUGCUUUU | | | |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 600% (e.g., at least 6500, at least 700%, at least 7500, at least 800%, at least 850%, at least 900%, at least 950%, at least 970%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 21); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 22); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 23); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 24); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 25); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 26); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 27); or GUUUUAGAGCUAGAAAUAGCAAGUUAAAAUAAGGCUAGUCCGUUAUCAACUUGG CACCGAGUCGGUGC (version 8; SEQ ID NO: 28). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide, and wherein the totality of N residues comprise a C5 DNA-targeting segment as described herein (e.g., the sequence set forth in SEQ ID NO: 29, wherein the N residues are replaced with the DNA-targeting segment of any one of SEQ ID NOS: 33-120. In another example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide, and wherein the totality of N residues comprise a C5 DNA-targeting segment as described herein (e.g., the sequence set forth in SEQ ID NO: 29, wherein the N residues are replaced with the DNA-targeting segment of any one of SEQ ID NOS: 60, 65, 67, 82, 85, 87, 97, and 119. In another example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide, and wherein the totality of N residues comprise a C5 DNA-targeting segment as described herein (e.g., the sequence set forth in SEQ ID NO: 29, wherein the N residues are replaced with the DNA-targeting segment of any one of SEQ ID NOS: 85 and 97. In another example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide, and wherein the totality of N residues comprise a C5 DNA-targeting segment as described herein (e.g., the sequence set forth in SEQ ID NO: 29, wherein the N residues are replaced with the DNA-targeting segment of SEQ ID NO: 85. In another example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide, and wherein the totality of N residues comprise a C5 DNA-targeting segment as described herein (e.g., the sequence set forth in SEQ ID NO: 29, wherein the N residues are replaced with the DNA-targeting segment of SEQ ID NO: 97. The terms “mA,” “mC,” “mU,” and “mG” denote a nucleotide (A, C, U, and G, respectively) that has been modified with 2′-O-Me. The symbol “*” depicts a phosphorothioate modification. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting a C5 gene can comprise, consist essentially of, or consist of the sequence set forth in any one of SEQ ID NOS: 297-312 and 316-331. Alternatively, a guide RNA targeting a C5 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 297-312 and 316-331. Alternatively, a guide RNA targeting a C5 gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 297-312 and 316-331. Alternatively, a guide RNA targeting a C5 gene can comprise, consist essentially of, or consist of a sequence that differs by no more than 3, no more than 2, or no more than 1 nucleotide from the sequence set forth in any one of SEQ ID NOS: 297-312 and 316-331.

As one example, a guide RNA targeting a C5 gene can comprise, consist essentially of, or consist of the sequence set forth in any one of SEQ ID NOS: 297-304 and 316-323. Alternatively, a guide RNA targeting a C5 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 297-304 and 316-323. Alternatively, a guide RNA targeting a C5 gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 297-304 and 316-323. Alternatively, a guide RNA targeting a C5 gene can comprise, consist essentially of, or consist of a sequence that differs by no more than 3, no more than 2, or no more than 1 nucleotide from the sequence set forth in any one of SEQ ID NOS: 297-304 and 316-323.

As one example, a guide RNA targeting a C5 gene can comprise, consist essentially of, or consist of the sequence set forth in any one of SEQ ID NOS: 299, 301, 318, and 320. Alternatively, a guide RNA targeting a C5 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 299, 301, 318, and 320. Alternatively, a guide RNA targeting a C5 gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 299, 301, 318, and 320. Alternatively, a guide RNA targeting a C5 gene can comprise, consist essentially of, or consist of a sequence that differs by no more than 3, no more than 2, or no more than 1 nucleotide from the sequence set forth in any one of SEQ ID NOS: 299, 301, 318, and 320.

As one example, a guide RNA targeting a C5 gene can comprise, consist essentially of, or consist of the sequence set forth in any one of SEQ ID NOS: 299 and 301. Alternatively, a guide RNA targeting a C5 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 299 and 301. Alternatively, a guide RNA targeting a C5 gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 299 and 301. Alternatively, a guide RNA targeting a C5 gene can comprise, consist essentially of, or consist of a sequence that differs by no more than 3, no more than 2, or no more than 1 nucleotide from the sequence set forth in any one of SEQ ID NOS: 299 and 301.

As one example, a guide RNA targeting a C5 gene can comprise, consist essentially of, or consist of the sequence set forth in any one of SEQ ID NOS: 318 and 320. Alternatively, a guide RNA targeting a C5 gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 318 and 320. Alternatively, a guide RNA targeting a C5 gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 318 and 320. Alternatively, a guide RNA targeting a C5 gene can comprise, consist essentially of, or consist of a sequence that differs by no more than 3, no more than 2, or no more than 1 nucleotide from the sequence set forth in any one of SEQ ID NOS: 318 and 320.

### D. Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 30) or N₂₀NGG (SEQ ID NO: 31). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 32) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 30-32, including the 5′ G or GG and the 3′ GG or NGG. Yet other guide RNA target sequences plus PAMs can have between 14 and 20 nucleotides in length of SEQ ID NOS: 30-32.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

A guide RNA targeting a C5 gene, such as a human C5 gene can target any desired location in the C5 gene. For example, a guide RNA target sequence can comprise any contiguous sequence in the C5 gene. The term C5 gene includes the genomic region encompassing the C5 regulatory promoters and enhancer sequences as well as the coding sequence. A guide RNA target sequence can comprise a coding sequence, a non-coding sequence (e.g., a regulatory element such as a promoter or enhancer region), or a combination thereof. As one example, a guide RNA target sequence can comprise a contiguous coding sequence in any of the C5 coding exons. In one example, the guide RNA target sequence is not in coding exon 16 and 17, as this region encodes anaphylatoxin C5a. As one example, the guide RNA target sequence can be in coding exon 1 of the C5 gene. As another example, the guide RNA target sequence can be in coding exon 2 of the C5 gene. As another example, the guide RNA target sequence can be in coding exon 3 of the C5 gene. As another example, the guide RNA target sequence can be in coding exon 4 of the C5 gene. As another example, the guide RNA target sequence can be in coding exon 5 of the C5 gene. As another example, the guide RNA target sequence can be in coding exon 6 of the C5 gene. As another example, the guide RNA target sequence can be in coding exon 7 of the C5 gene. As another example, the guide RNA target sequence can be in coding exon 8 of the C5 gene. As another example, the guide RNA target sequence can be in coding exon 9 of the C5 gene. As another example, the guide RNA target sequence can be in coding exon 10 of the C5 gene. As another example, the guide RNA target sequence can be in coding exon 11 of the C5 gene. As another example, the guide RNA target sequence can be in coding exon 12 of the C5 gene. As another example, the guide RNA target sequence can be in coding exon 13 of the C5 gene. As another example, the guide RNA target sequence can be in coding exon 14 of the C5 gene. As another example, the guide RNA target sequence can be in coding exon 15 of the C5 gene. As another example, the guide RNA target sequence can be in coding exon 16 of the C5 gene. As another example, the guide RNA target sequence can be in coding exon 17 of the C5 gene. As another example, the guide RNA target sequence can be in coding exon 18 of the C5 gene. As another example, the guide RNA target sequence can be in coding exon 19 of the C5 gene. As another example, the guide RNA target sequence can be in coding exon 20 of the C5 gene. As another example, the guide RNA target sequence can be in coding exon 21 of the C5 gene. As another example, the guide RNA target sequence can be in coding exon 22 of the C5 gene. As another example, the guide RNA target sequence can be in coding exon 23 of the C5 gene. As another example, the guide RNA target sequence can be in coding exon 24 of the C5 gene. As another example, the guide RNA target sequence can be in coding exon 25 of the C5 gene. As another example, the guide RNA target sequence can be in coding exon 26 of the C5 gene. As another example, the guide RNA target sequence can be in coding exon 27 of the C5 gene. As another example, the guide RNA target sequence can be in coding exon 28 of the C5 gene. As another example, the guide RNA target sequence can be in coding exon 29 of the C5 gene. As another example, the guide RNA target sequence can be in coding exon 30 of the C5 gene. As another example, the guide RNA target sequence can be in coding exon 31 of the C5 gene. As another example, the guide RNA target sequence can be in coding exon 32 of the C5 gene. As another example, the guide RNA target sequence can be in coding exon 33 of the C5 gene. As another example, the guide RNA target sequence can be in coding exon 34 of the C5 gene. As another example, the guide RNA target sequence can be in coding exon 35 of the C5 gene. As another example, the guide RNA target sequence can be in coding exon 36 of the C5 gene. As another example, the guide RNA target sequence can be in coding exon 37 of the C5 gene. As another example, the guide RNA target sequence can be in coding exon 38 of the C5 gene. As another example, the guide RNA target sequence can be in coding exon 39 of the C5 gene. As another example, the guide RNA target sequence can be in coding exon 40 of the C5 gene. As another example, the guide RNA target sequence can be in coding exon 41 of the C5 gene. A guide RNA target sequence is in coding exon X if at least a portion of the guide RNA target sequence (e.g., at least one nucleotide) is in coding exon X.

In a specific example, the guide RNA target sequence can be in coding exon 1, 12, 15, 21, 22, or 27. In another specific example, the guide RNA target sequence can be in coding exon 12 or 15.

The guide RNA target sequence can be selected to target a coding region of the C5 gene so that cleavage by the corresponding Cas protein will result in frameshift insertion/deletion (indel) mutations that result in a loss-of-function allele. An indel refer to insertion/deletion mutations consisting of a number of nucleotides that are either inserted or deleted at the site of double-stranded breaks (DSBs) in a target nucleic acid. Such frameshift mutations can be achieved through targeted DNA double strand breaks and subsequent mutagenic repair via the non-homologous end joining (NHEJ) pathway, which produces indels at the site of break. The indel being introduced into the DSB is random, with some indels leading to frameshift mutations that cause premature termination of the C5 gene. As another example, a guide RNA target sequence can be in a promoter region or enhancer region of the C5 gene so that cleavage by the corresponding Cas protein will result in disruption of the promoter region or enhancer region. Such mutations can be achieved through targeted DNA double strand breaks and subsequent mutagenic repair via the non-homologous end joining (NHEJ) pathway, which produces indels at the site of break.

The guide RNA target sequence can be in a constitutive exon of the C5 gene. For example, a guide RNA target sequence can be in a 5′ constitutive exon. Constitutive exons are coding exons that are consistently conserved after splicing. Exons expressed across all tissues in which C5 is expressed can be considered constitutive exons for gRNA targeting. In some examples, a guide RNA targeting a C5 gene does not target any exon containing an alternative splicing site. Because there is only a single C5 coding transcript, all 41 coding exons of C5 are considered constitutive.

As another example, the guide RNA target sequence can be within about 10, about 20, about 30, about 40, about 50, about 100, about 200, about 300, about 400, about 500, or about 1,000 nucleotides of the start codon or can comprise the start codon.

The guide RNA target sequence can also be selected to minimize off-target modification or avoid off-target effects (e.g., by avoiding two or fewer mismatches to off-target genomic sequences).

As one example, a guide RNA targeting a C5 gene can target the guide RNA target sequence set forth in any one of SEQ ID NOS: 209-296. As another example, a guide RNA targeting a C5 gene can target at least 17, at least 18, at least 19, or at least 20 contiguous nucleotides of the guide RNA target sequence set forth in any one of SEQ ID NOS: 209-296.

As another example, a guide RNA targeting a C5 gene can target the guide RNA target sequence set forth in any one of SEQ ID NOS: 236, 241, 243, 258, 261, 263, 273, and 295. As another example, a guide RNA targeting a C5 gene can target at least 17, at least 18, at least 19, or at least 20 contiguous nucleotides of the guide RNA target sequence set forth in any one of SEQ ID NOS: 236, 241, 243, 258, 261, 263, 273, and 295.

As another example, a guide RNA targeting a C5 gene can target the guide RNA target sequence set forth in any one of SEQ ID NOS: 261 and 273. As another example, a guide RNA targeting a C5 gene can target at least 17, at least 18, at least 19, or at least 20 contiguous nucleotides of the guide RNA target sequence set forth in any one of SEQ ID NOS: 261 and 273.

As another example, a guide RNA targeting a C5 gene can target the guide RNA target sequence set forth in SEQ ID NO: 261. As another example, a guide RNA targeting a C5 gene can target at least 17, at least 18, at least 19, or at least 20 contiguous nucleotides of the guide RNA target sequence set forth in SEQ ID NO: 261.

As another example, a guide RNA targeting a C5 gene can target the guide RNA target sequence set forth in SEQ ID NO: 273. As another example, a guide RNA targeting a C5 gene can target at least 17, at least 18, at least 19, or at least 20 contiguous nucleotides of the guide RNA target sequence set forth in SEQ ID NO: 273.

TABLE 4
C5 Guide RNA Target Sequences and Chromosomal Coordinates.

| | Coding | | | |
|---|---|---|---|---|
| ID | Exon | Target Sequence | SEQ ID | Location |
| CR005628 | 41 | CCTTGACCTGGATTGAATAC | 209 | chr9:120952828-120952848 |
| CR005629 | 41 | ACCCTTTAGATTCCTTGACC | 210 | chr9:120952840-120952860 |
| CR005630 | 40 | GGAAGACAGTACTTAATTAT | 211 | chr9:120953771-120953791 |
| CR005631 | 40 | AGGAAGACAGTACTTAATTA | 212 | chr9:120953772-120953792 |
| CR005632 | 39 | AGTTTTGTAGATATCCAGAA | 213 | chr9:120957285-120957305 |
| CR005633 | 39 | CAGTTTTGTAGATATCCAGA | 214 | chr9:120957284-120957304 |
| CR005634 | 38 | AACTCACCATATGCAATCTC | 215 | chr9:120960240-120960260 |
| CR005635 | 37 | GACTTTCTGAATTTTGATAT | 216 | chr9:120961512-120961532 |
| CR005636 | 36 | TACACAAAGGAAATCACTGG | 217 | chr9:120962749-120962769 |
| CR005637 | 36 | GGAGTTGATTACCTGGTCTG | 218 | chr9:120962658-120962678 |
| CR005638 | 36 | TTAGCATGGAGTTGATTACC | 219 | chr9:120962651-120962671 |
| CR005639 | 35 | CTCTTTTCCCCCTTAGCTTG | 220 | chr9:120962963-120962983 |
| CR005640 | 35 | TTCCCCCTTAGCTTGTGGAA | 221 | chr9:120962958-120962978 |
| CR005641 | 34 | ATGGACATCTCCTTGCCTAC | 222 | chr9:120963669-120963689 |
| CR005642 | 33 | GCATCCCACTACAGAGGCTA | 223 | chr9:120969099-120969119 |
| CR005643 | 32 | TCACAAAACCAGTACCTCTG | 224 | chr9:120970217-120970237 |
| CR005644 | 32 | AAAGCTGCAAACTTCCTCAG | 225 | chr9:120970200-120970220 |
| CR005645 | 31 | TACAGGATTTGGCAGTGGCT | 226 | chr9:120971943-120971963 |
| CR005646 | 31 | GTCAGTACAGGATTTGGCAG | 227 | chr9:120971948-120971968 |
| CR005647 | 30 | CAGACAAGAATTTCCTTGGG | 228 | chr9:120974790-120974810 |
| CR005648 | 30 | AGTTCTTCATTTACCTCTAC | 229 | chr9:120974764-120974784 |
| CR005649 | 29 | CTATCAGAAGAGCAGAGGTA | 230 | chr9:120976724-120976744 |
| CR005650 | 29 | GACAGCTCTGTACCTAACAC | 231 | chr9:120976838-120976858 |
| CR005651 | 29 | ATCCACCCATTTATCGTTTT | 232 | chr9:120976882-120976902 |
| CR005652 | 28 | GAAACAATTGAACGAAACTG | 233 | chr9:120980111-120980131 |
| CR005653 | 28 | AGCCCAGAGCACCTTTACAT | 234 | chr9:120980178-120980198 |
| CR005654 | 27 | CTGTTCTCTCGGGCTTCAAC | 235 | chr9:120981907-120981927 |
| CR005655 | 27 | CTCTCGGGCTTCAACAGGCA | 236 | chr9:120981912-120981932 |
| CR005656 | 24 | TGCACTCCCTTTGGGGAGGT | 237 | chr9:120989697-120989717 |
| CR005657 | 24 | ATTCTATGTTTTTCACTACC | 238 | chr9:120989648-120989668 |
| CR005658 | 23 | GACGAAAGGAGTTCCCATAC | 239 | chr9:120991248-120991268 |
| CR005659 | 23 | AGGTACCATTAGCAGACGAA | 240 | chr9:120991262-120991282 |
| CR005660 | 22 | TGCCTACCATAAATACCCCT | 241 | chr9:120996232-120996252 |
| CR005661 | 22 | TTAGCCAGAAGGTGTCAAAA | 242 | chr9:120996284-120996304 |
| CR005662 | 21 | TGCCCTGATGATCAATGACT | 243 | chr9:120997704-120997724 |
| CR005663 | 21 | GTGCCCTGATGATCAATGAC | 244 | chr9:120997703-120997723 |
| CR005664 | 21 | AGCCCAGTCATTGATCATCA | 245 | chr9:120997709-120997729 |
| CR005665 | 21 | AAGCCCAGTCATTGATCATC | 246 | chr9:120997710-120997730 |
| CR005666 | 20 | TTTACAACTATAGGACTTCT | 247 | chr9:121005927-121005947 |
| CR005667 | 20 | TCTCCTCGTACAACAGAATA | 248 | chr9:121005970-121005990 |
| CR005668 | 20 | AAGGAACTGTTTACAACTAT | 249 | chr9:121005936-121005956 |
| CR005669 | 20 | GTTTACAACTATAGGACTTC | 250 | chr9:121005928-121005948 |
| CR005670 | 20 | ATACCATATTCTGTTGTACG | 251 | chr9:121005976-121005996 |
| CR005671 | 18 | CGAATTTCTGGCTTGCTTAC | 252 | chr9:121008458-121008478 |
| CR005672 | 18 | CAGTAAGCAAGCCAGAAATT | 253 | chr9:121008460-121008480 |
| CR005673 | 18 | TGGCTTGCTTACTGGTAACA | 254 | chr9:121008466-121008486 |
| CR005674 | 18 | CTGGCTTGCTTACTGGTAAC | 255 | chr9:121008465-121008485 |
| CR005675 | 17 | CCGTGCAGCTCGCTGCTCAC | 256 | chr9:121013978-121013998 |
| CR005676 | 17 | GTGAAGAAATGTTGTTACGA | 257 | chr9:121014027-121014047 |
| CR005677 | 15 | TTCTTAGAGAAGAGTGATCT | 258 | chr9:121016354-121016374 |
| CR005678 | 15 | ATTCTTAGAGAAGAGTGATC | 259 | chr9:121016355-121016375 |
| CR005679 | 15 | GCCAATGTGTTCCACCTAGC | 260 | chr9:121016303-121016323 |
| CR005680 | 15 | TGAGGAAGGTAAGTCCAGCT | 261 | chr9:121016286-121016306 |
| CR005681 | 15 | GGAAGGTAAGTCCAGCTAGG | 262 | chr9:121016289-121016309 |
| CR005682 | 15 | TCCAGCTAGGTGGAACACAT | 263 | chr9:121016299-121016319 |
| CR005683 | 14 | AACTCTTTCCAAGGGCTTTT | 264 | chr9:121017358-121017378 |
| CR005684 | 14 | CATTACTTAACTCTTTCCAA | 265 | chr9:121017350-121017370 |
| CR005685 | 14 | CCATATTAAGAGACACAGTT | 266 | chr9:121017450-121017470 |
| CR005686 | 13 | ATTTGTGGCTTACCTGGAGC | 267 | chr9:121017629-121017649 |
| CR005687 | 13 | AAGGGCAAAATTATCCACTT | 268 | chr9:121017823-121017843 |
| CR005688 | 13 | AATTAGTGTCTGATTCAGTC | 269 | chr9:121017681-121017701 |
| CR005689 | 12 | TTTGTCAATATATGGGCTTT | 270 | chr9:121019996-121020016 |
| CR005690 | 12 | GAGTTATTTTGTCAATATAT | 271 | chr9:121019989-121020009 |
| CR005691 | 12 | TTGTCAATATATGGGCTTTT | 272 | chr9:121019997-121020017 |
| CR005692 | 12 | GAGATGAGTATGCTATTGCT | 273 | chr9:121020103-121020123 |
| CR005693 | 11 | TTTGTGCATTCAGTGTTACT | 274 | chr9:121021630-121021650 |
| CR005694 | 11 | GTTTGTGCATTCAGTGTTAC | 275 | chr9:121021629-121021649 |
| CR005695 | 11 | AGTGTAACACGTGTTGATGA | 276 | chr9:121021566-121021586 |
| CR005696 | 11 | CCAGCACCGTCACTCCAGAT | 277 | chr9:121021516-121021536 |
| CR005697 | 11 | TTGTTTTGACAAACAGGTGC | 278 | chr9:121021690-121021710 |
| CR005698 | 11 | CCCATCTGGAGTGACGGTGC | 279 | chr9:121021519-121021539 |
| CR005699 | 11 | TCCAGCACCGTCACTCCAGA | 280 | chr9:121021515-121021535 |
| CR005700 | 10 | CTTAGGTGGATTTTCTGAAG | 281 | chr9:121023504-121023524 |
| CR005701 | 10 | ATGCTTGCCATTTCCTTAGG | 282 | chr9:121023518-121023538 |
| CR005702 | 10 | CTCTCCCTACAAACTGAATT | 283 | chr9:121023453-121023473 |
| CR005703 | 10 | GAGAGGACATATTTGATGCC | 284 | chr9:121023470-121023490 |
| CR005704 | 10 | GCAACCAAATTCAGTTTGTA | 285 | chr9:121023446-121023466 |
| CR005705 | 6 | GCTTCCACAGATATGGTATG | 286 | chr9:121032185-121032205 |
| CR005706 | 5 | TTAGACGGAATCTTGAAGTC | 287 | chr9:121034808-121034828 |
| CR005707 | 4 | AAGACAGTTTCTCTTTTGGC | 288 | chr9:121037890-121037910 |
| CR005708 | 3 | AATCCATTGTCATAGGTTAT | 289 | chr9:121043050-121043070 |
| CR005709 | 2 | TCTCTGAGGATAAATGAACA | 290 | chr9:121046219-121046239 |
| CR005710 | 2 | GGATAAATGAACATGGCCTG | 291 | chr9:121046226-121046246 |
| CR005711 | 2 | GCACCAAAAATATTCCGTGT | 292 | chr9:121046350-121046370 |
| CR005712 | 2 | GCTCCAACACGGAATATTTT | 293 | chr9:121046344-121046364 |
| CR005713 | 1 | TTTCCTGCTACCTCCAACCA | 294 | chr9:121050245-121050265 |
| CR005714 | 1 | CCTCCAACCATGGGCCTTTT | 295 | chr9:121050235-121050255 |
| CR005715 | 1 | ACCTCCAACCATGGGCCTTT | 296 | chr9:121050236-121050256 |

### E. Lipid Nanoparticles Comprising CRISPR/Cas Systems Targeting a C5 Locus or Gene

Lipid nanoparticles comprising the CRISPR/Cas systems targeting a C5 locus or gene are also provided. The lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an exogenous donor sequence. In certain LNPs, the cargo can include an mRNA encoding a Cas nuclease, such as Cas9, a guide RNA or a nucleic acid encoding a guide RNA, and an exogenous donor sequence. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be about 4.5 or can be about 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, about 25:1, about 10:1, about 5:1, about 3:1, about 1:1, about 1:3, about 1:5, about 1:10, or about 1:25. Alternatively, the LNP formulation can include a ratio of Cas mRNA to gRNA nucleic acid of from about 1:1 to about 1:2. In specific examples, the ratio of Cas mRNA to gRNA can be about 1:1 or about 1:2. Alternatively, the LNP formulation can include a ratio of Cas mRNA to gRNA nucleic acid of from about 2:1 to about 1:2. In specific examples, the ratio of Cas mRNA to gRNA can be about 2:1 or about 1:1 or about 1:2.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, about 25:1, about 10:1, about 5:1, about 3:1, about 1:1, about 1:3, about 1:5, about 1:10, or about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT© GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### F. Viral Vectors Comprising CRISPR/Cas Systems Targeting a C5 Locus or Gene

Viral vectors, such as adeno-associated virus (AAV) vectors, comprising the CRISPR/Cas systems targeting a C5 locus or gene are also provided. Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas and/or gRNA). Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 706, SEQ ID NO: 707, or SEQ ID NO: 708. Other examples of ITRs comprise one or more mutations compared to SEQ ID NO: 706, SEQ ID NO: 707, or SEQ ID NO: 708 and can be at least 90%, at least 91%, at least 92%, at least 93%, at least 94%, at least 95%, at least 96%, at least 97%, at least 98%, or at least 99% identical to SEQ ID NO: 706, SEQ ID NO: 707, or SEQ ID NO: 708. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end). In one example, the ITR on each end can comprise, consist essentially of, or consist of SEQ ID NO: 706. In another example, the ITR on each end can comprise, consist essentially of, or consist of SEQ ID NO: 707. In one example, the ITR on at least one end comprises, consists essentially of, or consists of SEQ ID NO: 708. In one example, the ITR on the 5′ end comprises, consists essentially of, or consists of SEQ ID NO: 708. In one example, the ITR on the 3′ end comprises, consists essentially of, or consists of SEQ ID NO: 708. In one example, the ITR on each end can comprise, consist essentially of, or consist of SEQ ID NO: 708. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 706, and the ITR on the other end comprises, consists essentially of, or consists of SEQ ID NO: 707. In another example, the ITR on one end comprises, consists essentially of, or consists of SEQ ID NO: 706, and the ITR on the other end comprises, consists essentially of, or consists of SEQ ID NO: 708. In one example, the ITR on one end comprises, consists essentially of, or consists of SEQ ID NO: 707, and the ITR on the other end comprises, consists essentially of, or consists of SEQ ID NO: 708.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include an exogenous donor sequence. In certain AAVs, the cargo can include a nucleic acid (e.g., DNA) encoding a Cas nuclease, such as Cas9, a DNA encoding a guide RNA (or multiple guide RNAs), and an exogenous donor sequence.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV8-mediated delivery). For example, a Cas9 mRNA and a gRNA targeting a CS gene can be delivered via LNP-mediated delivery, or DNA encoding Cas9 and DNA encoding a gRNA targeting a CS gene can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### III. C5 Antigen-Binding Proteins

The methods and compositions disclosed herein can utilize C5 antigen-binding proteins (i.e., anti-C5 antigen binding proteins) such as C5 antibodies (i.e., anti-C5 antibodies) or an antigen-binding fragment thereof. Examples of C5 antigen-binding proteins include, but are not limited to, those disclosed in WO 2021/034639 A1, US 2021-0046182, WO 2021/081277 A1, US 2021-0139573, WO 2017/218515 A1, US 2020-0262901, US 2017-0355757, or US 2020-0262900, each of which is herein incorporated by reference in its entirety for all purposes.

The term “antibody,” as used herein, refers to immunoglobulin molecules comprising four polypeptide chains, two heavy chains (HCs) and two light chains (LCs), inter-connected by disulfide bonds (e.g., IgG), for example H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; eculizumab; tesidolumab; mubodina; or ravulizumab; preferably, pozelimab. In some embodiments, each antibody heavy chain (HC) comprises a heavy chain variable region (“HCVR” or “V_{H}”) (e.g., SEQ ID NO: 341; 357; 373; 389; 405; 421; 437; 461; 477; 485; 493; 509; 525; 541; 557; 573; 589; 605; 613; 629; 645; 661; or 677; or a variant thereof) and a heavy chain constant region; and each antibody light chain (LC) comprises a light chain variable region (“LCVR or “V_{L}”) (e.g., SEQ ID NO: 349; 365; 381; 397; 413; 429; 445; 453; 469; 501; 517; 533; 549; 565; 581; 597; 621; 637; 653; 669; or 685; or a variant thereof) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. In some embodiments, an antibody or antigen-binding fragment thereof used in the methods and combinations disclosed herein was expressed and isolated from a mammalian host cell such as a Chinese hamster ovary (CHO) cell.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)₂ fragments; (iii) Fd fragments (heavy chain portion of a Fab fragment cleaved with papain); (iv) Fv fragments (a V_{H} or V_{L}); and (v) single-chain Fv (scFv) molecules; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein. In some embodiments, the antigen-binding fragment comprises three or more CDRs of H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N (e.g., CDR-H1, CDR-H2 and CDR-H3; and/or CDR-L1, CDR-L2 and CDR-L3.

The term “recombinant” antigen-binding proteins, such as antibodies or antigen-binding fragments thereof, refers to such molecules created, expressed, isolated, or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. Recombinant antigen-binding proteins as set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N) can be used in the compositions and methods disclosed herein.

Antibodies as set forth herein include, for example, monoclonal, recombinant, chimeric, human and/or humanized antibodies. For example, included are monoclonal anti-C5 antigen-binding proteins (e.g., antibodies and antigen-binding fragments thereof). The term “monoclonal antibody” or “mAb”, as used herein, refers to an antibody from a population of substantially homogeneous antibodies, i.e., the antibody molecules comprising the population are identical in amino acid sequence except for possible naturally occurring mutations that may be present in minor amounts. The modifier “monoclonal” is not to be construed as requiring production of the antibody by any particular method. Monoclonal antibodies may be made by the hybridoma method of Kohler et al. (1975) Nature 256:495, herein incorporated by reference in its entirety for all purposes, or may be made by recombinant DNA methods (see, e.g., U.S. Pat. No. 4,816,567, herein incorporated by reference in its entirety for all purposes).

In some embodiments, the assignment of amino acids to each framework or CDR domain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat et al. (1977) J. Biol. Chem. 252:6609-6616; Chothia et al. (1987) J. Mol. Biol. 196:901-917; or Chothia et al. (1989) Nature 342:878-883, each of which is herein incorporated by reference in its entirety for all purposes. Thus, included are antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

In some embodiments, a C5 antigen-binding protein, e.g., antibody or antigen-binding fragment, comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. See, e.g., Silva et al. (2015) J Biol Chem. 290(9):5462-9, herein incorporated by reference in its entirety for all purposes. In some embodiments, an antigen-binding protein, e.g., antibody or antigen-binding fragment, comprises a light chain constant domain, e.g., of the type kappa or lambda. In some embodiments, included are antigen-binding proteins comprising the variable domains set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; crovalimab; eculizumab, tesidolumab, mubodina, IFX-1 (see, e.g., US 2017/0137499, herein incorporated by reference in its entirety for all purposes), olendalizumab, or ravulizumab; preferably, pozelimab) which are linked to a heavy and/or light chain constant domain, e.g., as set forth above.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments.

In some embodiments, an antibody or antigen-binding fragment thereof that binds specifically to complement factor 5 (C5) protein, interacts with one or more amino acids contained within NMATGMDSW (SEQ ID NO: 692) (or at least 1, 2, 3, 4 or 5 amino acids therein); or WEVHLVPRRKQLQFALPDSL (SEQ ID NO: 693) (or at least 1, 2, 3, 4 or 5 amino acids therein), as determined by hydrogen/deuterium exchange. In some embodiments, an antibody or antigen-binding fragment thereof that binds specifically to complement factor 5 (C5) protein interacts with one or more amino acids contained within the alpha chain and/or the beta chain of C5, as determined by hydrogen/deuterium exchange. For example, in some embodiments, the antibody or antigen-binding fragment does not interact with an amino acid of the C5a anaphylatoxin region of C5, as determined by hydrogen/deuterium exchange. In some embodiments, an antibody or antigen-binding fragment thereof that binds specifically to complement factor 5 (C5) protein interacts with an amino acid sequence selected from the group consisting of: (a) NMATGMDSW (SEQ ID NO: 692); (b) ATGMDSW (SEQ ID NO: 694); (c) WEVHLVPRRKQLQ (SEQ ID NO: 695); (d) WEVHLVPRRKQLQFALPDSL (SEQ ID NO: 693); and (e) LVPRRKQLQ (SEQ ID NO: 696).

In some embodiments, the C5 antigen-binding protein binds to the beta chain or the alpha chain of C5 or both, e.g., at residues 591-599 and/or 775-794, e.g., NMATGMDSW (SEQ ID NO: 692) and/or WEVHLVPRRKQLQFALPDSL (SEQ ID NO: 693). In some embodiments, the C5 antigen-binding protein does not bind C5a.

In some embodiments, the C5 antigen-binding protein binds C5 at residues KDMQLGRLHMKTLLPVSK (SEQ ID NO: 699).

In some embodiments, the C5 antigen-binding protein binds the beta chain of C5 thereof, e.g., at residues 332-398, 332-378, 332-364, 332-348, 350-420, 369-409, 379-398 and/or 386-392.

In some embodiments, the C5 antigen-binding protein binds C5a, e.g., at residues NDETCEQRA (SEQ ID NO: 700) and/or SHKDMQL (SEQ ID NO: 701).

In some embodiments, the C5 antigen-binding protein binds the beta chain of C5, e.g., residues 19-180. In some embodiments, binding to C5 is reduced by E48A, D51A and/or K109A C5 mutations.

Sequences of examples of C5 antibodies and antigen-binding fragments thereof that may be used in the methods and combinations disclosed herein are set forth in Table 5 below.

TABLE 5
Anti-C5 Antibody Chain Amino Acid Sequences*

| Antibody | SEQ ID NOS | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 341 | 343 | 345 | 347 | 349 | 351 | 353 | 355 |
| H2M11686N | 357 | 359 | 361 | 363 | 365 | 367 | 369 | 371 |
| H4H12159P | 373 | 375 | 377 | 379 | 381 | 383 | 385 | 387 |
| H4H12161P | 389 | 391 | 393 | 395 | 397 | 399 | 401 | 403 |
| H4H12163P | 405 | 407 | 409 | 411 | 413 | 415 | 417 | 419 |
| H4H12164P | 421 | 423 | 425 | 427 | 429 | 431 | 433 | 435 |
| H4H12166P | 437 | 439 | 441 | 443 | 445 | 447 | 449 | 451 |
| H4H12166P2 | 437 | 439 | 441 | 443 | 453 | 455 | 457 | 459 |
| H4H12166P3 | 461 | 463 | 465 | 467 | 445 | 447 | 449 | 451 |
| H4H12166P4 | 437 | 439 | 441 | 443 | 469 | 471 | 473 | 475 |
| H4H12166P5 | 477 | 479 | 481 | 483 | 445 | 447 | 449 | 451 |
| H4H12166P6 | 485 | 487 | 489 | 491 | 445 | 447 | 449 | 451 |
| H4H12166P7 | 461 | 463 | 465 | 467 | 469 | 471 | 473 | 475 |
| H4H12166P8 | 485 | 487 | 489 | 491 | 453 | 455 | 457 | 459 |
| H4H12166P9 | 485 | 487 | 489 | 491 | 469 | 471 | 473 | 475 |
| H4H12166P10 | 477 | 479 | 481 | 483 | 469 | 471 | 473 | 475 |
| H4H12167P | 493 | 495 | 497 | 499 | 501 | 503 | 505 | 507 |
| H4H12168P | 509 | 511 | 513 | 515 | 517 | 519 | 521 | 523 |
| H4H12169P | 525 | 527 | 529 | 531 | 533 | 535 | 537 | 539 |
| H4H12170P | 541 | 543 | 545 | 547 | 549 | 551 | 553 | 555 |
| H4H12171P | 557 | 559 | 561 | 563 | 565 | 567 | 569 | 571 |
| H4H12175P | 573 | 575 | 577 | 579 | 581 | 583 | 585 | 587 |
| H4H12176P2 | 589 | 591 | 593 | 595 | 597 | 599 | 601 | 603 |
| H4H12177P2 | 605 | 607 | 609 | 611 | 597 | 599 | 601 | 603 |
| H4H12183P2 | 613 | 615 | 617 | 619 | 621 | 623 | 625 | 627 |
| H2M11682N | 629 | 631 | 633 | 635 | 637 | 639 | 641 | 643 |
| H2M11684N | 645 | 647 | 649 | 651 | 653 | 655 | 657 | 659 |
| H2M11694N | 661 | 663 | 665 | 667 | 669 | 671 | 673 | 675 |
| H2M11695N | 677 | 679 | 681 | 683 | 685 | 687 | 689 | 691 |

*Antibodies and fragments may include one or more variants of said sequences.
See WO 2017/218515, herein incorporated by reference in its entirety for all purposes. See also WO 2021/034639 A1, US 2021-0046182, WO 2021/081277 A1, US 2021-0139573, US 2020-0262901, US 2017-0355757, or US 2020-0262900, each of which is herein incorporated by reference in its entirety for all purposes.

Polynucleotides encoding the chains set forth in Table 5 are set forth below in Table 6.

TABLE 6
Anti-C5 Antibody Chain Nucleotide Sequences*

| Antibody | SEQ ID NOS | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 340 | 342 | 344 | 346 | 348 | 350 | 352 | 354 |
| H2M11686N | 356 | 358 | 360 | 362 | 364 | 366 | 368 | 370 |
| H4H12159P | 372 | 374 | 376 | 378 | 380 | 382 | 384 | 386 |
| H4H12161P | 388 | 390 | 392 | 394 | 396 | 398 | 400 | 402 |
| H4H12163P | 404 | 406 | 408 | 410 | 412 | 414 | 416 | 418 |
| H4H12164P | 420 | 422 | 424 | 426 | 428 | 430 | 432 | 434 |
| H4H12166P | 436 | 438 | 440 | 442 | 444 | 446 | 448 | 450 |
| H4H12166P2 | 436 | 438 | 440 | 442 | 452 | 454 | 456 | 458 |
| H4H12166P3 | 460 | 462 | 464 | 466 | 444 | 446 | 448 | 450 |
| H4H12166P4 | 436 | 438 | 440 | 442 | 468 | 470 | 472 | 474 |
| H4H12166P5 | 476 | 478 | 480 | 482 | 444 | 446 | 448 | 450 |
| H4H12166P6 | 484 | 486 | 488 | 490 | 444 | 446 | 448 | 450 |
| H4H12166P7 | 460 | 462 | 464 | 466 | 468 | 470 | 472 | 474 |
| H4H12166P8 | 484 | 486 | 488 | 490 | 452 | 454 | 456 | 458 |
| H4H12166P9 | 484 | 486 | 488 | 490 | 468 | 470 | 472 | 474 |
| H4H12166P10 | 476 | 478 | 480 | 482 | 468 | 470 | 472 | 474 |
| H4H12167P | 492 | 494 | 496 | 498 | 500 | 502 | 504 | 506 |
| H4H12168P | 508 | 510 | 512 | 514 | 516 | 518 | 520 | 522 |
| H4H12169P | 524 | 526 | 528 | 530 | 532 | 534 | 536 | 538 |
| H4H12170P | 540 | 542 | 544 | 546 | 548 | 550 | 552 | 554 |
| H4H12171P | 556 | 558 | 560 | 562 | 564 | 566 | 568 | 570 |
| H4H12175P | 572 | 574 | 576 | 578 | 580 | 582 | 584 | 586 |
| H4H12176P2 | 588 | 590 | 592 | 594 | 596 | 598 | 600 | 602 |
| H4H12177P2 | 604 | 606 | 608 | 610 | 596 | 598 | 600 | 602 |
| H4H12183P2 | 612 | 614 | 616 | 618 | 620 | 622 | 624 | 626 |
| H2M11682N | 628 | 630 | 632 | 634 | 636 | 638 | 640 | 642 |
| H2M11684N | 644 | 646 | 648 | 650 | 652 | 654 | 656 | 658 |
| H2M11694N | 660 | 662 | 664 | 666 | 668 | 670 | 672 | 674 |
| H2M11695N | 676 | 678 | 680 | 682 | 684 | 686 | 688 | 690 |

*Antibodies and fragments may include one or more variants of said sequences.
See WO 2017/218515, herein incorporated by reference in its entirety for all purposes. See also WO 2021/034639 A1, US 2021-0046182, WO 2021/081277 A1, US 2021-0139573, US 2020-0262901, US 2017-0355757, or US 2020-0262900, each of which is herein incorporated by reference in its entirety for all purposes

(SEQ ID NO: 340)
CAGGTGCAGCTGGTGGAGTCTGGGGGAGGCGTGGTCCAGCCTGGAAGGTCCCTGAGACTCTCCTGTGT
AGCGTCTGGATTCACCTTCAGTAGTTATGGCATTCACTGGGTCCGCCAGGCTCCAGGCAAGGGGCTGG
AGTGGGTGGCAGTTATATGGGATGATGGAAATAATATAAACTATTCAGACTCCGTGAAGGGCCGATTC
ATCATCTCCAGAGACAATTCCAGGAAGACAGTGTATCTGCAAATGAACAGCCTGAGAGGCGAGGACA
CGGCTGTTTATTACTGTGCGAGAGATGCCCCCATAGCACCAGTCCCTGACTATTGGGGCCAGGGAACC
CTGGTCACCGTCTCCTCA
(SEQ ID NO: 341)
QVQLVESGGGVVQPGRSLRLSCVASGFTFSSYGIHWVRQAPGKGLEWVAVIWDDGNNINYSDSVKGRFIIS
RDNSRKTVYLQMNSLRGEDTAVYYCARDAPIAPVPDYWGQGTLVTVSS
(SEQ ID NO: 342)
GGATTCACCTTCAGTAGTTATGGC
(SEQ ID NO: 343)
GFTFSSYG
(SEQ ID NO: 344)
ATATGGGATGATGGAAATAATATA
(SEQ ID NO: 345)
IWDDGNNI
(SEQ ID NO: 346)
GCGAGAGATGCCCCCATAGCACCAGTCCCTGACTAT
(SEQ ID NO: 347)
ARDAPIAPVPDY
(SEQ ID NO: 348)
GACATCCAGATGACCCAGTCTCCTTCCACCCTGTCTGCATCTGTAGGAGACAGAGTCACCATCACTTGC
CGGGCCAGTCAGAGTATTAGTAGTTGGTTGGCCTGGTATCAGCAGAAACCAGGGAAAGCCCCTAAGCT
CCTGATCTATAAGGCGTCTAGTTTAGACACTGGGGTCCCATCAAGGTTCAGCGGCAGTGGATCTGGGA
CAGAGTTCACTCTCACCATCAGCAGCCTGCAGCCTGATGATTTTGCAACTTATTACTGCCAACAGTATA
ATACTTATTCGTACACTTTTGGCCTGGGGACCAAACTGGAGATCAAA
(SEQ ID NO: 349)
DIQMTQSPSTLSASVGDRVTITCRASQSISSWLAWYQQKPGKAPKLLIYKASSLDTGVPSRFSGSGSGTEFTL
TISSLQPDDFATYYCQQYNTYSYTFGLGTKLEIK
(SEQ ID NO: 350)
CAGAGTATTAGTAGTTGG
(SEQ ID NO: 351)
QSISSW
(SEQ ID NO: 352)
AAGGCGTCT
(SEQ ID NO: 353)
KAS
(SEQ ID NO: 354)
CAACAGTATAATACTTATTCGTACACT
(SEQ ID NO: 355)
QQYNTYSYT
(SEQ ID NO: 356)
CAGGTGCAACTGGTGGAGTCTGGGGGAGGCTTGGTCAAGCCTGGAGGGTCCCTGAGACTCTCCTGTGC
AGCTTCTGGATTCACCTTCAGTGACTACTACATGAGCTGGATCCGCCAGGCTCCAGGGAAGGGGCTGG
AGTGGGTTTCATATATTAGCAGTAGTGGTAATACCATAAAATATGCAGACTCTATGAAGGGCCGATTC
ACCATCTCCAGGGACAACGCCAAGAAATCACTGTTTGTGGAAATGAACAGCCTGAGAGCCGAGGACA
CGGCCGTGTATTACTGTGCGAGGTATAAAAGTTCGTCCGACTACTTTGACCACTGGGGCCAGGGAACC
CTGGTCACCGTCTCCTCA
(SEQ ID NO: 357)
QVQLVESGGGLVKPGGSLRLSCAASGFTFSDYYMSWIRQAPGKGLEWVSYISSSGNTIKYADSMKGRFTIS
RDNAKKSLFVEMNSLRAEDTAVYYCARYKSSSDYFDHWGQGTLVTVSS
(SEQ ID NO: 358)
GGATTCACCTTCAGTGACTACTAC
(SEQ ID NO: 359)
GFTFSDYY
(SEQ ID NO: 360)
ATTAGCAGTAGTGGTAATACCATA
(SEQ ID NO: 361)
ISSSGNTI
(SEQ ID NO: 362)
GCGAGGTATAAAAGTTCGTCCGACTACTTTGACCAC
(SEQ ID NO: 363)
ARYKSSSDYFDH
(SEQ ID NO: 364)
GAAATTGTGTTGACACAGTCTCCAGCCACCCTGTCTTTGTCTCCAGGGGAAAGAGCCACCCTCTCCTGC
AGGGCCAGTCAGAGTGTTAGGAGTTACTTAGCCTGGTACCAACAGAAACCTGGCCAGGCTCCCAGGCT
CCTCATCTATGATGCATCCAACAGGGCCACTGCCATCCCAGCCAGGTTCAGTGGCAGTGGGTCTGGGA
CAGACTTCACTCTCACCATCAGCAGCCTAGAGCCTGAAGATTTAGCAGTTTATTACTGTCAGCAGTCTG
GCAACTGGCCGCTCACTTTCGGCGGAGGGACCAAGGTGGAGATCAAA
(SEQ ID NO: 365)
EIVLTQSPATLSLSPGERATLSCRASQSVRSYLAWYQQKPGQAPRLLIYDASNRATAIPARFSGSGSGTDFTL
TISSLEPEDLAVYYCQQSGNWPLTFGGGTKVEIK
(SEQ ID NO: 366)
CAGAGTGTTAGGAGTTAC
(SEQ ID NO: 367)
QSVRSY
(SEQ ID NO: 368)
GATGCATCC
(SEQ ID NO: 369)
DAS
(SEQ ID NO: 370)
CAGCAGTCTGGCAACTGGCCGCTCACT
(SEQ ID NO: 371)
QQSGNWPLT
(SEQ ID NO: 372)
CAGGTGCAGCTGGTGGAGTCTGGGGGAGGCGTGGTCCAGCCTGGGAGGTCCTTGAGACTCTCCTGTGG
AGCGTCTGGATTCACCTTCAGTACTTATGGCATGCACTGGGTCCGCCAGGCTCCAGGCAAGGGGCTGG
AGTGGGTGGCAGTTATCTGGGATGATGGAAATAATAAATATTATGCAGACTCCGTGAAGGGCCGATTC
ACCATCTCCAGAGACAATTCGAAGAACACGCTGTATCTGCAGATGAACAGCCTGAGAGCCGAGGACA
CGGCTGTTTATTACTGTGCGAGAGATTCAGAGGTCGCCCCAGTTGGGGACTACTGGGGCCAGGGCACC
CTGGTCACCGTCTCCTCA
(SEQ ID NO: 373)
QVQLVESGGGVVQPGRSLRLSCGASGFTFSTYGMHWVRQAPGKGLEWVAVIWDDGNNKYYADSVKGRF
TISRDNSKNTLYLQMNSLRAEDTAVYYCARDSEVAPVGDYWGQGTLVTVSS
(SEQ ID NO: 374)
GGATTCACCTTCAGTACTTATGGC
(SEQ ID NO: 375)
GFTFSTYG
(SEQ ID NO: 376)
ATCTGGGATGATGGAAATAATAAA
(SEQ ID NO: 377)
IWDDGNNK
(SEQ ID NO: 378)
GCGAGAGATTCAGAGGTCGCCCCAGTTGGGGACTAC
(SEQ ID NO: 379)
ARDSEVAPVGDY
(SEQ ID NO: 380)
GACATCCAGATGACCCAGTCTCCTTCCACCCTGTCTGCATCTGTAGGAGACAGAGTCACTATCATTTGC
CGGGCCAGTCAGAGTATTAACAGGTGGTTGGCCTGGTATCAGCAGAAACCAGGGAAGGCCCCTAAAC
TCCTGATCTATAAGGCGTCTAGTTTAGAAAGTGGGGTCCCATCAAGGTTCAGCGGCAGTGGATCTGGG
ACAGAATTCACTCTCACCATCAGCAGCCTGCAGCCTGATGATTTTGCAGCTTATTACTGCCAACAGTAT
AATGATTATTCGTACACTTTTGGCCAGGGGACCAAGCTGGAGATCAAA
(SEQ ID NO: 381)
DIQMTQSPSTLSASVGDRVTIICRASQSINRWLAWYQQKPGKAPKLLIYKASSLESGVPSRFSGSGSGTEFTL
TISSLQPDDFAAYYCQQYNDYSYTFGQGTKLEIK
(SEQ ID NO: 382)
CAGAGTATTAACAGGTGG
(SEQ ID NO: 383)
QSINRW
(SEQ ID NO: 384)
AAGGCGTCT
(SEQ ID NO: 385)
KAS
(SEQ ID NO: 386)
CAACAGTATAATGATTATTCGTACACT
(SEQ ID NO: 387)
QQYNDYSYT
(SEQ ID NO: 388)
GAGGTGCAGCTGGTGGAGTCTGGGGGAGACTTGGTCCAGCCTGGAGGGTCCCTGAGACTCTCCTGTGC
AGCCTCTGGATTCACCTTCAGTGACCACTATATGGACTGGGTCCGCCAGGCTCCAGGGAAGGGGCTGG
ACTGGATTGGCCGTATTAGAAACAAAGCTAACGCTTATAACACAGAATACGCCGCGTCTGTGAGAGGC
AGATTCACCATCTCAAGAGATGATTCACAGAATTTACTGTATCTGCAAATGAACAGCCTGAAAACCGA
TGACACGGCCGTATATTATTGTGTTAGAGTCTGGAACTACGCCTACTTCGCTATGGACGTCTGGGGCCA
AGGGACCACGGTCACCGTCTCCTCA
(SEQ ID NO: 389)
EVQLVESGGDLVQPGGSLRLSCAASGFTFSDHYMDWVRQAPGKGLDWIGRIRNKANAYNTEYAASVRGR
FTISRDDSQNLLYLQMNSLKTDDTAVYYCVRVWNYAYFAMDVWGQGTTVTVSS
(SEQ ID NO: 390)
GGATTCACCTTCAGTGACCACTAT
(SEQ ID NO: 391)
GFTFSDHY
(SEQ ID NO: 392)
ATTAGAAACAAAGCTAACGCTTATAACACA
(SEQ ID NO: 393)
IRNKANAYNT
(SEQ ID NO: 394)
GTTAGAGTCTGGAACTACGCCTACTTCGCTATGGACGTC
(SEQ ID NO: 395)
VRVWNYAYFAMDV
(SEQ ID NO: 396)
GACATCCAGATGACCCAGTCTCCATCCTCCCTATCTGCATCTGTGGGAGACAGAGTCACCATCACTTGC
CGGTCAAGTCAGAACATTGGAATCTTTTTAAACTGGTATCAACAAAAACCAGGGGAAGCCCCTAACCT
CCTGATCTCCGCTGCATCCAGTTTACACAGTGGGGTCCCTTCAAGGTTCAGTGGCAGTGGGTCTGGGAC
AGATTTCACTCTCACCATCGGCAGTCTGCAGCCTGAAGATTTTGCGACTTACTACTGTCAACAGACGTA
CAATACCATATTCACTTTCGGCCCTGGGACCAAAGTGGATATCAAA
(SEQ ID NO: 397)
DIQMTQSPSSLSASVGDRVTITCRSSQNIGIFLNWYQQKPGEAPNLLISAASSLHSGVPSRFSGSGSGTDFTLT
IGSLQPEDFATYYCQQTYNTIFTFGPGTKVDIK
(SEQ ID NO: 398)
CAGAACATTGGAATCTTT
(SEQ ID NO: 399)
QNIGIF
(SEQ ID NO: 400)
GCTGCATCC
(SEQ ID NO: 401)
AAS
(SEQ ID NO: 402)
CAACAGACGTACAATACCATATTCACT
(SEQ ID NO: 403)
QQTYNTIFT
(SEQ ID NO: 404)
GAGGTGCAGCTGGTGGAGTCTGGGGGAGACTTGGTACAGCCTGGGGGGTCCCTGAGACTCTCCTGTGC
AGCCTCTGGATTCACCTTTAGCAGCTATGCCATGAACTGGGTCCGCCAGGGTCCAGGGAAGGGACTGG
AGTGGGTCTCAGCTATTAGTGGTCGTGGTGATAGTACATACTACGCAGACTCCGTGAAGGGCCGGCTC
ACCATCTCCAGAGACAATTCCAAGAACACGCTGTATCTGCAAATGAACAGCCTGAGAGCCGAGGACA
CGGCCGTATATTACTGTGTGAAAGAGGGGGAGCAACTCGTCTACTGGTACTTCGATCTCTGGGGCCGT
GGCACCCTGGTCACCGTCTCCTCA
(SEQ ID NO: 405)
EVQLVESGGDLVQPGGSLRLSCAASGFTFSSYAMNWVRQGPGKGLEWVSAISGRGDSTYYADSVKGRLTI
SRDNSKNTLYLQMNSLRAEDTAVYYCVKEGEQLVYWYFDLWGRGTLVTVSS
(SEQ ID NO: 406)
GGATTCACCTTTAGCAGCTATGCC
(SEQ ID NO: 407)
GFTFSSYA
(SEQ ID NO: 408)
ATTAGTGGTCGTGGTGATAGTACA
(SEQ ID NO: 409)
ISGRGDST
(SEQ ID NO: 410)
GTGAAAGAGGGGGAGCAACTCGTCTACTGGTACTTCGATCTC
(SEQ ID NO: 411)
VKEGEQLVYWYFDL
(SEQ ID NO: 412)
GACATCCAGATGACCCAGTCTCCATCCTCCCTGTCTGCATCTGTAGGAGACAGAGTCACCATCACTTGC
CGGGCAAGTCAGACCATTAGCAACTTTTTACATTGGTATCAGCAGAAACCAGGGAAAGCCCCTAAGCT
CCTGATCTATGCTGCATCCAGTTTGCAAAGTGGGGTCCCATCAAGGTTCAGTGGCAGTGGATCTGGGA
CAGATTTCACTCTCACCATCAGCAGTCTGCAACCTGAAGATTTTTCAACTTACTTCTGTCAACAGAGTT
ACACTACCCCGCTCACTTTCGGCGGAGGGACCAAGGTGGAGATCAAA
(SEQ ID NO: 413)
DIQMTQSPSSLSASVGDRVTITCRASQTISNFLHWYQQKPGKAPKLLIYAASSLQSGVPSRFSGSGSGTDFTL
TISSLQPEDFSTYFCQQSYTTPLTFGGGTKVEIK
(SEQ ID NO: 414)
CAGACCATTAGCAACTTT
(SEQ ID NO: 415)
QTISNF
(SEQ ID NO: 416)
GCTGCATCC
(SEQ ID NO: 417)
AAS
(SEQ ID NO: 418)
CAACAGAGTTACACTACCCCGCTCACT
(SEQ ID NO: 419)
QQSYTTPLT
(SEQ ID NO: 420)
GAGGTGCAGCTGGTGGAGTCTGGGGGAGGCTTGGTGAGGTCGGGGGGGTCCCTGAGACTCTCCTGTGC
AGCCTCTGGATTCACCTTTAACAGATATGCCATGACCTGGGTCCGCCAGGCTCCAGGGAAGGGGCTGG
AGTGGGTCTCAGCTATAAGTGGTAGTGGTAGCAGCACATACTACACAGACTCCGTGAAAGGCCGGTTC
ACCATCTCCAGAGACAATTCCAAGAATTCGGTGGATCTGCAAATGCACAGCCTGAGAGTCGAAGACAC
GGCCATATATTATTGTGCGAGAGGGACTACAGTCACTACGGGGTACGGTATGGACGTCTGGGGCCAAG
GGACCACGGTCACCGTCTCCTCA
(SEQ ID NO: 421)
EVQLVESGGGLVRSGGSLRLSCAASGFTFNRYAMTWVRQAPGKGLEWVSAISGSGSSTYYTDSVKGRFTIS
RDNSKNSVDLQMHSLRVEDTAIYYCARGTTVTTGYGMDVWGQGTTVTVSS
(SEQ ID NO: 422)
GGATTCACCTTTAACAGATATGCC
(SEQ ID NO: 423)
GFTFNRYA
(SEQ ID NO: 424)
ATAAGTGGTAGTGGTAGCAGCACA
(SEQ ID NO: 425)
ISGSGSST
(SEQ ID NO: 426)
GCGAGAGGGACTACAGTCACTACGGGGTACGGTATGGACGTC
(SEQ ID NO: 427)
ARGTTVTTGYGMDV
(SEQ ID NO: 428)
GACATCCAGATGACCCAGTCTCCATCCTCCCTGTCTGCATCTGTAGGAGACAGAGTCACCTTCACTTGC
CAGGCGAGTCAGGACATTACCAATTCTTTAAATTGGTATCAACAGAAACCTGGGAGAGCCCCTAAGCT
CCTGATCTACGATGCATCGTATTTGAAGGCAGGGGTCCCATCAAGATTCAGTGGAAGTGGATCTGGGA
CAGATTTTACTTTCACCATCAGCAGCCTGCAGCCTGAAGATATTGCAACATATTACTGTCAACAATATG
ATGATCTCCCATACACTTTTGGCCAGGGGACCAAGCTGGAGATCAAA
(SEQ ID NO: 429)
DIQMTQSPSSLSASVGDRVTFTCQASQDITNSLNWYQQKPGRAPKLLIYDASYLKAGVPSRFSGSGSGTDFT
FTISSLQPEDIATYYCQQYDDLPYTFGQGTKLEIK
(SEQ ID NO: 430)
CAGGACATTACCAATTCT
(SEQ ID NO: 431)
QDITNS
(SEQ ID NO: 432)
GATGCATCG
(SEQ ID NO: 433)
DAS
(SEQ ID NO: 434)
CAACAATATGATGATCTCCCATACACT
(SEQ ID NO: 435)
QQYDDLPYT
(SEQ ID NO: 436)
CAGGTGCAGCTGCAGGAGTCGGGCCCAGGACTGGTGAAGCCTTCGGAGACCCTGTCCCTCACCTGCAC
TGTCTCTGGTGACTCCGTCAGTAGTTCCTACTGGACCTGGATCCGGCAGCCCCCAGGGAAGGGACTGG
AGTGGATTGGCTATATCTATTACAGTGGGAGTTCCAACTACAACCCCTCCCTCAAGAGTCGAGCCACC
ATTTCAGTAGACACGTCCAAGAACCAGTTCTCCCTGAAGCTGAGTTCTGTGACCGCTGCGGACACGGC
CGTATATTACTGTGCGAGAGAAGGGAACGTGGATACAACTATGATATTTGACTACTGGGGCCAGGGAA
CCCTGGTCACCGTCTCCTCA
(SEQ ID NO: 437)
QVQLQESGPGLVKPSETLSLTCTVSGDSVSSSYWTWIRQPPGKGLEWIGYIYYSGSSNYNPSLKSRATISVD
TSKNQFSLKLSSVTAADTAVYYCAREGNVDTTMIFDYWGQGTLVTVSS
(SEQ ID NO: 438)
GGTGACTCCGTCAGTAGTTCCTAC
(SEQ ID NO: 439)
GDSVSSSY
(SEQ ID NO: 440)
ATCTATTACAGTGGGAGTTCC
(SEQ ID NO: 441)
IYYSGSS
(SEQ ID NO: 442)
GCGAGAGAAGGGAACGTGGATACAACTATGATATTTGACTAC
(SEQ ID NO: 443)
AREGNVDTTMIFDY
(SEQ ID NO: 444)
GCCATCCAGATGACCCAGTCTCCATCCTCCCTGTCTGCATCTGTAGGAGACAGAGTCACCATCACTTGC
CGGGCAAGTCAGGGCATTAGAAATGATTTAGGCTGGTATCAACAGAAACCAGGGAAAGCCCCTAAAC
TCCTGATCTATGCTGCATCCAGTTTACAAAGTGGGGTCCCATCGAGGTTCGCCGGCCGTGGATCTGGCA
CAGATTTCACTCTCACCATCAGCAGCCTGCAGCCTGAAGATTTTGCAACTTATTACTGTCTACAAGATT
TCAATTACCCGTGGACGTTCGGCCAAGGGACCAAGGTGGAAATCAAA
(SEQ ID NO: 445)
AIQMTQSPSSLSASVGDRVTITCRASQGIRNDLGWYQQKPGKAPKLLIYAASSLQSGVPSRFAGRGSGTDFT
LTISSLQPEDFATYYCLQDFNYPWTFGQGTKVEIK
(SEQ ID NO: 446)
CAGGGCATTAGAAATGAT
(SEQ ID NO: 447)
QGIRND
(SEQ ID NO: 448)
GCTGCATCC
(SEQ ID NO: 449)
AAS
(SEQ ID NO: 450)
CTACAAGATTTCAATTACCCGTGGACG
(SEQ ID NO: 451)
LQDFNYPWT
(SEQ ID NO: 452)
GCCATCCAGATGACCCAGTCTCCATCCTCCCTGTCTGCATCTGTAGGAGACAGAGTCACCATCACTTGC
CGGGCAAGTCAGGGCATTAGAAATGATTTAGGCTGGTATCAACAGAAACCAGGGAAAGCCCCTAAAC
TCCTGATCTATGCTGCATCCAGTTTACAAAGTGGGGTCCCATCGAGGTTCGCCGGCCGTGGATCTGGCA
CAGATTTCACTCTCACCATCAGCAGCCTGCAGCCTGAAGATTTTGCAACTTATTACTGTCATCAAGATT
TCAATTACCCGTGGACGTTCGGCCAAGGGACCAAGGTGGAAATCAAA
(SEQ ID NO: 453)
AIQMTQSPSSLSASVGDRVTITCRASQGIRNDLGWYQQKPGKAPKLLIYAASSLQSGVPSRFAGRGSGTDFT
LTISSLQPEDFATYYCHQDFNYPWTFGQGTKVEIK
(SEQ ID NO: 454)
CAGGGCATTAGAAATGAT
(SEQ ID NO: 455)
QGIRND
(SEQ ID NO: 456)
GCTGCATCC
(SEQ ID NO: 457)
AAS
(SEQ ID NO: 458)
CATCAAGATTTCAATTACCCGTGGACG
(SEQ ID NO: 459)
HQDFNYPWT
(SEQ ID NO: 460)
CAGGTGCAGCTGCAGGAGTCGGGCCCAGGACTGGTGAAGCCTTCGGAGACCCTGTCCCTCACCTGCAC
TGTCTCTGGTGACTCCGTCAGTAGTTCCTACTGGACCTGGATCCGGCAGCCCCCAGGGAAGGGACTGG
AGTGGATTGGCTATATCTATTACAGTGGGAGTTCCAACTACAACCCCTCCCTCAAGAGTCGAGCCACC
ATTTCAGTAGACACGTCCAAGAACCAGTTCTCCCTGAAGCTGAGTTCTGTGACCGCTGCGGACACGGC
CGTATATTACTGTGCGAGAGAACATAACGTGGATACAACTATGATATTTGACTACTGGGGCCAGGGAA
CCCTGGTCACCGTCTCCTCA
(SEQ ID NO: 461)
QVQLQESGPGLVKPSETLSLTCTVSGDSVSSSYWTWIRQPPGKGLEWIGYIYYSGSSNYNPSLKSRATISVD
TSKNQFSLKLSSVTAADTAVYYCAREHNVDTTMIFDYWGQGTLVTVSS
(SEQ ID NO: 462)
GGTGACTCCGTCAGTAGTTCCTAC
(SEQ ID NO: 463)
GDSVSSSY
(SEQ ID NO: 464)
ATCTATTACAGTGGGAGTTCC
(SEQ ID NO: 465)
IYYSGSS
(SEQ ID NO: 466)
GCGAGAGAACATAACGTGGATACAACTATGATATTTGACTAC
(SEQ ID NO: 467)
AREHNVDTTMIFDY
(SEQ ID NO: 468)
GCCATCCAGATGACCCAGTCTCCATCCTCCCTGTCTGCATCTGTAGGAGACAGAGTCACCATCACTTGC
CGGGCAAGTCAGGGCATTAGAAATGATTTAGGCTGGTATCAACAGAAACCAGGGAAAGCCCCTAAAC
TCCTGATCTATGCTGCATCCAGTTTACAAAGTGGGGTCCCATCGAGGTTCGCCGGCCGTGGATCTGGCA
CAGATTTCACTCTCACCATCAGCAGCCTGCAGCCTGAAGATTTTGCAACTTATTACTGTCTACAAGATT
TCAATTACCCGTGGCACTTCGGCCAAGGGACCAAGGTGGAAATCAAA
(SEQ ID NO: 469)
AIQMTQSPSSLSASVGDRVTITCRASQGIRNDLGWYQQKPGKAPKLLIYAASSLQSGVPSRFAGRGSGTDFT
LTISSLQPEDFATYYCLQDFNYPWHFGQGTKVEIK
(SEQ ID NO: 470)
CAGGGCATTAGAAATGAT
(SEQ ID NO: 471)
QGIRND
(SEQ ID NO: 472)
GCTGCATCC
(SEQ ID NO: 473)
AAS
(SEQ ID NO: 474)
CTACAAGATTTCAATTACCCGTGGCAC
(SEQ ID NO: 475)
LQDFNYPWH
(SEQ ID NO: 476)
CAGGTGCAGCTGCAGGAGTCGGGCCCAGGACTGGTGAAGCCTTCGGAGACCCTGTCCCTCACCTGCAC
TGTCTCTGGTGACTCCGTCAGTAGTTCCTACTGGACCTGGATCCGGCAGCCCCCAGGGAAGGGACTGG
AGTGGATTGGCTATATCTATTACAGTGGGAGTTCCAACTACAACCCCTCCCTCAAGAGTCGAGCCACC
ATTTCAGTAGACACGTCCAAGAACCAGTTCTCCCTGAAGCTGAGTTCTGTGACCGCTGCGGACACGGC
CGTATATTACTGTGCGAGAGAAGGGAACGTGGATACAACTATGATACATGACTACTGGGGCCAGGGA
ACCCTGGTCACCGTCTCCTCA
(SEQ ID NO: 477)
QVQLQESGPGLVKPSETLSLTCTVSGDSVSSSYWTWIRQPPGKGLEWIGYIYYSGSSNYNPSLKSRATISVD
TSKNQFSLKLSSVTAADTAVYYCAREGNVDTTMIHDYWGQGTLVTVSS
(SEQ ID NO: 478)
GGTGACTCCGTCAGTAGTTCCTAC
(SEQ ID NO: 479)
GDSVSSSY
(SEQ ID NO: 480)
ATCTATTACAGTGGGAGTTCC
(SEQ ID NO: 481)
IYYSGSS
(SEQ ID NO: 482)
GCGAGAGAAGGGAACGTGGATACAACTATGATACATGACTAC
(SEQ ID NO: 483)
AREGNVDTTMIHDY
(SEQ ID NO: 484)
CAGGTGCAGCTGCAGGAGTCGGGCCCAGGACTGGTGAAGCCTTCGGAGACCCTGTCCCTCACCTGCAC
TGTCTCTGGTGACTCCGTCAGTAGTTCCTACTGGACCTGGATCCGGCAGCCCCCAGGGAAGGGACTGG
AGTGGATTGGCTATATCTATTACAGTGGGAGTTCCAACTACAACCCCTCCCTCAAGAGTCGAGCCACC
ATTTCAGTAGACACGTCCAAGAACCAGTTCTCCCTGAAGCTGAGTTCTGTGACCGCTGCGGACACGGC
CGTATATTACTGTGCGAGAGAAGGGAACGTGGATCACACTATGATATTTGACTACTGGGGCCAGGGAA
CCCTGGTCACCGTCTCCTCA
(SEQ ID NO: 485)
QVQLQESGPGLVKPSETLSLTCTVSGDSVSSSYWTWIRQPPGKGLEWIGYIYYSGSSNYNPSLKSRATISVD
TSKNQFSLKLSSVTAADTAVYYCAREGNVDHTMIFDYWGQGTLVTVSS
(SEQ ID NO: 486)
GGTGACTCCGTCAGTAGTTCCTAC
(SEQ ID NO: 487)
GDSVSSSY
(SEQ ID NO: 488)
ATCTATTACAGTGGGAGTTCC
(SEQ ID NO: 489)
IYYSGSS
(SEQ ID NO: 490)
GCGAGAGAAGGGAACGTGGATCACACTATGATATTTGACTAC
(SEQ ID NO: 491)
AREGNVDHTMIFDY
(SEQ ID NO: 492)
CAGGTGCAGCTGGTGGAGTCTGGGGGAGGCTTGGTCAAGCCTGGAGGGTCCCTGAGACTCTCCTGTGC
AGCCTCTGGATTCACCTTCAGTGACTCCTACATGTCCTGGATCCGTCAGGCTCCAGGGAAGGGACTAG
AGTGGATTTCATACATTGGTAGTAGTGGTAATACCTTTTACTACGCAGACTCTGTGAAGGGCCGGTTCA
CCATTTCCAGAGACAACGCCAACAATTTACTGTATCTGCAAATGACCAGCCTGAGAGCCGAGGACACG
GCCGTGTATTACTGTGCGAGAGAAGAAGGCGATTTTTGGAGTGCCGTTGACTCCTGGGGCCAGGGAAC
CCTGGTCACCGTCTCCTCA
(SEQ ID NO: 493)
QVQLVESGGGLVKPGGSLRLSCAASGFTFSDSYMSWIRQAPGKGLEWISYIGSSGNTFYYADSVKGRFTISR
DNANNLLYLQMTSLRAEDTAVYYCAREEGDFWSAVDSWGQGTLVTVSS
(SEQ ID NO: 494)
GGATTCACCTTCAGTGACTCCTAC
(SEQ ID NO: 495)
GFTFSDSY
(SEQ ID NO: 496)
ATTGGTAGTAGTGGTAATACCTTT
(SEQ ID NO: 497)
IGSSGNTF
(SEQ ID NO: 498)
GCGAGAGAAGAAGGCGATTTTTGGAGTGCCGTTGACTCC
(SEQ ID NO: 499)
AREEGDFWSAVDS
(SEQ ID NO: 500)
GACATCCAGTTGACCCAGTCTCCATCCTTCCTGTCTGCATCTGTAGGAGACAGAGTCACCATCACTTGC
TGGGCCAGTCAGGGCATTAGCAGTTATTTAGCCTGGTATCAGCAAAAACCAGGTAAAGCCCCTAAACT
CCTGATCCATACTGCATCCACTTTGCAAAGTGGGGTCCCATCAAGGTTCAGCGGCAGTGGATCTGGGA
CAGAATTCACTCTCACAATCAGCAACCTGCAGCCTGAAGATTTTGCAACTTATTACTGTCAACAGCTTA
ATAGTTACCCATTCACTTTCGGCCCTGGGACCAAAGTGGATATCAAA
(SEQ ID NO: 501)
DIQLTQSPSFLSASVGDRVTITCWASQGISSYLAWYQQKPGKAPKLLIHTASTLQSGVPSRFSGSGSGTEFTL
TISNLQPEDFATYYCQQLNSYPFTFGPGTKVDIK
(SEQ ID NO: 502)
CAGGGCATTAGCAGTTAT
(SEQ ID NO: 503)
QGISSY
(SEQ ID NO: 504)
ACTGCATCC
(SEQ ID NO: 505)
TAS
(SEQ ID NO: 506)
CAACAGCTTAATAGTTACCCATTCACT
(SEQ ID NO: 507)
QQLNSYPFT
(SEQ ID NO: 508)
CAGGTGCAGCTGGTGGAGTCTGGGGGAGGCGTGGTCCAGCCTGGGGGGTCCCTGAGACTCTCCTGTGC
AGCCTCTGGATTCACCTTCGGTGGCCATGCCATGCACTGGGTCCGCCAGGCTCCAGGCAAGGGGCTGG
AGTGGCTGGCAGTTATATCATCTGATGGCAGTAATAAACAGTATGCAGATTCTGTGAAGGGCCGATTC
ACCATCTCCAGGGACAATCCCAAGAACACGCTGTATCTGCAAATGAACAGTCTGAGAGTTGGGGACAC
GGCTATTTATTACTGTGCGAAAGAGGTGGCACCTCGTTATTATTATTACGGTCTGGACGTCTGGGGCCA
AGGGACCACGGTCACCGTCTCCTCA
(SEQ ID NO: 509)
QVQLVESGGGVVQPGGSLRLSCAASGFTFGGHAMHWVRQAPGKGLEWLAVISSDGSNKQYADSVKGRFT
ISRDNPKNTLYLQMNSLRVGDTAIYYCAKEVAPRYYYYGLDVWGQGTTVTVSS
(SEQ ID NO: 510)
GGATTCACCTTCGGTGGCCATGCC
(SEQ ID NO: 511)
GFTFGGHA
(SEQ ID NO: 512)
ATATCATCTGATGGCAGTAATAAA
(SEQ ID NO: 513)
ISSDGSNK
(SEQ ID NO: 514)
GCGAAAGAGGTGGCACCTCGTTATTATTATTACGGTCTGGACGTC
(SEQ ID NO: 515)
AKEVAPRYYYYGLDV
(SEQ ID NO: 516)
GACATCCAGATGACCCAGTCTCCATCCTCCCTGTCTGCATCTGTGGGAGACAGAGTCACCATCACTTGC
CGGGCGAGTCAGGACATTAGCAATTTTTTAGCCTGGTATCAGCAGAAACCAGGGAAGGTTCCTAAACT
CCTGATCTATACTGCATCCACTTTACAATCAGGGGTCCCATCTCGGTTCAGTGGCAGTGGATCTGGGAC
AGATTTCACTCTCACCGTCAGCAGCCTACAGCCTGAAGATGTTGCAACTTATTACTGTCAAAAGTATGC
CGGCGCCCTCACTTTCGGCCCTGGGACCAAAGTGGATATCAAA
(SEQ ID NO: 517)
DIQMTQSPSSLSASVGDRVTITCRASQDISNFLAWYQQKPGKVPKLLIYTASTLQSGVPSRFSGSGSGTDFTL
TVSSLQPEDVATYYCQKYAGALTFGPGTKVDIK
(SEQ ID NO: 518)
CAGGACATTAGCAATTTT
(SEQ ID NO: 519)
QDISNF
(SEQ ID NO: 520)
ACTGCATCC
(SEQ ID NO: 521)
TAS
(SEQ ID NO: 522)
CAAAAGTATGCCGGCGCCCTCACT
(SEQ ID NO: 523)
QKYAGALT
(SEQ ID NO: 524)
GAGGTGCAGCTGGTGGAGTCTGGGGGAGGCTTGGCACAGCCTGGGGGGTCCCTGAGACTCTCCTGTGC
AGCCTCTGGATTCACGTTTAGAAGCTATGCCATGAGCTGGGTCCGCCAGGCTCCAGGGAAGGGGCCGG
AGTGGGTCTCAGGTATAGGTGGTAATGGTGTTACCACATACTACGCAGACTCCGTGAAGGGCCGGTTC
ACCATCTCCAGAGACAATTCCAAGAACACGCTGTTTCTGCAAATGAATAGCCTGAGAGCCGAGGACAC
GGCCGTATATTATTGTGTGCAGGGGGGTTTAGGTGGTTATTTTACAGGCTACTGGGGCCAGGGAACCC
TGGTCACCGTCTCCTCA
(SEQ ID NO: 525)
EVQLVESGGGLAQPGGSLRLSCAASGFTFRSYAMSWVRQAPGKGPEWVSGIGGNGVTTYYADSVKGRFTI
SRDNSKNTLFLQMNSLRAEDTAVYYCVQGGLGGYFTGYWGQGTLVTVSS
(SEQ ID NO: 526)
GGATTCACGTTTAGAAGCTATGCC
(SEQ ID NO: 527)
GFTFRSYA
(SEQ ID NO: 528)
ATAGGTGGTAATGGTGTTACCACA
(SEQ ID NO: 529)
IGGNGVTT
(SEQ ID NO: 530)
GTGCAGGGGGGTTTAGGTGGTTATTTTACAGGCTAC
(SEQ ID NO: 531)
VQGGLGGYFTGY
(SEQ ID NO: 532)
GACATCCAGATGACCCAGTCTCCATCCTCCCTGTCTGCATCTGTAGGAGACAGAGTCACCATCACTTGC
CGGGCAAGTCAGAGTATTAGTACCTATTTAAATTGGTATCAGCAGAATCCAGGGAAAGCCCCTAAACT
CCTGATCTTTGATGCATCCAGTTTGCAAAGTGGGGTCCCATCAAGGTTCAGTGGCAGTGGATCTGGGA
CAGATTTCACTCTCACCATCAGAGGTCTGCAACCTGAAGATTTTGCAACTTACTACTGTCAACAGAGTT
ACAGTGCCCCGCTCACTTTCGGCGGAGGGACCAAGGTGGAGATCAAA
(SEQ ID NO: 533)
DIQMTQSPSSLSASVGDRVTITCRASQSISTYLNWYQQNPGKAPKLLIFDASSLQSGVPSRFSGSGSGTDFTL
TIRGLQPEDFATYYCQQSYSAPLTFGGGTKVEIK
(SEQ ID NO: 534)
CAGAGTATTAGTACCTAT
(SEQ ID NO: 535)
QSISTY
(SEQ ID NO: 536)
GATGCATCC
(SEQ ID NO: 537)
DAS
(SEQ ID NO: 538)
CAACAGAGTTACAGTGCCCCGCTCACT
(SEQ ID NO: 539)
QQSYSAPLT
(SEQ ID NO: 540)
CAGGTGCAGCTGGTGGAGTCTGGGGGAGGCGTGGTCCAGCCTGGGAGGTCCCTGAGACTCTCCTGTGC
AGCGTCTGGATTCACCTTCAGTGGTTATGGCATGCACTGGGTCCGCCAGGCTCCAGGCAAGGGGCTGG
AGTGGGTGGCACTTATATGGCTTGATGGAAGTAATGACTACTATGCAGACTCCGTGAAGGGCCGATTC
ACCATCTCCAGAGACAATTCCAAGAACACGTTATATCTGCAAATGAACAGACTGAGAGCCGAGGACA
CGGCTGTGTATTACTGTGCGAGAGATGGCCCGGTTGCTGCTATACCCGACTACTGGGGCCAGGGAACC
CTGGTCACCGTCTCCTCA
(SEQ ID NO: 541)
QVQLVESGGGVVQPGRSLRLSCAASGFTFSGYGMHWVRQAPGKGLEWVALIWLDGSNDYYADSVKGRF
TISRDNSKNTLYLQMNRLRAEDTAVYYCARDGPVAAIPDYWGQGTLVTVSS
(SEQ ID NO: 542)
GGATTCACCTTCAGTGGTTATGGC
(SEQ ID NO: 543)
GFTFSGYG
(SEQ ID NO: 544)
ATATGGCTTGATGGAAGTAATGAC
(SEQ ID NO: 545)
IWLDGSND
(SEQ ID NO: 546)
GCGAGAGATGGCCCGGTTGCTGCTATACCCGACTAC
(SEQ ID NO: 547)
ARDGPVAAIPDY
(SEQ ID NO: 548)
GACATCCAGATGACCCAGTCTCCTTCCACCCTGTCTGCATCTGTAGGAGACAGAGTCACCATCACTTGC
CGGGCCAGTCAGAGTATTAGTAGGTGGTTGGCCTGGTATCAGCTGAAACCAGGGAAAGCCCCTAAGCT
CCTGATCTATAAGGCGTCTAGTTTAGAAAGTGGGGTCCCATCAAGGTTCAGCGGCAGTGGATCTGGGA
CAGACTTCACTCTCACCATCAGCAGCCTGCAACCTGATGATTTTGCAACTTATTACTGCCAACAGTATA
ATACTTATTCGTACACTTTTGGCCAGGGGACCAAGCTGGAGATCAAA
(SEQ ID NO: 549)
DIQMTQSPSTLSASVGDRVTITCRASQSISRWLAWYQLKPGKAPKLLIYKASSLESGVPSRFSGSGSGTDFTL
TISSLQPDDFATYYCQQYNTYSYTFGQGTKLEIK
(SEQ ID NO: 550)
CAGAGTATTAGTAGGTGG
(SEQ ID NO: 551)
QSISRW
(SEQ ID NO: 552)
AAGGCGTCT
(SEQ ID NO: 553)
KAS
(SEQ ID NO: 554)
CAACAGTATAATACTTATTCGTACACT
(SEQ ID NO: 555)
QQYNTYSYT
(SEQ ID NO: 556)
GAGGTGCAGCTGGTGGAGTCTGGGGGAGGTGTGGTACGGCCTGGGGGGTCCCTGAGACTCTCCTGTGC
AGCCTCTGGATTCACCTTTGATGAATATGGCATGACTTGGGTCCGCCAAGTTCCAGGGAAGGGGCTGG
AGTGGGTCTCTGGTATTACTTGGAATGGTGGTTTCACAGATTATACAGACTCTGTGAAGGGCCGATTCA
CCAGCTCCAGAGACAACGCCAAGAACTCCCTGTATCTGCAAATGAACAGTCTGAGAGCCGAGGACAC
GGCCTTGTATTACTGTGCGAGAGATGGATATAGCAGCTCGTGGGGGGCTTATGATATATGGGGCCAAG
GGACAATGGTCACCGTCTCTTCA
(SEQ ID NO: 557)
EVQLVESGGGVVRPGGSLRLSCAASGFTFDEYGMTWVRQVPGKGLEWVSGITWNGGFTDYTDSVKGRFT
SSRDNAKNSLYLQMNSLRAEDTALYYCARDGYSSSWGAYDIWGQGTMVTVSS
(SEQ ID NO: 558)
GGATTCACCTTTGATGAATATGGC
(SEQ ID NO: 559)
GFTFDEYG
(SEQ ID NO: 560)
ATTACTTGGAATGGTGGTTTCACA
(SEQ ID NO: 561)
ITWNGGFT
(SEQ ID NO: 562)
GCGAGAGATGGATATAGCAGCTCGTGGGGGGCTTATGATATA
(SEQ ID NO: 563)
ARDGYSSSWGAYDI
(SEQ ID NO: 564)
GACATCCAGATGACCCAGTCTCCATCATCCCTGTCTGCATCTGTGGGAGACAGAGTCACCATCACTTGC
CGGGCAAGTCAGAGCATTAGCACCTATTTAAATTGGTATCAGCAGAAACCAGGGAAAGCCCCTAAGCT
CCTGATCTATGCTGCATCCAGTTTGCAAAGTGGGGTCCCATTAAGGTTCAGTGGCAGTGGATCTGGGA
CTGATTTCACTCTCACCATCAGCAGTCTGCAACCTGAAGATTTTGCAAGTTATTTCTGTCAACAGAGTT
ACAGTACCCCGTACACTTTTGGCCAGGGGACCAAGCTGGAGATCAAA
(SEQ ID NO: 565)
DIQMTQSPSSLSASVGDRVTITCRASQSISTYLNWYQQKPGKAPKLLIYAASSLQSGVPLRFSGSGSGTDFTL
TISSLQPEDFASYFCQQSYSTPYTFGQGTKLEIK
(SEQ ID NO: 566)
CAGAGCATTAGCACCTAT
(SEQ ID NO: 567)
QSISTY
(SEQ ID NO: 568)
GCTGCATCC
(SEQ ID NO: 569)
AAS
(SEQ ID NO: 570)
CAACAGAGTTACAGTACCCCGTACACT
(SEQ ID NO: 571)
QQSYSTPYT
(SEQ ID NO: 572)
GAAGTGCAGCTGGTGGAGTCTGGGGGAGGCGTGGTACAGCCTGGGGGGTCCCTGAGACTCTCCTGTGC
AGCCTCTGGATTCACCTTTAATGATTATGCCATGCACTGGGTCCGTCAAGCTCCAGGGAAGGGTCTGG
AGTGGGTCTCTCTTATTAGTGGAGATGGTGGTAACACATACTATGCAGACTCTGTGAAGGGCCGACTC
ACCATCTCCAGAGACAACAGCAAAAACTCCCTGTATCTGCAAATGAACAGTCTGAGAACAGAGGACA
CCGCCTTATATTACTGTGCAAAAGATAAGGGCTGGAACTTCGGTTACTTCGATCTCTGGGGCCGTGGC
ACCCTGGTCACTGTCTCCTCA
(SEQ ID NO: 573)
EVQLVESGGGVVQPGGSLRLSCAASGFTFNDYAMHWVRQAPGKGLEWVSLISGDGGNTYYADSVKGRLT
ISRDNSKNSLYLQMNSLRTEDTALYYCAKDKGWNFGYFDLWGRGTLVTVSS
(SEQ ID NO: 574)
GGATTCACCTTTAATGATTATGCC
(SEQ ID NO: 575)
GFTFNDYA
(SEQ ID NO: 576)
ATTAGTGGAGATGGTGGTAACACA
(SEQ ID NO: 577)
ISGDGGNT
(SEQ ID NO: 578)
GCAAAAGATAAGGGCTGGAACTTCGGTTACTTCGATCTC
(SEQ ID NO: 579)
AKDKGWNFGYFDL
(SEQ ID NO: 580)
GACATCCAGATGACCCAGTCTCCATCCTCCCTGTCTACATCTGTGGGAGACAGAGTCACCATCACTTGC
CGGGCAAGTCAGAACATTGACACCTATTTAAATTGGTATCAGCAGAAACCAGGGAAAGCCCCTAAACT
CCTGATCTATGATGCATCCAGTTTACAAAGTGGGGTCCCATCACGGTTCAGTGGCAGCGGATCTGGGA
CAGATTTCACTCTCACCATCACCAGTCTGCAACCTGAAGATTTTGCCACTTACTACTGTCAACAGAATG
ACAATATTCTTCACCCTCTCACTTTCGGCGGAGGGACCAAGGTGGAGATCAAA
(SEQ ID NO: 581)
DIQMTQSPSSLSTSVGDRVTITCRASQNIDTYLNWYQQKPGKAPKLLIYDASSLQSGVPSRFSGSGSGTDFTL
TITSLQPEDFATYYCQQNDNILHPLTFGGGTKVEIK
(SEQ ID NO: 582)
CAGAACATTGACACCTAT
(SEQ ID NO: 583)
QNIDTY
(SEQ ID NO: 584)
GATGCATCC
(SEQ ID NO: 585)
DAS
(SEQ ID NO: 586)
CAACAGAATGACAATATTCTTCACCCTCTCACT
(SEQ ID NO: 587)
QQNDNILHPLT
(SEQ ID NO: 588)
GAGGTGCAGCTGGTGGAGTCTGGGGGAGGCTTGGTCCAACCGGGGGGGTCCCTGAGACTCTCCTGTGC
AGCCTCTGGATTCCACTCTAATAGATATTGGATGGACTGGGTCCGCCAGGCTCCAGGGAAGGGGCTGG
AGTGGGTGGCCAACATAAAGCAAGATGGAAGTGAGGAAAACTATGTGGACTCTGTGAAGGGCCGATT
CACCATCTCCAGAGACAACGCCAAGAACTCACTTTATCTGCAAATGAACAGCCTGAGAGCCGAGGAC
ACGGCTGTGTATTACTGTGCGAGAGATCGAAGCACCTCGTGGGTCCCTTACTGGTTCTTCGATCTCTGG
GGCCGTGGCACCCTGGTCACTGTCTCCTCA
(SEQ ID NO: 589)
EVQLVESGGGLVQPGGSLRLSCAASGFHSNRYWMDWVRQAPGKGLEWVANIKQDGSEENYVDSVKGRF
TISRDNAKNSLYLQMNSLRAEDTAVYYCARDRSTSWVPYWFFDLWGRGTLVTVSS
(SEQ ID NO: 590)
GGATTCCACTCTAATAGATATTGG
(SEQ ID NO: 591)
GFHSNRYW
(SEQ ID NO: 592)
ATAAAGCAAGATGGAAGTGAGGAA
(SEQ ID NO: 593)
IKQDGSEE
(SEQ ID NO: 594)
GCGAGAGATCGAAGCACCTCGTGGGTCCCTTACTGGTTCTTCGATCTC
(SEQ ID NO: 595)
ARDRSTSWVPYWFFDL
(SEQ ID NO: 596)
GACATCCAGATGACCCAGTCTCCATCCTCCCTGTCTGCATCTGTAGGAGACAGAGTCACCATCACTTGC
CGGGCAAGTCAGAGCATTAGCAGCTATTTAAATTGGTATCAGCAGAAACCAGGGAAAGCCCCTAAGC
TCCTGATCTATGCTGCATCCAGTTTGCAAAGTGGGGTCCCGTCAAGGTTCAGTGGCAGTGGATCTGGG
ACAGATTTCACTCTCACCATCAGCAGTCTGCAACCTGAAGATTTTGCAACTTACTACTGTCAACAGAGT
TACAGTACCCCTCCGATCACCTTCGGCCAAGGGACACGACTGGAGATTAAA
(SEQ ID NO: 597)
DIQMTQSPSSLSASVGDRVTITCRASQSISSYLNWYQQKPGKAPKLLIYAASSLQSGVPSRFSGSGSGTDFTL
TISSLQPEDFATYYCQQSYSTPPITFGQGTRLEIK
(SEQ ID NO: 598)
CAGAGCATTAGCAGCTAT
(SEQ ID NO: 599)
QSISSY
(SEQ ID NO: 600)
GCTGCATCC
(SEQ ID NO: 601)
AAS
(SEQ ID NO: 602)
CAACAGAGTTACAGTACCCCTCCGATCACC
(SEQ ID NO: 603)
QQSYSTPPIT
(SEQ ID NO: 604)
GAAGTGCAGCTGGTGGAGTCTGGGGGAGGCGTGGTACAGCGGGGGGAGTCCCTGAGACTCTCCTGTTC
AGCCTCTGACTTCATCTTTAAAGATTATGCCATGTACTGGGTCCGTCAAATTCCAGGGAAGGGTCTAGA
GTGGATCTCTCTTATTAGTGGTGATGGTGACACTACATGGTATGGAGACTCTGTGAAGGGCCGATTCA
CCATCTCCAGAGACAACAACGAAAACTCCCTCTTTCTGCAAATGAACGATCTGAGAACTGAGGACACC
GCCATGTACTACTGTGCAAGAGATATGGGGTGGAACTTCTTTCAGTTGCAATACTGGGGCCAGGGAAC
CCTGGTCACCGTCTCCTCA
(SEQ ID NO: 605)
EVQLVESGGGVVQRGESLRLSCSASDFIFKDYAMYWVRQIPGKGLEWISLISGDGDTTWYGDSVKGRFTIS
RDNNENSLFLQMNDLRTEDTAMYYCARDMGWNFFQLQYWGQGTLVTVSS
(SEQ ID NO: 606)
GACTTCATCTTTAAAGATTATGCC
(SEQ ID NO: 607)
DFIFKDYA
(SEQ ID NO: 608)
ATTAGTGGTGATGGTGACACTACA
(SEQ ID NO: 609)
ISGDGDTT
(SEQ ID NO: 610)
GCAAGAGATATGGGGTGGAACTTCTTTCAGTTGCAATAC
(SEQ ID NO: 611)
ARDMGWNFFQLQY
(SEQ ID NO: 612)
CAGGTGCAGCTGCAGGAGTCGGGCCCCGCACTGGTGAAGCCTTCACAGACCCTGTCCCTCACCTGCAC
TGTCTCTGGTGGCTCCATCATCAGAGGTAGTACCTACTGGAGTTGGGTCCGCCAATTCCCAGGGAAGG
GCCTGGAGTGGATTGGATACAGTTATTACAGTGGGACCGCCTACTATAATCCGTCCCTCGAGAGTCGA
GCTACCATTTCTGTAGACACGTCTAAGAACCAGTTCTCCCTGAACCTGAAGTCTGTGACGGCCGCGGA
CACGGCCGTGTATTATTGTACAAGAGAAATAGGAGTGGCTGGTCTCTTTGACATCTGGGGCCAGGGAA
CCCTGGTCACCGTCTCCTCA
(SEQ ID NO: 613)
QVQLQESGPALVKPSQTLSLTCTVSGGSIIRGSTYWSWVRQFPGKGLEWIGYSYYSGTAYYNPSLESRATIS
VDTSKNQFSLNLKSVTAADTAVYYCTREIGVAGLFDIWGQGTLVTVSS
(SEQ ID NO: 614)
GGTGGCTCCATCATCAGAGGTAGTACCTAC
(SEQ ID NO: 615)
GGSIIRGSTY
(SEQ ID NO: 616)
AGTTATTACAGTGGGACCGCC
(SEQ ID NO: 617)
SYYSGTA
(SEQ ID NO: 618)
ACAAGAGAAATAGGAGTGGCTGGTCTCTTTGACATC
(SEQ ID NO: 619)
TREIGVAGLFDI
(SEQ ID NO: 620)
GAAATAGTTTTGACACAGAGTCCCGGCACACTGTCACTCTCTCCCGGGGAAAGAGCCACCTTGTCATG
TAGAGCAAGTCAGTCAGTCTCTAGCTCTTATCTCGCCTGGTACCAGCAGAAGCCGGGACAGGCCCCTA
GACTGCTGATCTACGGGGCAAGTTCCAGGGCCACCGGAATCCCCGACCGGTTCAGTGGAAGCGGAAG
CGGAACCGATTTTACTTTGACGATTTCTAGACTGGAGCCAGAGGATTTCGCCGTTTACTATTGTCAACA
GTACGGAAGCAGCCCGTGGACGTTTGGCCAGGGCACGAAGGTAGAAATCAAG
(SEQ ID NO: 621)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSPWTFGQGTKVEIK
(SEQ ID NO: 622)
AGAGCAAGTCAGTCAGTCTCTAGCTCTTATCTCGCC
(SEQ ID NO: 623)
RASQSVSSSYLA
(SEQ ID NO: 624)
GGGGCAAGTTCCAGGGCCACC
(SEQ ID NO: 625)
GASSRAT
(SEQ ID NO: 626)
CAACAGTACGGAAGCAGCCCGTGGACG
(SEQ ID NO: 627)
QQYGSSPWT
(SEQ ID NO: 628)
CAGGAGCAGTTGGTGCAGTCTGGGGCTGAGGTGAAGAAGCCTGGGGCCTCAGTGAAGGTCTCCTGTA
AGGCTTCTGGATACACCTTCACCGGCTACTATATACATTGGGTGCGACAGGCCCCTGGACTAGGGCTT
GAATGGATGGGATGGATCAACCCTAACAGTGGTGGCACAAAATATGCACAGAAGTTTCAGGGCAGGG
TCACCATGACCAGGGACACGTCCATCAATACAGCCTACATGGAGCTGAAAAGACTGAAATCTGACGA
CTCGGCCGTATATTACTGTGCGAGAGACGCCCCTCCCCATGATGTTTTTGATATCTGGGGCCAAGGGAC
ATTGGTCACCGTCTCTTCA
(SEQ ID NO: 629)
QEQLVQSGAEVKKPGASVKVSCKASGYTFTGYYIHWVRQAPGLGLEWMGWINPNSGGTKYAQKFQGRV
TMTRDTSINTAYMELKRLKSDDSAVYYCARDAPPHDVFDIWGQGTLVTVSS
(SEQ ID NO: 630)
GGATACACCTTCACCGGCTACTAT
(SEQ ID NO: 631)
GYTFTGYY
(SEQ ID NO: 632)
ATCAACCCTAACAGTGGTGGCACA
(SEQ ID NO: 633)
INPNSGGT
(SEQ ID NO: 634)
GCGAGAGACGCCCCTCCCCATGATGTTTTTGATATC
(SEQ ID NO: 635)
ARDAPPHDVFDI
(SEQ ID NO: 636)
GACATCCAGATGACCCAGTCTCCATCCTCCCTGTCTGCATCTGTAGGAGACAGAGTCACCATCACTTGC
CGGGCAAGTCAGGGCATTAGAAATGATTTAGGCTGGTATCAGCAGAAACCAGGGAAAGCCCCTAAGC
GCCTGATCTATGCTGCATCCAGTTTGCAAATTGGGGTCCCATCAAGGTTCAGCGGCAGTGGATCTGGG
ACAGAATTCACTCTCACAATCAGCAGCCTGCAGCCTGAAGATTTTGCAACTTATTACTGTCTACAGCAT
AATAGTTACCCGCTCACTTTCGGCGGAGGGACCAAGGTGGAGATCAAA
(SEQ ID NO: 637)
DIQMTQSPSSLSASVGDRVTITCRASQGIRNDLGWYQQKPGKAPKRLIYAASSLQIGVPSRFSGSGSGTEFTL
TISSLQPEDFATYYCLQHNSYPLTFGGGTKVEIK
(SEQ ID NO: 638)
CAGGGCATTAGAAATGAT
(SEQ ID NO: 639)
QGIRND
(SEQ ID NO: 640)
GCTGCATCC
(SEQ ID NO: 641)
AAS
(SEQ ID NO: 642)
CTACAGCATAATAGTTACCCGCTCACT
(SEQ ID NO: 643)
LQHNSYPLT
(SEQ ID NO: 644)
CAGGTGCAGCTGCAAGAGTCGGGCCCAGGACTGGTGAAGCCTTCACAGACCCTGTCCCTCACCTGCAC
TGTCTCTGGTGGCTCCATCAGTAGTGGTGCTTACCACTGGAGCTGGATCCGCCAGCACCCAGGGAAGG
GCCTAGAGTGGATTGGATACATCTATTACAATGGGGACACCTACTATAATCCGTCCCTCAAGAGTCGC
GTTACCATTTCAGTGGACACGTCTAAGAACCAATTCTTCCTGAAGGTGACCTCTGTGACTGCCGCGGAC
ACGGCCATGTATTACTGTGCGGGAGAAAAGCAGCTGACTGCTTTTGATATCTGGGGCCAAGGGACATT
GGTCACCGTCTCTTCA
(SEQ ID NO: 645)
QVQLQESGPGLVKPSQTLSLTCTVSGGSISSGAYHWSWIRQHPGKGLEWIGYIYYNGDTYYNPSLKSRVTIS
VDTSKNQFFLKVTSVTAADTAMYYCAGEKQLTAFDIWGQGTLVTVSS
(SEQ ID NO: 646)
GGTGGCTCCATCAGTAGTGGTGCTTACCAC
(SEQ ID NO: 647)
GGSISSGAYH
(SEQ ID NO: 648)
ATCTATTACAATGGGGACACC
(SEQ ID NO: 649)
IYYNGDT
(SEQ ID NO: 650)
GCGGGAGAAAAGCAGCTGACTGCTTTTGATATC
(SEQ ID NO: 651)
AGEKQLTAFDI
(SEQ ID NO: 652)
GTCATCCAGATGACCCAGTCTCCATCCTCCCTGTCTGCATCTGTTGGAGACAGAGTCACCATAACTTGC
CGGGCGAGTCAGGACATTAATAATTTTTTAAATTGGTATCAACAGAAATTAGGGAAAGCCCCTAAACT
CCTGATCTCCGATGCATCCAATTTGCAGACAGGAGTCCCGTCAAGGTTCAGTGGAAGTGGATCTGGGA
CAGATTTTACTTTCACCATCAGCAGCCTGCAGCCTGAAGATATTGCTGCATATTACTGTCAACAATATG
ATCATTTCCCGTATACTTTTGGCCAGGGGACCAGACTGGAGAACAAT
(SEQ ID NO: 653)
VIQMTQSPSSLSASVGDRVTITCRASQDINNFLNWYQQKLGKAPKLLISDASNLQTGVPSRFSGSGSGTDFT
FTISSLQPEDIAAYYCQQYDHFPYTFGQGTRLENN
(SEQ ID NO: 654)
CAGGACATTAATAATTTT
(SEQ ID NO: 655)
QDINNF
(SEQ ID NO: 656)
GATGCATCC
(SEQ ID NO: 657)
DAS
(SEQ ID NO: 658)
CAACAATATGATCATTTCCCGTATACT
(SEQ ID NO: 659)
QQYDHFPYT
(SEQ ID NO: 660)
GAGGTGCAGTTGGTGGAGTCTGGGGGAGGTGTGGTTCGGCCTGGGGGGTCCCTGAGACTCTCCTGTGC
AGCCTCTGGATTCACCTTTGATGATTATGGCATGACCTGGGTCCGCCAAGCTCCAGGGAAGGGGCTGG
AGTGGGTCTCTGGTATTAATTGGAATGGCGATAGCACAGAGTATTCAGACTCTGTGAAGGGCCGATTC
ACCATCTCCAGAGACAACGCCAAGAACTCCCTGTATCTGCAAATGAACAGTCTGAGAGCCGAGGACA
CGGCCTTCTATCACTGTGCGAGAGAGAATAACTGGAACTTCTACTTTGACTACTGGGGCCAGGGAACC
CTGGTCACCGTCTCCTCA
(SEQ ID NO: 661)
EVQLVESGGGVVRPGGSLRLSCAASGFTFDDYGMTWVRQAPGKGLEWVSGINWNGDSTEYSDSVKGRFT
ISRDNAKNSLYLQMNSLRAEDTAFYHCARENNWNFYFDYWGQGTLVTVSS
(SEQ ID NO: 662)
GGATTCACCTTTGATGATTATGGC
(SEQ ID NO: 663)
GFTFDDYG
(SEQ ID NO: 664)
ATTAATTGGAATGGCGATAGCACA
(SEQ ID NO: 665)
INWNGDST
(SEQ ID NO: 666)
GCGAGAGAGAATAACTGGAACTTCTACTTTGACTAC
(SEQ ID NO: 667)
ARENNWNFYFDY
(SEQ ID NO: 668)
GAAATAGTGATGACGCAGTCTCCAGCCACCCTGTCTGTGTCTCGAGGGGAAAGAGCCACCCTCTCCTG
TAGGGCCAGTCAGAGTGTTAGCAGCAACTTAGCCTGGTACCAGCAGAAACTTGGCCAGGCTCCCAGGC
TCCTCATCTATGGTGCATCCACCAGGGCCACTGGTATCCCAGCCAGGTTCAGTGGCAGTGGGTCTGGG
ACAGAGTTCACTCTCACCATCAGCAGCCTGCAGTCTGAAGATTTTGCAGTTTATTATTGTCAGCAGTAT
AATAACTGGCCGTGGACGTTCGGCCAAGGGACCAAGGTGGAAATCAAA
(SEQ ID NO: 669)
EIVMTQSPATLSVSRGERATLSCRASQSVSSNLAWYQQKLGQAPRLLIYGASTRATGIPARFSGSGSGTEFT
LTISSLQSEDFAVYYCQQYNNWPWTFGQGTKVEIK
(SEQ ID NO: 670)
CAGAGTGTTAGCAGCAAC
(SEQ ID NO: 671)
QSVSSN
(SEQ ID NO: 672)
GGTGCATCC
(SEQ ID NO: 673)
GAS
(SEQ ID NO: 674)
CAGCAGTATAATAACTGGCCGTGGACG
(SEQ ID NO: 675)
QQYNNWPWT
(SEQ ID NO: 676)
CAGGTCCACCTGGTACAGTCTGGGGCTGAGGTGAAGAAGCCTGGGGCCTCAGTGAAGGTCTCCTGCAA
GGTTTCCGGAAACACCCTCACTGAATTATCCATGCACTGGGTGCGACAGGCTCCTGGAAAAGGGCTTG
AGTGGATGGGAGGTTTTGATCCTGAAGATGGTGACACAATCTACTCACAGAAGTTCCAGGGCAGAGTC
ACCTTGACCGAGGACACATCTACAGACACAGCCTACATGGAGCTGAGCAGCCTGAGATCTGAGGACA
CGGCCGTGTATTACTGTTCAACAGTGGGGGGACCTACCTCTGACTGCTGGGGCCAGGGAACCCTGGTC
ACCGTCTCCTCA
(SEQ ID NO: 677)
QVHLVQSGAEVKKPGASVKVSCKVSGNTLTELSMHWVRQAPGKGLEWMGGFDPEDGDTIYSQKFQGRV
TLTEDTSTDTAYMELSSLRSEDTAVYYCSTVGGPTSDCWGQGTLVTVSS
(SEQ ID NO: 678)
GGAAACACCCTCACTGAATTATCC
(SEQ ID NO: 679)
GNTLTELS
(SEQ ID NO: 680)
TTTGATCCTGAAGATGGTGACACA
(SEQ ID NO: 681)
FDPEDGDT
(SEQ ID NO: 682)
TCAACAGTGGGGGGACCTACCTCTGACTGC
(SEQ ID NO: 683)
STVGGPTSDC
(SEQ ID NO: 684)
GACATCCAGATGACCCAGTCTCCATCCTCCCTGTCTGCATCTGTAGGAGACAGAGTCACCATCACTTGC
CAGGCGAGTCAGGACATTAGCAACTATTTAAATTGGTATCAGCAGAAACCAGGGAAAGCCCCTAAGG
TCCTGATCTTCGATGCATCCAATTTAGAACCAGGGGTCCCATCAAGGTTCAGTGGAAGTGGATCTGGG
ACAGATTTTACTTTCACCATCATCAGCCTGCAGCCTGAAGATATTGCAACATATTACTGTCAACAATAT
GATAATCTCCCGATCACCTTCGGCCAGGGGACACGACTGGACATTAAA
(SEQ ID NO: 685)
DIQMTQSPSSLSASVGDRVTITCQASQDISNYLNWYQQKPGKAPKVLIFDASNLEPGVPSRFSGSGSGTDFTF
TIISLQPEDIATYYCQQYDNLPITFGQGTRLDIK
(SEQ ID NO: 686)
CAGGACATTAGCAACTAT
(SEQ ID NO: 687)
QDISNY
(SEQ ID NO: 688)
GATGCATCC
(SEQ ID NO: 689)
DAS
(SEQ ID NO: 690)
CAACAATATGATAATCTCCCGATCACC
(SEQ ID NO: 691)
QQYDNLPIT

In some embodiments, an antibody or antigen-binding fragment thereof that binds specifically to C5 (e.g., which is used in a combination described herein) comprises: (1) a heavy chain variable region (HCVR) that comprises the HCDR1, HCDR2 and HCDR3 of a HCVR that comprises the amino acid sequence set forth in SEQ ID NO: 341 (or a variant thereof), and a light chain variable region (LCVR) that comprises the LCDR1, LCDR2 and LCDR3 of a LCVR that comprises the amino acid sequence set forth in SEQ ID NO: 349 (or a variant thereof); (2) a heavy chain variable region (HCVR) that comprises the HCDR1, HCDR2 and HCDR3 of a HCVR that comprises the amino acid sequence set forth in SEQ ID NO: 357 (or a variant thereof), and a light chain variable region (LCVR) that comprises the LCDR1, LCDR2 and LCDR3 of a LCVR that comprises the amino acid sequence set forth in SEQ ID NO: 365 (or a variant thereof); (3) a heavy chain variable region (HCVR) that comprises the HCDR1, HCDR2 and HCDR3 of a HCVR that comprises the amino acid sequence set forth in SEQ ID NO: 373 (or a variant thereof), and a light chain variable region (LCVR) that comprises the LCDR1, LCDR2 and LCDR3 of a LCVR that comprises the amino acid sequence set forth in SEQ ID NO: 381 (or a variant thereof); (4) a heavy chain variable region (HCVR) that comprises the HCDR1, HCDR2 and HCDR3 of a HCVR that comprises the amino acid sequence set forth in SEQ ID NO: 389 (or a variant thereof), and a light chain variable region (LCVR) that comprises the LCDR1, LCDR2 and LCDR3 of a LCVR that comprises the amino acid sequence set forth in SEQ ID NO: 397 (or a variant thereof); (5) a heavy chain variable region (HCVR) that comprises the HCDR1, HCDR2 and HCDR3 of a HCVR that comprises the amino acid sequence set forth in SEQ ID NO: 405 (or a variant thereof), and a light chain variable region (LCVR) that comprises the LCDR1, LCDR2 and LCDR3 of a LCVR that comprises the amino acid sequence set forth in SEQ ID NO: 413 (or a variant thereof); (6) a heavy chain variable region (HCVR) that comprises the HCDR1, HCDR2 and HCDR3 of a HCVR that comprises the amino acid sequence set forth in SEQ ID NO: 421 (or a variant thereof), and a light chain variable region (LCVR) that comprises the LCDR1, LCDR2 and LCDR3 of a LCVR that comprises the amino acid sequence set forth in SEQ ID NO: 429 (or a variant thereof); (7) a heavy chain variable region (HCVR) that comprises the HCDR1, HCDR2 and HCDR3 of a HCVR that comprises the amino acid sequence set forth in SEQ ID NO: 437 (or a variant thereof), and a light chain variable region (LCVR) that comprises the LCDR1, LCDR2 and LCDR3 of a LCVR that comprises the amino acid sequence set forth in SEQ ID NO: 445 (or a variant thereof); (8) a heavy chain variable region (HCVR) that comprises the HCDR1, HCDR2 and HCDR3 of a HCVR that comprises the amino acid sequence set forth in SEQ ID NO: 437 (or a variant thereof), and a light chain variable region (LCVR) that comprises the LCDR1, LCDR2 and LCDR3 of a LCVR that comprises the amino acid sequence set forth in SEQ ID NO: 453 (or a variant thereof); (9) a heavy chain variable region (HCVR) that comprises the HCDR1, HCDR2 and HCDR3 of a HCVR that comprises the amino acid sequence set forth in SEQ ID NO: 461 (or a variant thereof), and a light chain variable region (LCVR) that comprises the LCDR1, LCDR2 and LCDR3 of a LCVR that comprises the amino acid sequence set forth in SEQ ID NO: 445 (or a variant thereof); (10) a heavy chain variable region (HCVR) that comprises the HCDR1, HCDR2 and HCDR3 of a HCVR that comprises the amino acid sequence set forth in SEQ ID NO: 437 (or a variant thereof), and a light chain variable region (LCVR) that comprises the LCDR1, LCDR2 and LCDR3 of a LCVR that comprises the amino acid sequence set forth in SEQ ID NO: 469 (or a variant thereof); (11) a heavy chain variable region (HCVR) that comprises the HCDR1, HCDR2 and HCDR3 of a HCVR that comprises the amino acid sequence set forth in SEQ ID NO: 477 (or a variant thereof), and a light chain variable region (LCVR) that comprises the LCDR1, LCDR2 and LCDR3 of a LCVR that comprises the amino acid sequence set forth in SEQ ID NO: 445 (or a variant thereof); (12) a heavy chain variable region (HCVR) that comprises the HCDR1, HCDR2 and HCDR3 of a HCVR that comprises the amino acid sequence set forth in SEQ ID NO: 485 (or a variant thereof), and a light chain variable region (LCVR) that comprises the LCDR1, LCDR2 and LCDR3 of a LCVR that comprises the amino acid sequence set forth in SEQ ID NO: 445 (or a variant thereof); (13) a heavy chain variable region (HCVR) that comprises the HCDR1, HCDR2 and HCDR3 of a HCVR that comprises the amino acid sequence set forth in SEQ ID NO: 461 (or a variant thereof), and a light chain variable region (LCVR) that comprises the LCDR1, LCDR2 and LCDR3 of a LCVR that comprises the amino acid sequence set forth in SEQ ID NO: 469 (or a variant thereof); (14) a heavy chain variable region (HCVR) that comprises the HCDR1, HCDR2 and HCDR3 of a HCVR that comprises the amino acid sequence set forth in SEQ ID NO: 485 (or a variant thereof), and a light chain variable region (LCVR) that comprises the LCDR1, LCDR2 and LCDR3 of a LCVR that comprises the amino acid sequence set forth in SEQ ID NO: 453 (or a variant thereof); (15) a heavy chain variable region (HCVR) that comprises the HCDR1, HCDR2 and HCDR3 of a HCVR that comprises the amino acid sequence set forth in SEQ ID NO: 485 (or a variant thereof), and a light chain variable region (LCVR) that comprises the LCDR1, LCDR2 and LCDR3 of a LCVR that comprises the amino acid sequence set forth in SEQ ID NO: 469 (or a variant thereof); (16) a heavy chain variable region (HCVR) that comprises the HCDR1, HCDR2 and HCDR3 of a HCVR that comprises the amino acid sequence set forth in SEQ ID NO: 477, and a light chain variable region (LCVR) that comprises the LCDR1, LCDR2 and LCDR3 of a LCVR that comprises the amino acid sequence set forth in SEQ ID NO: 469 (or a variant thereof); (17) a heavy chain variable region (HCVR) that comprises the HCDR1, HCDR2 and HCDR3 of a HCVR that comprises the amino acid sequence set forth in SEQ ID NO: 493 (or a variant thereof), and a light chain variable region (LCVR) that comprises the LCDR1, LCDR2 and LCDR3 of a LCVR that comprises the amino acid sequence set forth in SEQ ID NO: 501 (or a variant thereof); (18) a heavy chain variable region (HCVR) that comprises the HCDR1, HCDR2 and HCDR3 of a HCVR that comprises the amino acid sequence set forth in SEQ ID NO: 509 (or a variant thereof), and a light chain variable region (LCVR) that comprises the LCDR1, LCDR2 and LCDR3 of a LCVR that comprises the amino acid sequence set forth in SEQ ID NO: 517 (or a variant thereof); (19) a heavy chain variable region (HCVR) that comprises the HCDR1, HCDR2 and HCDR3 of a HCVR that comprises the amino acid sequence set forth in SEQ ID NO: 525 (or a variant thereof), and a light chain variable region (LCVR) that comprises the LCDR1, LCDR2 and LCDR3 of a LCVR that comprises the amino acid sequence set forth in SEQ ID NO: 533 (or a variant thereof); (20) a heavy chain variable region (HCVR) that comprises the HCDR1, HCDR2 and HCDR3 of a HCVR that comprises the amino acid sequence set forth in SEQ ID NO: 541 (or a variant thereof), and a light chain variable region (LCVR) that comprises the LCDR1, LCDR2 and LCDR3 of a LCVR that comprises the amino acid sequence set forth in SEQ ID NO: 549 (or a variant thereof); (21) a heavy chain variable region (HCVR) that comprises the HCDR1, HCDR2 and HCDR3 of a HCVR that comprises the amino acid sequence set forth in SEQ ID NO: 557 (or a variant thereof), and a light chain variable region (LCVR) that comprises the LCDR1, LCDR2 and LCDR3 of a LCVR that comprises the amino acid sequence set forth in SEQ ID NO: 565 (or a variant thereof); (22) a heavy chain variable region (HCVR) that comprises the HCDR1, HCDR2 and HCDR3 of a HCVR that comprises the amino acid sequence set forth in SEQ ID NO: 573 (or a variant thereof), and a light chain variable region (LCVR) that comprises the LCDR1, LCDR2 and LCDR3 of a LCVR that comprises the amino acid sequence set forth in SEQ ID NO: 581 (or a variant thereof); (23) a heavy chain variable region (HCVR) that comprises the HCDR1, HCDR2 and HCDR3 of a HCVR that comprises the amino acid sequence set forth in SEQ ID NO: 589 (or a variant thereof), and a light chain variable region (LCVR) that comprises the LCDR1, LCDR2 and LCDR3 of a LCVR that comprises the amino acid sequence set forth in SEQ ID NO: 597 (or a variant thereof); (24) a heavy chain variable region (HCVR) that comprises the HCDR1, HCDR2 and HCDR3 of a HCVR that comprises the amino acid sequence set forth in SEQ ID NO: 605 (or a variant thereof), and a light chain variable region (LCVR) that comprises the LCDR1, LCDR2 and LCDR3 of a LCVR that comprises the amino acid sequence set forth in SEQ ID NO: 597 (or a variant thereof); (25) a heavy chain variable region (HCVR) that comprises the HCDR1, HCDR2 and HCDR3 of a HCVR that comprises the amino acid sequence set forth in SEQ ID NO: 613 (or a variant thereof), and a light chain variable region (LCVR) that comprises the LCDR1, LCDR2 and LCDR3 of a LCVR that comprises the amino acid sequence set forth in SEQ ID NO: 621 (or a variant thereof); (26) a heavy chain variable region (HCVR) that comprises the HCDR1, HCDR2 and HCDR3 of a HCVR that comprises the amino acid sequence set forth in SEQ ID NO: 629 (or a variant thereof), and a light chain variable region (LCVR) that comprises the LCDR1, LCDR2 and LCDR3 of a LCVR that comprises the amino acid sequence set forth in SEQ ID NO: 637 (or a variant thereof); (27) a heavy chain variable region (HCVR) that comprises the HCDR1, HCDR2 and HCDR3 of a HCVR that comprises the amino acid sequence set forth in SEQ ID NO: 645 (or a variant thereof), and a light chain variable region (LCVR) that comprises the LCDR1, LCDR2 and LCDR3 of a LCVR that comprises the amino acid sequence set forth in SEQ ID NO: 653 (or a variant thereof); (28) a heavy chain variable region (HCVR) that comprises the HCDR1, HCDR2 and HCDR3 of a HCVR that comprises the amino acid sequence set forth in SEQ ID NO: 661 (or a variant thereof), and a light chain variable region (LCVR) that comprises the LCDR1, LCDR2 and LCDR3 of a LCVR that comprises the amino acid sequence set forth in SEQ ID NO: 669 (or a variant thereof); and/or (29) a heavy chain variable region (HCVR) that comprises the HCDR1, HCDR2 and HCDR3 of a HCVR that comprises the amino acid sequence set forth in SEQ ID NO: 677 (or a variant thereof), and a light chain variable region (LCVR) that comprises the LCDR1, LCDR2 and LCDR3 of a LCVR that comprises the amino acid sequence set forth in SEQ ID NO: 685 (or a variant thereof).

In some embodiments, an antibody or antigen-binding fragment thereof that binds specifically to C5 (e.g., which is used in a combination disclosed herein) comprises: (a) a heavy chain variable region comprising an HCDR1 that comprises the amino acid sequence set forth in SEQ ID NO: 343 (or a variant thereof), an HCDR2 that comprises the amino acid sequence set forth in SEQ ID NO: 345 (or a variant thereof), an HCDR3 that comprises the amino acid sequence set forth in SEQ ID NO: 347 (or a variant thereof), and a light chain variable region comprising an LCDR1 that comprises the amino acid sequence set forth in SEQ ID NO: 351 (or a variant thereof), an LCDR2 that comprises the amino acid sequence set forth in SEQ ID NO:353 (or a variant thereof), an LCDR3 that comprises the amino acid sequence set forth in SEQ ID NO:355 (or a variant thereof); (b) a heavy chain variable region comprising an HCDR1 that comprises the amino acid sequence set forth in SEQ ID NO: 359 (or a variant thereof), an HCDR2 that comprises the amino acid sequence set forth in SEQ ID NO: 361 (or a variant thereof), an HCDR3 that comprises the amino acid sequence set forth in SEQ ID NO: 363 (or a variant thereof), and a light chain variable region comprising an LCDR1 that comprises the amino acid sequence set forth in SEQ ID NO: 367 (or a variant thereof), an LCDR2 that comprises the amino acid sequence set forth in SEQ ID NO: 369 (or a variant thereof), an LCDR3 that comprises the amino acid sequence set forth in SEQ ID NO: 371 (or a variant thereof); (c) a heavy chain variable region comprising an HCDR1 that comprises the amino acid sequence set forth in SEQ ID NO: 375 (or a variant thereof), an HCDR2 that comprises the amino acid sequence set forth in SEQ ID NO: 377 (or a variant thereof), an HCDR3 that comprises the amino acid sequence set forth in SEQ ID NO: 379 (or a variant thereof), and a light chain variable region comprising an LCDR1 that comprises the amino acid sequence set forth in SEQ ID NO: 383 (or a variant thereof), an LCDR2 that comprises the amino acid sequence set forth in SEQ ID NO:385 (or a variant thereof), an LCDR3 that comprises the amino acid sequence set forth in SEQ ID NO:387 (or a variant thereof); (d) a heavy chain variable region comprising an HCDR1 that comprises the amino acid sequence set forth in SEQ ID NO: 391 (or a variant thereof), an HCDR2 that comprises the amino acid sequence set forth in SEQ ID NO: 393 (or a variant thereof), an HCDR3 that comprises the amino acid sequence set forth in SEQ ID NO: 395 (or a variant thereof), and a light chain variable region comprising an LCDR1 that comprises the amino acid sequence set forth in SEQ ID NO: 399 (or a variant thereof), an LCDR2 that comprises the amino acid sequence set forth in SEQ ID NO: 401 (or a variant thereof), an LCDR3 that comprises the amino acid sequence set forth in SEQ ID NO: 403 (or a variant thereof); (e) a heavy chain variable region comprising an HCDR1 that comprises the amino acid sequence set forth in SEQ ID NO: 407 (or a variant thereof), an HCDR2 that comprises the amino acid sequence set forth in SEQ ID NO: 409 (or a variant thereof), an HCDR3 that comprises the amino acid sequence set forth in SEQ ID NO: 411 (or a variant thereof), and a light chain variable region comprising an LCDR1 that comprises the amino acid sequence set forth in SEQ ID NO: 415 (or a variant thereof), an LCDR2 that comprises the amino acid sequence set forth in SEQ ID NO: 417 (or a variant thereof), an LCDR3 that comprises the amino acid sequence set forth in SEQ ID NO: 419 (or a variant thereof); (f) a heavy chain variable region comprising an HCDR1 that comprises the amino acid sequence set forth in SEQ ID NO: 423 (or a variant thereof), an HCDR2 that comprises the amino acid sequence set forth in SEQ ID NO: 425 (or a variant thereof), an HCDR3 that comprises the amino acid sequence set forth in SEQ ID NO: 427 (or a variant thereof), and a light chain variable region comprising an LCDR1 that comprises the amino acid sequence set forth in SEQ ID NO: 431 (or a variant thereof), an LCDR2 that comprises the amino acid sequence set forth in SEQ ID NO: 433 (or a variant thereof), an LCDR3 that comprises the amino acid sequence set forth in SEQ ID NO: 435 (or a variant thereof); (h) a heavy chain variable region comprising an HCDR1 that comprises the amino acid sequence set forth in SEQ ID NO: 439 (or a variant thereof), an HCDR2 that comprises the amino acid sequence set forth in SEQ ID NO: 441 (or a variant thereof), an HCDR3 that comprises the amino acid sequence set forth in SEQ ID NO: 443 (or a variant thereof), and a light chain variable region comprising an LCDR1 that comprises the amino acid sequence set forth in SEQ ID NO: 447 (or a variant thereof), an LCDR2 that comprises the amino acid sequence set forth in SEQ ID NO: 449 (or a variant thereof), an LCDR3 that comprises the amino acid sequence set forth in SEQ ID NO: 451 (or a variant thereof); (j) a heavy chain variable region comprising an HCDR1 that comprises the amino acid sequence set forth in SEQ ID NO: 439 (or a variant thereof), an HCDR2 that comprises the amino acid sequence set forth in SEQ ID NO: 441 (or a variant thereof), an HCDR3 that comprises the amino acid sequence set forth in SEQ ID NO: 443 (or a variant thereof), and a light chain variable region comprising an LCDR1 that comprises the amino acid sequence set forth in SEQ ID NO: 455 (or a variant thereof), an LCDR2 that comprises the amino acid sequence set forth in SEQ ID NO:457 (or a variant thereof), an LCDR3 that comprises the amino acid sequence set forth in SEQ ID NO:459 (or a variant thereof); (k) a heavy chain variable region comprising an HCDR1 that comprises the amino acid sequence set forth in SEQ ID NO: 463 (or a variant thereof), an HCDR2 that comprises the amino acid sequence set forth in SEQ ID NO: 465 (or a variant thereof), an HCDR3 that comprises the amino acid sequence set forth in SEQ ID NO: 467 (or a variant thereof), and a light chain variable region comprising an LCDR1 that comprises the amino acid sequence set forth in SEQ ID NO: 447 (or a variant thereof), an LCDR2 that comprises the amino acid sequence set forth in SEQ ID NO: 449 (or a variant thereof), an LCDR3 that comprises the amino acid sequence set forth in SEQ ID NO: 451 (or a variant thereof); (m) a heavy chain variable region comprising an HCDR1 that comprises the amino acid sequence set forth in SEQ ID NO: 439 (or a variant thereof), an HCDR2 that comprises the amino acid sequence set forth in SEQ ID NO: 441 (or a variant thereof), an HCDR3 that comprises the amino acid sequence set forth in SEQ ID NO: 443 (or a variant thereof), and a light chain variable region comprising an LCDR1 that comprises the amino acid sequence set forth in SEQ ID NO: 471 (or a variant thereof), an LCDR2 that comprises the amino acid sequence set forth in SEQ ID NO: 473 (or a variant thereof), an LCDR3 that comprises the amino acid sequence set forth in SEQ ID NO: 475 (or a variant thereof); (n) a heavy chain variable region comprising an HCDR1 that comprises the amino acid sequence set forth in SEQ ID NO: 479 (or a variant thereof), an HCDR2 that comprises the amino acid sequence set forth in SEQ ID NO: 481 (or a variant thereof), an HCDR3 that comprises the amino acid sequence set forth in SEQ ID NO: 483 (or a variant thereof), and a light chain variable region comprising an LCDR1 that comprises the amino acid sequence set forth in SEQ ID NO: 447 (or a variant thereof), an LCDR2 that comprises the amino acid sequence set forth in SEQ ID NO: 449 (or a variant thereof), an LCDR3 that comprises the amino acid sequence set forth in SEQ ID NO: 451 (or a variant thereof); (p) a heavy chain variable region comprising an HCDR1 that comprises the amino acid sequence set forth in SEQ ID NO: 487 (or a variant thereof), an HCDR2 that comprises the amino acid sequence set forth in SEQ ID NO: 489 (or a variant thereof), an HCDR3 that comprises the amino acid sequence set forth in SEQ ID NO: 491 (or a variant thereof), and a light chain variable region comprising an LCDR1 that comprises the amino acid sequence set forth in SEQ ID NO: 447 (or a variant thereof), an LCDR2 that comprises the amino acid sequence set forth in SEQ ID NO: 449 (or a variant thereof), an LCDR3 that comprises the amino acid sequence set forth in SEQ ID NO: 451 (or a variant thereof); (q) a heavy chain variable region comprising an HCDR1 that comprises the amino acid sequence set forth in SEQ ID NO: 463 (or a variant thereof), an HCDR2 that comprises the amino acid sequence set forth in SEQ ID NO: 465 (or a variant thereof), an HCDR3 that comprises the amino acid sequence set forth in SEQ ID NO: 467 (or a variant thereof), and a light chain variable region comprising an LCDR1 that comprises the amino acid sequence set forth in SEQ ID NO: 471 (or a variant thereof), an LCDR2 that comprises the amino acid sequence set forth in SEQ ID NO: 473 (or a variant thereof), an LCDR3 that comprises the amino acid sequence set forth in SEQ ID NO: 475 (or a variant thereof); (r) a heavy chain variable region comprising an HCDR1 that comprises the amino acid sequence set forth in SEQ ID NO: 487 (or a variant thereof), an HCDR2 that comprises the amino acid sequence set forth in SEQ ID NO: 489 (or a variant thereof), an HCDR3 that comprises the amino acid sequence set forth in SEQ ID NO: 491 (or a variant thereof), and a light chain variable region comprising an LCDR1 that comprises the amino acid sequence set forth in SEQ ID NO: 455 (or a variant thereof), an LCDR2 that comprises the amino acid sequence set forth in SEQ ID NO: 457 (or a variant thereof), an LCDR3 that comprises the amino acid sequence set forth in SEQ ID NO: 459 (or a variant thereof); (s) a heavy chain variable region comprising an HCDR1 that comprises the amino acid sequence set forth in SEQ ID NO: 487 (or a variant thereof), an HCDR2 that comprises the amino acid sequence set forth in SEQ ID NO: 489 (or a variant thereof), an HCDR3 that comprises the amino acid sequence set forth in SEQ ID NO: 491 (or a variant thereof), and a light chain variable region comprising an LCDR1 that comprises the amino acid sequence set forth in SEQ ID NO: 471 (or a variant thereof), an LCDR2 that comprises the amino acid sequence set forth in SEQ ID NO: 473 (or a variant thereof), an LCDR3 that comprises the amino acid sequence set forth in SEQ ID NO: 475 (or a variant thereof); (t) a heavy chain variable region comprising an HCDR1 that comprises the amino acid sequence set forth in SEQ ID NO: 479 (or a variant thereof), an HCDR2 that comprises the amino acid sequence set forth in SEQ ID NO: 481 (or a variant thereof), an HCDR3 that comprises the amino acid sequence set forth in SEQ ID NO: 483 (or a variant thereof), and a light chain variable region comprising an LCDR1 that comprises the amino acid sequence set forth in SEQ ID NO: 471 (or a variant thereof), an LCDR2 that comprises the amino acid sequence set forth in SEQ ID NO: 473 (or a variant thereof), an LCDR3 that comprises the amino acid sequence set forth in SEQ ID NO: 475 (or a variant thereof); (u) a heavy chain variable region comprising an HCDR1 that comprises the amino acid sequence set forth in SEQ ID NO: 495 (or a variant thereof), an HCDR2 that comprises the amino acid sequence set forth in SEQ ID NO: 497 (or a variant thereof), an HCDR3 that comprises the amino acid sequence set forth in SEQ ID NO: 499 (or a variant thereof), and a light chain variable region comprising an LCDR1 that comprises the amino acid sequence set forth in SEQ ID NO: 503 (or a variant thereof), an LCDR2 that comprises the amino acid sequence set forth in SEQ ID NO: 505 (or a variant thereof), an LCDR3 that comprises the amino acid sequence set forth in SEQ ID NO: 507 (or a variant thereof); (v) a heavy chain variable region comprising an HCDR1 that comprises the amino acid sequence set forth in SEQ ID NO: 511 (or a variant thereof), an HCDR2 that comprises the amino acid sequence set forth in SEQ ID NO: 513 (or a variant thereof), an HCDR3 that comprises the amino acid sequence set forth in SEQ ID NO: 515 (or a variant thereof), and a light chain variable region comprising an LCDR1 that comprises the amino acid sequence set forth in SEQ ID NO: 519 (or a variant thereof), an LCDR2 that comprises the amino acid sequence set forth in SEQ ID NO: 521 (or a variant thereof), an LCDR3 that comprises the amino acid sequence set forth in SEQ ID NO: 523 (or a variant thereof); (w) a heavy chain variable region comprising an HCDR1 that comprises the amino acid sequence set forth in SEQ ID NO: 527 (or a variant thereof), an HCDR2 that comprises the amino acid sequence set forth in SEQ ID NO: 529 (or a variant thereof), an HCDR3 that comprises the amino acid sequence set forth in SEQ ID NO: 531 (or a variant thereof), and a light chain variable region comprising an LCDR1 that comprises the amino acid sequence set forth in SEQ ID NO: 535 (or a variant thereof), an LCDR2 that comprises the amino acid sequence set forth in SEQ ID NO: 537 (or a variant thereof), an LCDR3 that comprises the amino acid sequence set forth in SEQ ID NO: 539 (or a variant thereof); (x) a heavy chain variable region comprising an HCDR1 that comprises the amino acid sequence set forth in SEQ ID NO: 543 (or a variant thereof), an HCDR2 that comprises the amino acid sequence set forth in SEQ ID NO: 545 (or a variant thereof), an HCDR3 that comprises the amino acid sequence set forth in SEQ ID NO: 547 (or a variant thereof), and a light chain variable region comprising an LCDR1 that comprises the amino acid sequence set forth in SEQ ID NO: 551 (or a variant thereof), an LCDR2 that comprises the amino acid sequence set forth in SEQ ID NO: 553 (or a variant thereof), an LCDR3 that comprises the amino acid sequence set forth in SEQ ID NO: 555 (or a variant thereof); (y) a heavy chain variable region comprising an HCDR1 that comprises the amino acid sequence set forth in SEQ ID NO: 559 (or a variant thereof), an HCDR2 that comprises the amino acid sequence set forth in SEQ ID NO: 561 (or a variant thereof), an HCDR3 that comprises the amino acid sequence set forth in SEQ ID NO: 563 (or a variant thereof), and a light chain variable region comprising an LCDR1 that comprises the amino acid sequence set forth in SEQ ID NO: 567 (or a variant thereof), an LCDR2 that comprises the amino acid sequence set forth in SEQ ID NO: 569 (or a variant thereof), an LCDR3 that comprises the amino acid sequence set forth in SEQ ID NO: 571 (or a variant thereof); (z) a heavy chain variable region comprising an HCDR1 that comprises the amino acid sequence set forth in SEQ ID NO: 575 (or a variant thereof), an HCDR2 that comprises the amino acid sequence set forth in SEQ ID NO: 577 (or a variant thereof), an HCDR3 that comprises the amino acid sequence set forth in SEQ ID NO: 579 (or a variant thereof), and a light chain variable region comprising an LCDR1 that comprises the amino acid sequence set forth in SEQ ID NO: 583 (or a variant thereof), an LCDR2 that comprises the amino acid sequence set forth in SEQ ID NO: 585 (or a variant thereof), an LCDR3 that comprises the amino acid sequence set forth in SEQ ID NO: 587 (or a variant thereof); (aa) a heavy chain variable region comprising an HCDR1 that comprises the amino acid sequence set forth in SEQ ID NO: 591 (or a variant thereof), an HCDR2 that comprises the amino acid sequence set forth in SEQ ID NO: 593 (or a variant thereof), an HCDR3 that comprises the amino acid sequence set forth in SEQ ID NO: 595 (or a variant thereof), and a light chain variable region comprising an LCDR1 that comprises the amino acid sequence set forth in SEQ ID NO: 599 (or a variant thereof), an LCDR2 that comprises the amino acid sequence set forth in SEQ ID NO: 601 (or a variant thereof), an LCDR3 that comprises the amino acid sequence set forth in SEQ ID NO: 603 (or a variant thereof); (ab) a heavy chain variable region comprising an HCDR1 that comprises the amino acid sequence set forth in SEQ ID NO: 607 (or a variant thereof), an HCDR2 that comprises the amino acid sequence set forth in SEQ ID NO: 609 (or a variant thereof), an HCDR3 that comprises the amino acid sequence set forth in SEQ ID NO: 611 (or a variant thereof), and a light chain variable region comprising an LCDR1 that comprises the amino acid sequence set forth in SEQ ID NO: 599 (or a variant thereof), an LCDR2 that comprises the amino acid sequence set forth in SEQ ID NO: 601 (or a variant thereof), an LCDR3 that comprises the amino acid sequence set forth in SEQ ID NO: 603 (or a variant thereof); (ac) a heavy chain variable region comprising an HCDR1 that comprises the amino acid sequence set forth in SEQ ID NO: 615 (or a variant thereof), an HCDR2 that comprises the amino acid sequence set forth in SEQ ID NO: 617 (or a variant thereof), an HCDR3 that comprises the amino acid sequence set forth in SEQ ID NO: 619 (or a variant thereof), and a light chain variable region comprising an LCDR1 that comprises the amino acid sequence set forth in SEQ ID NO: 623 (or a variant thereof), an LCDR2 that comprises the amino acid sequence set forth in SEQ ID NO: 625 (or a variant thereof), an LCDR3 that comprises the amino acid sequence set forth in SEQ ID NO: 627 (or a variant thereof); (ad) a heavy chain variable region comprising an HCDR1 that comprises the amino acid sequence set forth in SEQ ID NO: 631 (or a variant thereof), an HCDR2 that comprises the amino acid sequence set forth in SEQ ID NO: 633 (or a variant thereof), an HCDR3 that comprises the amino acid sequence set forth in SEQ ID NO: 635 (or a variant thereof), and a light chain variable region comprising an LCDR1 that comprises the amino acid sequence set forth in SEQ ID NO: 639 (or a variant thereof), an LCDR2 that comprises the amino acid sequence set forth in SEQ ID NO: 641 (or a variant thereof), an LCDR3 that comprises the amino acid sequence set forth in SEQ ID NO: 643 (or a variant thereof); (ae) a heavy chain variable region comprising an HCDR1 that comprises the amino acid sequence set forth in SEQ ID NO: 647 (or a variant thereof), an HCDR2 that comprises the amino acid sequence set forth in SEQ ID NO: 649 (or a variant thereof), an HCDR3 that comprises the amino acid sequence set forth in SEQ ID NO: 651 (or a variant thereof), and a light chain variable region comprising an LCDR1 that comprises the amino acid sequence set forth in SEQ ID NO: 655 (or a variant thereof), an LCDR2 that comprises the amino acid sequence set forth in SEQ ID NO: 657 (or a variant thereof), an LCDR3 that comprises the amino acid sequence set forth in SEQ ID NO: 659 (or a variant thereof); (af) a heavy chain variable region comprising an HCDR1 that comprises the amino acid sequence set forth in SEQ ID NO: 663 (or a variant thereof), an HCDR2 that comprises the amino acid sequence set forth in SEQ ID NO: 665 (or a variant thereof), an HCDR3 that comprises the amino acid sequence set forth in SEQ ID NO: 667 (or a variant thereof), and a light chain variable region comprising an LCDR1 that comprises the amino acid sequence set forth in SEQ ID NO: 671 (or a variant thereof), an LCDR2 that comprises the amino acid sequence set forth in SEQ ID NO: 673 (or a variant thereof), an LCDR3 that comprises the amino acid sequence set forth in SEQ ID NO: 675 (or a variant thereof); and/or (ag) a heavy chain variable region comprising an HCDR1 that comprises the amino acid sequence set forth in SEQ ID NO: 679 (or a variant thereof), an HCDR2 that comprises the amino acid sequence set forth in SEQ ID NO: 681 (or a variant thereof), an HCDR3 that comprises the amino acid sequence set forth in SEQ ID NO: 683 (or a variant thereof), and a light chain variable region comprising an LCDR1 that comprises the amino acid sequence set forth in SEQ ID NO: 687 (or a variant thereof), an LCDR2 that comprises the amino acid sequence set forth in SEQ ID NO: 689 (or a variant thereof), an LCDR3 that comprises the amino acid sequence set forth in SEQ ID NO: 691 (or a variant thereof).

In some embodiments, an antibody or antigen-binding fragment thereof that binds specifically to C5 (e.g., which is used in a combination described herein) comprises: (i) a heavy chain variable region that comprises the amino acid sequence set forth in SEQ ID NO: 341 (or a variant thereof), and a light chain variable region that comprises the amino acid sequence set forth in SEQ ID NO: 349 (or a variant thereof); (ii) a heavy chain variable region that comprises the amino acid sequence set forth in SEQ ID NO: 357 (or a variant thereof), and a light chain variable region that comprises the amino acid sequence set forth in SEQ ID NO: 365 (or a variant thereof); (iii) a heavy chain variable region that comprises the amino acid sequence set forth in SEQ ID NO: 373 (or a variant thereof), and a light chain variable region that comprises the amino acid sequence set forth in SEQ ID NO: 381 (or a variant thereof); (iv) a heavy chain variable region that comprises the amino acid sequence set forth in SEQ ID NO: 389 (or a variant thereof), and a light chain variable region that comprises the amino acid sequence set forth in SEQ ID NO: 397 (or a variant thereof); (v) a heavy chain variable region that comprises the amino acid sequence set forth in SEQ ID NO: 405 (or a variant thereof), and a light chain variable region that comprises the amino acid sequence set forth in SEQ ID NO: 413 (or a variant thereof); (vi) a heavy chain variable region that comprises the amino acid sequence set forth in SEQ ID NO: 421 (or a variant thereof), and a light chain variable region that comprises the amino acid sequence set forth in SEQ ID NO: 429 (or a variant thereof); (vii) a heavy chain variable region that comprises the amino acid sequence set forth in SEQ ID NO: 437 (or a variant thereof), and a light chain variable region that comprises the amino acid sequence set forth in SEQ ID NO: 445 (or a variant thereof); (viii) a heavy chain variable region that comprises the amino acid sequence set forth in SEQ ID NO: 437 (or a variant thereof), and a light chain variable region that comprises the amino acid sequence set forth in SEQ ID NO: 453 (or a variant thereof); (ix) a heavy chain variable region that comprises the amino acid sequence set forth in SEQ ID NO: 461 (or a variant thereof), and a light chain variable region that comprises the amino acid sequence set forth in SEQ ID NO: 445 (or a variant thereof); (x) a heavy chain variable region that comprises the amino acid sequence set forth in SEQ ID NO: 437 (or a variant thereof), and a light chain variable region that comprises the amino acid sequence set forth in SEQ ID NO: 469 (or a variant thereof); (xi) a heavy chain variable region that comprises the amino acid sequence set forth in SEQ ID NO: 477 (or a variant thereof), and a light chain variable region that comprises the amino acid sequence set forth in SEQ ID NO: 445 (or a variant thereof); (xii) a heavy chain variable region that comprises the amino acid sequence set forth in SEQ ID NO: 485 (or a variant thereof), and a light chain variable region that comprises the amino acid sequence set forth in SEQ ID NO: 445 (or a variant thereof); (xiii) a heavy chain variable region that comprises the amino acid sequence set forth in SEQ ID NO: 461 (or a variant thereof), and a light chain variable region that comprises the amino acid sequence set forth in SEQ ID NO: 469 (or a variant thereof); (xiv) a heavy chain variable region that comprises the amino acid sequence set forth in SEQ ID NO: 485 (or a variant thereof), and a light chain variable region that comprises the amino acid sequence set forth in SEQ ID NO: 453 (or a variant thereof); (xv) a heavy chain variable region that comprises the amino acid sequence set forth in SEQ ID NO: 485 (or a variant thereof), and a light chain variable region that comprises the amino acid sequence set forth in SEQ ID NO: 469 (or a variant thereof); (xvi) a heavy chain variable region that comprises the amino acid sequence set forth in SEQ ID NO: 477 (or a variant thereof), and a light chain variable region that comprises the amino acid sequence set forth in SEQ ID NO: 469 (or a variant thereof); (xvii) a heavy chain variable region that comprises the amino acid sequence set forth in SEQ ID NO: 493 (or a variant thereof), and a light chain variable region that comprises the amino acid sequence set forth in SEQ ID NO: 501 (or a variant thereof); (xviii) a heavy chain variable region that comprises the amino acid sequence set forth in SEQ ID NO: 509 (or a variant thereof), and a light chain variable region that comprises the amino acid sequence set forth in SEQ ID NO: 517 (or a variant thereof); (xix) a heavy chain variable region that comprises the amino acid sequence set forth in SEQ ID NO: 525 (or a variant thereof), and a light chain variable region that comprises the amino acid sequence set forth in SEQ ID NO: 533 (or a variant thereof); (xx) a heavy chain variable region that comprises the amino acid sequence set forth in SEQ ID NO: 541 (or a variant thereof), and a light chain variable region that comprises the amino acid sequence set forth in SEQ ID NO: 549 (or a variant thereof); (xxi) a heavy chain variable region that comprises the amino acid sequence set forth in SEQ ID NO: 557 (or a variant thereof), and a light chain variable region that comprises the amino acid sequence set forth in SEQ ID NO: 565 (or a variant thereof); (xxii) a heavy chain variable region that comprises the amino acid sequence set forth in SEQ ID NO: 573 (or a variant thereof), and a light chain variable region that comprises the amino acid sequence set forth in SEQ ID NO: 581 (or a variant thereof); (xxiii) a heavy chain variable region that comprises the amino acid sequence set forth in SEQ ID NO: 589 (or a variant thereof), and a light chain variable region that comprises the amino acid sequence set forth in SEQ ID NO: 597 (or a variant thereof); (xxiv) a heavy chain variable region that comprises the amino acid sequence set forth in SEQ ID NO: 605 (or a variant thereof), and a light chain variable region that comprises the amino acid sequence set forth in SEQ ID NO: 597 (or a variant thereof); (xxv) a heavy chain variable region that comprises the amino acid sequence set forth in SEQ ID NO: 613 (or a variant thereof), and a light chain variable region that comprises the amino acid sequence set forth in SEQ ID NO: 621 (or a variant thereof); (xxvi) a heavy chain variable region that comprises the amino acid sequence set forth in SEQ ID NO: 629 (or a variant thereof), and a light chain variable region that comprises the amino acid sequence set forth in SEQ ID NO: 637 (or a variant thereof); (xxvii) a heavy chain variable region that comprises the amino acid sequence set forth in SEQ ID NO: 645 (or a variant thereof), and a light chain variable region that comprises the amino acid sequence set forth in SEQ ID NO: 653 (or a variant thereof); (xxviii) a heavy chain variable region that comprises the amino acid sequence set forth in SEQ ID NO: 661 (or a variant thereof), and a light chain variable region that comprises the amino acid sequence set forth in SEQ ID NO: 669 (or a variant thereof); and/or (xxix) a heavy chain variable region that comprises the amino acid sequence set forth in SEQ ID NO: 677 (or a variant thereof), and a light chain variable region that comprises the amino acid sequence set forth in SEQ ID NO: 685 (or a variant thereof).

In some embodiments, any antigen-binding protein that binds specifically to C5 (anti-C5) which is discussed herein is an antagonist. Such an antagonist (e.g., an antagonist antigen-binding protein that binds specifically to C5) binds to C5 and inhibits at least one biological activity of C5; for example, preventing or blocking complement-mediated hemolysis by classical pathway or alternative pathway and/or inhibits cleavage of C5 to C5a and C5b and/or inhibits complement mediated lysis of red blood cells and/or inhibits formation of membrane attack complex (MAC) and/or inhibits formation of C5b-6 complex.

In some embodiments, the C5 antigen-binding protein is eculizumab (sold as Soliris), crovalimab, ravulizumab (ALXN1210; sold as Ultomiris), tesidolumab (see U.S. Pat. No. 8,241,628; WO 2010/015608; or WO 2017/212375, each of which is herein incorporated by reference in its entirety for all purposes), or mubodina (see U.S. Pat. No. 7,999,081, herein incorporated by reference in its entirety for all purposes).

In some embodiments, an antibody or antigen-binding fragment thereof that binds specifically to C5 is pozelimab (REGN3918; H4H12166P) antibody. Pozelimab (REGN3918; H4H12166P) antibody comprises a heavy chain immunoglobulin comprising the amino acid sequence:

(SEQ ID NO: 697)
QVQLQESGPGLVKPSETLSLTCTVSGDSVSSSYWTWIRQPPGKGLEWIG
YIYYSGSSNYNPSLKSRATISVDTSKNQFSLKLSSVTAADTAVYYCARE
GNVDTTMIFDYWGQGTLVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK

and a light chain comprising the amino acid sequence:

(SEQ ID NO: 698)
AIQMTQSPSSLSASVGDRVTITCRASQGIRNDLGWYQQKPGKAPKLLIY
AASSLQSGVPSRFAGRGSGTDFTLTISSLQPEDFATYYCLQDFNYPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the C5 antigen-binding protein comprises a heavy chain immunoglobulin comprising the amino acid sequence: QVQLVESGGGLVQPGRSLRLSCAASGFTVHSSYYMAWVRQAPGKGLEWVGAIFTGSG AEYKAEWAKGRVTISKDTSKNQVVLTMTNMDPVDTATYYCASDAGYDYPTHAMHYW GQGTLVTVSSASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKKVEPKSCDKTHTC PPCPAPELRRGPKVFLFPPKPKDTLMISRTPEVTCVVVDVSHEDPEVKFNWYVDGVEVH NAKTKPREEQYNSTYRVVSVLTVLHQDWLNGKEYKCKVSNKGLPSSIEKTISKAKGQP REPQVYTLPPSREEMTKNQVSLTCLVKGFYPSDIAVEWESNGQPENNYKTTPPVLDSDG SFFLYSKLTVDKSRWQQGNVFSCSVLHEALHAHYTRKELSLSP (SEQ ID NO: 702) or the HCDR1, HCDR2 and HCDR3 thereof, or the V_{H} thereof (or a variant thereof); and a light chain immunoglobulin comprising the amino acid sequence: DIQMTQSPSSLSASVGDRVTITCRASQGISSSLAWYQQKPGKAPKLLIYGASETESGVPSR FSGSGSGTDFTLTISSLQPEDFATYYCQNTKVGSSYGNTFGGGTKVEIKRTVAAPSVFIFP PSDEQLKSGTASVVCLLNNFYPREAKVQWKVDNALQSGNSQESVTEQDSKDSTYSLSST LTLSKADYEKHKVYACEVTHQGLSSPVTKSFNRGEC (SEQ ID NO: 703) or the LCDR1, LCDR2 and LCDR3 thereof, or the V_{L} thereof (or a variant thereof).

In some embodiments, human C5 (including the signal sequence) comprises the amino acid sequence set forth in SEQ ID NO: 704; and mature human C5 comprising the mutation R885H comprises the amino acid sequence set forth in SEQ ID NO: 705.

“H2M11683N”; “H2M11686N”; “H4H12159P”; “H4H12161P”; “H4H12163P”; “H4H12164P”; “H4H12166P”; “H4H12166P2”; “H4H12166P3”; “H4H12166P4”; “H4H12166P5”; “H4H12166P6”; “H4H12166P7”; “H4H12166P8”; “H4H12166P9”; “H4H12166P10”; “H4H12167P”; “H4H12168P”; “H4H12169P”; “H4H12170P”; “H4H12171P”; “H4H12175P”; “H4H12176P2”; “H4H12177P2”; “H4H12183P2”; “H2M11682N”; “H2M11684N”; “H2M11694N” or “H2M11695N”, unless otherwise stated, refer to C5 antigen-binding proteins, e.g., antibodies and antigen-binding fragments thereof (including multispecific antigen-binding proteins), that bind specifically to C5, comprising an immunoglobulin heavy chain or variable region thereof (V_{H}) comprising the amino acid sequence specifically set forth herein corresponding, in Table 5 herein or Table 1 of WO 2017/218515 (and the sequences set forth therein), herein incorporated by reference in its entirety for all purposes, to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 341; 357; 373; 389; 405; 421; 437; 461; 477; 485; 493; 509; 525; 541; 557; 573; 589; 605; 613; 629; 645; 661; or 677) (or a variant thereof), and/or an immunoglobulin light chain or variable region thereof (V_{L}) comprising the amino acid sequence specifically set forth herein corresponding, in Table 5 herein or Table 1 of WO 2017/218515 (and the sequences set forth therein), herein incorporated by reference in its entirety for all purposes, to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 349; 365; 381; 397; 413; 429; 445; 453; 469; 501; 517; 533; 549; 565; 581; 597; 621; 637; 653; 669; or 685) (or a variant thereof); and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In some embodiments, the V_{H} is linked to an IgG constant heavy chain domain (e.g., IgG1 or IgG4 (e.g., IgG4 (S228P mutant))) and/or the V_{L} is linked to a lambda or kappa constant light chain domain.

A C5 antigen-binding protein (i.e., anti-C5 antigen-binding protein) or C5 antibody or antigen-binding fragment or antibody or antigen-binding fragment that “binds specifically” to C5 binds to human C5 with a K_{D} of at least 1 nM (i.e., 1 nM or a higher affinity), e.g., about 0.1 or 0.2 nM.

The C5 antigen-binding proteins and antibodies (i.e., “anti-C5” antigen-binding proteins and antibodies) can be in pharmaceutical formulations, such as aqueous formulations. The pharmaceutical formulation can be, for example, any of those disclosed in WO 2021/034639 A1, US 2021-0046182, WO 2021/081277 A1, US 2021-0139573, WO 2017/218515 A1, US 2020-0262901, US 2017-0355757, or US 2020-0262900, each of which is herein incorporated by reference in its entirety for all purposes. In some embodiments, they can be in combination or co-formulation with CRISPR/Cas systems targeting a C5 locus or gene as disclosed herein. In some embodiments, they can be in combination with but not in a co-formulation with CRISPR/Cas systems targeting a C5 locus or gene as disclosed herein. Such pharmaceutical formulations or co-formulations, as used herein, refer to formulations comprising a C5 antigen-binding protein or antibody and a pharmaceutically acceptable carrier. A pharmaceutically acceptable carrier includes, for example, one or more excipients. In some embodiments, a pharmaceutical formulation or a co-formulation is aqueous, i.e., includes water.

Pharmaceutical formulations including C5 antigen-binding proteins may be prepared by admixing the antigen-binding protein with one or more excipients (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). Pharmaceutical formulations may include, for example, a viscosity reducer, a stabilizer, a non-ionic surfactant, a buffer, or any combination thereof.

Various viscosity reducers are known in the art for use with antibody containing formulations. Such viscosity reducers include an amino acid (e.g., D- or L-isomer), such as, for example, (D- or L-) arginine (e.g., L-arginine, e.g., L-arginine HCl or D-arginine), (D- or L-) alanine, proline, (D- or L-) valine, glycine, (D- or L-) serine, (D- or L-) phenylalanine, (D- or L-) lysine, and (D- or L-) glutamate, and salts thereof (e.g., Na or HCl salts); an inorganic salt (e.g., NaCl or MgCl₂), pyridoxamine; L-Ornithine; thiamine phosphoric acid ester chloride dihydrate, benzenesulfonic acid and/or pyridoxine. In some embodiments, the amino acid is an L-amino acid such as L-arginine.

Stabilizers include agents, such as sugars or polyols, that aid in the reduction of antibody or antigen-binding fragment degradation, e.g., aggregation. Polyols are sugar alcohols having multiple hydroxyl groups. Stabilizers include a sugar or polyol, e.g., trehalose, sorbitol, mannitol, taurine, propane sulfonic acid, L-proline, sucrose, glycerol, threitol, maltitol, and/or polyethylene glycol (PEG; such as PEG3350).

Non-ionic surfactants contain molecules with head groups that are uncharged. Non-ionic surfactants include a non-ionic surfactant including a polyoxyethylene moiety; a sorbitan; a polyoxyethylene glycol alkyl ether, such as octaethylene glycol monododecyl ether; pentaethylene glycol monododecyl ether; polyoxypropylene glycol alkyl ether; glucoside alkyl ether, such as decyl glucoside, lauryl glucoside, octyl glucoside; polyoxyethylene glycol octylphenol ether, such as triton X-100; polyoxyethylene glycol alkylphenol ether, such as nonoxynol-9; glycerol alkyl ester, such as glyceryl laurate; polyoxyethylene glycol sorbitan alkyl ester, such as polysorbate; sorbitan alkyl ester, such as spans; cocamide MEA, cocamide DEA, dodecyldimethylamine oxide; block copolymer of polyethylene glycol and polypropylene glycol, such as poloxamer; and polyethoxylated tallow amine (POEA); poloxamer 188, polyethylene glycol 3350, a polyethylene glycol (e.g., PEG3350) or a polysorbate such as polysorbate 80 (PS80) or polysorbate 20 (PS20). In some embodiments, the non-ionic detergent is polysorbate-20 (PS20), polysorbate-80 (PS80).

A buffer is an aqueous solution consisting of a mixture of a weak acid and its conjugate base or vice versa which resists changes in its pH and therefore keeps the pH at a nearly constant value. Various buffers may be used in the pharmaceutical formulations or co-formulations, for example, histidine-based buffer, phosphate buffer or citrate buffer. A histidine-based buffer is a buffer comprising histidine. Examples of histidine buffers include histidine chloride, histidine hydrochloride, histidine acetate, histidine phosphate, and histidine sulphate.

In some embodiments, the C5 antigen-binding protein or C5 antibody can be in a pharmaceutical formulation that comprises high concentrations (at least 150 mg/mL or at least 200 mg/mL) of C5 antigen-binding protein or C5 antibody having a low viscosity (e.g., less than about 15 cP, e.g., about 14 or 14.3). For example, such pharmaceutical formulations can comprise, consist of, or consist essentially of: 200 mg/mL pozelimab; 20 mM histidine buffer; 100 mM L-arginine hydrochloride; 2% (w/v) sucrose; 0.15% (w/v) polysorbate-80; and water, pH 5.8.

In some embodiments, the pharmaceutical formulation can comprise C5 antigen-binding protein (e.g., ≥150 mg/mL, ≥200 mg/mL, ≥250 mg/mL, ≥274 mg/ml or ≥275 mg/mL of anti-C5 antigen-binding protein); buffer (e.g., about 20 mM); an amino acid (e.g., about 100 mM); an optional sugar (e.g., about 2%); an optional non-ionic detergent (e.g., about 0.15%); and water; pH about 5-6 (e.g., about pH. 5.8).

In some embodiments, the pharmaceutical formulation can comprise about 150 or 200 mg/mL or more C5 antigen-binding protein and a pharmaceutically acceptable carrier comprising: buffer (e.g., phosphate buffer, acetate buffer, citrate buffer, histidine buffer or imidazole buffer); arginine (for example, L-arginine HCl, e.g., 50-100 mM, e.g., 100 mM); water; and, optionally, an oligosaccharide (for example, sucrose, mannitol, dextrose, glycerol, TMAO (trimethylamine N-oxide), trehalose, ethylene glycol, glycine betaine, xylitol or sorbitol, e.g., 2%); and optionally, a non-ionic detergent (e.g., a polyoxyethylene-based detergent or a glycosidic compound-based detergent, polysorbate-20, polysorbate-80 or tween-20), pH of up to about 6.1, e.g., 5-6, e.g., 5.8; and a viscosity of about 14, 14.3 or 15 cP (20° C.) or less.

In some embodiments, the pharmaceutical formulation is aqueous (e.g., suitable for intravenous and/or subcutaneous administration) and comprises a C5 antigen-binding protein or antibody or antigen-binding fragment thereof (e.g., pozelimab) (e.g., about 200 mg/mL or about 180-210 mg/mL), histidine (e.g., histidine-HCl; e.g., about 20 mM or 20 mM±4 mM), pH about 5.8 or 5.8±0.3, arginine (e.g., about 100 mM or 100 mM±20 mM; e.g., L-arginine, L-arginine HCl or L-arginine monohydrochloride), a polyol such as sucrose (e.g., about 2% or 2%±0.4% (w/v)), and a non-ionic surfactant such as polysorbate (e.g., polysorbate 80; e.g., about 0.15% or 0.15% 0.075% (w/v). For example, the pharmaceutical formulation can comprise 200 mg/mL pozelimab, 20 mM histidine buffer, 100 mM L-arginine hydrochloride, 2% (w/v) sucrose, 0.15% (w/v) polysorbate-80, and water, pH 5.8.

“Arginine” or “L-arginine” includes any pharmaceutically acceptable salt form thereof, e.g., L-arginine hydrochloride.

Buffers control the pH of formulations and in some cases contribute to the overall stability of a protein product. In some embodiments, the buffer is a phosphate buffer, acetate buffer, citrate buffer, histidine buffer or imidazole buffer.

An amino acid can be any one of the 20 essential amino acids. In some embodiments, the amino acid is glycine, arginine, aspartic acid, glutamic acid, lysine, asparagine, glutamine, proline, or histidine.

In some embodiments, the oligosaccharide is sucrose, mannitol, dextrose, glycerol, TMAO (trimethylamine N-oxide), trehalose, ethylene glycol, glycine betaine, xylitol or sorbitol.

Non-ionic detergents contain molecules with head groups that are uncharged. In an embodiment of the invention, the non-ionic detergent is polyoxyethylene-based or glycosidic compound-based. In some embodiments, the non-ionic detergent is polysorbate-20 (PS20), polysorbate-80 (PS80) or tween-20.

In some embodiments, the pharmaceutical formulation is one described in WO 2021/034639 A1 or US 2021-0046182, each of which is herein incorporated by reference in its entirety for all purposes.

### IV. Combinations

Also disclosed herein are combinations of CRISPR/Cas systems targeting a C5 locus in combination with or in association with C5 antigen-binding proteins and/or other therapeutic agents. CRISPR/Cas systems targeting a C5 locus and C5 antigen-binding proteins are described in detail elsewhere herein. As used herein, the term “in combination with” means that additional therapeutically active component(s) may be administered prior to, concurrent with, or after the administration of the CRISPR/Cas system. The term “in combination with” also includes sequential or concomitant administration of a CRISPR/Cas system and a second therapeutic agent.

The CRISPR/Cas systems disclosed herein may be combined synergistically with one or more drugs or therapy used to treat a disease or disorder associated with C5. In some embodiments, the CRISPR/Cas systems described herein may be combined with a second therapeutic agent to ameliorate one or more symptoms of the disease.

Depending upon the C5-associated disease or disorder, the CRISPR/Cas systems may be used in combination with one or more additional therapeutic agents including, but not limited to, a C5 antigen-binding protein, an anti-coagulant (e.g., warfarin, aspirin, heparin, phenindione, fondaparinux, idraparinux, and thrombin inhibitors such as argatroban, lepirudin, bivalirudin, or dabigatran), an anti-inflammatory drug (e.g., corticosteroids, and non-steroidal anti-inflammatory drugs (NSAIDs)), an antihypertensive (e.g., an angiotensin-converting enzyme inhibitor), an immunosuppressive agent (e.g., vincristine, cyclosporine A, or methotrexate), a fibrinolytic agent (e.g., ancrod, E-aminocaproic acid, antiplasmin-a1, prostacyclin, and defibrotide), a lipid-lowering agent such as an inhibitor of hydroxymethylglutaryl CoA reductase, an anti-CD20 agent such as rituximab, an anti-TNFα agent such as infliximab, an anti-seizure agent (e.g., magnesium sulfate), a C3 inhibitor, or an anti-thrombotic agent. In some embodiments, the further therapeutic agent is acetaminophen, albumin (e.g., in the form of an infusion), ancrod, an angiotensin-converting enzyme inhibitor, an antibiotic (e.g. an oral antibiotic), a further antibody, an anti-CD20 agent, rituximab, an anti-coagulant, an anti-fungal agent, an antihypertensive, an anti-inflammatory drug, antiplasmin-a1, an anti-seizure agent, anti-thrombotic agent, an anti-TNFα agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a meningococcal vaccine (e.g., serotypes A, C, Y, W and serotype B), methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin, and/or warfarin.

In certain embodiments, the second therapeutic agent is a C5 antigen-binding protein (e.g., a C5 antibody) or a combination of C5 antigen-binding proteins. It is contemplated herein to use a combination (“cocktail”) of antibodies with broad neutralization or inhibitory activity against C5. In some embodiments, non-competing antibodies may be combined and administered to a subject in need thereof. In some embodiments, the antibodies comprising the combination bind to distinct non-overlapping epitopes on the protein. The antibodies comprising the combination may block the C5 binding to C5 convertase and/or may prevent/inhibit cleavage of C5 into C5a and C5b. In certain embodiments, a second antibody may possess longer half-life in human serum.

The additional therapeutically active component(s) may be administered to a subject prior to administration of a CRISPR/Cas system targeting a C5 locus or gene. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of a CRISPR/Cas system targeting a C5 locus or gene. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of a CRISPR/Cas system targeting a C5 locus or gene. “Concurrent” administration includes, e.g., administration of a CRISPR/Cas system targeting a C5 locus or gene and an additional therapeutically active component to a subject in a single dosage form, or in separate dosage forms administered to the subject within about 30 minutes or less of each other. If administered in separate dosage forms, each dosage form may be administered via the same route (e.g., both the CRISPR/Cas system and the additional therapeutically active component (e.g., anti-C5 antibody) may be administered intravenously, etc.); alternatively, each dosage form may be administered via a different route (e.g., a CRISPR/Cas system targeting a C5 locus or gene may be administered intravenously, and the additional therapeutically active component may be administered subcutaneously). In any event, administering the components in a single dosage from, in separate dosage forms by the same route, or in separate dosage forms by different routes are all considered “concurrent administration,” for purposes of the present disclosure. For purposes of the present disclosure, administration of a CRISPR/Cas system targeting a C5 locus or gene “prior to,” “concurrent with,” or “after” (as those terms are defined herein above) administration of an additional therapeutically active component is considered administration of a CRISPR/Cas system targeting a C5 locus or gene “in combination with” an additional therapeutically active component.

In some embodiments, pharmaceutical compositions in which a CRISPR/Cas system is co-formulated with one or more of the additional therapeutically active component(s) as described elsewhere herein (e.g., an C5 antigen-binding protein or antibody) is used. In some embodiments, pharmaceutical compositions comprising a CRISPR/Cas system are used in combination with but not in a co-formulation with one or more of the additional therapeutically active component(s) as described elsewhere herein (e.g., an C5 antigen-binding protein or antibody). For example, provided are pharmaceutical formulations that include a CRISPR/Cas system targeting a C5 locus or gene in association with one or more further therapeutic agents (e.g., a C5 antigen-binding protein disclosed herein). The term “in association with” indicates that components of a pharmaceutical formulation, (1) a CRISPR/Cas system and pharmaceutically acceptable carrier components, along with (2) one or more further therapeutic agents, such as a C5 antigen-binding protein or antibody, can be formulated into a single composition, e.g., for simultaneous delivery, or formulated separately into two or more compositions (e.g., a kit including each component, for example, wherein the further therapeutic agent is in a separate formulation). Components administered in association with each another can be administered to a subject at the same time or at a different time than when the other component is administered; for example, each administration may be given simultaneously (e.g., together in a single composition or essentially simultaneously during the same administration session) or non-simultaneously at one or more intervals over a given period of time. Moreover, the separate components administered in association with each another may be administered to a subject by the same or by a different route.

### V. Methods for Targeting C5

Also disclosed herein are methods of modifying or targeting or knocking down or knocking out a C5 locus or gene using the CRISPR/Cas systems described herein, as well as use of the CRISPR/Cas systems in prophylactic and therapeutic applications for treatment and/or prevention of a disease, disorder, or condition associated with C5 and/or for ameliorating at least one symptom associated with such disease, disorder, or condition either alone or in combination with other therapeutic agents such as the C5 antigen-binding proteins or antibodies disclosed herein.

### A. Methods of Modifying or Knocking Out a C5 Locus

Various methods are provided for modifying or inactivating (e.g., knocking down or knocking out) a C5 gene or locus (e.g., endogenous C5 gene or C5 genomic locus) using the CRISPR/Cas reagents (e.g., Cas protein or a nucleic acid encoding and one or more C5-targeting gRNAs or DNAs encoding) described elsewhere herein to generate a targeted genetic modification in the C5 gene or locus (e.g., a target genomic locus within the C5 gene or locus). Optionally, an exogenous donor nucleic acid (e.g., targeting vector) targeting the C5 gene or locus can be used together with the CRISPR/Cas reagents. Alternatively, the CRISPR/Cas reagents can be used without any exogenous donor nucleic acid. Knockdown refers to a decrease in expression of a C5 gene product (e.g., C5 protein, C5 mRNA, or both). Knockdown of a C5 protein can be measured by detecting total cellular amount of the protein from a tissue or cell population of interest or in the serum. Methods for measuring knockdown of C5 mRNA are known and include sequencing of mRNA isolated from a tissue or cell population of interest. Knockdown may refer to some loss of expression of a C5 gene product, for example a decrease in the amount of C5 mRNA transcribed or a decrease in the amount of C5 protein expressed by a population of cells (including in vivo populations such as those found in tissues). Knockout refers to a loss of expression of a C5 protein in a cell. Knockout can be measured either by detecting total cellular amount of a C5 protein in a cell, a tissue, or a population of cells or in the serum. The methods described herein can knock out C5 in one or more cells (e.g., in a population of cells including in vivo populations such as those found in tissues). In some methods, a knockout is not the formation of mutant C5 protein (e.g., created by indels), but rather the complete loss of expression of C5 protein in a cell. Such methods can also be in combination with administering a C5 antigen-binding protein or C5 antibody. For example, any of the C5 antigen-binding proteins or C5 antibodies disclosed herein can be used.

The C5 gene or locus can be in an animal or cell, and the methods can occur in vitro, ex vivo, or in vivo. Animals include mammals, fishes, and birds. A mammal can be, for example, a non-human mammal, a human, a rodent, a rat, a mouse, or a hamster. In one example, the animal is a human. Other non-human mammals include, for example, non-human primates (e.g., cynomolgus), monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The animals in the methods disclosed herein can be humans or they can be non-human animals. The term “non-human” excludes humans. Particular examples of non-human animals include rodents, such as mice and rats, or non-human primates, such as cynomolgus.

Cells used in the methods can be from any type of animal, and they can be any type of undifferentiated or differentiated state. The cells can be in vitro, ex vivo, or in vivo. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types.

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, kidney cells, hematopoietic cells, endothelial cells, epithelial cells, fibroblasts, mesenchymal cells, keratinocytes, blood cells, melanocytes, monocytes, mononuclear cells, monocytic precursors, B cells, erythroid-megakaryocytic cells, eosinophils, macrophages, T cells, islet beta cells, exocrine cells, pancreatic progenitors, endocrine progenitors, adipocytes, preadipocytes, neurons, glial cells, neural stem cells, neurons, hepatoblasts, hepatocytes, cardiomyocytes, skeletal myoblasts, smooth muscle cells, ductal cells, acinar cells, alpha cells, beta cells, delta cells, PP cells, cholangiocytes, white or brown adipocytes, or ocular cells (e.g., trabecular meshwork cells, retinal pigment epithelial cells, retinal microvascular endothelial cells, retinal pericyte cells, conjunctival epithelial cells, conjunctival fibroblasts, iris pigment epithelial cells, keratocytes, lens epithelial cells, non-pigment ciliary epithelial cells, ocular choroid fibroblasts, photoreceptor cells, ganglion cells, bipolar cells, horizontal cells, or amacrine cells). For example, the cells can be liver cells, such as hepatoblasts or hepatocytes. The cells provided herein can be normal, healthy cells, or can be diseased or mutant-bearing cells.

Non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. In some cases, suitable rats can be from an inbred rat strain. See, e.g., US 2014/0235933, herein incorporated by reference in its entirety for all purposes.

The CRISPR/Cas reagents (and optionally exogenous donor nucleic acid) can be introduced into a cell or an animal in any form and by any means as described elsewhere herein, and all or some can be introduced simultaneously or sequentially in any combination as described elsewhere herein. Likewise, any additional agents (e.g., C5 antigen-binding proteins) can be introduced in any form and by any means as described elsewhere herein.

In some methods, one guide RNA is used. In other methods, one or more additional guide RNAs that target additional guide RNA target sequences within the C5 gene or locus can be used. By using one or more additional guide RNAs (e.g., a second guide RNA that target a second guide RNA target sequence), cleavage by the Cas protein can create two or more double-strand breaks or two or more single-strand breaks (e.g., if the Cas protein is a nickase). In some methods, two or more guide RNAs targeting a guide RNA target sequence in the C5 gene or locus can be used (e.g., two guide RNAs can be used). In some methods, three or more guide RNAs targeting a guide RNA target sequence in the C5 gene or locus can be used (e.g., three guide RNAs can be used). In some methods, four or more guide RNAs targeting a guide RNA target sequence in the C5 gene or locus can be used (e.g., four guide RNAs can be used).

Optionally, one or more exogenous donor sequences which recombine with a target genomic locus in the C5 gene or locus can be used together with the CRISPR/Cas reagents to generate a targeted genetic modification. Examples and variations of exogenous donor sequences that can be used in the methods are disclosed elsewhere herein.

Targeted genetic modifications in a C5 gene or locus in a genome can be generated by contacting the genome with a complex comprising a Cas protein (e.g., a Cas9 protein) and one or more guide RNAs, each targeting a different guide RNA target sequence in the C5 gene, such that Cas protein creates one or more nicks or double-strand breaks at the guide RNA target sequence. Optionally, the genome can be further contacted with one or more exogenous donor nucleic acids. For example, targeted genetic modifications to a CS gene in a genome can be generated by introducing into a cell or an animal a Cas protein (or a nucleic acid encoding the Cas protein, such as an mRNA or a DNA) and one or more guide RNAs (or DNAs encoding the one or more guide RNAs), each targeting a different guide RNA target sequence in the CS gene, such that Cas protein creates one or more nicks or double-strand breaks at the guide RNA target sequence. Optionally, one or more exogenous donor nucleic acids can also be introduced into the cell. The Cas protein forms a different complex with each guide RNA, and the Cas protein cleaves the guide RNA target sequence. An exogenous donor nucleic acid, if used, can recombine with the target genomic locus. Cleavage by the Cas protein can create a double-strand break or a single-strand break (e.g., if the Cas protein is a nickase). Examples and variations of Cas proteins and guide RNAs that can be used in the methods are described elsewhere herein.

A guide RNA can be introduced into an animal or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the animal. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into an animal or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or an animal, the Cas protein can be transiently, conditionally, or constitutively expressed in the cell or in a cell in the animal.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle).

Guide RNAs can be modified as disclosed elsewhere herein. Likewise, Cas mRNAs can be modified as disclosed elsewhere herein.

A guide RNA targeting a C5 gene can target any desired location in the C5 gene. Guide RNAs targeting a C5 gene, such as a human C5 gene, can target any contiguous sequence in the C5 gene. The term C5 gene includes the genomic region encompassing the C5 regulatory promoters and enhancer sequences as well as the C5 coding sequence. Guide RNAs targeting a C5 gene can target a coding sequence, a non-coding sequence (e.g., a regulatory element such as a promoter or enhancer region), or a combination thereof. As one example, a guide RNA targeting a C5 gene can target a contiguous coding sequence in any of the coding exons. In one example, the guide RNA target sequence is not in coding exon 16 and 17, as this region encodes anaphylatoxin C5a. As one example, a guide RNA targeting a C5 gene can target coding exon 1 of the C5 gene. As another example, a guide RNA targeting a C5 gene can target coding exon 2 of the C5 gene. As another example, a guide RNA targeting a C5 gene can target coding exon 3 of the C5 gene. As another example, a guide RNA targeting a C5 gene can target coding exon 4 of the C5 gene. As another example, a guide RNA targeting a C5 gene can target coding exon 5 of the C5 gene. As another example, a guide RNA targeting a C5 gene can target coding exon 6 of the C5 gene. As another example, a guide RNA targeting a C5 gene can target coding exon 7 of the C5 gene. As another example, a guide RNA targeting a C5 gene can target coding exon 8 of the C5 gene. As another example, a guide RNA targeting a C5 gene can target coding exon 9 of the C5 gene. As another example, a guide RNA targeting a C5 gene can target coding exon 10 of the C5 gene. As another example, a guide RNA targeting a C5 gene can target coding exon 11 of the C5 gene. As another example, a guide RNA targeting a C5 gene can target coding exon 12 of the C5 gene. As another example, a guide RNA targeting a C5 gene can target coding exon 13 of the C5 gene. As another example, a guide RNA targeting a C5 gene can target coding exon 14 of the C5 gene. As another example, a guide RNA targeting a C5 gene can target coding exon 15 of the C5 gene. As another example, a guide RNA targeting a C5 gene can target coding exon 16 of the C5 gene. As another example, a guide RNA targeting a C5 gene can target coding exon 17 of the C5 gene. As another example, a guide RNA targeting a C5 gene can target coding exon 18 of the C5 gene. As another example, a guide RNA targeting a C5 gene can target coding exon 19 of the C5 gene. As another example, a guide RNA targeting a C5 gene can target coding exon 20 of the C5 gene. As another example, a guide RNA targeting a C5 gene can target coding exon 21 of the C5 gene. As another example, a guide RNA targeting a C5 gene can target coding exon 22 of the C5 gene. As another example, a guide RNA targeting a C5 gene can target coding exon 23 of the C5 gene. As another example, a guide RNA targeting a C5 gene can target coding exon 24 of the C5 gene. As another example, a guide RNA targeting a C5 gene can target coding exon 25 of the C5 gene. As another example, a guide RNA targeting a C5 gene can target coding exon 26 of the C5 gene. As another example, a guide RNA targeting a C5 gene can target coding exon 27 of the C5 gene. As another example, a guide RNA targeting a C5 gene can target coding exon 28 of the C5 gene. As another example, a guide RNA targeting a C5 gene can target coding exon 29 of the C5 gene. As another example, a guide RNA targeting a C5 gene can target coding exon 30 of the C5 gene. As another example, a guide RNA targeting a C5 gene can target coding exon 31 of the C5 gene. As another example, a guide RNA targeting a C5 gene can target coding exon 32 of the C5 gene. As another example, a guide RNA targeting a C5 gene can target coding exon 33 of the C5 gene. As another example, a guide RNA targeting a C5 gene can target coding exon 34 of the C5 gene. As another example, a guide RNA targeting a C5 gene can target coding exon 35 of the C5 gene. As another example, a guide RNA targeting a C5 gene can target coding exon 36 of the C5 gene. As another example, a guide RNA targeting a C5 gene can target coding exon 37 of the C5 gene. As another example, a guide RNA targeting a C5 gene can target coding exon 38 of the C5 gene. As another example, a guide RNA targeting a C5 gene can target coding exon 39 of the C5 gene. As another example, a guide RNA targeting a C5 gene can target coding exon 40 of the C5 gene. As another example, a guide RNA targeting a C5 gene can target coding exon 41 of the C5 gene. A guide RNA target sequence is in coding exon X if at least a portion of the guide RNA target sequence (e.g., at least one nucleotide) is in coding exon X.

In a specific example, a guide RNA targeting a C5 gene can target coding exon 1, 12, 15, 21, 22, or 27. In another specific example, a guide RNA targeting a C5 gene can target coding exon 12 or 15.

A guide RNA can be designed to target a coding region of the C5 gene so that cleavage by the corresponding Cas protein will result in frameshift insertion/deletion (indel) mutations that result in a loss-of-function allele. Such frameshift mutations can be achieved through targeted DNA double strand breaks and subsequent mutagenic repair via the non-homologous end joining (NHEJ) pathway, which produces indels at the site of break. The indel being introduced into the DSB is random, with some indels leading to frameshift mutations that cause premature termination of the C5 gene. As another example, a guide RNA can be designed to target a promoter region or enhancer region of the C5 gene so that cleavage by the corresponding Cas protein will result in disruption of the promoter region or enhancer region. Such mutations can be achieved through targeted DNA double strand breaks and subsequent mutagenic repair via the non-homologous end joining (NHEJ) pathway, which produces indels at the site of break.

A guide RNA targeting a C5 gene can target a constitutive exon of the C5 gene. For example, a guide RNA targeting a C5 gene can target a 5′ constitutive exon. Constitutive exons are coding exons that are consistently conserved after splicing. Exons expressed across all relevant tissues can be considered constitutive exons for guide RNA targeting. In some examples, a guide RNA targeting a C5 gene does not target any exon containing an alternative splicing site. Because there is only a single C5 coding transcript, all 41 coding exons of C5 are considered constitutive.

As another example, the guide RNA target sequence can be within about 10, about 20, about 30, about 40, about 50, about 100, about 200, about 300, about 400, about 500, or about 1,000 nucleotides of the start codon or can comprise the start codon.

Guide RNA target sequences can also be selected to minimize off-target modification or avoid off-target effects (e.g., by avoiding two or fewer mismatches to off-target genomic sequences).

In methods in which two guide RNAs are used, each can target the same region of the C5 gene or locus or different regions of the C5 gene or locus. For example, each guide RNA can target a different guide RNA target sequence within about 10, about 20, about 30, about 40, about 50, about 100, about 200, about 300, about 400, about 500, or about 1,000 nucleotides of the start codon or comprising the start codon (e.g., such that the start codon is disrupted following cleavage by the Cas protein). Alternatively or additionally, each guide RNA can target a different contiguous coding sequence. For example, each guide RNA can target a different guide RNA target sequence in a constitutive exon of the C5 gene or a 5′ constitutive exon of the C5 gene. As another example, each guide RNA can target a different guide RNA target sequence in a coding exon selected from coding exons 1-41, coding exons 1, 12, 15, 21, 22, and 27, or coding exons 12 and 15. As another example, each guide RNA can be designed to target a different guide RNA target sequence in a promoter region or enhancer region of the C5 gene. As another example, at least one guide RNA can target a contiguous coding sequence of the C5 gene and at least one guide RNA can target a guide RNA target sequence in a promoter region or enhancer region of the C5 gene. As another example, at least one guide RNA can target a guide RNA target sequence within about 10, about 20, about 30, about 40, about 50, about 100, about 200, about 300, about 400, about 500, or about 1,000 nucleotides of the start codon or comprising the start codon, and at least one guide RNA can target a guide RNA target sequence within about 10, about 20, about 30, about 40, about 50, about 100, about 200, about 300, about 400, about 500, or about 1,000 nucleotides of the stop codon or comprising the stop codon (e.g., wherein cleavage by the Cas protein at both guide RNA target sequences can result in deletion of the coding region between the two guide RNA target sequences).

In methods in which one or more exogenous donor nucleic acids are used to modify the C5 gene or locus following cleavage by the Cas protein, the Cas protein can cleave the target genomic locus to create a single-strand break (nick) or double-strand break, and the cleaved or nicked locus can be repaired by the exogenous donor nucleic acid via non-homologous end joining (NHEJ)-mediated insertion or homology-directed repair. Optionally, repair with the exogenous donor nucleic acid removes or disrupts the guide RNA target sequence(s) so that alleles that have been targeted cannot be re-targeted by the CRISPR/Cas reagents.

The exogenous donor nucleic acid can target any sequence in C5 gene or locus. Some exogenous donor nucleic acids comprise homology arms. Other exogenous donor nucleic acids do not comprise homology arms. The exogenous donor nucleic acids can be capable of insertion into a C5 gene or locus by homology-directed repair, and/or they can be capable of insertion into a C5 gene or locus by non-homologous end joining.

Exogenous donor nucleic acids can comprise deoxyribonucleic acid (DNA) or ribonucleic acid (RNA), they can be single-stranded or double-stranded, and they can be in linear or circular form. For example, an exogenous donor nucleic acid can be a single-stranded oligodeoxynucleotide (ssODN). See, e.g., Yoshimi et al. (2016) Nat. Commun. 7:10431, herein incorporated by reference in its entirety for all purposes. Exogenous donor nucleic acids can be naked nucleic acids or can be delivered by viruses, such as AAV. In a specific example, the exogenous donor nucleic acid can be delivered via AAV and can be capable of insertion into a the C5 gene or locus by non-homologous end joining (e.g., the exogenous donor nucleic acid can be one that does not comprise homology arms).

An exemplary exogenous donor nucleic acid is between about 50 nucleotides to about 5 kb in length or between about 50 nucleotides to about 3 kb in length. Alternatively, an exogenous donor nucleic acid can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, an exogenous donor nucleic acid can be, for example, no more than 5 kb, 4.5 kb, 4 kb, 3.5 kb, 3 kb, or 2.5 kb in length.

In one example, an exogenous donor nucleic acid is an ssODN that is between about 80 nucleotides and about 3 kb in length. Such an ssODN can have homology arms or short single-stranded regions at the 5′ end and/or the 3′ end that are complementary to one or more overhangs created by Cas-agent-mediated cleavage at the target genomic locus, for example, that are each between about 40 nucleotides and about 60 nucleotides in length. Such an ssODN can also have homology arms or complementary regions, for example, that are each between about 30 nucleotides and 100 nucleotides in length. The homology arms or complementary regions can be symmetrical (e.g., each 40 nucleotides or each 60 nucleotides in length), or they can be asymmetrical (e.g., one homology arm or complementary region that is 36 nucleotides in length, and one homology arm or complementary region that is 91 nucleotides in length).

Exogenous donor nucleic acids can include modifications or sequences that provide for additional desirable features (e.g., modified or regulated stability; tracking or detecting with a fluorescent label; a binding site for a protein or protein complex; and so forth). Exogenous donor nucleic acids can comprise one or more fluorescent labels, purification tags, epitope tags, or a combination thereof. For example, an exogenous donor nucleic acid can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). Such fluorescent labels (e.g., internal fluorescent labels) can be used, for example, to detect an exogenous donor nucleic acid that has been directly integrated into a cleaved target nucleic acid having protruding ends compatible with the ends of the exogenous donor nucleic acid. The label or tag can be at the 5′ end, the 3′ end, or internally within the exogenous donor nucleic acid. For example, an exogenous donor nucleic acid can be conjugated at 5′ end with the IR700 fluorophore from Integrated DNA Technologies (5′IRDYE©700).

The exogenous donor nucleic acids disclosed herein also comprise nucleic acid inserts including segments of DNA to be integrated at target genomic loci. Integration of a nucleic acid insert at a target genomic locus can result in addition of a nucleic acid sequence of interest to the target genomic locus or replacement of a nucleic acid sequence of interest at the target genomic locus (i.e., deletion and insertion). Some exogenous donor nucleic acids are designed for deletion of a nucleic acid sequence at a target genomic locus without any corresponding insertion at the target genomic locus. Some exogenous donor nucleic acids are designed for insertion of a nucleic acid insert at a target genomic locus without any corresponding deletion at the target genomic locus. Other exogenous donor nucleic acids are designed to delete a nucleic acid sequence of interest at a target genomic locus and replace it with a nucleic acid insert.

The nucleic acid insert or the corresponding nucleic acid at the target genomic locus being deleted and/or replaced can be various lengths. An exemplary nucleic acid insert or corresponding nucleic acid at the target genomic locus being deleted and/or replaced is between about 1 nucleotide to about 5 kb in length or is between about 1 nucleotide to about 3 kb nucleotides in length. For example, a nucleic acid insert or a corresponding nucleic acid at the target genomic locus being deleted and/or replaced can be between about 1 to about 100, about 100 to about 200, about 200 to about 300, about 300 to about 400, about 400 to about 500, about 500 to about 600, about 600 to about 700, about 700 to about 800, about 800 to about 900, or about 900 to about 1,000 nucleotides in length. Likewise, a nucleic acid insert or a corresponding nucleic acid at the target genomic locus being deleted and/or replaced can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, about 4.5 kb to about 5 kb in length, or longer.

The nucleic acid insert or the corresponding nucleic acid at the target genomic locus being deleted and/or replaced can be a coding region such as an exon; a non-coding region such as an intron, an untranslated region, or a regulatory region (e.g., a promoter or an enhancer); or any combination thereof.

The nucleic acid insert can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acid inserts can also comprise a polynucleotide encoding a selection marker. Alternatively, the nucleic acid inserts can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo), hygromycin B phosphotransferase (hygr), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acid insert can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

The nucleic acid insert can also comprise one or more expression cassettes or deletion cassettes. A given cassette can comprise one or more of a nucleotide sequence of interest, a polynucleotide encoding a selection marker, and a reporter gene, along with various regulatory components that influence expression. Examples of selectable markers and reporter genes that can be included are discussed in detail elsewhere herein.

The nucleic acid insert can comprise a nucleic acid flanked with site-specific recombination target sequences. Alternatively, the nucleic acid insert can comprise one or more site-specific recombination target sequences. Although the entire nucleic acid insert can be flanked by such site-specific recombination target sequences, any region or individual polynucleotide of interest within the nucleic acid insert can also be flanked by such sites. Site-specific recombination target sequences, which can flank the nucleic acid insert or any polynucleotide of interest in the nucleic acid insert can include, for example, loxP, lox511, lox2272, lox66, lox71, loxM2, lox5171, FRT, FRT11, FRT71, attp, att, FRT, rox, or a combination thereof. In one example, the site-specific recombination sites flank a polynucleotide encoding a selection marker and/or a reporter gene contained within the nucleic acid insert. Following integration of the nucleic acid insert at a targeted locus, the sequences between the site-specific recombination sites can be removed. Optionally, two exogenous donor nucleic acids can be used, each with a nucleic acid insert comprising a site-specific recombination site. The exogenous donor nucleic acids can be targeted to 5′ and 3′ regions flanking a nucleic acid of interest. Following integration of the two nucleic acid inserts into the target genomic locus, the nucleic acid of interest between the two inserted site-specific recombination sites can be removed.

Nucleic acid inserts can also comprise one or more restriction sites for restriction endonucleases (i.e., restriction enzymes), which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res. 31:418-420; Roberts et al., (2003)Nucleic Acids Res. 31:1805-1812; and Belfort et al. (2002) in Mobile DNA II, pp. 761-783, Eds. Craigie et al., (ASM Press, Washington, DC)).

Some exogenous donor nucleic acids are capable of insertion into a C5 gene or locus by non-homologous end joining. In some cases, such exogenous donor nucleic acids do not comprise homology arms. For example, such exogenous donor nucleic acids can be inserted into a blunt end double-strand break following cleavage with a Cas protein. In a specific example, the exogenous donor nucleic acid can be delivered via AAV and can be capable of insertion into a C5 gene or locus by non-homologous end joining (e.g., the exogenous donor nucleic acid can be one that does not comprise homology arms).

In a specific example, the exogenous donor nucleic acid can be inserted via homology-independent targeted integration. For example, the nucleic acid insert in the exogenous donor nucleic acid is flanked on each side by a guide RNA target sequence (e.g., the same target site as in the C5 gene locus, and the CRISPR/Cas reagent (Cas protein and guide RNA) being used to cleave the target site in the C5 gene or locus). The Cas protein can then cleave the target sites flanking the nucleic acid insert. In a specific example, the exogenous donor nucleic acid is delivered AAV-mediated delivery, and cleavage of the target sites flanking the nucleic acid insert can remove the inverted terminal repeats (ITRs) of the AAV. In some methods, the target site in the C5 gene or locus (e.g., a guide RNA target sequence including the flanking protospacer adjacent motif) is no longer present if the nucleic acid insert is inserted into the C5 gene or locus in the correct orientation but it is reformed if the nucleic acid insert is inserted into the C5 gene or locus in the opposite orientation.

Other exogenous donor nucleic acids have short single-stranded regions at the 5′ end and/or the 3′ end that are complementary to one or more overhangs created by Cas-mediated cleavage at the C5 gene or locus. For example, some exogenous donor nucleic acids have short single-stranded regions at the 5′ end and/or the 3′ end that are complementary to one or more overhangs created by Cas-mediated cleavage at 5′ and/or 3′ target sequences at the C5 gene or locus. Some such exogenous donor nucleic acids have a complementary region only at the 5′ end or only at the 3′ end. For example, some such exogenous donor nucleic acids have a complementary region only at the 5′ end complementary to an overhang created at a 5′ target sequence at the C5 gene or locus or only at the 3′ end complementary to an overhang created at a 3′ target sequence at the C5 gene or locus. Other such exogenous donor nucleic acids have complementary regions at both the 5′ and 3′ ends. For example, other such exogenous donor nucleic acids have complementary regions at both the 5′ and 3′ ends (e.g., complementary to first and second overhangs, respectively) generated by Cas-mediated cleavage at the C5 gene or locus. For example, if the exogenous donor nucleic acid is double-stranded, the single-stranded complementary regions can extend from the 5′ end of the top strand of the donor nucleic acid and the 5′ end of the bottom strand of the donor nucleic acid, creating 5′ overhangs on each end. Alternatively, the single-stranded complementary region can extend from the 3′ end of the top strand of the donor nucleic acid and from the 3′ end of the bottom strand of the template, creating 3′ overhangs.

The complementary regions can be of any length sufficient to promote ligation between the exogenous donor nucleic acid and the target nucleic acid. Exemplary complementary regions are between about 1 to about 5 nucleotides in length, between about 1 to about 25 nucleotides in length, or between about 5 to about 150 nucleotides in length. For example, a complementary region can be at least about 1, 2, 3, 4, 5, 6, 7, 8, 9, 10, 11, 12, 13, 14, 15, 16, 17, 18, 19, 20, 21, 22, 23, 24, or 25 nucleotides in length. Alternatively, the complementary region can be about 5 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, about 120 to about 130, about 130 to about 140, about 140 to about 150 nucleotides in length, or longer.

Such complementary regions can be complementary to overhangs created by two pairs of nickases. Two double-strand breaks with staggered ends can be created by using first and second nickases that cleave opposite strands of DNA to create a first double-strand break, and third and fourth nickases that cleave opposite strands of DNA to create a second double-strand break. For example, a Cas protein can be used to nick first, second, third, and fourth guide RNA target sequences corresponding with first, second, third, and fourth guide RNAs. The first and second guide RNA target sequences can be positioned to create a first cleavage site such that the nicks created by the first and second nickases on the first and second strands of DNA create a double-strand break (i.e., the first cleavage site comprises the nicks within the first and second guide RNA target sequences). Likewise, the third and fourth guide RNA target sequences can be positioned to create a second cleavage site such that the nicks created by the third and fourth nickases on the first and second strands of DNA create a double-strand break (i.e., the second cleavage site comprises the nicks within the third and fourth guide RNA target sequences). The nicks within the first and second guide RNA target sequences and/or the third and fourth guide RNA target sequences can be off-set nicks that create overhangs. The offset window can be, for example, at least about 5 bp, 10 bp, 20 bp, 30 bp, 40 bp, 50 bp, 60 bp, 70 bp, 80 bp, 90 bp, 100 bp or more. See Ran et al. (2013) Cell 154:1380-1389; Mali et al. (2013) Nat. Biotechnol. 31:833-838; and Shen et al. (2014) Nat. Methods 11:399-404, each of which is herein incorporated by reference in its entirety for all purposes. In such cases, a double-stranded exogenous donor nucleic acid can be designed with single-stranded complementary regions that are complementary to the overhangs created by the nicks within the first and second guide RNA target sequences and by the nicks within the third and fourth guide RNA target sequences. Such an exogenous donor nucleic acid can then be inserted by non-homologous-end-joining-mediated ligation.

Some exogenous donor nucleic acids comprise homology arms. If the exogenous donor nucleic acid also comprises a nucleic acid insert, the homology arms can flank the nucleic acid insert. For ease of reference, the homology arms are referred to herein as 5′ and 3′ (i.e., upstream and downstream) homology arms. This terminology relates to the relative position of the homology arms to the nucleic acid insert within the exogenous donor nucleic acid. The 5′ and 3′ homology arms correspond to regions within the target genomic locus in the C5 gene or locus, which are referred to herein as “5′ target sequence” and “3′ target sequence,” respectively.

A homology arm and a target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. Exemplary homology arms are between about 25 nucleotides to about 2.5 kb in length, are between about 25 nucleotides to about 1.5 kb in length, or are between about 25 to about 500 nucleotides in length. For example, a given homology arm (or each of the homology arms) and/or corresponding target sequence can comprise corresponding regions of homology that are between about 25 to about 30, about 30 to about 40, about 40 to about 50, about 50 to about 60, about 60 to about 70, about 70 to about 80, about 80 to about 90, about 90 to about 100, about 100 to about 150, about 150 to about 200, about 200 to about 250, about 250 to about 300, about 300 to about 350, about 350 to about 400, about 400 to about 450, or about 450 to about 500 nucleotides in length, such that the homology arms have sufficient homology to undergo homologous recombination with the corresponding target sequences within the target nucleic acid. Alternatively, a given homology arm (or each homology arm) and/or corresponding target sequence can comprise corresponding regions of homology that are between about 0.5 kb to about 1 kb, about 1 kb to about 1.5 kb, about 1.5 kb to about 2 kb, or about 2 kb to about 2.5 kb in length. For example, the homology arms can each be about 750 nucleotides in length. The homology arms can be symmetrical (each about the same size in length), or they can be asymmetrical (one longer than the other).

In some methods, the exogenous donor nucleic acid can be a “large targeting vector” or “LTVEC,” which includes targeting vectors that comprise homology arms that correspond to and are derived from nucleic acid sequences larger than those typically used by other approaches intended to perform homologous recombination in cells. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. Generation and use of large targeting vectors (LTVECs) derived from bacterial artificial chromosome (BAC) DNA through bacterial homologous recombination (BHR) reactions using VELOCIGENE© genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes. CRISPR/Cas-assisted LTVEC targeting is described, e.g., in U.S. Pat. No. 9,546,384; US 2015-0159174; U.S. Pat. No. 9,228,208; US 2015-0159175; US 2016-0060657; U.S. Pat. No. 10,208,317; US 2017-0067078; U.S. Pat. No. 10,711,280; US 2019-0112619; and WO 2015/088643, each of which is herein incorporated by reference in its entirety for all purposes.

When a CRISPR/Cas system is used in combination with an exogenous donor nucleic acid, the 5′ and 3′ target sequences can be located in sufficient proximity to the Cas cleavage site (e.g., within sufficient proximity to a guide RNA target sequence) so as to promote the occurrence of a homologous recombination event between the target sequences and the homology arms upon a single-strand break (nick) or double-strand break at the Cas cleavage site. The term “Cas cleavage site” includes a DNA sequence at which a nick or double-strand break is created by a Cas protein complexed with a guide RNA. The target sequences within the targeted locus that correspond to the 5′ and 3′ homology arms of the exogenous donor nucleic acid are “located in sufficient proximity” to a Cas cleavage site if the distance is such as to promote the occurrence of a homologous recombination event between the 5′ and 3′ target sequences and the homology arms upon a single-strand break or double-strand break at the Cas cleavage site. Thus, the target sequences corresponding to the 5′ and/or 3′ homology arms of the exogenous donor nucleic acid can be, for example, within at least 1 nucleotide of a given Cas cleavage site or within at least 10 nucleotides to about 1,000 nucleotides of a given Cas cleavage site. As an example, the Cas cleavage site can be immediately adjacent to at least one or both of the target sequences.

The spatial relationship of the target sequences that correspond to the homology arms of the exogenous donor nucleic acid and the Cas cleavage site can vary. For example, target sequences can be located 5′ to the Cas cleavage site, target sequences can be located 3′ to the Cas cleavage site, or the target sequences can flank the Cas cleavage site.

Repair in response to cleavage of the C5 gene or locus by a Cas protein in the methods described herein can occur through any repair pathway. For example, repair can occur via homologous recombination (HR) or non-homologous end joining (NHEJ). Repair in response to double-strand breaks (DSBs) occurs principally through two conserved DNA repair pathways: homologous recombination (HR) and non-homologous end joining (NHEJ). See Kasparek & Humphrey (2011) Semin. Cell Dev. Biol. 22(8):886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLoS ONE 7:e45768:1-9; and Wang et al. (2013) Nat. Biotechnol. 31:530-532, each of which is herein incorporated by reference in its entirety for all purposes.

Non-homologous end joining (NHEJ) includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

Various types of targeted genetic modifications in a C5 gene or locus can be introduced using the methods described herein. Such targeted modifications can include, for example, additions of one or more nucleotides, deletions of one or more nucleotides, substitutions of one or more nucleotides, a point mutation, or a combination thereof. For example, at least 1, 2, 3, 4, 5, 7, 8, 9, 10 or more nucleotides can be changed (e.g., deleted, inserted, or substituted) to form the targeted genomic modification. The deletions, insertions, or substitutions can be of any size, as disclosed elsewhere herein. See, e.g., Wang et al. (2013) Cell 153:910-918; Mandalos et al. (2012) PLOS ONE 7:e45768:1-9; and Wang et al. (2013) Nat Biotechnol. 31:530-532, each of which is herein incorporated by reference in its entirety for all purposes.

Such targeted genetic modifications can result in disruption of the C5 gene or locus. Disruption can include alteration of a regulatory element (e.g., promoter or enhancer), a missense mutation, a nonsense mutation, a frame-shift mutation, a truncation mutation, a null mutation, or an insertion or deletion of small number of nucleotides (e.g., causing a frameshift mutation), and it can result in inactivation (i.e., loss of function) or loss of an allele. For example, a targeted modification can comprise disruption of the start codon of a C5 gene such that the start codon is no longer functional, or introduction of a frameshift mutation such that a functional C5 protein is no longer expressed. In some methods, the C5 gene or locus is inactivated or knocked out such that it no longer expresses a functional C5 protein. In some methods, the C5 gene or locus is modified such that it expresses a decreased amount of functional C5 protein.

In a specific example, a targeted modification can comprise a deletion between first and second guide RNA target sequences or Cas cleavage sites. If an exogenous donor sequence (e.g., repair template or targeting vector) is used, the modification can comprise a deletion between first and second guide RNA target sequences or Cas cleavage sites as well as an insertion of a nucleic acid insert between the 5′ and 3′ target sequences.

Alternatively, if an exogenous donor sequence is used in combination with CRISPR/Cas reagents, the modification can comprise a deletion between the 5′ and 3′ target sequences as well as an insertion of a nucleic acid insert between the 5′ and 3′ target sequences in the pair of first and second homologous chromosomes, thereby resulting in a homozygous modified genome. Alternatively, if the exogenous donor sequence comprises 5′ and 3′ homology arms with no nucleic acid insert, the modification can comprise a deletion between the 5′ and 3′ target sequences.

The deletion between the first and second guide RNA target sequences or the deletion between the 5′ and 3′ target sequences can be a precise deletion wherein the deleted nucleic acid consists of only the nucleic acid sequence between the first and second nuclease cleavage sites or only the nucleic acid sequence between the 5′ and 3′ target sequences such that there are no additional deletions or insertions at the modified genomic target locus. The deletion between the first and second guide RNA target sequences can also be an imprecise deletion extending beyond the first and second nuclease cleavage sites, consistent with imprecise repair by non-homologous end joining (NHEJ), resulting in additional deletions and/or insertions at the modified genomic locus. For example, the deletion can extend about 1 bp, about 2 bp, about 3 bp, about 4 bp, about 5 bp, about 10 bp, about 20 bp, about 30 bp, about 40 bp, about 50 bp, about 100 bp, about 200 bp, about 300 bp, about 400 bp, about 500 bp, or more beyond the first and second Cas protein cleavage sites. Likewise, the modified genomic locus can comprise additional insertions consistent with imprecise repair by NHEJ, such as insertions of about 1 bp, about 2 bp, about 3 bp, about 4 bp, about 5 bp, about 10 bp, about 20 bp, about 30 bp, about 40 bp, about 50 bp, about 100 bp, about 200 bp, about 300 bp, about 400 bp, about 500 bp, or more.

The targeted genetic modification can be, for example, a biallelic modification or a monoallelic modification. Biallelic modifications include events in which the same modification is made to the same locus on corresponding homologous chromosomes (e.g., in a diploid cell), or in which different modifications are made to the same locus on corresponding homologous chromosomes. In some methods, the targeted genetic modification is a monoallelic modification. A monoallelic modification includes events in which a modification is made to only one allele (i.e., a modification to the C5 gene in only one of the two homologous chromosomes). Homologous chromosomes include chromosomes that have the same genes at the same loci but possibly different alleles (e.g., chromosomes that are paired during meiosis). The term allele includes any of one or more alternative forms of a genetic sequence. In a diploid cell or organism, the two alleles of a given sequence typically occupy corresponding loci on a pair of homologous chromosomes.

A monoallelic mutation can result in a cell that is heterozygous for the targeted C5 modification. Heterozygosity includes situation in which only one allele of the C5 gene (i.e., corresponding alleles on both homologous chromosomes) have the targeted modification.

A biallelic modification can result in homozygosity for a targeted modification. Homozygosity includes situations in which both alleles of the CS gene (i.e., corresponding alleles on both homologous chromosomes) have the targeted modification. Alternatively, a biallelic modification can result in compound heterozygosity (e.g., hemizygosity) for the targeted modification. Compound heterozygosity includes situations in which both alleles of the target locus (i.e., the alleles on both homologous chromosomes) have been modified, but they have been modified in different ways (e.g., a targeted modification in one allele and inactivation or disruption of the other allele). For example, in the allele without the targeted modification, a double-strand break created by the Cas protein may have been repaired by non-homologous end joining (NHEJ)-mediated DNA repair, which generates a mutant allele comprising an insertion or a deletion of a nucleic acid sequence and thereby causes disruption of that genomic locus. For example, a biallelic modification can result in compound heterozygosity if the cell has one allele with the targeted modification and another allele that is not capable of being expressed. Compound heterozygosity includes hemizygosity. Hemizygosity includes situations in which only one allele (i.e., an allele on one of two homologous chromosomes) of the target locus is present. For example, a biallelic modification can result in hemizygosity for a targeted modification if the targeted modification occurs in one allele with a corresponding loss or deletion of the other allele.

Methods for assessing activity of CRISPR/Cas systems disclosed herein for targeting a CS gene or CS locus are well-known and are provided elsewhere herein. Assessment of activity can be in any cell type (e.g., liver cells, such as hepatocytes), any tissue type (e.g., liver), or any organ type (e.g., liver).

For example, the methods can comprise assessing modification of the CS locus. As one example, the assessing can comprise measuring non-homologous end joining (NHEJ) activity at the CS locus. This can comprise, for example, measuring the frequency of insertions or deletions within the CS locus. For example, in in vivo methods, the assessing can comprise sequencing the CS locus in one or more cells isolated from the animal (e.g., next-generation sequencing). Assessment can comprise isolating a target organ or tissue (e.g., liver) from the non-human animal and assessing modification of CS locus in the target organ or tissue. Assessment can also comprise assessing modification of CS locus in two or more different cell types within the target organ or tissue. Similarly, assessment can comprise isolating a non-target organ or tissue (e.g., two or more non-target organs or tissues) from the animal and assessing modification of C5 locus in the non-target organ or tissue. As one specific example, percent editing (e.g., total number of insertions or deletions observed over the total number of sequences read in the PCR reaction from a pool of lysed cells) at the C5 locus can be assessed (e.g., in liver cells, such as hepatocytes).

Such methods can also comprise measuring expression levels of the mRNA produced by the C5 locus, or by measuring expression levels of the protein encoded by the C5 locus. For example, protein levels can be measured in a particular cell, tissue, or organ type (e.g., liver), or secreted levels can be measured in the serum. Methods for assessing expression of C5 mRNA or C5 protein expressed from the C5 locus are provided elsewhere herein and are well-known.

In some methods, the percent editing (e.g., percent reads with indel) of the C5 gene in a target population of cells (e.g., hepatocytes, such as primary human hepatocytes) in vitro, ex vivo, or in vivo can be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% or can be between about 30% and about 99% of the population of cells or between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, between about 95% and about 99% of the population of cells. For example, the percent editing can be at least 50%, at least 60%, or at least 70% (e.g., in vivo at an LNP dose of 2 mg/kg).

In some methods, the percent reduction in plasma or serum C5 levels as compared to before administration of the CRISPR/Cas system or as compared to a negative control is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% or between about 50% and about 99%, between about 55% and about 99%, between about 60% and about 99%, between about 55% and about 99%, between about 60% and about 99%, between about 65% and about 99%, between about 70% and about 99%, between about 75% and about 99%, between about 80% and about 99%, between about 85% and about 99%, between about 90% and about 99%, or between about 95% and about 99% at 1 week post-administration, 2 weeks post-administration, 3 weeks post-administration, or 4 weeks post-administration. As one example, the percent reduction in plasma or serum C5 levels can be at least 50% at 3 weeks post-administration (e.g., with the CRISPR/Cas system delivered at an LNP dose of 0.3 mg/kg). As another example, the percent reduction in plasma or serum C5 levels can be at least 90% or at least 95% at 3 weeks post-administration (e.g., with the CRISPR/Cas system delivered at an LNP dose of 1 mg/kg). As another example, the percent reduction in plasma or serum C5 levels can be at least 95% or at least 98% at 3 weeks post-administration (e.g., with the CRISPR/Cas system delivered at an LNP dose of 2 mg/kg).

Such methods can also comprise measuring C5 activity. For example, classical pathway hemolysis can be measured ex vivo using sensitized sheep red blood cells. In some methods, the percent inhibition in classical pathway hemolysis as compared to before administration of the CRISPR/Cas system or as compared to a negative control is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% or between about 50% and about 99%, between about 55% and about 99%, between about 60% and about 99%, between about 65% and about 99%, between about 70% and about 99%, between about 75% and about 99%, between about 80% and about 99%, between about 85% and about 99%, between about 90% and about 99%, or between about 95% and about 99% at 1 week post-administration, 2 weeks post-administration, 3 weeks post-administration, or 4 weeks post-administration. As one example, the percent inhibition in classical pathway hemolysis can be at least 70% or at least 75% (e.g., at 3 weeks post-administration (e.g., with the CRISPR/Cas system delivered at an LNP dose of 2 mg/kg)).

In the methods described herein, introduction or administration of the CRISPR/Cas system can result in a durable effect in editing of the C5 gene or locus, and/or in reduction of plasma or serum C5 levels, and/or in reduction of C5 activity. For example, the durable effect can extend at least 3, at least 4, at least 5, at least 6, at least 7, at least 8, at least 9, at least 10, at least 11, at least 12, at least 13, at least 14, or at least 15 weeks, or it can extend at least 1, at least 2, at least 3, at least 4, at least 5, at least 6, at least 7, at least 8, at least 9, at least 10, at least 11, at least 12, at least 18, at least 24, at least 30, or at least 36 months, or it can extend at least 1, at least 2, at least 3, at least 4, at least 5, at least 6, at least 7, at least 8, at least 9, or at least 10 years. In one example, a single dose of the CRISPR/Cas system can result in a durable effect in editing of the C5 gene or locus, and/or in reduction of plasma or serum C5 levels, and/or in reduction of C5 activity.

### B. Prophylactic or Therapeutic Applications

The CRISPR/Cas systems disclosed herein for targeting a C5 gene or C5 locus are useful for the treatment and/or prevention of a disease, disorder, or condition associated with C5 and/or for ameliorating at least one symptom associated with such disease, disorder, or condition, either alone or in combination with or in association with other therapeutic agents such as the C5 antigen-binding proteins or antibodies. For example, any of the C5 antigen-binding proteins or antibodies disclosed herein can be used. In some methods, a CRISPR/Cas system disclosed herein for targeting a C5 gene or C5 locus may be administered at a therapeutic dose to a patient with a disease or disorder or condition associated with C5. A CRISPR/Cas system disclosed herein for targeting a C5 gene or C5 locus can also be used for the preparation of a pharmaceutical composition or medicament for treating patients suffering from a disease or disorder associated with C5 (e.g., a disease or disorder that would benefit from a decrease in C5 expression or activity) either alone or in combination with or in association with other therapeutic agents such as the C5 antigen-binding proteins or antibodies disclosed herein. Therapeutic or pharmaceutical compositions comprising a CRISPR/Cas system disclosed herein for targeting a C5 gene or C5 locus can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311.

The term “C5-associated disease” refers to a disease, disorder, condition or syndrome which is caused, maintained or exacerbated, or whose signs and/or symptoms are caused, maintained or exacerbated, directly or indirectly, by complement system activity wherein the complement system activity can be reduced or stabilized or eliminated by inhibition of C5 activity. See, e.g., WO 2021/034639 A1, US 2021-0046182, WO 2021/081277 A1, US 2021-0139573, WO 2017/218515 A1, US 2020-0262901, US 2017-0355757, or US 2020-0262900, each of which is herein incorporated by reference in its entirety for all purposes. Such C5 activity can be inhibited by preventing, for example, cleavage of C5 precursor into C5a and C5b chains, formation of membrane attack complex (MAC) and/or binding of the MAC to the surface of a target cell (e.g., a red blood cell). In some embodiments, C5 activity inhibition is as measured in a CH50 assay.

CH50 (50% Hemolytic Complement) is an assay to determine the level of classical complement pathway and it is sensitive to the reduction, absence and/or inactivity of any component of the pathway which is well known in the art. CH50 tests the functional capability of serum complement components of the classical pathway to lyse, for example, sheep red blood cells (SRBC) pre-coated with rabbit anti-sheep red blood cell antibody (haemolysin). For example, when antibody-coated SRBC are incubated with test serum, the classical pathway of complement is activated and hemolysis results. If a complement component is absent, the CH50 level will be zero; if one or more components of the classical pathway are decreased, the CH50 will be decreased. A fixed volume of optimally sensitized SRBC is added to each serum dilution. For example, after incubation, the mixture is centrifuged and the degree of hemolysis is quantified by measuring the absorbance of the hemoglobin released into the supernatant at 540 nm. The amount of complement activity is determined by examining the capacity of various dilutions of test serum to lyse antibody coated SRBC. See Costabile, Measuring the 50% haemolytic complement (CH50) activity of serum, J Vis Exp. 2010 (37): 1923; and Mayer, Complement and complement fixation, 1 p. 133-240. In E. A. 2 Kabat and M. M. Mayer (ed.), Experimental immunochemistry. Thomas, Springfield. AH50 is an analogous test to measure alternate-pathway function. See e.g., Mayer, Complement and complement fixation, p. 133-240. In E. Kabat and M. M. Mayer (ed.), Experimental immunochemistry. C. C. Thomas, Springfield, Ill. 1961; and Rapp & Borsos. Molecular basis of complement action. Appton Century Crofts, New York, N.Y. 1970. Tests evaluating the functional activity of the alternative pathway (AH50) use guinea pig, rabbit, or chicken erythrocytes as target cells. The AP has weak hemolytic activity for sheep erythrocytes. Here, activation of the classical pathway has to be blocked by adding EGTA to chelate 2⁺, and an optimal concentration of Mg²⁺ is required. Detection of low or absent hemolytic activity in CH50 and/or AH50 directs further complement analysis. See e.g., Joiner et al., 1983. A study of optimal reaction conditions for an assay of the human alternative complement pathway. Am. J. Clin. Pathol. 79:65-72.

C5-associated diseases include, for example: adult respiratory distress syndrome, age-related macular degeneration (AMD), allergy, Alport's syndrome, Alzheimer's disease, amyotrophic lateral sclerosis (ALS), antiphospholipid syndrome (APS), asthma, atherosclerosis, atypical hemolytic uremic syndrome (aHUS), an autoimmune disease, autoimmune hemolytic anemia (AIHA), balloon angioplasty, bronchoconstriction, bullous pemphigoid, burns, C3 glomerulopathy, capillary leak syndrome, a cardiovascular disorder, catastrophic antiphospholipid syndrome (CAPS), a cerebrovascular disorder, CHAPLE disease (CD55 deficiency with hyperactivation of complement, angiopathic thrombosis, and protein-losing enteropathy), a chemical injury, chronic obstructive pulmonary disease (COPD), cold agglutinin disease (CAD), corneal and/or retinal tissue, Crohn's disease, Degos disease, dense deposit disease (DDD), dermatomyositis, diabetes, diabetic angiopathy, diabetic macular edema (DME), diabetic nephropathy, diabetic retinopathy, dilated cardiomyopathy, disorder of inappropriate or undesirable complement activation, dyspnea, eclampsia, emphysema, epidermolysis bullosa, epilepsy, fibrogenic dust disease, frostbite, geographic atrophy (GA), glomerulonephritis, glomerulopathy, Goodpasture's Syndrome, Graves' disease, Guillain-Barre Syndrome, Hashimoto's thyroiditis, hemodialysis complications, hemolysis-elevated liver enzymes- and low platelets (HELLP) syndrome, hemolytic anemia, hemoptysis, Henoch-Schonlein purpura nephritis, hereditary angioedema, hyperacute allograft rejection, hypersensitivity pneumonitis, idiopathic thrombocytopenic purpura (ITP), IgA nephropathy, an immune complex disorder, immune complex vasculitis, immune complex-associated inflammation, an infectious disease, inflammation caused by an autoimmune disease, an inflammatory disorder, inherited CD59 deficiency, injury due to inert dusts and/or minerals, interleukin-2 induced toxicity during IL-2 therapy, ischemia-reperfusion injury, Kawasaki's disease, a lung disease or disorder, lupus nephritis, membrane proliferative glomerulonephritis, membranoproliferative nephritis, mesenteric artery reperfusion after aortic reconstruction, mesenteric/enteric vascular disorder, multifocal motor neuropathy (MMN), multiple sclerosis, myasthenia gravis, myocardial infarction, myocarditis, neurological disorder, neuromyelitis optica, obesity, ocular angiogenesis, ocular neovascularization affecting choroidal, organic dust disease, parasitic disease, Parkinson's disease, paroxysmal nocturnal hemoglobinuria (PNH), e.g., active PNH, pauci-immune vasculitis, pemphigus, percutaneous transluminal coronary angioplasty (PTCA), peripheral (e.g., musculoskeletal) vascular disorder, pneumonia, post-ischemic reperfusion condition, post-pump syndrome in cardiopulmonary bypass, post-pump syndrome in renal bypass, pre-eclampsia, progressive kidney failure, proliferative nephritis, proteinuric kidney disease, psoriasis, pulmonary embolism, pulmonary fibrosis, pulmonary infarction, pulmonary vasculitis, recurrent fetal loss, a renal disorder, renal ischemia, renal ischemia-reperfusion injury, a renovascular disorder, restenosis following stent placement, rheumatoid arthritis (RA), rotational atherectomy, schizophrenia, sepsis, septic shock, SLE nephritis, smoke injury, spinal cord injury, spontaneous fetal loss, stroke, systemic inflammatory response to sepsis, systemic lupus erythematosus (SLE), systemic lupus erythematosus-associated vasculitis, Takayasu's disease, thermal injury, thrombotic thrombocytopenic purpura (TTP), traumatic brain injury, type I diabetes, typical hemolytic uremic syndrome (tHUS), uveitis, vasculitis, vasculitis associated with rheumatoid arthritis, venous gas embolus (VGE); or xenograft rejection.

Likewise, the methods for modifying or knocking at a C5 gene or locus disclosed herein can be used for treating a disease or disorder associated with C5 in a subject using the C5-targeting CRISPR/Cas reagents disclosed herein either alone or in combination with or in association with other therapeutic agents such as the C5 antigen-binding proteins or antibodies disclosed herein. Such therapeutic methods can comprise administering a therapeutically effective amount of a pharmaceutical composition comprising a CRISPR/Cas system disclosed herein for targeting a C5 gene or C5 locus to the subject in need thereof. Such therapeutic methods can comprise administering a therapeutically effective amount of a pharmaceutical composition comprising a CRISPR/Cas system disclosed herein for targeting a C5 gene or C5 locus and a pharmaceutical composition comprising a C5 antigen-binding protein or antibody to the subject in need thereof. Such therapeutic methods can comprise administering a therapeutically effective amount of a pharmaceutical composition comprising a CRISPR/Cas system disclosed herein for targeting a C5 gene or C5 locus and comprising a C5 antigen-binding protein or antibody to the subject in need thereof. The disorder treated can be any disease or condition which is improved, ameliorated, inhibited, or prevented by inhibition of or reduction of C5 activity. Some such methods prevent, treat, or ameliorate at least one symptom of atypical hemolytic uremic syndrome (aHUS), the method comprising administering a therapeutically effective amount of a CRISPR/Cas system disclosed herein for targeting a C5 gene or C5 locus to a subject in need thereof. Some such methods ameliorate or reduce the severity of at least one symptom or indication of paroxysmal nocturnal hemoglobinuria (PNH) in a subject by administering a CRISPR/Cas system disclosed herein for targeting a C5 gene or C5 locus. In some methods, the CRISPR/Cas system disclosed herein for targeting a C5 gene or C5 locus may be administered prophylactically or therapeutically to a subject having or at risk of having a C5-associated disease or disorder. The CRISPR/Cas system disclosed herein for targeting a C5 gene or C5 locus (either alone or in combination with other therapeutic agents such as the C5 antigen-binding proteins or antibodies disclosed herein) may be administered subcutaneously, intravenously, intradermally, intraperitoneally, orally, or intramuscularly, or by any other suitable means.

A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

A subject can be an animal, preferably a mammal, more preferably a human, in need of amelioration, prevention, and/or treatment of a C5-associated disease or disorder (e.g., such as atypical hemolytic uremic syndrome (aHUS) or paroxysmal nocturnal hemoglobinuria (PNH)). The term includes human subjects who have or are at risk of having such a disease or disorder. In some embodiments, amino acid Arginine 885 is mutated in the subject's C5 to another amino acid, e.g., R885H or R885C.

The terms treat, treating, or treatment refer to the reduction or amelioration of the severity of at least one symptom or indication of a C5-associated disease or disorder due to the administration of a therapeutic agent such as a CRISPR/Cas system disclosed herein for targeting a C5 gene or C5 locus (either alone or in combination with other therapeutic agents such as the C5 antigen-binding proteins or antibodies disclosed herein) to a subject in need thereof. The terms include inhibition of progression of disease or of worsening of a symptom/indication. The terms also include positive prognosis of disease (i.e., the subject may be free of disease or may have reduced disease upon administration of a therapeutic agent such as a CRISPR/Cas system disclosed herein for targeting a C5 gene or C5 locus (either alone or in combination with other therapeutic agents such as the C5 antigen-binding proteins or antibodies disclosed herein)). The therapeutic agent may be administered at a therapeutic dose to the subject. The terms prevent, preventing, or prevention refer to inhibition of manifestation of a C5-associated disease or disorder or any symptoms or indications of such a disease or disorder upon administration of a CRISPR/Cas system disclosed herein for targeting a C5 gene or C5 locus.

A C5-associated disease or disorder refers to a disease or disorder which is caused (directly or indirectly) by inflammation, cell injury and/or cell killing that is mediated by C5a and/or C5b. Non-limiting examples of C5-associated diseases include atypical hemolytic uremic syndrome (aHUS), paroxysmal nocturnal hemoglobinuria (PNH), refractory myasthenia gravis (rMG), neuromyelitis optica (NMO), IgA nephropathy, membranous nephropathy, lupus nephritis, C3 glomerulopathy, and ANCA-vasculitis. In a specific example, the C5-associated disease or disorder is atypical hemolytic uremic syndrome (aHUS). In a specific example, the C5-associated disease or disorder is paroxysmal nocturnal hemoglobinuria (PNH).

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of atypical hemolytic uremic syndrome (aHUS). aHUS is a rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Signs and symptoms of aHUS can include, for example, feelings of illness, fatigue, irritability, and lethargy, anemia, thrombocytopenia, acute kidney failure, hypertension and organ damage. Symptoms and indications of aHUS include, but are not limited to, platelet activation, hemolysis, systemic thrombotic microangiopathy (formation of blood clots in small blood vessels throughout the body) leading to stroke, heart attack, kidney failure and/or death, end-stage renal disease, permanent renal damage, abdominal pain, confusion, edema, fatigue, nausea/vomiting, diarrhea, and microangiopathic anemia.

Atypical hemolytic uremic syndrome (aHUS) is a rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Most aHUS are caused by complement system defects impairing ordinary regulatory mechanisms. Activating events therefore lead to unbridled, ongoing complement activity producing widespread endothelial injury. Signs and symptoms of aHUS can include, for example, feelings of illness, fatigue, irritability, and lethargy, anemia, thrombocytopenia, acute kidney failure, hypertension and organ damage.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of paroxysmal nocturnal hemoglobinuria (PNH). PNH is a rare acquired, life-threatening disease of the blood. The disease is characterized by destruction of red blood cells (hemolytic anemia), blood clots (thrombosis), and impaired bone marrow function (not making enough of the three blood components). Signs and symptoms of PNH can include significant fatigue or weakness, bruising or bleeding easily, shortness of breath, recurring infections and/or flu-like symptoms, difficulty in controlling bleeding, even from very minor wounds, the appearance of small red dots on the skin that indicates bleeding under the skin, severe headache, fever due to infection and blood clots (thrombosis). Symptoms and indications of PNH include, but are not limited to, destruction of red blood cells, thrombosis (including deep vein thrombosis, pulmonary embolism), intravascular hemolytic anemia, red discoloration of urine, symptoms of anemia such as tiredness, shortness of breath, and palpitations, abdominal pain and difficulty swallowing.

Paroxysmal nocturnal hemoglobinuria (PNH) originates from a multipotent, hematopoietic stem cell (HSC) that acquires a mutation of the phosphatidylinositol glycan anchor biosynthesis class A (PIGA) gene. The PIGA gene product is required for the biosynthesis of the glycophosphatidylinositol (GPI) anchor, a glycolipid moiety that attaches dozens of proteins to the plasma membrane of cells. Consequently, the PNH stem cell and all of its progeny have a reduction or absence of GPI-anchored proteins. The mature blood cells derived from the hematopoietic clone can have a complete deficiency (type III) or a partial deficiency (type II) of GPI-linked proteins (Hillmen et al., Effect of eculizumab on hemolysis and transfusion requirements in patients with paroxysmal nocturnal hemoglobinuria. N Engl J Med 2004; 350(6):552-9). Two of the proteins that are affected by the absence of GPI anchors are CD55 and CD59, complement regulatory proteins. CD55 regulates complement activation by inhibiting complement component 3 (C3) convertases, whereas CD59 inhibits the assembly of the membrane-attack complex (MAC) C5b-C9 by interacting with C8 and C9 (Brodsky, How I treat paroxysmal nocturnal hemoglobinuria. Blood 2009; 113(26):6522-7). Their absence renders PNH erythrocytes susceptible to complement-mediated intravascular hemolysis. This intravascular hemolysis in patients with PNH causes anemia (frequently requiring blood transfusion) and hemoglobinuria. Complications of PNH include thrombosis, abdominal pain, dysphagia, erectile dysfunction, and pulmonary hypertension (Hillmen et al., The complement inhibitor eculizumab in paroxysmal nocturnal hemoglobinuria. N Engl J Med 2006; 355(12):1233-43). Thromboembolism is a common cause of mortality in patients with PNH. Potential mechanisms for thromboembolism include platelet activation, toxicity of free hemoglobin, nitric oxide depletion, absence of other GPI-linked proteins, and endothelial dysfunction (Hill et al., Thrombosis in paroxysmal nocturnal hemoglobinuria. Blood 2013; 121(25):4985-96). PNH frequently occurs with autoimmune aplastic anemia (Luzzatto & Risitano, Advances in understanding the pathogenesis of acquired aplastic anaemia. Br J Haematol 2018; 182(6):758-76). Included herein are methods for reducing the need for blood transfusions to address anemia secondary to hemolysis that is caused by PNH, reducing the need for erythropoietin, iron supplements and/or folic acid, reducing the incidence of anemia, reducing the incidence of hemoglobinuria or reducing the incidence of hemolysis, in a subject suffering from PNH, by administering, to the subject, an antagonist antigen-binding protein that binds specifically to C5 such as REGN3918 by a dosing regimen set forth herein.

The diagnosis of PNH can be established using an internationally accepted definition of presence of PNH granulocyte clone size of >10% measured in peripheral blood by flow cytometry. An accepted definition of “active disease” (active PNH) is the presence of 1 or more of the following PNH-related signs or symptoms within 3 months: fatigue, hemoglobinuria, abdominal pain, shortness of breath (dyspnea), anemia (hemoglobin <10 g/dL), history of a major adverse vascular event (MAVE; including thrombosis), dysphagia, or erectile dysfunction. Alternatively, activity can be established by a history of RBC transfusion due to PNH within 3 months. Methods for treating active PNH are also included.

In certain applications, the CRISPR/Cas systems disclosed herein for targeting a CS gene or CS locus (either alone or in combination with other therapeutic agents such as the C5 antigen-binding proteins or antibodies disclosed herein) are useful in treating or preventing a symptom or indication of CHAPLE (CD55 deficiency with hyperactivation of complement, angiopathic thrombosis, and protein-losing enteropathy). CHAPLE disease is an autosomal recessive disorder caused by loss of function mutations in CD55 (also known as decay accelerating factor, DAF). Signs and symptoms of CHAPLE can include hypoproteinemia (low serum levels of albumin and immunoglobulins)-hypoproteinemia leads to facial and extremity edema and recurrent infections, malabsorption syndrome (chronic diarrhea, failure to thrive, anemia, and micronutrient deficiencies), complement overactivation, intestinal lymphangiectasia (IL) and bowel inflammation; and/or increased susceptibility to visceral thrombosis.

CHAPLE disease (CD55 deficiency with hyperactivation of complement, angiopathic thrombosis, and protein-losing enteropathy) is an autosomal recessive disorder caused by loss of function mutations in CD55 (also known as decay accelerating factor, DAF). Signs and symptoms of CHAPLE can include hypoproteinemia (low serum levels of albumin and immunoglobulins)-hypoproteinemia leads to facial and extremity edema and recurrent infections, malabsorption syndrome (chronic diarrhea, failure to thrive, anemia, and micronutrient deficiencies), complement overactivation, intestinal lymphangiectasia (IL) and bowel inflammation; and/or increased susceptibility to visceral thrombosis. CHAPLE disease is caused by biallelic loss-of-function mutations in the CD55 gene. Clinically, it manifests as a familial form of protein-losing enteropathy (PLE) caused by primary intestinal lymphangiectasia (PIL) or Waldmann's disease that is frequently severe and can be accompanied by lethal systemic manifestations. CD55 is a glycophosphatidylinositol (GPI)-anchored membrane protein that inhibits the enzymatic activity of C3b and C4b, thus preventing the formation of C3 and C5 convertases that lead ultimately to the assembly of the membrane-attack complex (C5b-C9). Thus, the absence of CD55 causes overactivation of the complement system, causing the production of various complement products including anaphylatoxins and the membrane-attack complex. When absent due to somatic mutation of the PIGA gene (required for the biosynthesis of GPI anchors) in hematopoietic stem cells, CD55 loss, as well as CD59 loss, is specific to hematopoietic cells (CD59 is another GPI-linked complement regulatory protein). Typically, the resultant complement-mediated lysis of red cells and platelets gives rise to intravascular hemolysis and thrombosis in PNH. In CHAPLE, isolated germ line loss of CD55 expression in all tissues manifests in the GI tract, as primary intestinal lymphangiectasia, which causes PLE. In general, unlike PNH, hemolysis is not observed in CHAPLE patients. Also included methods for reducing the need for the administration of corticosteroids, immunoglobulin, albumin, biological therapeutic agents (e.g., antibodies or antigen-binding fragments thereof such as anti-TNFalpha, or vedolizumab), immunomodulators (e.g., azathioprine or mesalazine), micronutrients, enteral or parenteral supplementation, anti-coagulants (e.g., low-molecular-weight heparin), antibiotics and/or anti-platelet agents (e.g., aspirin, such as low-dose aspirin), in a subject suffering from CHAPLE, by administering, to the subject, an antagonist antigen-binding protein that binds specifically to C5, such as REGN3918, by a dosing regimen set forth herein. See Kurolap et al., Loss of CD55 in Eculizumab-Responsive Protein-Losing Enteropathy. N Engl J Med 2017; 377(1):87-9.; and Ozen et al., CD55 Deficiency and Protein-Losing Enteropathy. N Engl J Med 2017b; 377(15):1499-500. CD55 mutations associated with CHAPLE disease include, for example, 149-150delAA; 149-150insCCTT; 109delC; 800G>C; 287-1G>C; 149-150delAAinsCCTT; (as set forth in WO2018/053039) or a CD55 mutation that results in the same mutant amino acid sequence.

Diagnosis of CHAPLE can be done by genetic analysis to identify a CD55 loss-of-function mutation. Diagnosis can be confirmed by flow cytometry or Western blotting of peripheral blood cells to identify decreased presence of CD55. Active CHAPLE disease is, in some embodiments, characterized by hypoalbuminemia of less than or equal to 3.2 g/dL, and one or more of the following signs or symptoms which are attributable to CHAPLE: diarrhea, vomiting, abdominal pain, peripheral or facial edema, or an episode of infection with concomitant hypogammaglobulinemia, or a new thromboembolic event. The normal range of serum albumin is typically about 3.5-5.5 g/dL.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of antiphospholipid syndrome (APS). APS is an autoimmune disease characterized by arterial and venous thrombosis due to antiphospholipid antibodies. The disorder is referred to as primary when it occurs in the absence of another autoimmune disease. Secondary APS occurs in the context of an autoimmune disorder such as systemic lupus erythematosus. The catastrophic APS (CAPS) is a rare life-threatening form of APS in which widespread intravascular thrombosis results in multiorgan ischemia and failure.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of myasthenia gravis (MG). MG is a chronic autoimmune neuromuscular disease that causes weakness in the skeletal muscles, which are responsible for breathing and moving parts of the body, including the arms and legs.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of typical hemolytic uremic syndrome (tHUS). tHUS may follow a gastrointestinal infection with Shiga toxin-producing Escherichia coli (STEC). Typical HUS (STEC-HUS; Shiga toxin-producing Escherichia coli (STEC)-hemolytic uremic syndrome (HUS)) can be initiated when the Shiga toxin (or Shiga-like toxin), a known potent cytotoxin, binds to cell membrane glycolipid Gb3 (via domain B). Domain A is internalized and subsequently halts protein synthesis and induces apoptosis of the affected cell. The Shiga toxin has several additional effects on endothelial cells, one of which is enhanced expression of functional tissue factor that could contribute to microvascular thrombosis. The toxin causes damage to or activation of endothelium, red cells, and platelets.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in treating or preventing a symptom or indication of a C5-associated ophthalmologic disease, e.g., age-related macular degeneration (AMD; e.g., wet or dry), diabetic retinopathy (DR), non-infections uveitis, geographic atrophy, Stargardt Macular Dystrophy or optic neuritis. In such methods, the CRISPR/Cas systems and/or other therapeutic agents can be administered, e.g., by intraocular or intravitreal injection.

AMD is the progressive degeneration of the macula (central part of the retina), typically, in people aged over 55 years. Various complement components, including C3, C5b-9, CFB, and CFH, have been detected in drusen as well as in AMD lesions. In addition, increased plasma levels of C3a, C3d, Bb, and C5a have been observed in AMD patients. These results suggest increased local and systemic complement activation in AMD.

DR is a progressive degeneration of retinal vasculature and neurons as a result of diabetes. Choriocapillaris of DR eyes contain significant levels of C3d and the C5b-9 complex. C5b-9 deposition may also be detectable in retinal vessels of patients with >9-year type-2 diabetes and increased C5a may be detected in the vitreous of patients with proliferative DR suggesting that complement activation is involved in retinal vascular damage in DR.

Non-infectious uveitis is inflammation—heat, redness, pain, and swelling—in one or both eyes which is not due to infection.

Geographic atrophy (GA) is a chronic progressive degeneration of the macula, as part of late-stage age-related macular degeneration (AMD). The disease is characterized by localized sharply demarcated atrophy of outer retinal tissue, retinal pigment epithelium and choriocapillaris. It typically starts typically in the perifoveal region and expands to involve the fovea with time, leading to central scotomas and permanent loss of visual acuity. It is bilateral in most cases.

Autosomal recessive Stargardt macular dystrophy (STGD1) is a dystrophy resulting from mutations in the ABCA4 (ABCR) gene. Mutations in ABCA4 also result in cone-rod dystrophy. The age of onset of juvenile and early adult STGD1 is usually 8-25 years with some cases occurring in older adults (late adult onset STGD1). A hallmark of the disease is premature accumulation of lipofuscin (a brown-yellow autofluorescent pigment associated with aging) in the retinal pigment epithelia (RPE) of the eye, causing a pattern of yellowish flecks that extend outward from the macula.

Optic neuritis is an inflammation that damages the optic nerve. Pain and temporary vision loss in one eye are common symptoms of optic neuritis.

In certain applications, the CRISPR/Cas systems disclosed herein for targeting a C5 gene or C5 locus (either alone or in combination with other therapeutic agents such as the C5 antigen-binding proteins or antibodies disclosed herein) are useful in reducing complement activity (e.g., C5-mediated complement activity) in the body of a subject. For example, in some embodiments, the complement activity is complement-mediated hemolysis (e.g., classical pathway mediated or alternative pathway mediated) or C5 activity (e.g., binding of C5a to C5aR1, generation of C5a and/or C5b from C5 precursor; or formation or deposition of membrane attack complex (MAC) in cells, e.g., endothelial cells). In some embodiments, complement activity is the capacity of serum taken from a subject's body to lyse sheep erythrocytes coated with anti-sheep antibodies.

In certain applications, the CRISPR/Cas systems disclosed herein for targeting a C5 gene or C5 locus are useful (either alone or in combination with other therapeutic agents such as the C5 antigen-binding proteins or antibodies disclosed herein) for treating or preventing at least one symptom or indication of a C5-associated disease or disorder selected from the group consisting of neurological disorders, renal disorders, multiple sclerosis, stroke, Guillain Bane Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, myocardial infarction, capillary leak syndrome, obesity, diabetes, Alzheimer's disease, schizophrenia, stroke, epilepsy, atherosclerosis, vasculitis, bullous pemphigoid, C3 glomerulopathy, membranoproliferative glomerulonephritis, balloon angioplasty, post-pump syndrome in cardiopulmonary bypass or renal bypass, hemodialysis, renal ischemia, mesenteric artery reperfusion after aortic reconstruction, infectious disease or sepsis, immune complex disorders and autoimmune diseases, diabetic nephropathy, Alport's syndrome, progressive kidney failure, proteinuric kidney diseases, renal ischemia-reperfusion injury, lupus nephritis, glomerulopathy, rheumatoid arthritis, systemic lupus erythematosus (SLE), SLE nephritis, membranoproliferative nephritis, hemolytic anemia, neuromyelitis optica, renal transplant, inherited CD59 deficiency, psoriasis, and myasthenia gravis. In some embodiments, the CRISPR/Cas systems are useful (either alone or in combination with other therapeutic agents such as the C5 antigen-binding proteins or antibodies disclosed herein) for treating or preventing at least one symptom or indication of a C5-associated disease or disorder selected from the group consisting of lung disease and disorders such as dyspnea, hemoptysis, ARDS, asthma, chronic obstructive pulmonary disease (COPD), emphysema, pulmonary embolisms and infarcts, pneumonia, fibrogenic dust diseases, injury due to inert dusts and minerals (e.g., silicon, coal dust, beryllium, and asbestos), pulmonary fibrosis, organic dust diseases, chemical injury (due to irritant gasses and chemicals, e.g., chlorine, phosgene, sulfur dioxide, hydrogen sulfide, nitrogen dioxide, ammonia, and hydrochloric acid), smoke injury, thermal injury (e.g., burn, freeze), asthma, allergy, bronchoconstriction, hypersensitivity pneumonitis, parasitic diseases, Goodpasture's Syndrome, pulmonary vasculitis, hereditary angioedema, and immune complex-associated inflammation.

In some embodiments, the CRISPR/Cas systems are useful (either alone or in combination with other therapeutic agents such as the C5 antigen-binding proteins or antibodies disclosed herein) for treating or preventing a C5-associated disease which is one or more of: adult respiratory distress syndrome, age-related macular degeneration (AMD), allergy, Alport's syndrome, Alzheimer's disease, antiphospholipid syndrome (APS), asthma, atherosclerosis, atypical hemolytic uremic syndrome (aHUS), an autoimmune disease, autoimmune hemolytic anemia (AIHA), balloon angioplasty, bronchoconstriction, bullous pemphigoid, burns, C3 glomerulopathy, capillary leak syndrome, a cardiovascular disorder, catastrophic antiphospholipid syndrome (CAPS), a cerebrovascular disorder, CHAPLE disease (CD55 deficiency with hyperactivation of complement, angiopathic thrombosis, and protein-losing enteropathy), a chemical injury, chronic obstructive pulmonary disease (COPD), cold agglutinin disease (CAD), corneal and/or retinal tissue, Crohn's disease, Degos disease, dense deposit disease (DDD), dermatomyositis, diabetes, diabetic angiopathy, diabetic, macular edema (DME), diabetic nephropathy, diabetic retinopathy, dilated cardiomyopathy, disorder of inappropriate or undesirable complement activation, dyspnea, emphysema, epidermolysis bullosa, epilepsy, fibrogenic dust disease, frostbite, geographic atrophy (GA), glomerulonephritis, glomerulopathy, Goodpasture's Syndrome, Graves' disease, Guillain-Barre Syndrome, Hashimoto's thyroiditis, hemodialysis complications, hemolysis-elevated liver enzymes- and low platelets (HELLP) syndrome, hemolytic anemia, hemoptysis, Henoch-Schonlein purpura nephritis, hereditary angioedema, hyperacute allograft rejection, hypersensitivity pneumonitis, idiopathic thrombocytopenic purpura (ITP), IgA nephropathy, an immune complex disorder, immune complex vasculitis, immune complex-associated inflammation, an infectious disease, inflammation caused by an autoimmune disease, an inflammatory disorder, inherited CD59 deficiency, injury due to inert dusts and/or minerals, interleukin-2 induced toxicity during IL-2 therapy, ischemia-reperfusion injury, Kawasaki's disease, a lung disease or disorder, lupus nephritis, membrane proliferative glomerulonephritis, membrano-proliferative nephritis, mesenteric artery reperfusion after aortic reconstruction, mesenteric/enteric vascular disorder, multifocal motor neuropathy (MMN), multiple sclerosis, myasthenia gravis, myocardial infarction, myocarditis, neurological disorder, neuromyelitis optica, obesity, ocular angiogenesis, ocular neovascularization affecting choroidal, organic dust disease, parasitic disease, Parkinson's disease, paroxysmal nocturnal hemoglobinuria (PNH), pauci-immune vasculitis, pemphigus, percutaneous transluminal coronary angioplasty (PTCA), peripheral (e.g., musculoskeletal) vascular disorder, pneumonia, post-ischemic reperfusion condition, post-pump syndrome in cardiopulmonary bypass, post-pump syndrome in renal bypass, progressive kidney failure, proliferative nephritis, proteinuric kidney disease, psoriasis, pulmonary embolism, pulmonary fibrosis, pulmonary infarction, pulmonary vasculitis, recurrent fetal loss, a renal disorder, renal ischemia, renal ischemia-reperfusion injury, a renovascular disorder, restenosis following stent placement, rheumatoid arthritis (RA), rotational atherectomy, schizophrenia, sepsis, septic shock, SLE nephritis, smoke injury, spinal cord injury, spontaneous fetal loss, stroke, systemic inflammatory response to sepsis, systemic lupus erythematosus (SLE), systemic lupus erythematosus-associated vasculitis, Takayasu's disease, thermal injury, thrombotic thrombocytopenic purpura (TTP), traumatic brain injury, type I diabetes, typical hemolytic uremic syndrome (tHUS), uveitis, vasculitis, vasculitis associated with rheumatoid arthritis, venous gas embolus (VGE); and/or xenograft rejection.

In certain applications, the CRISPR/Cas systems disclosed herein for targeting a CS gene or CS locus (either alone or in combination with other therapeutic agents such as the C5 antigen-binding proteins or antibodies disclosed herein) are useful to treat subjects suffering from an ocular disease such as age-related macular degeneration (AMD), diabetic macular edema (DME), diabetic retinopathy, ocular angiogenesis (ocular neovascularization affecting choroidal, corneal or retinal tissue), geographic atrophy (GA), uveitis and neuromyelitis optica. The CRISPR/Cas systems disclosed herein for targeting a CS gene or CS locus (either alone or in combination with other therapeutic agents such as the C5 antigen-binding proteins or antibodies disclosed herein) may be used to treat or to ameliorate at least one symptom or indication of dry AMD or wet AMD. In some methods, the CRISPR/Cas systems disclosed herein for targeting a CS gene or CS locus are useful in preventing or slowing rate of loss of vision. In some methods, the CRISPR/Cas systems disclosed herein for targeting a CS gene or CS locus (either alone or in combination with other therapeutic agents such as the C5 antigen-binding proteins or antibodies disclosed herein) are useful in reducing drusen in the eye of a subject with dry AMD. In some methods, the CRISPR/Cas systems disclosed herein for targeting a CS gene or CS locus (either alone or in combination with other therapeutic agents such as the C5 antigen-binding proteins or antibodies disclosed herein) are useful in preventing or reducing/slowing loss of vision in a subject with AMD.

The CRISPR/Cas systems disclosed herein for targeting a C5 gene or C5 locus may be administered (either alone or in combination with other therapeutic agents such as the C5 antigen-binding proteins or antibodies disclosed herein) to relieve or prevent or decrease the severity of one or more of the symptoms or conditions/indications of the ocular disease or disorder. The CRISPR/Cas systems disclosed herein for targeting a C5 gene or C5 locus may be used (either alone or in combination with other therapeutic agents such as the C5 antigen-binding proteins or antibodies disclosed herein) to ameliorate or reduce the severity of at least one symptom including, but not limited to loss of vision, visual distortion, difficulty adapting to low light levels, crooked central vision, increase in haziness of central/overall vision, presence of drusen (tiny accumulations of extracellular material that build up on the retina), pigmentary changes, distorted vision in the form of metamorphopsia, in which a grid of straight lines appears wavy and parts of the grid may appear blank, exudative changes (hemorrhages in the eye, hard exudates, subretinal/sub-RPE/intraretinal fluid), slow recovery of visual function after exposure to bright light (photostress test), incipient and geographic atrophy, visual acuity drastically decreasing (two levels or more), e.g., 20/20 to 20/80, preferential hyperacuity perimetry changes (for wet AMD), blurred vision, gradual loss of central vision (for those with non-exudative macular degeneration, rapid onset of vision loss (often caused by leakage and bleeding of abnormal blood vessels in subjects with exudative macular degeneration, central scotomas (shadows or missing areas of vision), trouble discerning colors, specifically dark ones from dark ones and light ones from light ones, loss in contrast sensitivity, straight lines appear curved in an Amsler grid.

The CRISPR/Cas systems disclosed herein for targeting a C5 gene or C5 locus may also be used (either alone or in combination with other therapeutic agents such as the C5 antigen-binding proteins or antibodies disclosed herein) prophylactically in subjects at risk for developing macular degeneration such as subjects over the age of 50, subjects with a family history of macular degeneration, smokers, and subjects with obesity, high cholesterol, cardiovascular disease, or unhealthy diet.

C. Administering CRISPR/Cas Reagents and/or C5 Antigen-Binding Proteins and/or Other Reagents Targeting C5 to Animals or Cells

The methods disclosed herein can comprise introducing into an animal (e.g., mammal, such as a human) or cell various CRISPR/Cas reagents targeting C5 (and optionally exogenous donor nucleic acids targeting C5), including in the form of nucleic acids (e.g., DNA or RNA), proteins, or nucleic-acid-protein complexes. The methods disclosed herein can also comprise introducing into an animal (e.g., mammal, such as a human) or cell various C5 antigen-binding proteins or C5 antibodies or other therapeutic agents. “Introducing” includes presenting to the cell or animal the molecule(s) (e.g., nucleic acid(s) or protein(s)) in such a manner that it gains access to the interior of the animal or cell or to the interior of cells within the animal. The introducing can be accomplished by any means, and two or more of the components (e.g., two of the components, or all of the components) can be introduced into the cell or animal simultaneously or sequentially in any combination. For example, a Cas protein can be introduced into a cell or animal before introduction of a guide RNA, or it can be introduced following introduction of the guide RNA. As another example, an exogenous donor nucleic acid can be introduced prior to the introduction of a Cas protein and a guide RNA, or it can be introduced following introduction of the Cas protein and the guide RNA (e.g., the exogenous donor nucleic acid can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or animal by the same delivery method or different delivery methods. Similarly, two or more of the components can be introduced into an animal by the same route of administration or different routes of administration.

A guide RNA can be introduced into an animal or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the animal. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or an animal, the Cas protein can be transiently, conditionally, or constitutively expressed in the cell or in a cell in the animal.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery.

Molecules (e.g., Cas proteins or guide RNAs or nucleic acids encoding or C5 antigen-binding proteins) introduced into the animal or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or animal. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to an animal include hydrodynamic delivery, virus-mediated delivery (e.g., adeno-associated virus (AAV)-mediated delivery), and lipid-nanoparticle-mediated delivery.

Introduction of nucleic acids and proteins into cells or animals can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 706, SEQ ID NO: 707, or SEQ ID NO: 708. Other examples of ITRs comprise one or more mutations compared to SEQ ID NO: 706, SEQ ID NO: 707, or SEQ ID NO: 708 and can be at least 90%, at least 91%, at least 92%, at least 93%, at least 94%, at least 95%, at least 96%, at least 97%, at least 98%, or at least 99% identical to SEQ ID NO: 706, SEQ ID NO: 707, or SEQ ID NO: 708. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end). In one example, the ITR on each end can comprise, consist essentially of, or consist of SEQ ID NO: 706. In another example, the ITR on each end can comprise, consist essentially of, or consist of SEQ ID NO: 707. In one example, the ITR on at least one end comprises, consists essentially of, or consists of SEQ ID NO: 708. In one example, the ITR on the 5′ end comprises, consists essentially of, or consists of SEQ ID NO: 708. In one example, the ITR on the 3′ end comprises, consists essentially of, or consists of SEQ ID NO: 708. In one example, the ITR on each end can comprise, consist essentially of, or consist of SEQ ID NO: 708. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 706, and the ITR on the other end comprises, consists essentially of, or consists of SEQ ID NO: 707. In another example, the ITR on one end comprises, consists essentially of, or consists of SEQ ID NO: 706, and the ITR on the other end comprises, consists essentially of, or consists of SEQ ID NO: 708. In one example, the ITR on one end comprises, consists essentially of, or consists of SEQ ID NO: 707, and the ITR on the other end comprises, consists essentially of, or consists of SEQ ID NO: 708.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include a guide RNA or a nucleic acid encoding a guide RNA. In certain AAVs, the cargo can include an mRNA encoding a Cas nuclease, such as Cas9, and a guide RNA or a nucleic acid encoding a guide RNA. In certain AAVs, the cargo can include an exogenous donor sequence. In certain AAVs, the cargo can include an mRNA encoding a Cas nuclease, such as Cas9, a guide RNA or a nucleic acid encoding a guide RNA, and an exogenous donor sequence.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an exogenous donor sequence. In certain LNPs, the cargo can include an mRNA encoding a Cas nuclease, such as Cas9, a guide RNA or a nucleic acid encoding a guide RNA, and an exogenous donor sequence. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA to gRNA nucleic acid of from about 1:1 to about 1:2. In specific examples, the ratio of Cas mRNA to gRNA can be about 1:1 or about 1:2.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 1:1, 1:3, 1:5, 1:10, or 1:25.

A specific example of a suitable LNP has a nitrogen-to-phosphate (N/P) ratio of 4.5 and contains biodegradable cationic lipid, cholesterol, DSPC, and PEG2k-DMG in a 45:44:9:2 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in a 1:1 ratio by weight to the guide RNA. Another specific example of a suitable LNP contains Dlin-MC3-DMA (MC3), cholesterol, DSPC, and PEG-DMG in a 50:38.5:10:1.5 molar ratio.

Another specific example of a suitable LNP has a nitrogen-to-phosphate (N/P) ratio of 6 and contains biodegradable cationic lipid, cholesterol, DSPC, and PEG2k-DMG in a 50:38:9:3 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. The Cas9 mRNA can be in a 1:2 ratio by weight to the guide RNA.

Another specific example of a suitable LNP has a nitrogen-to-phosphate (N/P) ratio of 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT© GM-020(DMG-PEG)) in a 50:10:38.5:1.5 ratio or a 47:10:42:1 ratio. The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)).

Another specific example of a suitable LNP contains Dlin-MC3-DMA, DSPC, cholesterol, and a PEG lipid in a 45:9:44:2 ratio. Another specific example of a suitable LNP contains Dlin-MC3-DMA, DOPE, cholesterol, and PEG lipid or PEG DMG in a 50:10:39:1 ratio. Another specific example of a suitable LNP has Dlin-MC3-DMA, DSPC, cholesterol, and PEG2k-DMG at a 55:10:32.5:2.5 ratio. Another specific example of a suitable LNP has Dlin-MC3-DMA, DSPC, cholesterol, and PEG-DMG in a 50:10:38.5:1.5 ratio. Another specific example of a suitable LNP has Dlin-MC3-DMA, DSPC, cholesterol, and PEG-DMG in a 50:10:38.5:1.5 ratio.

Other examples of suitable LNPs can be found, e.g., in WO 2019/067992, herein incorporated by reference in its entirety for all purposes.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or exogenous donor nucleic acid/repair template). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous. Routes of administration can include, for example, parenteral, non-parenteral, oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The frequency of administration and the number of dosages can depend on the half-life of the exogenous donor nucleic acids, guide RNAs, or Cas proteins (or nucleic acids encoding the guide RNAs or Cas proteins) and the route of administration among other factors. The introduction of nucleic acids or proteins into the cell or animal can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

In some methods, a single dose of a CRISPR/Cas system disclosed herein for targeting a CS gene or CS locus may be administered (either alone or in combination with or in association with other therapeutic agents such as the C5 antigen-binding proteins or antibodies disclosed herein) to a subject in need thereof. In other methods, multiple doses of a CRISPR/Cas system disclosed herein for targeting a CS gene or CS locus may be administered (either alone or in combination with or in association with other therapeutic agents such as the C5 antigen-binding proteins or antibodies disclosed herein) to a subject over a defined time course. Such methods can comprise sequentially administering to a subject multiple doses of a CRISPR/Cas system disclosed herein for targeting a CS gene or CS locus (either alone or in combination with or in association with other therapeutic agents such as the C5 antigen-binding proteins or antibodies disclosed herein). Sequentially administering means that each dose of the CRISPR/Cas system is administered to the subject at a different point in time, such as on different days separated by a predetermined interval (e.g., hours, days, weeks, or months). Some methods comprise sequentially administering to the patient a single initial dose of a CRISPR/Cas system disclosed herein for targeting a CS gene or CS locus (either alone or in combination with other therapeutic agents such as the C5 antigen-binding proteins or antibodies disclosed herein), followed by one or more secondary doses of the CRISPR/Cas system (either alone or in combination with other therapeutic agents such as the C5 antigen-binding proteins or antibodies disclosed herein), and optionally followed by one or more tertiary doses of the CRISPR/Cas system (either alone or in combination with other therapeutic agents such as the C5 antigen-binding proteins or antibodies disclosed herein).

Initial dose, secondary doses, and tertiary doses refer to the temporal sequence of administration of the CRISPR/Cas system disclosed herein for targeting a C5 gene or C5 locus. Thus, the initial dose is the dose which is administered at the beginning of the treatment regimen (also referred to as the baseline dose), the secondary doses are the doses which are administered after the initial dose, and the tertiary doses are the doses which are administered after the secondary doses. The initial, secondary, and tertiary doses may all contain the same amount of CRISPR/Cas system, but generally may differ from one another in terms of frequency of administration. In some methods, however, the amount of CRISPR/Cas system contained in the initial, secondary, and/or tertiary doses varies from one another (e.g., adjusted up or down as appropriate) during the course of treatment. In some methods, two or more (e.g., 2, 3, 4, or 5) doses are administered at the beginning of the treatment regimen as loading doses followed by subsequent doses that are administered on a less frequent basis (e.g., maintenance doses).

Such methods may comprise administering to a patient any number of secondary and/or tertiary doses of a CRISPR/Cas system disclosed herein for targeting a C5 gene or C5 locus (either alone or in combination with other therapeutic agents such as the C5 antigen-binding proteins or antibodies disclosed herein). In one example, only a single secondary dose is administered to the patient. In another example, two or more (e.g., 2, 3, 4, 5, 6, 7, 8, or more) secondary doses are administered to the patient. Likewise, in another example, only a single tertiary dose is administered to the patient. In other examples, two or more (e.g., 2, 3, 4, 5, 6, 7, 8, or more) tertiary doses are administered to the patient.

The frequency at which the secondary and/or tertiary doses are administered to a patient can vary over the course of the treatment regimen. The frequency of administration may also be adjusted during the course of treatment by a physician depending on the needs of the individual patient following clinical examination.

Therapeutic compositions comprising the C5 antigen-binding proteins or antibodies or antigen-binding fragments thereof described herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding protein or antibody may vary depending upon the age and the size of a subject to be administered, target disease, conditions, route of administration, and the like. When an antigen-binding protein or antibody disclosed herein is used for treating a disease or disorder in an adult patient, or for preventing such a disease, it can be advantageous to administer the antigen-binding protein or antibody normally at a dose (e.g., single dose) of about 0.1 to about 100 mg/kg body weight, more preferably about 5 to about 80, about 10 to about 70, or about 20 to about 50 mg/kg body weight. In some embodiments, the antigen-binding protein or antibody can be administered at a dose (e.g., intravenous dose) of about 1, about 3, about 10, about 15, or about 30 mg/kg. Depending on the severity of the condition, the frequency and the duration of the treatment can be adjusted. In certain embodiments, the antigen-binding protein or antibody or antigen-binding fragment thereof can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the antigen-binding protein or antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432, herein incorporated by reference in its entirety for all purposes). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, e.g., Langer (1990) Science 249:1527-1533, herein incorporated by reference in its entirety for all purposes).

The use of nanoparticles to deliver the antigen-binding protein or antibody is also contemplated herein. Antibody-conjugated nanoparticles may be used both for therapeutic and diagnostic applications. Antibody-conjugated nanoparticles and methods of preparation and use are described in detail by Arruebo et al. (2009) (“Antibody-conjugated nanoparticles for biomedical applications” in J. Nanomat. Volume 2009, Article ID 439389, 24 pages, doi: 10.1155/2009/439389), incorporated herein by reference in its entirety for all purposes. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each of which is herein incorporated by reference in its entirety for all purposes.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition comprising an antigen-binding protein or antibody can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition disclosed herein. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.) and the HUMIRA™ Pen (Abbott Labs, Abbott Park, Ill.), to name only a few.

Pharmaceutical compositions for oral or parenteral use described above can be prepared into dosage forms in a unit dose suited to fit a dose of the active ingredients. Such dosage forms in a unit dose include, for example, tablets, pills, capsules, injections (ampoules), suppositories, etc. The amount of the antigen-binding protein or antibody contained is generally about 5 to about 500 mg per dosage form in a unit dose; especially in the form of injection, it is preferred that the antigen-binding protein or antibody is contained in about 5 to about 300 mg and in about 10 to about 300 mg for the other dosage forms.

In some embodiments, a single dose of a C5 antigen-binding protein or C5 antibody (i.e., anti-C5 antigen-binding protein or antibody) (or a pharmaceutical composition comprising a combination of a C5 antigen-binding protein or C5 antibody and any of the additional therapeutically active agents mentioned herein) may be administered to a subject in need thereof. In some embodiments, a single dose of a C5 antigen-binding protein or C5 antibody (i.e., anti-C5 antigen-binding protein or antibody) (or a pharmaceutical composition comprising a combination of a C5 antigen-binding protein or C5 antibody and a CRISPR/Cas system disclosed herein) may be administered to a subject in need thereof. In some embodiments, multiple doses of a C5 antigen-binding protein or C5 antibody (or a pharmaceutical composition comprising a combination of a C5 antigen-binding protein or C5 antibody and any of the additional therapeutically active agents mentioned herein) may be administered to a subject over a defined time course. Some methods comprise sequentially administering to a subject multiple doses of a C5 antigen-binding protein or C5 antibody. Some methods comprise administering a first dose of a C5 antigen-binding protein or C5 antibody in combination with a CRISPR/Cas system disclosed herein, and then sequentially administering additional doses of the C5 antigen-binding protein or C5 antibody (e.g., alone or in combination with the CRISPR/Cas system). As used herein, “sequentially administering” means that each dose is administered to the subject at a different point in time, e.g., on different days separated by a predetermined interval (e.g., hours, days, weeks or months).

Some embodiments include methods which comprise sequentially administering to the patient a single initial dose of a C5 antigen-binding protein or C5 antibody (e.g., intravenously), followed by one or more secondary doses of the C5 antigen-binding protein or C5 antibody (e.g., subcutaneously), and optionally followed by one or more tertiary doses of the C5 antigen-binding protein or C5 antibody (e.g., subcutaneously). In some embodiments, the initial dose is in combination with a CRISPR/Cas system disclosed herein, and the secondary and tertiary doses are optionally not in combination with a CRISPR/Cas system disclosed herein. In some embodiments, the CRISPR/Cas system and the C5 antigen-binding protein are in separate formulations and not a co-formulation. In some embodiments, the initial dose of C5 antigen-binding protein is higher than subsequent secondary and/or tertiary doses. In some embodiments, the initial dose of C5 antigen-binding protein is the same as subsequent secondary and/or tertiary doses. In some embodiments, doses of the C5 antigen-binding protein or more frequent and/or higher until a steady-state effect of CRISPR/Cas-mediated C5 knockdown is achieved, after which point lower doses and/or less frequent doses of C5 antigen-binding protein are used. For example, the steady-state effect of CRISPR/Cas-mediated C5 knockdown may be achieved between about 2 weeks and about 6 weeks, between about 3 weeks and about 5 weeks, or about 1 month following administration of the CRISPR/Cas system.

The terms “initial dose,” “secondary doses,” and “tertiary doses,” refer to the temporal sequence of administration of a C5 antigen-binding protein or C5 antibody. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of a C5 antigen-binding protein or C5 antibody, but generally may differ from one another in terms of frequency of administration. In certain embodiments, however, the amount of a C5 antigen-binding protein or C5 antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In some embodiments, each secondary and/or tertiary dose is administered 1 to 48 hours (e.g., 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, 15, 15%, 16, 16%, 17, 17%, 18, 18%, 19, 19%, 20, 20%, 21, 21%, 22, 22%, 23, 23%, 24, 24%, 25, 25%, 26, 26%, or more) after the immediately preceding dose. The phrase “the immediately preceding dose,” as used herein, means, in a sequence of multiple administrations, the dose of a C5 antigen-binding protein or C5 antibody which is administered to a patient prior to the administration of the very next dose in the sequence with no intervening doses.

The methods may comprise administering to a patient any number of secondary and/or tertiary doses of a C5 antigen-binding protein or C5 antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, a C5 antigen-binding protein (e.g., REGN3918) is administered to a subject as follows: (i) administering one or more doses (e.g., 1 dose) of about 30 mg/kg (body weight (BW)) of the antigen-binding protein intravenously (IV) (e.g., in combination with a CRISPR/Cas system disclosed herein); then (ii) administering either one or more doses (e.g., 2 or more) of about 800 mg of the antigen-binding protein (e.g., subcutaneously (SC)) (this may be referred to, herein, as the 30+800 dosing regimen), or one or more SC doses according to body weight as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg. Such SC dose(s) may be given on a weekly basis following the initial IV dose(s). The weekly doses can be continued indefinitely, for example, as long as a therapeutic effect or prevention of an undesired outcome (e.g., loss of serum albumin, or increase in serum LDH levels) is desired. See, e.g., WO 2021/081277 A1 or US 2021-0139573, each of which is herein incorporated by reference in its entirety for all purposes.

In some embodiments, one or more doses (e.g., one or more than one) of about 30 mg/kg (body weight (BW)) of C5 antigen-binding protein (e.g., REGN3918) or pharmaceutical formulation thereof, intravenously (IV) (e.g., in combination with a CRISPR/Cas system disclosed herein). An intravenous dose of 30 mg/kg has been demonstrated to help to quickly achieve the steady-state trough concentrations of the antigen-binding protein (e.g., antibody) required for sustained maximal CH50 inhibition which, thus, would lead to a therapeutic effect in the subject. Optional, further subcutaneous doses of antigen-binding protein may be given to the subject, e.g., weekly, e.g., following the IV dose(s).

In some embodiments, the subject (e.g., who suffers from PNH) is administered: (i) about 30 mg/kg of C5 antigen-binding protein intravenously (IV) initially (day 1) (e.g., in combination with a CRISPR/Cas system disclosed herein); then (ii) about 800 mg of the antigen-binding protein (e.g., subcutaneously (SC)) once a week (e.g., ±1, ±2 or ±3 days), e.g., on about day 8 (e.g., ±1, ±2 or ±3 days), 15 (e.g., ±1, ±2 or ±3 days), 22 (e.g., ±1, ±2 or ±3 days), etc., and every week (e.g., ±1, ±2 or ±3 days) thereafter.

In some embodiments, the subject is administered a therapeutically effective dose of a C5 antigen-binding protein or antibody (e.g., 30 mg/kg intravenous) (e.g., in combination with a CRISPR/Cas system disclosed herein). In some embodiments, the subject (e.g., who suffers from CHAPLE) is administered: (i) about 30 mg/kg of the antigen-binding protein intravenously (IV) (on day 1); then (ii) starting at about day 8 (e.g., day 8, day 8±1 day, day 8±2 days or day 8±3 days), one or more doses administered subcutaneously (SC), and continuing thereafter on a weekly basis, at doses depending on body weight (BW) as follows: for body weight (BW) <10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg.

Dosing once a week or weekly dosing or QW dosing refers to administering one or more doses where each occurs about 7 (e.g., ±1, ±2 or ±3) days after the immediately preceding dose.

In some embodiments, the IV and first SC dose are given on the same day.

In some embodiments, the C5 antigen-binding protein or antibody, when administered subcutaneously (SC), is delivered in less than 7 ml volume, about 0.625 mL, about 1 mL, about 1.75 mL, about 2.5 mL, about 4 mL, about 0.5-4.0 mL, or about 0.625-4.0 mL. In some embodiments, each SC dose is delivered in a single injection. In some embodiments, the SC injection is delivered in about 60 seconds or less.

In some embodiments, a subject (e.g., who suffers from a C5-associated disease) is administered one or more doses of C5 antigen-binding protein or antibody as follows: 1 mg/kg IV; 3 mg/kg IV; 300 mg SC; 800 mg SC; 10 mg/kg IV; 600 mg SC; or 30 mg/kg IV; or a loading dose of 15 mg/kg IV followed by one or more SC doses of 400 mg administered once weekly.

A serum concentration of about 100 mg/liter C5 antigen-binding protein or antibody (e.g., REGN3918) in a human subject maximally suppresses C5 activity (e.g., alternative, classical and lectin pathways) (e.g., as measured by AH50 and/or CH50 assay). Thus, included herein are methods for suppressing complement activity or C5 activity (e.g., alternative pathway (AP)) (e.g., suppressing C5 activity to about its maximal level (e.g., at least about 80%, 81%, 82%, 83%, 84%, 85%, 86%, 87%, 88%, 89%, 90%, 91%, 92%, 93%, 94%, 95%, 96%, 97%, 98%, 99% or 100%); e.g., which is measured as AH50 and/or CH50 activity) in a subject comprising administering one or more doses of the antigen-binding protein at sufficient levels so as to maintain the serum concentration of the anti-C5 antigen-binding protein at about 100 mg/liter or more (e.g., 150, 400, 600 or 700 mg/liter). In some embodiments, the dosing regimen comprises (i) administering one or more doses (e.g., 1 dose) of about 30 mg/kg (body weight (BW)) of the antigen-binding protein intravenously (IV); then, optionally, (ii) administering one or more weekly doses (e.g., 2 or more) of about 800 mg of the antigen-binding protein (e.g., subcutaneously (SC)); or, one or more weekly doses, administered subcutaneously (SC) depending on body weight (BW), as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; or for BW ≥60 kg: about 800 mg. The weekly doses can be continued indefinitely, for example, as long as maintenance of the serum concentration of the anti-C5 antigen-binding protein and/or suppression of the C5 activity is desired.

Also included are methods for achieving or achieving and maintaining a serum concentration (e.g., a steady state serum trough concentration over time) of about 100 mg/liter or more C5 antigen-binding protein or antibody in a subject comprising: (i) administering one or more doses (e.g., 1 dose) of about 30 mg/kg (body weight (BW)) of the antigen-binding protein intravenously (IV) (e.g., in combination with a CRISPR/Cas system disclosed herein); then, optionally, (ii) administering one or more weekly doses (e.g., 2 or more) of about 800 mg of the antigen-binding protein (e.g., subcutaneously (SC)); or one or more weekly doses, administered subcutaneously (SC) depending on body weight (BW) as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; or for BW ≥60 kg: about 800 mg. The weekly doses can be continued indefinitely, for example, as long as maintenance of the anti-C5 antigen-binding protein serum concentration is desired.

In some embodiments, the intravenous infusion of C5 antigen-binding protein or antibody is interrupted and restarted at 50% of the original infusion rate if, during the infusion, the subject suffers from one or more adverse events, such as, for example: cough, rigors/chills, Rash, pruritus (itching), urticaria (hives, welts, wheals), diaphoresis (sweating), hypotension, dyspnea (shortness of breath), vomiting, or flushing.

Also included are methods of administering C5 antigen-binding protein or antibody 5 (e.g., REGN3918) to a subject comprising (i) administering one or more doses (e.g., 1 dose) of about 30 mg/kg (body weight (BW)) of the antigen-binding protein (e.g., REGN3918) intravenously (IV) (e.g., in combination with a CRISPR/Cas system disclosed herein); then, optionally, (ii) administering one or more doses (e.g., 2 or more) of about 800 mg of the antigen-binding protein (e.g., subcutaneously (SC)). Such SC dose(s) may be given on a weekly basis following the initial IV dose(s). Also included are methods for administering an antigen-binding protein (e.g., REGN3918) to a subject comprising (i) administering one or more doses (e.g., 1 dose) of about 30 mg/kg (body weight (BW)) of the antigen-binding protein intravenously (IV) (e.g., in combination with a CRISPR/Cas system disclosed herein); then, optionally, (ii) administering one or more SC doses according to body weight as follows: for body weight (BW) <10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg. Such SC dose(s) may be given on a weekly basis following the initial IV dose(s). In some embodiments, the subject suffers from a C5-associated disease such as, for example, CHAPLE, PNH, aHUS or MG.

### D. Measuring Delivery, Activity, or Efficacy of CRISPR/Cas Systems Targeting a C5 Gene or C5 Locus

The methods disclosed herein can further comprise detecting or measuring activity of CRISPR/Cas systems targeting a C5 gene or C5 locus.

The measuring can comprise assessing the C5 locus for modifications. Various methods can be used to identify cells having a targeted genetic modification. The screening can comprise a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER© Probes, TAQMAN© Molecular Beacon probes, or ECLIPSE™ probe technology (see, e.g., US 2005/0144655, herein incorporated by reference in its entirety for all purposes). Next-generation sequencing (NGS) can also be used for screening. Next-generation sequencing can also be referred to as “NGS” or “massively parallel sequencing” or “high throughput sequencing.” NGS can be used as a screening tool in addition to the MOA assays to define the exact nature of the targeted genetic modification and whether it is consistent across cell types or tissue types or organ types.

The measuring can also comprise assessing C5 mRNA or C5 protein expression. Methods of measuring mRNA and protein expression are well-known.

The measuring can also comprise assessing C5 activity. For example, classical pathway hemolysis can be measured ex vivo using sensitized sheep red blood cells as disclosed elsewhere herein.

The assessing in an animal can be in any cell type from any tissue or organ. For example, the assessment can be in multiple cell types from the same tissue or organ (e.g., liver) or in cells from multiple locations within the tissue or organ. This can provide information about which cell types within a target tissue or organ are being targeted or which sections of a tissue or organ are being reached by the CRISPR/Cas systems. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

One example of an assay that can be used are the RNASCOPE™ and BASESCOPE™ RNA in situ hybridization (ISH) assays, which are methods that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a ZZ probe, and it can differentially detect single nucleotide edits and mutations in intact fixed tissue.

In some methods, the efficacy of a guide RNA is measured by percent editing of C5. In some methods, the percent editing of C5 is compared to the percent editing necessary to achieve knockdown of C5 protein (e.g., in the serum).

In some methods, the number of off-target sites at which a deletion or insertion occurs in an in vitro model (e.g., cultured cells) is determined, for example, by analyzing genomic DNA from cells transfected in vitro with Cas mRNA and guide RNA.

In some methods, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type, or which produce a frequency of off-target indel formation of <5% in a cell population and/or relative to the frequency of indel creation at the target site. In some methods, the disclosure provides guide RNAs which do not exhibit any off target indel formation in the target cell type. In some methods, guide RNAs are provided which produce indels at less than 5 off-target sites (e.g., as evaluated by one or more known methods or methods described herein). In some methods, guide RNAs are provided which produce indels at less than or equal to 4, 3, 2, or 1 off-target site(s) (e.g., as evaluated by one or more known methods or methods described herein). In some embodiments, the off-target site(s) does not occur in a protein coding region in the target cell genome.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 7
Description of Sequences.

| SEQ | | |
|---|---|---|
| ID | | |
| NO | Type | Description |
| 1 | Protein | Human C5 Protein NCBI Accession No. NP_ |
| | | 0017626.2 |
| 2 | DNA | Human C5 mRNA (cDNA) NCBI Accession |
| | | No. NM_001735.3 |
| 3 | DNA | Human C5 CDS CCDS ID CCDS6826.1 |
| 4 | Protein | Human C5 Protein NCBI Accession No. NP_ |
| | | 001304092.1 |
| 5 | DNA | Human C5 mRNA (cDNA) NCBI Accession |
| | | No. NM_001317163.2 |
| 6 | DNA | C5 Coding Sequence in Humanized C5 Mice |
| 7 | DNA | Monkey C5 CDS (Gene ID 102125658) |
| 8 | Protein | SpCas9 Protein V1 |
| 9 | DNA | SpCas9 DNA V1 |
| 10 | DNA | SpCas9 mRNA (cDNA) |
| 11 | Protein | SpCas9 Protein V2 |
| 12 | RNA | SpCas9 mRNA V2 |
| 13 | Protein | SV40 NLS v1 |
| 14 | Protein | SV40 NLS v2 |
| 15 | Protein | Nucleoplasmin NLS |
| 16 | RNA | crRNA Tail v1 |
| 17 | RNA | crRNA Tail v2 |
| 18 | RNA | TracrRNA vl |
| 19 | RNA | TracrRNA v2 |
| 20 | RNA | TracrRNA v3 |
| 21 | RNA | gRNA Scaffold v1 |
| 22 | RNA | gRNA Scaffold v2 |
| 23 | RNA | gRNA Scaffold v3 |
| 24 | RNA | gRNA Scaffold v4 |
| 25 | RNA | gRNA Scaffold v5 |
| 26 | RNA | gRNA Scaffold v6 |
| 27 | RNA | gRNA Scaffold v7 |
| 28 | RNA | gRNA Scaffold v8 |
| 29 | RNA | Modified gRNA Scaffold |
| 30 | DNA | Guide RNA Target Sequence Plus PAM v1 |
| 31 | DNA | Guide RNA Target Sequence Plus PAM v2 |
| 32 | DNA | Guide RNA Target Sequence Plus PAM v3 |
| 33-120 | RNA | C5 Guide Sequences |
| 121-208 | RNA | C5 crRNA Sequences |
| 209-296 | DNA | C5 Guide RNA Target Sequences |
| 297-334 | RNA | C5 and Control sgRNAs |
| 335-336 | DNA | Cas9 ORFs |
| 337 | DNA | ORF for Cas9 with Hibit Tag |
| 338 | RNA | Cas9 mRNA |
| 339 | RNA | Cas9 mRNA CDS |
| 341-703 | DNA or | C5 Antibody-Related Sequences |
| | Protein | |
| 704 | Protein | Human C5 |
| 705 | Protein | Mature Human C5 R885H |
| 706 | DNA | ITR 145 |
| 707 | DNA | ITR 141 |
| 708 | DNA | ITR 130 |

### EXAMPLES

### Example 1. Screening of Guide RNAs in Primary Human Hepatocytes (PHH)—Editing and Protein Knockdown

Several C5 CRISPR guide RNAs were designed in silico with the goal of knocking out the C5 gene, thereby reducing circulatory C5 levels. Primary human hepatocytes (PHH) (Invitrogen) were thawed and resuspended in hepatocyte thawing medium with supplements (Gibco, Cat. CM7500) followed by centrifugation. The supernatant was discarded and the pelleted cells resuspended in hepatocyte plating medium (William's E Medium (Invitrogen, Cat. A1217601) with Dexamethasone+cocktail supplement, FBS content, and Plating Supplements (Gibco, Cat. CM3000)). Cells were counted and plated on Bio-coat collagen I coated 96-well plates (ThermoFisher, Cat. 877272) at a density of 30,000-35,000 cells/well for PHH. Plated cells were allowed to settle and adhere for 4-6 hours in a tissue culture incubator at 37° C. and 5% CO₂ atmosphere. After incubation, cells were checked for monolayer formation and were washed once with hepatocyte maintenance medium (William's E Medium with maintenance supplements (Gibco, Cat. CM4000)).

Six hours after thaw, individual crRNA as described in Table 8 and tracrRNA (trRNA) was pre-annealed by mixing equivalent amounts of reagent and incubating at 95° C. for 2 min and cooling to room temperature. The dual guide (dgRNA) consisting of pre-annealed crRNA and trRNA, was incubated with Streptococcus pyogenes Cas9 (Spy Cas9) protein to form a ribonucleoprotein (RNP) complex. Cells were transfected with Lipofectamine RNAiMAX (ThermoFisher, Cat. 13778150) according to the manufacturer's protocol. Cells were transfected with an RNP containing Spy Cas9 (10 nM), crRNA (10 nM), trRNA (10 nM), Lipofectamine RNAiMAX (1.0 μL/well), and OptiMem media.

The cells were lysed 48 hours post-transfection and genomic DNA was isolated using 50 μL/well BuccalAmp DNA Extraction solution (Epicentre, Cat. QE09050) according to manufacturer's protocol. PCR primers were designed around the target site within the gene of interest (e.g., C5), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated. The editing percentage (e.g., the “editing efficiency” or “percent editing”) is defined as the total number of sequence reads with insertions or deletions (“indels”) over the total number of sequence reads, including wild type. Editing results with tested guides are shown in Table 8.

TABLE 8
Mean Percent Editing Following Treatment
with RNP and Listed dgRNAs.

| | |
|---|---|
| Primer Set A | Primer Set B |

| Guide ID | Mean | SD | n | Mean | SD | n |
|---|---|---|---|---|---|---|
| CR005628 | 25.00 | 0.00 | 1 | 14.70 | 2.27 | 3 |
| CR005629 | no data | | | 14.50 | 2.08 | 3 |
| CR005630 | 22.63 | 0.45 | 3 | 19.93 | 1.08 | 3 |
| CR005631 | 8.47 | 0.25 | 3 | 8.37 | 0.29 | 3 |
| CR005632 | 28.07 | 3.20 | 3 | 25.10 | 1.39 | 3 |
| CR005633 | 36.00 | 1.85 | 3 | 32.97 | 0.99 | 3 |
| CR005634 | 8.17 | 1.56 | 3 | 7.83 | 1.72 | 3 |
| CR005635 | 4.50 | 0.10 | 3 | 3.30 | 0.00 | 1 |
| CR005636 | 19.00 | 0.52 | 3 | 17.30 | 1.14 | 3 |
| CR005637 | 7.73 | 1.46 | 3 | 6.40 | 2.46 | 3 |
| CR005638 | 18.67 | 1.21 | 3 | 18.40 | 3.90 | 3 |
| CR005639 | 5.07 | 0.71 | 3 | 6.43 | 1.53 | 3 |
| CR005640 | 12.53 | 1.94 | 3 | 11.87 | 1.50 | 3 |
| CR005641 | 16.77 | 1.36 | 3 | 15.53 | 1.19 | 3 |
| CR005642 | 19.00 | 1.41 | 3 | 19.17 | 1.46 | 3 |
| CR005643 | 30.57 | 4.96 | 3 | 28.07 | 7.71 | 3 |
| CR005644 | 31.80 | 5.02 | 3 | 16.23 | 14.25 | 3 |
| CR005645 | 29.83 | 1.33 | 3 | 27.47 | 1.72 | 3 |
| CR005646 | 18.17 | 0.06 | 3 | 15.37 | 2.08 | 3 |
| CR005647 | 8.73 | 1.86 | 3 | 7.77 | 0.91 | 3 |
| CR005648 | 13.17 | 2.54 | 3 | 11.87 | 1.85 | 3 |
| CR005649 | 28.97 | 5.99 | 3 | 27.63 | 3.72 | 3 |
| CR005650 | 2.90 | 0.79 | 3 | 2.97 | 1.14 | 3 |
| CR005651 | 1.30 | 0.35 | 3 | 1.10 | 0.56 | 3 |
| CR005652 | 13.43 | 1.25 | 3 | 12.53 | 2.14 | 3 |
| CR005653 | 16.30 | 1.47 | 3 | 13.80 | 2.33 | 3 |
| CR005654 | 8.57 | 1.29 | 3 | 7.57 | 1.25 | 3 |
| CR005655 | 66.67 | 3.59 | 3 | 62.97 | 6.62 | 3 |
| CR005656 | 2.17 | 0.21 | 3 | 1.70 | 0.17 | 3 |
| CR005657 | 14.80 | 2.34 | 3 | no data | | |
| CR005658 | 6.73 | 2.84 | 3 | no data | | |
| CR005659 | 8.87 | 1.47 | 3 | no data | | |
| CR005660 | 68.03 | 0.85 | 3 | 67.33 | 9.88 | 3 |
| CR005661 | 32.20 | 6.43 | 3 | no data | | |
| CR005662 | 44.33 | 0.76 | 3 | 38.70 | 4.83 | 3 |
| CR005663 | 26.70 | 3.20 | 3 | 28.03 | 1.74 | 3 |
| CR005664 | 52.83 | 4.28 | 3 | 53.27 | 1.91 | 3 |
| CR005665 | 39.77 | 2.22 | 3 | 38.40 | 2.75 | 3 |
| CR005666 | 24.30 | 5.75 | 3 | 39.20 | 19.25 | 3 |
| CR005667 | 7.63 | 2.58 | 3 | 5.93 | 1.59 | 3 |
| CR005668 | 19.23 | 1.36 | 3 | 14.40 | 2.97 | 2 |
| CR005669 | 6.90 | 0.44 | 3 | 18.57 | 18.62 | 3 |
| CR005670 | 7.87 | 1.53 | 3 | 8.37 | 1.38 | 3 |
| CR005671 | 6.93 | 0.84 | 3 | 6.67 | 0.85 | 3 |
| CR005672 | 7.83 | 1.62 | 3 | 7.57 | 0.72 | 3 |
| CR005673 | 4.33 | 0.78 | 3 | 4.37 | 1.12 | 3 |
| CR005674 | 7.17 | 1.63 | 3 | 7.40 | 0.95 | 3 |
| CR005675 | 12.20 | 1.56 | 3 | 11.40 | 0.56 | 3 |
| CR005676 | 8.67 | 1.33 | 3 | 7.70 | 2.35 | 3 |
| CR005677 | 36.67 | 10.05 | 3 | 33.80 | 4.79 | 3 |
| CR005678 | 10.40 | 0.60 | 3 | 8.47 | 1.19 | 3 |
| CR005679 | 6.20 | 0.79 | 3 | 5.37 | 1.11 | 3 |
| CR005680 | 46.50 | 0.60 | 3 | 41.47 | 3.81 | 3 |
| CR005681 | 38.23 | 2.80 | 3 | 36.67 | 2.41 | 3 |
| CR005682 | 54.33 | 9.55 | 3 | 53.77 | 8.18 | 3 |
| CR005683 | 22.57 | 2.14 | 3 | 20.27 | 2.02 | 3 |
| CR005684 | 26.70 | 2.69 | 3 | 24.60 | 3.90 | 3 |
| CR005685 | 34.60 | 1.39 | 3 | 32.07 | 1.15 | 3 |
| CR005686 | 21.47 | 2.10 | 3 | 23.87 | 6.01 | 3 |
| CR005687 | 42.27 | 3.24 | 3 | 40.50 | 4.33 | 3 |
| CR005688 | 21.27 | 3.46 | 3 | 18.63 | 3.19 | 3 |
| CR005689 | 2.33 | 0.64 | 3 | 8.30 | 0.00 | 1 |
| CR005690 | 11.63 | 0.12 | 3 | 25.40 | 22.51 | 3 |
| CR005691 | 12.80 | 0.78 | 3 | 17.50 | 10.61 | 2 |
| CR005692 | 60.70 | 2.96 | 3 | 58.43 | 4.24 | 3 |
| CR005693 | 25.50 | 2.26 | 3 | 25.83 | 1.78 | 3 |
| CR005694 | no data | | | 9.93 | 0.61 | 3 |
| CR005695 | 31.37 | 1.55 | 3 | 31.07 | 2.65 | 3 |
| CR005696 | 24.80 | 5.20 | 3 | 23.23 | 2.54 | 3 |
| CR005697 | 24.50 | 2.43 | 3 | 25.87 | 5.17 | 3 |
| CR005698 | 29.53 | 2.87 | 3 | 29.40 | 2.76 | 3 |
| CR005699 | 33.03 | 1.07 | 3 | 30.27 | 3.56 | 3 |
| CR005700 | 28.97 | 1.25 | 3 | 24.70 | 6.30 | 3 |
| CR005701 | 5.57 | 1.16 | 3 | 6.63 | 0.99 | 3 |
| CR005702 | 12.60 | 0.46 | 3 | 11.73 | 2.40 | 3 |
| CR005703 | 6.00 | 1.81 | 3 | 5.20 | 0.75 | 3 |
| CR005704 | 17.07 | 3.65 | 3 | 16.87 | 2.54 | 3 |
| CR005705 | 11.97 | 1.29 | 3 | no data | | |
| CR005706 | 8.93 | 0.40 | 3 | 8.77 | 0.12 | 3 |
| CR005707 | 5.33 | 0.31 | 3 | no data | | |
| CR005708 | 10.20 | 1.66 | 3 | 11.47 | 1.99 | 3 |
| CR005709 | 37.63 | 2.14 | 3 | 35.70 | 3.60 | 3 |
| CR005710 | 24.17 | 1.25 | 3 | 27.00 | 1.75 | 3 |
| CR005713 | 45.27 | 0.70 | 3 | 44.93 | 3.29 | 3 |
| CR005714 | 42.50 | 3.62 | 3 | no data | | |
| CR005715 | 26.27 | 2.32 | 3 | no data | | |

### Example 2. Off-Target Analysis of C5 Guide RNAs

Screening for potential off-target genomic sites cleaved by Cas9 targeting HLA-A was performed. See, e.g., Cameron et al. (2017) Nature Methods 6, 600-606, herein incorporated by reference in its entirety for all purposes. In this experiment, eight sgRNAs targeting human C5 and control guides targeting EMX1 and VEGFA, with known off-target profiles were screened using purified genomic DNA from lymphoblast cell line NA24385 (Coriell Institute). The number of potential off-target sites were detected using a sgRNA as shown in Table 9 at a concentration of 192 nM sgRNA and 64 nM RNP in the biochemical assay. The assay identified potential on target and off-target sites for the sgRNAs tested, as shown in Table 9.

TABLE 9
Off-Target Analysis.

| Guide | Target | Count of Potential Sites |
|---|---|---|
| G000644 | EMX1 | 304 |
| G000645 | VEGFA | 4227 |
| G012393 | C5 | 220 |
| G012394 | C5 | 51 |
| G012395 | C5 | 259 |
| G012396 | C5 | 224 |
| G012397 | C5 | 185 |
| G012398 | C5 | 359 |
| G012399 | C5 | 38 |
| G012400 | C5 | 277 |

TABLE 10
sgRNAs Tested.

| | Corresponding | | |
|---|---|---|---|
| sgRNA | crRNA | Modified sgRNA Sequence | SEQ ID |
| G012393 | CR005660 | mU*mG*mC*CUACCAUAAAUACCCCUGUUUUAGAmGmCmU | 316 |
| | | mAmGmAmAmAmUmAmGmCAAGUUAAAAUAAGGCUAGUC | |
| | | CGUUAUCAmAmCmUmUmGmAmAmAmAmAmGmUmGmGmC | |
| | | mAmCmCmGmAmGmUmCmGmGmUmGmCmU*mU*mU*mU | |
| G012394 | CR005655 | mC*mU*mC*UCGGGCUUCAACAGGCAGUUUUAGAmGmCm | 317 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G012395 | CR005692 | mG*mA*mG*AUGAGUAUGCUAUUGCUGUUUUAGAmGmCm | 318 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G012396 | CR005682 | mU*mC*mC*AGCUAGGUGGAACACAUGUUUUAGAmGmCm | 319 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G012397 | CR005680 | mU*mG*mA*GGAAGGUAAGUCCAGCUGUUUUAGAmGmCm | 320 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G012398 | CR005662 | mU*mG*mC*CCUGAUGAUCAAUGACUGUUUUAGAmGmCm | 321 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G012399 | CR005714 | mC*mC*mU*CCAACCAUGGGCCUUUUGUUUUAGAmGmCmU | 322 |
| | | mAmGmAmAmAmUmAmGmCAAGUUAAAAUAAGGCUAGUC | |
| | | CGUUAUCAmAmCmUmUmGmAmAmAmAmAmGmUmGmGmC | |
| | | mAmCmCmGmAmGmUmCmGmGmUmGmCmU*mU*mU*mU | |
| G012400 | CR005677 | mU*mU*mC*UUAGAGAAGAGUGAUCUGUUUUAGAmGmCm | 323 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G000395 | non-targeting | mG*mC*mA*AUGGUGUAGCGGGUUUUAGAmGmCmUmAmG | 332 |
| | guide in mouse | mAmAmAmUmAmGmCAAGUUAAAAUAAGGCUAGUCCGUU | |
| | | AUCAmAmCmUmUmGmAmAmAmAmAmGmUmGmGmCmAm | |
| | | CmCmGmAmGmUmCmGmGmUmGmCmU*mU*mU*mU | |
| G000644 | EMX1 | mG*mA*mG*UCCGAGCAGAAGAAGAAGUUUUAGAmGmCm | 333 |
| | | UmAmGmAmAmAmUmAmGmCAAGUUAAAAUAAGGCUAGU | |
| | | CCGUUAUCAmAmCmUmUmGmAmAmAmAmAmGmUmGmGm | |
| | | CmAmCmCmGmAmGmUmCmGmGmUmGmCmU*mU*mU*mU | |
| G000645 | VEGFA | mG*mA*mC*CCCCUCCACCCCGCCUCGUUUUAGAmGmCmU | 334 |
| | | mAmGmAmAmAmUmAmGmCAAGUUAAAAUAAGGCUAGUC | |
| | | CGUUAUCAmAmCmUmUmGmAmAmAmAmAmGmUmGmGmC | |
| | | mAmCmCmGmAmGmUmCmGmGmUmGmCmU*mU*mU*mU | |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods are validated on a guide-by-guide basis using targeted sequencing of the identified potential off-target sites.

### Example 3. Testing of Guide RNAs Targeting Human C5 In Vitro

Fifteen guide RNAs were tested for their efficacy in vitro in primary human hepatocytes and primary cynomolgus hepatocytes. The cynomolgus C5 coding sequence is set forth in SEQ ID NO: 7. The sequences for these fifteen guide RNAs are provided in Table 11.

TABLE 11
Human C5 Guide RNAs.

| crRNA | gRNA | | | SEQ |
|---|---|---|---|---|
| ID | ID | LNP ID | Guide Sequence | ID NO |
| CR005660 | G012393 | LNP0365 | UGCCUACCAUAAAUACCCCU | 65 |
| CR005655 | G012394 | LNP0366 | CUCUCGGGCUUCAACAGGCA | 60 |
| CR005692 | G012395 | LNP0367 | GAGAUGAGUAUGCUAUUGCU | 97 |
| CR005682 | G012396 | LNP0368 | UCCAGCUAGGUGGAACACAU | 87 |
| CR005680 | G012397 | LNP0369 | UGAGGAAGGUAAGUCCAGCU | 85 |
| CR005662 | G012398 | LNP0370 | UGCCCUGAUGAUCAAUGACU | 67 |
| CR005714 | G012399 | LNP0371 | CCUCCAACCAUGGGCCUUUU | 119 |
| CR005677 | G012400 | LNP0372 | UUCUUAGAGAAGAGUGAUCU | 82 |
| CR005664 | | | AGCCCAGUCAUUGAUCAUCA | 69 |
| CR005665 | | | AAGCCCAGUCAUUGAUCAUC | 70 |
| CR005681 | | | GGAAGGUAAGUCCAGCUAGG | 86 |
| CR005685 | | | CCAUAUUAAGAGACACAGUU | 90 |
| CR005687 | | | AAGGGCAAAAUUAUCCACUU | 92 |
| CR005709 | | | UCUCUGAGGAUAAAUGAACA | 114 |
| CR005713 | | | UUUCCUGCUACCUCCAACCA | 118 |

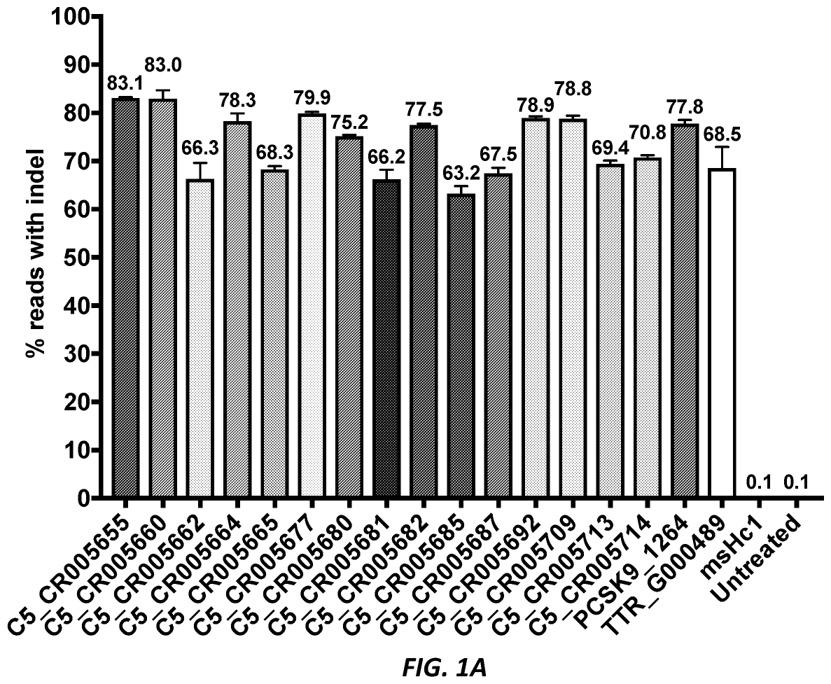

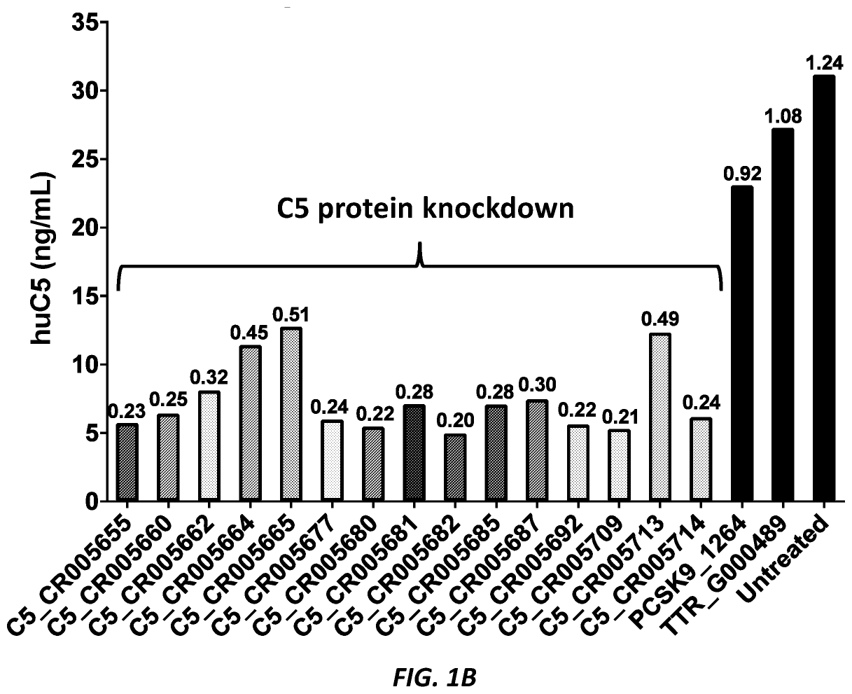

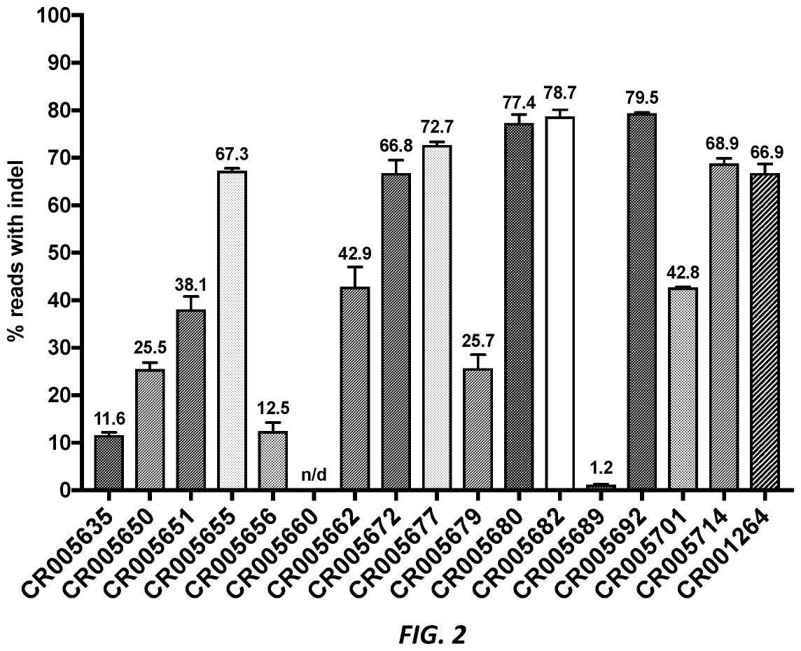

C5 indel frequency at Day 5 following administration of 10 nM Cas9 ribonucleoprotein complexes to primary human hepatocytes is shown in FIG. 1A. Cas9:crRNA:tracrRNA ribonucleoprotein complexes were transfected into cells at a final concentration of 10 nM in the culture media using CRISPRMAX (ThermoFisher) transfection reagent. The Cas9 protein used in the RNP experiments was purchased from Thermo Fisher. Five days post-transfection, cell lysates were prepared using QuickExtract™ DNA Extraction Solution (Epicentre). Lysates were used as genomic DNA templates for amplicon-based next-generation sequencing at each respective gRNA target site. Positive control sgRNAs PCSK9_1264 and TTR_G000489 target regions in PCSK9 and TTR genes, respectively. Negative control sgRNA msHcl is a human non-targeting sgRNA. The corresponding C5 protein expression is shown in FIG. 1B. Up to 80% knockdown of C5 protein in primary human hepatocyte conditioned medium was observed. C5 indel frequency at Day 3 following administration of Cas9 mRNA and 25 nM chemically modified sgRNA to primary human hepatocytes is shown in FIG. 2. Cas9 mRNA and sgRNA were co-transfected into cells using MessengerMax (ThermoFisher) transfection reagent. Three days post-transfection, cell lysates were prepared using QuickExtract™ DNA Extraction Solution (Epicentre). Lysates were used as genomic DNA templates for amplicon-based next-generation sequencing at each respective gRNA target site. This assay more closely mimics in vitro the LNP delivery modality used in vivo in that both approaches deliver Cas9 and sgRNA as ribonucleotides to the target cells.

### Example 4. Testing of Guide RNAs Targeting Human C5 In Vivo in Humanized C5 Mice

Eight top guide RNAs (sgRNAs corresponding to CR0005680, CR0005692, CR0005682, CR0005660, CR0005655, CR0005677, CR0005662, and CR0005714) were chosen for in vivo experiments and were synthesized with Cas9 mRNA (SEQ ID NO: 338, with the CDS set forth in SEQ ID NO: 339) in lipid nanoparticles (LNP) for in vivo delivery. The LNPs contained biodegradable cationic lipid, cholesterol, DSPC, and PEG2k-DMG in a 50:38:9:3 molar ratio. The biodegradable cationic lipid was (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. The Cas9 mRNA was in a 1:1 ratio by weight to the guide RNA.

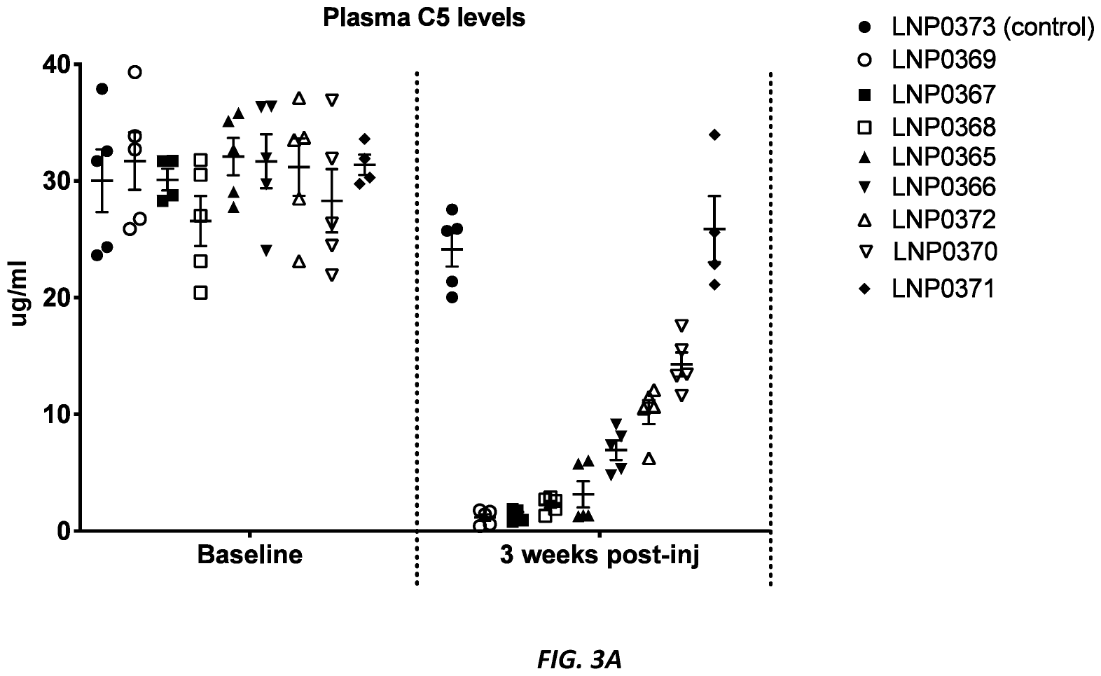

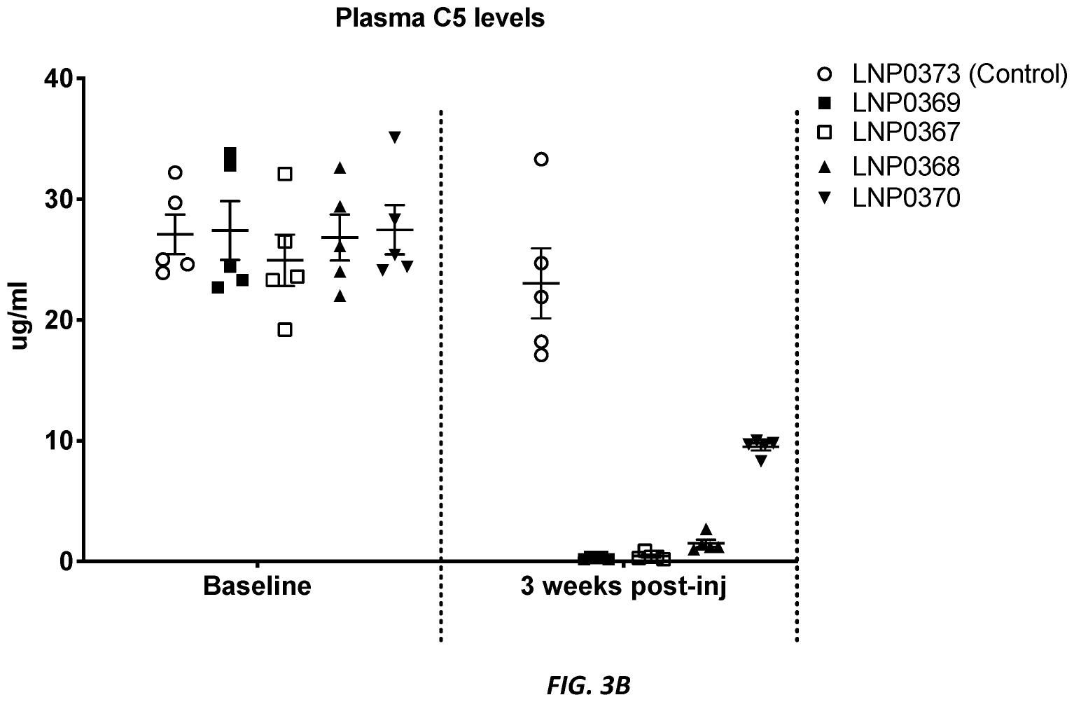

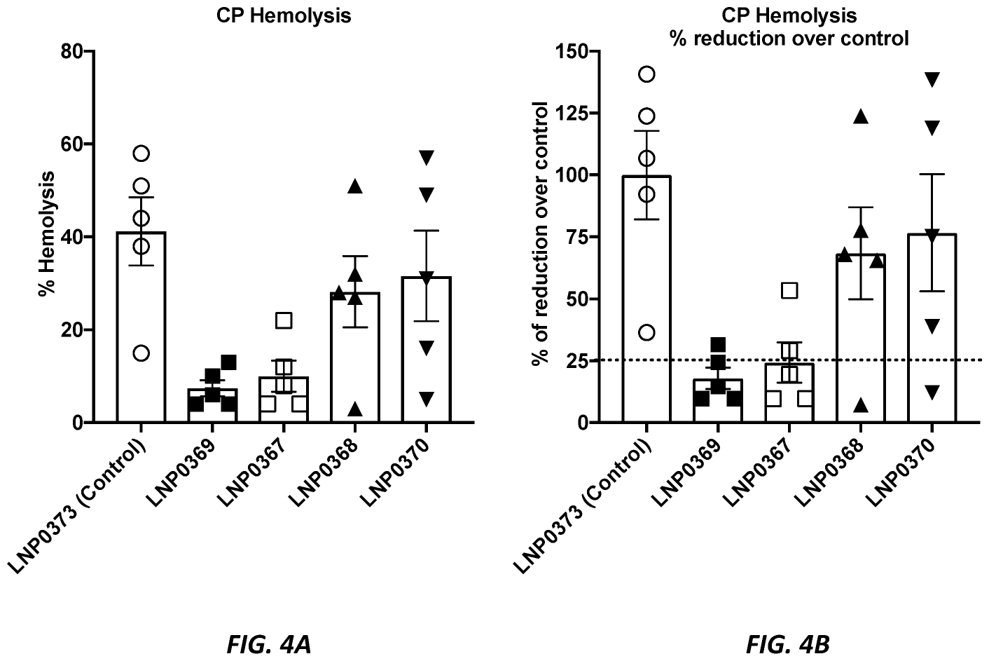

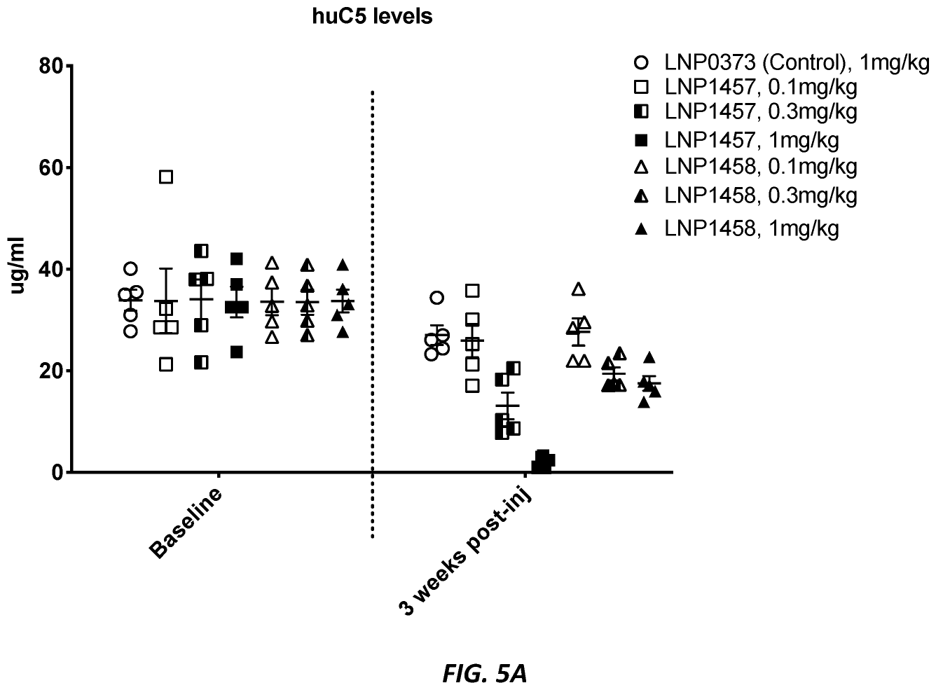

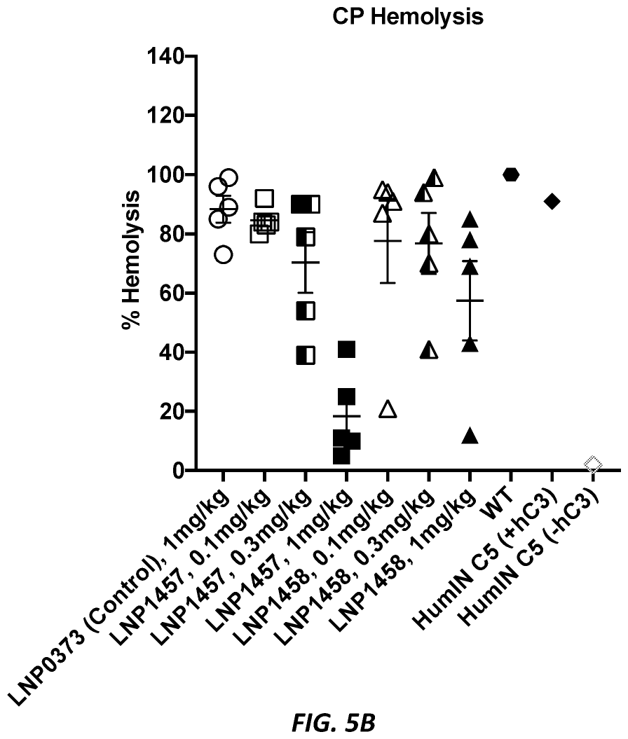

The eight C5 CRISPR/Cas9 formulated LNPs led to a dose-dependent reduction in circulatory C5 levels in humanized C5 mice. In the humanized C5 mice, coding exons 2 through 42 of the human C5 gene (97 kb) replaced a 75.8 kb portion of the murine C5 gene locus spanning coding exons 2 through 41 and including a portion of the 3′ UTR. See WO 2015/171523 A1, herein incorporated by reference in its entirety for all purposes. The coding sequence of the humanized C5 gene is set forth in SEQ ID NO: 6. A good correlation existed when comparing plasma C5 levels with percent editing of the C5 gene in vivo (data not shown). Two guides, sgRNAs corresponding to CR005680 and CR005692, reduced circulatory C5 levels more than 50% at 0.3 mg/kg (data not shown) and about 95% at 1 mg/kg at 3 weeks post-administration when delivered as a single intravenous injection in humanized C5 mice. See FIG. 3A. Humanized C5 mice were administered with a single intravenous of injection of C5 LNP formulated CRISPR/Cas9 guides at 1 mg/kg. The treatment groups were formed based on plasma C5 measured a week before the treatment. Eight guides (n=5 per group) were tested in the experiment: LNP0365; LNP0366; LNP0367; LNP0368; LNP0369; LNP0370; LNP0371; and LNP0372. LNP0373 was the control used in the experiment. Plasma C5 levels were measured weekly after guide administration for 3 weeks by using a commercial ELISA (Abcam). Three guides, LNP0369, LNP0367, and LNP0368, achieved a 90-95% reduction of plasma C5 levels (FIG. 3A). In another experiment, humanized C5 mice were administered with a single intravenous of injection of C5 LNP formulated CRISPR/Cas9 guides at 2 mg/kg. The treatment groups were formed based on plasma C5 measured a week before the treatment. Four guides (n=5 per group) were tested in the experiment: LNP0367; LNP0368; LNP0369; and LNP0370. LNP0373 (non-targeting mouse guide RNA) was the control used in the experiment. Plasma C5 levels were measured at 2 weeks and 3 weeks using an in-house developed ELISA with two C5 monoclonal antibodies that bind to C5 at different epitopes. To examine the effect on C5 function, classical pathway (CP) hemolysis using sensitized RBC was utilized at 3 weeks post-administration of the guides. See, e.g., Latuszek et al. (2020) PLoS One 15(5):e0231892, herein incorporated by reference in its entirety for all purposes. LNP0369 and LNP0367 achieved a reduction of plasma C5 levels of about 98% (FIG. 3B). About 75% reduction of hemolysis was observed with LNP0369 and LNP0367 (FIGS. 4A and 4B). The humanized C5 mice were administered with LNP1457 (comprising sgRNA corresponding to CR005692) and LNP1458 (comprising sgRNA corresponding to CR005662) at 0.1, 0.3, or 1 mg/kg. There was a dose-dependent decrease in plasma C5 levels with about 92% reduction with LNP1457 at 1 mg/kg (FIG. 5A) and a corresponding 80% decrease in classical pathway hemolysis ex vivo (FIG. 5B).

Both top leads, sgRNAs corresponding to CR005680 and CR005692, were highly specific for C5 with little or no off-target hits as done by GuideSeq and SiteSeq experiments. The results are summarized in Table 12. “HEK293 Off Targets” represents the number of potential off-target sites identified in HEK293 cells that stably overexpress Cas9. These experiments show the successful generation of CRISPR/Cas9 guide RNAs that are highly efficient and selective for C5.

TABLE 12
Summary of C5 gRNA Results.

| | | | In Vivo Plasma C5 Levels | | | | | HEK293 |
|---|---|---|---|---|---|---|---|---|
| Corresponding | | | (% Change vs. Control) | | | % Indel | % Indel | Off |
| crRNA ID | sgRNA ID | LNP ID | 2 mg/kg | 1 mg/kg | 0.3 mg/kg | in PHH | in PCH | Targets |
| | | LNP0373 | N/A | N/A | N/A | N/A | N/A | N/A |
| | | (Control) | | | | | | |
| CR005680 | G012397 | LNP0369 | −98.70 | −95.12 | −55.91 | 75.2 | 23.1 | 0 |
| CR005692 | G012395 | LNP0367 | −98.09 | −94.39 | −50.35 | 78.9 | 34.0 | 0 |
| CR005682 | G012396 | LNP0368 | −93.49 | −90.55 | −30.41 | 77.5 | 46.6 | 0 |
| CR005660 | G012393 | LNP0365 | ND | −86.88 | −43.55 | 83 | 28.2 | 5 |
| CR005655 | G012394 | LNP0366 | ND | −71.24 | −4.16 | 83.1 | 31.3 | 0 |
| CR005677 | G012400 | LNP0372 | ND | −57.74 | −0.69 | 79.9 | 9.9 | 1 |
| CR005662 | G012398 | LNP0370 | −58.77 | −40.77 | −16.17 | 68.3 | 16.9 | 0 |
| CR005714 | G012399 | LNP0371 | ND | 7.33 | 5.4 | 70.8 | 23.6 | 3 |

ND = not done
N/A = not applicable

We claim:

1. A composition comprising a guide RNA or a DNA encoding the guide RNA, wherein the guide RNA comprises a DNA-targeting segment that targets a guide RNA target sequence in a C5 gene, and wherein the guide RNA binds to a Cas protein and targets the Cas protein to the guide RNA target sequence in the C5 gene.

2. The composition of claim 1, wherein the guide RNA target sequence is in coding exon 27, 22, 21, 15, 12, or 1 of the C5 gene, or wherein the guide RNA target sequence is in coding exon 15 or 12 of the C5 gene.

3. The composition of claim 1 or 2, wherein the C5 gene is a human C5 gene.

4. The composition of any one of claims 1-3, wherein the DNA-targeting segment comprises at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97, or
wherein the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273, or wherein the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

5. The composition of any preceding claim, wherein the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97, or
wherein the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

6. The composition of any preceding claim, wherein the composition comprises the guide RNA in the form of RNA.

7. The composition of any one of claims 1-5, wherein the composition comprises the DNA encoding the guide RNA.

8. The composition of any one of claims 1-6, wherein the guide RNA comprises at least one modification.

9. The composition of claim 8, wherein the at least one modification comprises a 2′-O-methyl-modified nucleotide.

10. The composition of claim 8 or 9, wherein the at least one modification comprise a phosphorothioate bond between nucleotides.

11. The composition of any one of claims 8-10, wherein the at least one modification comprise a modification at one or more of the first five nucleotides at the 5′ end of the guide RNA.

12. The composition of any one of claims 8-11, wherein the at least one modification comprises a modification at one or more of the last five nucleotides at the 3′ end of the guide RNA.

13. The composition of any one of claims 8-12, wherein the at least one modification comprises phosphorothioate bonds between the first four nucleotides at the 5′ end of the guide RNA.

14. The composition of any one of claims 8-13, wherein the at least one modification comprises phosphorothioate bonds between the last four nucleotides at the 3′ end of the guide RNA.

15. The composition of any one of claims 8-14, wherein the at least one modification comprises 2′-O-methyl-modified nucleotides at the first three nucleotides at the 5′ end of the guide RNA.

16. The composition of any one of claims 8-15, wherein the at least one modification comprises 2′-O-methyl-modified nucleotides at the last three nucleotides at the 3′ end of the guide RNA.

17. The composition of any one of claims 8-16, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

18. The composition of any one of claims 8-17, wherein the guide RNA comprises the modified nucleotides of SEQ ID NO: 29.

19. The composition of any preceding claim, wherein the guide RNA is a single guide RNA (sgRNA).

20. The composition of claim 19, wherein the guide RNA comprises, consists essentially of, or consists of the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

21. The composition of any one of claims 1-17, wherein the guide RNA is a dual guide RNA (dgRNA) comprising two separate RNA molecules comprising a CRISPR RNA (crRNA) and a trans-activating crRNA (tracrRNA).

22. The composition of claim 21, wherein the crRNA comprises the sequence set forth in any one of SEQ ID NOS: 16-17.

23. The composition of claim 21 or 22, wherein the tracrRNA comprises the sequence set forth in any one of SEQ ID NOS: 18-20.

24. The composition of any preceding claim, wherein the composition is associated with a lipid nanoparticle.

25. The composition of claim 24, wherein the lipid nanoparticle comprises a cationic lipid, a neutral lipid, a helper lipid, and a stealth lipid.

26. The composition of claim 25, wherein the cationic lipid is Lipid A.

27. The composition of claim 25 or 26, wherein the neutral lipid is DSPC.

28. The composition of any one of claims 25-27, wherein the helper lipid is cholesterol.

29. The composition of any one of claims 25-28, wherein the stealth lipid is PEG2k-DMG.

30. The composition of any one of claims 25-29, wherein the cationic lipid is Lipid A, the neutral lipid is DSPC, the helper lipid is cholesterol, and the stealth lipid is PEG2k-DMG.

31. The composition of any preceding claim, wherein the composition is a pharmaceutical composition comprising a pharmaceutically acceptable carrier.

32. The composition of any preceding claim, further comprising the Cas protein or a nucleic acid encoding the Cas protein.

33. The composition of claim 32, wherein the Cas protein is a Cas9 protein.

34. The composition of claim 33, wherein the Cas protein is derived from a Streptococcus pyogenes Cas9 protein.

35. The composition of any one of claims 32-34, wherein the composition comprises the Cas protein in the form of a protein.

36. The composition of any one of claims 32-34, wherein the composition comprises the nucleic acid encoding the Cas protein, wherein the nucleic acid comprises a DNA encoding the Cas protein, optionally wherein the composition comprises the DNA encoding the guide RNA.

37. The composition of any one of claims 32-34, wherein the composition comprises the nucleic acid encoding the Cas protein, wherein the nucleic acid comprises an mRNA encoding the Cas protein, optionally wherein the composition comprises the guide RNA in the form of RNA.

38. The composition of claim 37, wherein the mRNA encoding the Cas protein comprises at least one modification.

39. The composition of claim 38, wherein the mRNA encoding the Cas protein is modified to comprise a modified uridine at one or more or all uridine positions.

40. The composition of claim 39, wherein the modified uridine is N1-methyl-pseudouridine.

41. The composition of claim 39 or 40, wherein the mRNA encoding the Cas protein is fully substituted with N1-methyl-pseudouridine.

42. The composition of any one of claims 38-41, wherein the mRNA encoding the Cas protein comprises a 5′ cap.

43. The composition of any one of claims 38-42, wherein the mRNA encoding the Cas protein comprises a poly(A) tail.

44. The composition of any one of claims 37-43, wherein the mRNA encoding the Cas protein comprises the sequence set forth in SEQ ID NO: 339, 338, or 12.

45. The composition of any one of claims 32-44, wherein the nucleic acid encoding the Cas protein is codon-optimized for expression in a mammalian cell or a human cell.

46. The composition of any one of claims 32-45, wherein the Cas protein comprises the sequence set forth in SEQ ID NO: 11 or 8.

47. The composition of any preceding claim, further comprising a second guide RNA or a DNA encoding the second guide RNA, wherein the second guide RNA comprises a DNA-targeting segment that targets a second guide RNA target sequence in the C5 gene, and wherein the second guide RNA binds to the Cas protein and targets the Cas protein to the second guide RNA target sequence in the C5 gene.

48. The composition of any preceding claim, in association with an antigen-binding protein that binds specifically to C5.

49. The composition of claim 48, wherein the antigen-binding protein that binds specifically to C5 is an antibody or an antigen-binding fragment thereof.

50. The composition of claim 48 or 49, wherein the antigen-binding protein that binds specifically to C5 comprises:
(1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof,
(2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof,
(3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof,
(4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof,
(5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof,
(6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof,
(7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof,
(8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof,
(9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof,
(10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof,
(11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof,
(12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof,
(13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof,
(14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof,
(15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof,
(16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof,
(17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof,
(18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof,
(19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof,
(20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof,
(21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof,
(22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof,
(23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof,
(24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof,
(25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof,
(26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof,
(27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof,
(28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or
(29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or
competes for binding to C5 with an antigen-binding protein selected from the group consisting of (1)-(29); or
binds to the same epitope on C5 as an antigen-binding protein selected from the group consisting of (1)-(29).

51. The composition of any one of claims 48-50, wherein the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof,
optionally wherein the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698.

52. The composition of any one of claims 48-50, wherein the antigen-binding protein that binds specifically to C5 is pozelimab.

53. A cell comprising the composition of any preceding claim.

54. A method of modifying a C5 gene in a cell, comprising introducing the composition of any one of claims 32-52 into the cell, wherein the guide RNA forms a complex with the Cas protein and targets the guide RNA target sequence in the C5 gene, and the Cas protein cleaves the guide RNA target sequence to generate a targeted genetic modification in the C5 gene.

55. The method of claim 54, wherein cleavage by the Cas protein creates a double-strand break in the C5 gene.

56. The method of claim 54, wherein cleavage by the Cas protein creates a single-strand break in the C5 gene.

57. The method of any one of claims 54-56, wherein the targeted genetic modification is generated by repair of the cleaved guide RNA target sequence by non-homologous end-joining.

58. The method of any one of claims 54-57, wherein the method results in reduced expression or activity of the C5 gene in the cell or wherein the method results in loss of function or inactivation of the C5 gene in the cell.

59. The method of any one of claims 54-58, wherein the cell is a hepatocyte.

60. The method of any one of claims 54-59, wherein the cell is a mammalian cell, and the C5 gene is a mammalian C5 gene.

61. The method of any one of claims 54-60, wherein the cell is a human cell, and the C5 gene is a human C5 gene.

62. The method of any one of claims 54-61, wherein the cell is in vitro or ex vivo.

63. The method of any one of claims 54-61, wherein the cell is in an animal in vivo.

64. The method of claim 63, wherein:
(I) the method results in at least 50%, at least 55%, at least 60%, at least 65%, at least 70%, at least 75%, at least 80%, at least 85%, at least 90%, or at least 95% percent editing of the C5 gene in a target population of cells in the animal; or
(II) the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the animal.

65. The method of claim 63 or 64, wherein the method results in reduced serum levels of complement C5 protein in the animal, optionally wherein serum levels of complement C5 protein are reduced by at least 50%, at least 55%, at least 60%, at least 65%, at least 70%, at least 75%, at least 80%, at least 85%, at least 90%, or at least 95%.

66. The method of any one of claims 63-65, wherein the method results in reduced complement C5 protein activity in the animal, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells.

67. A method of modifying a C5 gene in a cell, comprising contacting the genome of the cell with:
(a) a Cas protein; and
(b) a guide RNA that forms a complex with the Cas protein and targets a guide RNA target sequence in the C5 gene,
wherein the Cas protein cleaves the guide RNA target sequence to generate a targeted genetic modification in the C5 gene.

68. The method of claim 67, wherein the guide RNA target sequence is in coding exon 27, 22, 21, 15, 12, or 1 of the C5 gene, or wherein the guide RNA target sequence is in coding exon 15 or 12 of the C5 gene.

69. The method of claim 67 or 68, wherein the guide RNA comprises a DNA-targeting segment that targets the guide RNA target sequence, wherein the DNA-targeting segment comprises, consists essentially of, or consists of at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97, or
wherein the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273, or
wherein the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

70. The method of any one of claims 67-69, wherein the guide RNA comprises a DNA-targeting segment that targets the guide RNA target sequence, wherein the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97, or
wherein the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

71. The method of any one of claims 67-70, wherein the method comprises introducing into the cell:
(a) the Cas protein or a nucleic acid encoding the Cas protein; and
(b) the guide RNA or a DNA encoding the guide RNA.

72. The method of claim 71, wherein the Cas protein or the nucleic acid encoding the Cas protein and/or the guide RNA or the DNA encoding the guide RNA are introduced into the cell via lipid-nanoparticle-mediated delivery.

73. The method of claim 72, wherein the guide RNA in the form of RNA and the nucleic acid encoding the Cas protein are introduced into the cell via lipid-nanoparticle-mediated delivery, wherein the nucleic acid encoding the Cas protein is an mRNA.

74. The method of claim 72 or 73, wherein the lipid nanoparticle comprises a cationic lipid, a neutral lipid, a helper lipid, and a stealth lipid.

75. The method of claim 74, wherein the cationic lipid is Lipid A.

76. The method of claim 74 or 75, wherein the neutral lipid is DSPC.

77. The method of any one of claims 74-76, wherein the helper lipid is cholesterol.

78. The method of any one of claims 74-77, wherein the stealth lipid is PEG2k-DMG.

79. The method of any one of claims 74-78, wherein the cationic lipid is Lipid A, the neutral lipid is DSPC, the helper lipid is cholesterol, and the stealth lipid is PEG2k-DMG.

80. The method of claim 71, wherein the Cas protein or the nucleic acid encoding the Cas protein and/or the guide RNA or the DNA encoding the guide RNA are introduced into the cell via adeno-associated virus.

81. The method of any one of claims 71-80, wherein the method comprises introducing into the cell the nucleic acid encoding the Cas protein.

82. The composition of any one of claims 71-81, wherein the nucleic acid encoding the Cas protein is codon-optimized for expression in a mammalian cell or a human cell.

83. The method of any one of claims 71-82, wherein the nucleic acid encoding the Cas protein comprises DNA, optionally wherein the method comprises introducing into the cell the DNA encoding the guide RNA.

84. The method of any one of claims 71-82, wherein the nucleic acid encoding the Cas protein comprises RNA, optionally wherein the method comprises introducing into the cell the guide RNA in the form of RNA.

85. The method of claim 84, wherein the RNA encoding the Cas protein comprises at least one modification.

86. The method of claim 85, wherein the RNA encoding the Cas protein is modified to comprise a modified uridine at one or more or all uridine positions.

87. The method of claim 86, wherein the modified uridine is N1-methyl-pseudouridine.

88. The method of claim 86 or 87, wherein the RNA encoding the Cas protein is fully substituted with N1-methyl-pseudouridine.

89. The method of any one of claims 85-88, wherein the RNA encoding the Cas protein comprises a 5′ cap.

90. The method of any one of claims 85-89, wherein the RNA encoding the Cas protein comprises a poly(A) tail.

91. The method of any one of claims 84-90, wherein the RNA encoding the Cas protein comprises the sequence set forth in SEQ ID NO: 339, 338, or 12.

92. The method of any one of claims 71-91, wherein the method comprises introducing into the cell the guide RNA in the form of RNA.

93. The method of any one of claims 71-91, wherein the method comprises introducing into the cell the DNA encoding the guide RNA.

94. The method of any one of claims 71-92, wherein the guide RNA comprises at least one modification.

95. The method of claim 94, wherein the at least one modification comprises a 2′-O-methyl-modified nucleotide.

96. The method of claim 94 or 95, wherein the at least one modification comprise a phosphorothioate bond between nucleotides.

97. The method of any one of claims 94-96, wherein the at least one modification comprise a modification at one or more of the first five nucleotides at the 5′ end of the guide RNA.

98. The method of any one of claims 94-97, wherein the at least one modification comprises a modification at one or more of the last five nucleotides at the 3′ end of the guide RNA.

99. The method of any one of claims 94-98, wherein the at least one modification comprises phosphorothioate bonds between the first four nucleotides at the 5′ end of the guide RNA.

100. The method of any one of claims 94-99, wherein the at least one modification comprises phosphorothioate bonds between the last four nucleotides at the 3′ end of the guide RNA.

101. The method of any one of claims 94-100, wherein the at least one modification comprises 2′-O-methyl-modified nucleotides at the first three nucleotides at the 5′ end of the guide RNA.

102. The method of any one of claims 94-101, wherein the at least one modification comprises 2′-O-methyl-modified nucleotides at the last three nucleotides at the 3′ end of the guide RNA.

103. The method of any one of claims 94-102, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

104. The method of any one of claims 94-103, wherein the guide RNA comprises the modified nucleotides of SEQ ID NO: 29.

105. The method of any one of claims 67-104, wherein the guide RNA is a single guide RNA (sgRNA).

106. The method of claim 105, wherein the guide RNA comprises the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

107. The method of any one of claims 67-103, wherein the guide RNA is a dual guide RNA (dgRNA) comprising two separate RNA molecules comprising a CRISPR RNA (crRNA) and a trans-activating crRNA (tracrRNA).

108. The method of claim 107, wherein the crRNA comprises the sequence set forth in any one of SEQ ID NOS: 16-17.

109. The method of claim 107 or 108, wherein the tracrRNA comprises the sequence set forth in any one of SEQ ID NOS: 18-20.

110. The method of any one of claims 67-109, wherein the Cas protein is a Cas9 protein.

111. The method of claim 110, wherein the Cas protein is derived from a Streptococcus pyogenes Cas9 protein.

112. The method of any one of claims 67-111, wherein the Cas protein comprises the sequence set forth in SEQ ID NO: 11 or 8.

113. The method of any one of claims 67-112, further comprising introducing into the cell a second guide RNA or a DNA encoding the second guide RNA, wherein the second guide RNA forms a complex with the Cas protein and targets the Cas protein to a second guide RNA target sequence in the C5 gene, and wherein the Cas protein cleaves the second guide RNA target sequence to generate a targeted genetic modification in the C5 gene.

114. The method of any one of claims 67-113, wherein cleavage by the Cas protein creates a double-strand break in the C5 gene.

115. The method of any one of claims 67-113, wherein cleavage by the Cas protein creates a single-strand break in the C5 gene.

116. The method of any one of claims 67-115, wherein the targeted genetic modification is generated by repair of the cleaved guide RNA target sequence by non-homologous end-joining.

117. The method of any one of claims 67-116, wherein the method results in reduced expression or activity of the C5 gene in the cell or wherein the method results in loss of function or inactivation of the C5 gene in the cell.

118. The method of any one of claims 67-117, wherein the cell is a hepatocyte.

119. The method of any one of claims 67-118, wherein the cell is a mammalian cell, and the C5 gene is a mammalian C5 gene.

120. The method of any one of claims 67-119, wherein the cell is a human cell, and the C5 gene is a human C5 gene.

121. The method of any one of claims 67-120, wherein the cell is in vitro or ex vivo.

122. The method of any one of claims 67-120, wherein the cell is in an animal in vivo.

123. The method of claim 122, wherein:
(I) the method results in at least 50%, at least 55%, at least 60%, at least 65%, at least 70%, at least 75%, at least 80%, at least 85%, at least 90%, or at least 95% percent editing of the C5 gene in a target population of cells in the animal; or
(II) the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the animal.

124. The method of claim 122 or 123, wherein the method results in reduced serum levels of complement C5 protein in the animal, optionally wherein serum levels of complement C5 protein are reduced by at least 50%, at least 55%, at least 60%, at least 65%, at least 70%, at least 75%, at least 80%, at least 85%, at least 90%, or at least 95%.

125. The method of any one of claims 122-124, wherein the method results in reduced complement C5 protein activity in the animal, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells.

126. The method of any one of claims 122-125, wherein C5 complement activity is reduced by about 95-100% as measured by CH50 assay of complement-mediated sheep red blood cell lysis.

127. The method of any one of claims 122-126, further comprising administering to the animal a further therapeutic agent.

128. The method of claim 127, wherein the further therapeutic agent is an antigen-binding protein that binds specifically to C5, acetaminophen, an albumin infusion, ancrod, an angiotensin-converting enzyme inhibitor, an antibiotic, an anti-CD20 agent, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a meningococcal vaccine, methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin, and/or warfarin.

129. The method of claim 128, wherein the therapeutic agent is the antigen-binding protein that binds specifically to C5.

130. The method of claim 129, wherein the antigen-binding protein is administered to the animal intravenously or subcutaneously.

131. The method of claim 129 or 130, wherein a first dose of the antigen-binding protein is administered to the animal intravenously, and one or more additional doses of the antigen-binding protein are administered subcutaneously.

132. The method of any one of claims 129-131, wherein the antigen-binding protein that binds specifically to C5 is an antibody or an antigen-binding fragment thereof.

133. The method of any one of claims 129-132, wherein the antigen-binding protein that binds specifically to C5 comprises:
(1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof,
(2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof,
(3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof,
(4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof,
(5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof,
(6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof,
(7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof,
(8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof,
(9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof,
(10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof,
(11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof,
(12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof,
(13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof,
(14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof,
(15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof,
(16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof,
(17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof,
(18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof,
(19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof,
(20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof,
(21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof,
(22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof,
(23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof,
(24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof,
(25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof,
(26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof,
(27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof,
(28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or
(29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or
competes for binding to C5 with an antigen-binding protein selected from the group consisting of (1)-(29); or
binds to the same epitope on C5 as an antigen-binding protein selected from the group consisting of (1)-(29).

134. The method of any one of claims 129-133, wherein the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof,
optionally wherein the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698.

135. The method of any one of claims 129-134, wherein the antigen-binding protein that binds specifically to C5 is pozelimab.

136. A method of modifying a C5 gene or reducing expression of a C5 gene or reducing activity of complement C5 protein in a subject, comprising administering to a subject:
(a) a Cas protein or a nucleic acid encoding the Cas protein; and
(b) a guide RNA or a DNA encoding the guide RNA, wherein the guide RNA forms a complex with the Cas protein and targets a guide RNA target sequence in a C5 gene,
wherein the Cas protein cleaves the guide RNA target sequence to generate a targeted genetic modification in the C5 gene.

137. A method of preventing, treating, or ameliorating at least one symptom or indication of a disease or disorder associated with C5, comprising administering to a subject in need thereof a pharmaceutical composition comprising a therapeutically effective amount of:
(a) a Cas protein or a nucleic acid encoding the Cas protein; and
(b) a guide RNA or a DNA encoding the guide RNA, wherein the guide RNA forms a complex with the Cas protein and targets a guide RNA target sequence in a C5 gene,
wherein the Cas protein cleaves the guide RNA target sequence to generate a targeted genetic modification in the C5 gene.

138. The method of claim 137, wherein the disease or disorder is adult respiratory distress syndrome; age-related macular degeneration (AMD); allergy; Alport's syndrome; Alzheimer's disease; amyotrophic lateral sclerosis (ALS); antiphospholipid syndrome (APS); asthma; atherosclerosis; atypical hemolytic uremic syndrome (aHUS); an autoimmune disease; autoimmune hemolytic anemia (AIHA); balloon angioplasty; bronchoconstriction; bullous pemphigoid; burns; C3 glomerulopathy; capillary leak syndrome; a cardiovascular disorder; catastrophic antiphospholipid syndrome (CAPS); a cerebrovascular disorder; CHAPLE disease (CD55 deficiency with hyperactivation of complement, angiopathic thrombosis, and protein-losing enteropathy); a chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; eclampsia; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain-Barre Syndrome; Hashimoto's thyroiditis; hemodialysis complications; hemolysis-elevated liver enzymes- and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; an immune complex disorder; immune complex vasculitis; immune complex-associated inflammation; an infectious disease; inflammation caused by an autoimmune disease; an inflammatory disorder; inherited CD59 deficiency; injury due to inert dusts and/or minerals; interleukin-2 induced toxicity during IL-2 therapy; ischemia-reperfusion injury; Kawasaki's disease; a lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); pauci-immune vasculitis; pemphigus; percutaneous transluminal coronary angioplasty (PTCA); peripheral vascular disorder; pneumonia; post-ischemic reperfusion condition; post-pump syndrome in cardiopulmonary bypass; post-pump syndrome in renal bypass; pre-eclampsia; progressive kidney failure; proliferative nephritis; proteinuric kidney disease; psoriasis; pulmonary embolism; pulmonary fibrosis; pulmonary infarction; pulmonary vasculitis; recurrent fetal loss; a renal disorder; renal ischemia; renal ischemia-reperfusion injury; a renovascular disorder; restenosis following stent placement; rheumatoid arthritis (RA); rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome (tHUS); uveitis; vasculitis; vasculitis associated with rheumatoid arthritis; venous gas embolus (VGE); and/or xenograft rejection.

139. The method of claim 137, wherein the disease or disorder is selected from the group consisting of atypical hemolytic uremic syndrome (aHUS), paroxysmal nocturnal hemoglobinuria (PNH), age-related macular degeneration, geographic atrophy, uveitis, neuromyelitis optica, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, thermal injury including burns or frostbite, post-ischemic reperfusion conditions, myocardial infarction, capillary leak syndrome, obesity, diabetes, Alzheimer's disease, schizophrenia, stroke, epilepsy, atherosclerosis, vasculitis, bullous pemphigoid, C3 glomerulopathy, membranoproliferative glomerulonephritis, diabetic nephropathy, Alport's syndrome, progressive kidney failure, proteinuric kidney diseases, renal ischemia-reperfusion injury, lupus nephritis, balloon angioplasty, post-pump syndrome in cardiopulmonary bypass or renal bypass, hemodialysis, renal ischemia, mesenteric artery reperfusion after aortic reconstruction, infectious disease or sepsis, immune complex disorders and autoimmune diseases, renal disorders, rheumatoid arthritis, systemic lupus erythematosus (SLE), SLE nephritis, proliferative nephritis, hemolytic anemia, asthma, chronic obstructive pulmonary disease (COPD), emphysema, pulmonary embolisms and infarcts, pneumonia, and myasthenia gravis.

140. The method of claim 137, wherein the disease or disorder is atypical hemolytic uremic syndrome (aHUS), paroxysmal nocturnal hemoglobinuria (PNH), refractory myasthenia gravis (rMG), neuromyelitis optica (NMO), IgA nephropathy, membranous nephropathy, lupus nephritis, C3 glomerulopathy, and ANCA-vasculitis.

141. The method of claim 137, wherein the disease or disorder is aHUS or PNH.

142. The method of claim 137, wherein the disease or disorder is PNH, optionally wherein the method is for reducing serum lactate dehydrogenase (LDH) levels, intravascular hemolysis, and/or the need for transfusions of red blood cells in the subject.

143. The method of claim 137, wherein the disease or disorder is CD55-deficient protein-losing enteropathy (CHAPLE disease), optionally wherein the method is for (i) normalizing and/or increasing serum albumin or decreasing loss thereof through the gastrointestinal tract; increasing total serum protein level, or decreasing loss thereof through the gastrointestinal tract; increasing serum vitamin B12 or gastrointestinal absorption thereof; decreasing platelet counts or decreasing coagulation cascade activation or decreasing the incidence of thrombotic events; decreasing the loss of alpha-1-antitrypsin through the gastrointestinal tract; treating or preventing facial and/or peripheral edema; decreasing the frequency of bowel movements; treating or preventing diarrhea; treating or preventing abdominal pain; decreasing the use of corticosteroids; and/or decreasing the incidence of hospitalization in the subject; or (ii) reducing therapeutic interventions in the subject, wherein the therapeutic intervention is one or more selected from the group consisting of: administration of a corticosteroid; administration of an immunoglobulin; administration of albumin; administration of an anti-tumor necrosis factor alpha therapeutic agent; administration of an immunomodulator; administration of a micronutrient; administration of enteral or parenteral supplementation; administration of an anti-coagulant; administration of an antibiotic; and administration of an anti-platelet agent, and
optionally wherein the method is for increasing serum albumin by at least 1 g/dL and/or for normalizing serum albumin to about 3.5 to about 5.5 g/dL.

144. The method of any one of claims 137-143, wherein the pharmaceutical composition is administered prophylactically or therapeutically to the subject in need thereof.

145. The method of any one of claims 137-144, wherein the pharmaceutical composition is administered intravenously.

146. The method of any one of claims 136-145, wherein the guide RNA target sequence is in coding exon 27, 22, 21, 15, 12, or 1 of the C5 gene, or wherein the guide RNA target sequence is in coding exon 15 or 12 of the C5 gene.

147. The method of any one of claims 136-146, wherein the guide RNA comprises a DNA-targeting segment that targets the guide RNA target sequence, wherein the DNA-targeting segment comprises, consists essentially of, or consists of at least 17, at least 18, at least 19, or at least 20 contiguous nucleotides of the sequence set forth in any one of SEQ ID NOS: 33-120, any one of SEQ ID NOS: 60, 65, 67, 82, 85, 87, 97, and 119, or any one of SEQ ID NOS: 85 and 97, or wherein the guide RNA target sequence comprises at least 17, at least 18, at least 19, or at least 20 contiguous nucleotides of the sequence set forth in any one of SEQ ID NOS: 209-296, any one of SEQ ID NOS: 236, 241, 243, 258, 261, 263, 273, and 295, or any one of SEQ ID NOS: 261 and 273, or wherein the DNA-targeting segment is at least 90%, at least 91%, at least 92%, at least 93%, at least 94%, at least 95%, at least 96%, at least 97%, at least 98%, or at least 99% identical to the sequence set forth in any one of SEQ ID NOS: 33-120, any one of SEQ ID NOS: 60, 65, 67, 82, 85, 87, 97, and 119, or any one of SEQ ID NOS: 85 and 97.

148. The method of any one of claims 136-147, wherein the guide RNA comprises a DNA-targeting segment that targets the guide RNA target sequence, wherein the DNA-targeting segment comprises, consists essentially of, or consists of the sequence set forth in any one of SEQ ID NOS: 33-120, any one of SEQ ID NOS: 60, 65, 67, 82, 85, 87, 97, and 119, or any one of SEQ ID NOS: 85 and 97, or wherein the guide RNA target sequence comprises, consists essentially of, or consists of the sequence set forth in any one of SEQ ID NOS: 209-296, any one of SEQ ID NOS: 236, 241, 243, 258, 261, 263, 273, and 295, or any one of SEQ ID NOS: 261 and 273.

149. The method of any one of claims 136-148, wherein the Cas protein or the nucleic acid encoding the Cas protein and/or the guide RNA or the DNA encoding the guide RNA are administered to the subject via lipid-nanoparticle-mediated delivery.

150. The method of claim 149, wherein the guide RNA in the form of RNA and the nucleic acid encoding the Cas protein are administered to the subject via lipid-nanoparticle-mediated delivery, wherein the nucleic acid encoding the Cas protein is an mRNA.

151. The method of claim 149 or 150, wherein the lipid nanoparticle comprises a cationic lipid, a neutral lipid, a helper lipid, and a stealth lipid.

152. The method of claim 151, wherein the cationic lipid is Lipid A.

153. The method of claim 151 or 152, wherein the neutral lipid is DSPC.

154. The method of any one of claims 151-153, wherein the helper lipid is cholesterol.

155. The method of any one of claims 151-154, wherein the stealth lipid is PEG2k-DMG.

156. The method of any one of claims 151-155, wherein the cationic lipid is Lipid A, the neutral lipid is DSPC, the helper lipid is cholesterol, and the stealth lipid is PEG2k-DMG.

157. The method of any one of claims 136-148, wherein the Cas protein or the nucleic acid encoding the Cas protein and/or the guide RNA or the DNA encoding the guide RNA are administered to the subject via adeno-associated virus.

158. The method of any one of claims 136-157, wherein the method comprises administering the nucleic acid encoding the Cas protein.

159. The composition of any one of claims 136-158, wherein the nucleic acid encoding the Cas protein is codon-optimized for expression in a mammalian cell or a human cell.

160. The method of any one of claims 136-159, wherein the nucleic acid encoding the Cas protein comprises DNA, optionally wherein the method comprises administering the DNA encoding the guide RNA.

161. The method of any one of claims 136-160, wherein the nucleic acid encoding the Cas protein comprises RNA, optionally wherein the method comprises administering the guide RNA in the form of RNA.

162. The method of claim 161, wherein the RNA encoding the Cas protein comprises at least one modification.

163. The method of claim 162, wherein the RNA encoding the Cas protein is modified to comprise a modified uridine at one or more or all uridine positions.

164. The method of claim 163, wherein the modified uridine is N1-methyl-pseudouridine.

165. The method of claim 163 or 164, wherein the RNA encoding the Cas protein is fully substituted with N1-methyl-pseudouridine.

166. The method of any one of claims 162-165, wherein the RNA encoding the Cas protein comprises a 5′ cap.

167. The method of any one of claims 162-166, wherein the RNA encoding the Cas protein comprises a poly(A) tail.

168. The method of any one of claims 161-167, wherein the RNA encoding the Cas protein comprises the sequence set forth in SEQ ID NO: 339, 338, or 12.

169. The method of any one of claims 136-168, wherein the method comprises administering the guide RNA in the form of RNA.

170. The method of any one of claims 136-168, wherein the method comprises administering the DNA encoding the guide RNA.

171. The method of any one of claims 136-169, wherein the guide RNA comprises at least one modification.

172. The method of claim 171, wherein the at least one modification comprises a 2′-O-methyl-modified nucleotide.

173. The method of claim 171 or 172, wherein the at least one modification comprise a phosphorothioate bond between nucleotides.

174. The method of any one of claims 171-173, wherein the at least one modification comprise a modification at one or more of the first five nucleotides at the 5′ end of the guide RNA.

175. The method of any one of claims 171-174, wherein the at least one modification comprises a modification at one or more of the last five nucleotides at the 3′ end of the guide RNA.

176. The method of any one of claims 171-175, wherein the at least one modification comprises phosphorothioate bonds between the first four nucleotides at the 5′ end of the guide RNA.

177. The method of any one of claims 171-176, wherein the at least one modification comprises phosphorothioate bonds between the last four nucleotides at the 3′ end of the guide RNA.

178. The method of any one of claims 171-177, wherein the at least one modification comprises 2′-O-methyl-modified nucleotides at the first three nucleotides at the 5′ end of the guide RNA.

179. The method of any one of claims 171-178, wherein the at least one modification comprises 2′-O-methyl-modified nucleotides at the last three nucleotides at the 3′ end of the guide RNA.

180. The method of any one of claims 171-179, wherein the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

181. The method of any one of claims 171-180, wherein the guide RNA comprises the modified nucleotides of SEQ ID NO: 29.

182. The method of any one of claims 136-181, wherein the guide RNA is a single guide RNA (sgRNA).

183. The method of claim 182, wherein the guide RNA comprises the sequence set forth in any one of SEQ ID NOS: 21-29, wherein the guide RNA comprises, consists essentially of, or consists of the sequence set forth in any one of SEQ ID NOS:297-312 and 316-331, wherein the guide RNA comprises, consists essentially of, or consists of the sequence set forth in any one of SEQ ID NOS: 297-304 and 316-323, or wherein the guide RNA comprises, consists essentially of, or consists of the sequence set forth in any one of SEQ ID NOS: 299, 301, 318, and 320.

184. The method of any one of claims 136-180, wherein the guide RNA is a dual guide RNA (dgRNA) comprising two separate RNA molecules comprising a CRISPR RNA (crRNA) and a trans-activating crRNA (tracrRNA).

185. The method of claim 184, wherein the crRNA comprises the sequence set forth in any one of SEQ ID NOS: 16-17.

186. The method of claim 184 or 185, wherein the tracrRNA comprises the sequence set forth in any one of SEQ ID NOS: 18-20.

187. The method of any one of claims 136-186, wherein the Cas protein is a Cas9 protein.

188. The method of claim 187, wherein the Cas protein is derived from a Streptococcus pyogenes Cas9 protein.

189. The method of any one of claims 136-188, wherein the Cas protein comprises the sequence set forth in SEQ ID NO: 11 or 8.

190. The method of any one of claims 136-189, further comprising administering to the subject a second guide RNA or a DNA encoding the second guide RNA, wherein the second guide RNA forms a complex with the Cas protein and targets the Cas protein to a second guide RNA target sequence in the C5 gene, wherein the Cas protein cleaves the second guide RNA target sequence to generate a targeted genetic modification in the C5 gene.

191. The method of any one of claims 136-190, wherein cleavage by the Cas protein creates a double-strand break in the C5 gene.

192. The method of any one of claims 136-190, wherein cleavage by the Cas protein creates a single-strand break in the C5 gene.

193. The method of any one of claims 136-192, wherein the targeted genetic modification is generated by repair of the cleaved guide RNA target sequence by non-homologous end-joining.

194. The method of any one of claims 136-193, wherein the method results in reduced expression or activity of the C5 gene in the cell.

195. The method of any one of claims 136-194, wherein the method results in loss of function or inactivation of the C5 gene in the cell.

196. The method of any one of claims 136-195, wherein the subject is a mammal, and the C5 gene is a mammalian C5 gene.

197. The method of any one of claims 136-196, wherein the subject is a human, and the C5 gene is a human C5 gene.

198. The method of any one of claims 136-197, wherein:
(I) the method results in at least 50%, at least 55%, at least 60%, at least 65%, at least 70%, at least 75%, at least 80%, at least 85%, at least 90%, or at least 95% percent editing of the C5 gene in a target population of cells in the subject; or
(II) the method results in between about 30% and about 35%, between about 35% and about 40%, between about 40% and about 45%, between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, or between about 95% and about 99% editing of the C5 gene in a target population of cells in the subject.

199. The method of any one of claims 136-198, wherein the method results in reduced serum levels of complement C5 protein in the subject, optionally wherein serum levels of complement C5 protein are reduced by at least 50%, at least 55%, at least 60%, at least 65%, at least 70%, at least 75%, at least 80%, at least 85%, at least 90%, or at least 95%.

200. The method of any one of claims 136-199, wherein the method results in reduced complement C5 protein activity in the subject, optionally wherein the method results in at least 50%, at least 55%, at least 60%, at least 65%, at least 70%, at least 75%, at least 80%, at least 85%, at least 90%, or at least 95% percent inhibition of classical pathway hemolysis as measured ex vivo using sensitized sheep red blood cells.

201. The method of any one of claims 136-200, wherein C5 complement activity is reduced by about 95-100% as measured by CH50 assay of complement-mediated sheep red blood cell lysis.

202. The method of any one of claims 136-200, wherein the composition is administered in association with a further therapeutic agent.

203. The method of claim 202, wherein the further therapeutic agent is an antigen-binding protein that binds specifically to C5, acetaminophen, an albumin infusion, ancrod, an angiotensin-converting enzyme inhibitor, an antibiotic, an anti-CD20 agent, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a meningococcal vaccine, methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin, and/or warfarin.

204. The method of claim 203, wherein the further therapeutic agent is the antigen-binding protein that binds specifically to C5.

205. The method of claim 204, wherein the antigen-binding protein is administered to the subject intravenously or subcutaneously.

206. The method of claim 204 or 205, wherein a first dose of the antigen-binding protein is administered to the subject intravenously, and one or more additional doses of the antigen-binding protein are administered subcutaneously.

207. The method of any one of claims 204-206, wherein the antigen-binding protein that binds specifically to C5 is an antibody or an antigen-binding fragment thereof.

208. The method of any one of claims 204-207, wherein the antigen-binding protein that binds specifically to C5 comprises:
(1) a heavy chain variable region (HCVR) that comprises the amino acid sequence set forth in SEQ ID NO: 341 or HCDR1, HCDR2 and HCDR3 thereof, and a light chain variable region (LCVR) that comprises the amino acid sequence set forth in SEQ ID NO: 349 or LCDR1, LCDR2 and LCDR3 thereof,
(2) a HCVR that comprises the amino acid sequence set forth in SEQ ID NO: 357 or HCDR1, HCDR2 and HCDR3 thereof, and an LCVR that comprises the amino acid sequence set forth in SEQ ID NO: 365 or LCDR1, LCDR2 and LCDR3 thereof,
(3) a HCVR that comprises the amino acid sequence set forth in SEQ ID NO: 373 or HCDR1, HCDR2 and HCDR3 thereof, and an LCVR that comprises the amino acid sequence set forth in SEQ ID NO: 381 or LCDR1, LCDR2 and LCDR3 thereof,
(4) a HCVR that comprises the amino acid sequence set forth in SEQ ID NO: 389 or HCDR1, HCDR2 and HCDR3 thereof, and an LCVR that comprises the amino acid sequence set forth in SEQ ID NO: 397 or LCDR1, LCDR2 and LCDR3 thereof,
(5) a HCVR that comprises the amino acid sequence set forth in SEQ ID NO: 405 or HCDR1, HCDR2 and HCDR3 thereof, and an LCVR that comprises the amino acid sequence set forth in SEQ ID NO: 413 or LCDR1, LCDR2 and LCDR3 thereof,
(6) a HCVR that comprises the amino acid sequence set forth in SEQ ID NO: 421 or HCDR1, HCDR2 and HCDR3 thereof, and an LCVR that comprises the amino acid sequence set forth in SEQ ID NO: 429 or LCDR1, LCDR2 and LCDR3 thereof,
(7) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 445 or LCDR1, LCDR2 and LCDR3 thereof,
(8) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 453 or LCDR1, LCDR2 and LCDR3 thereof,
(9) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 445 or LCDR1, LCDR2 and LCDR3 thereof,
(10) a HCVR that comprises the amino acid sequence set forth in SEQ ID NO: 437 or HCDR1, HCDR2 and HCDR3 thereof, and an LCVR that comprises the amino acid sequence set forth in SEQ ID NO: 469 or LCDR1, LCDR2 and LCDR3 thereof,
(11) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 445 or LCDR1, LCDR2 and LCDR3 thereof,
(12) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 445 or LCDR1, LCDR2 and LCDR3 thereof,
(13) a HCVR that comprises the amino acid sequence set forth in SEQ ID NO: 461 or HCDR1, HCDR2 and HCDR3 thereof, and an LCVR that comprises the amino acid sequence set forth in SEQ ID NO: 469 or LCDR1, LCDR2 and LCDR3 thereof,
(14) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 453 or LCDR1, LCDR2 and LCDR3 thereof,
(15) a HCVR that comprises the amino acid sequence set forth in SEQ ID NO: 485 or HCDR1, HCDR2 and HCDR3 thereof, and an LCVR that comprises the amino acid sequence set forth in SEQ ID NO: 469 or LCDR1, LCDR2 and LCDR3 thereof,
(16) a HCVR that comprises the amino acid sequence set forth in SEQ ID NO: 477 or HCDR1, HCDR2 and HCDR3 thereof, and an LCVR that comprises the amino acid sequence set forth in SEQ ID NO: 469 or LCDR1, LCDR2 and LCDR3 thereof,
(17) a HCVR that comprises the amino acid sequence set forth in SEQ ID NO: 493 or HCDR1, HCDR2 and HCDR3 thereof, and an LCVR that comprises the amino acid sequence set forth in SEQ ID NO: 501 or LCDR1, LCDR2 and LCDR3 thereof,
(18) a HCVR that comprises the amino acid sequence set forth in SEQ ID NO: 509 or HCDR1, HCDR2 and HCDR3 thereof, and an LCVR that comprises the amino acid sequence set forth in SEQ ID NO: 517 or LCDR1, LCDR2 and LCDR3 thereof,
(19) a HCVR that comprises the amino acid sequence set forth in SEQ ID NO: 525 or HCDR1, HCDR2 and HCDR3 thereof, and an LCVR that comprises the amino acid sequence set forth in SEQ ID NO: 533 or LCDR1, LCDR2 and LCDR3 thereof,
(20) a HCVR that comprises the amino acid sequence set forth in SEQ ID NO: 541 or HCDR1, HCDR2 and HCDR3 thereof, and an LCVR that comprises the amino acid sequence set forth in SEQ ID NO: 549 or LCDR1, LCDR2 and LCDR3 thereof,
(21) a HCVR that comprises the amino acid sequence set forth in SEQ ID NO: 557 or HCDR1, HCDR2 and HCDR3 thereof, and an LCVR that comprises the amino acid sequence set forth in SEQ ID NO: 565 or LCDR1, LCDR2 and LCDR3 thereof,
(22) a HCVR that comprises the amino acid sequence set forth in SEQ ID NO: 573 or HCDR1, HCDR2 and HCDR3 thereof, and an LCVR that comprises the amino acid sequence set forth in SEQ ID NO: 581 or LCDR1, LCDR2 and LCDR3 thereof,
(23) a HCVR that comprises the amino acid sequence set forth in SEQ ID NO: 589 or HCDR1, HCDR2 and HCDR3 thereof, and an LCVR that comprises the amino acid sequence set forth in SEQ ID NO: 597 or LCDR1, LCDR2 and LCDR3 thereof,
(24) a HCVR that comprises the amino acid sequence set forth in SEQ ID NO: 605 or HCDR1, HCDR2 and HCDR3 thereof, and an LCVR that comprises the amino acid sequence set forth in SEQ ID NO: 597 or LCDR1, LCDR2 and LCDR3 thereof,
(25) a HCVR that comprises the amino acid sequence set forth in SEQ ID NO: 613 or HCDR1, HCDR2 and HCDR3 thereof, and an LCVR that comprises the amino acid sequence set forth in SEQ ID NO: 621 or LCDR1, LCDR2 and LCDR3 thereof,
(26) a HCVR that comprises the amino acid sequence set forth in SEQ ID NO: 629 or HCDR1, HCDR2 and HCDR3 thereof, and an LCVR that comprises the amino acid sequence set forth in SEQ ID NO: 637 or LCDR1, LCDR2 and LCDR3 thereof,
(27) a HCVR that comprises the amino acid sequence set forth in SEQ ID NO: 645 or HCDR1, HCDR2 and HCDR3 thereof, and an LCVR that comprises the amino acid sequence set forth in SEQ ID NO: 653 or LCDR1, LCDR2 and LCDR3 thereof,
(28) a HCVR that comprises the amino acid sequence set forth in SEQ ID NO: 661 or HCDR1, HCDR2 and HCDR3 thereof, and an LCVR that comprises the amino acid sequence set forth in SEQ ID NO: 669 or LCDR1, LCDR2 and LCDR3 thereof, or
(29) a HCVR that comprises the amino acid sequence set forth in SEQ ID NO: 677 or HCDR1, HCDR2 and HCDR3 thereof, and an LCVR that comprises the amino acid sequence set forth in SEQ ID NO: 685 or LCDR1, LCDR2 and LCDR3 thereof, or
competes for binding to C5 with an antigen-binding protein selected from the group consisting of (1)-(29); or
binds to the same epitope on C5 as an antigen-binding protein selected from the group consisting of (1)-(29).

209. The method of any one of claims 204-208, wherein the antigen-binding protein that binds specifically to C5 is a monoclonal antibody comprising an immunoglobulin heavy chain comprising the amino acid sequence set forth in SEQ ID NO: 697 or a variable region thereof or HCDR1, HCDR2 and HCDR3 thereof, and an immunoglobulin light chain comprising the amino acid sequence set forth in SEQ ID NO: 698 or a variable region thereof or LCDR1, LCDR2 and LCDR3 thereof, optionally wherein the antigen-binding protein that binds specifically to C5 is a monoclonal antibody comprising an immunoglobulin heavy chain comprising the amino acid sequence set forth in SEQ ID NO: 697, and an immunoglobulin light chain comprising the amino acid sequence set forth in SEQ ID NO: 698.

210. The method of any one of claims 204-209, wherein the antigen-binding protein that binds specifically to C5 is pozelimab.


SEQUENCE LISTING (708 sequences)

SEQ ID NO: 1 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 2 - DNA, 5464 bp, Homo sapiens
  source 1..5464: mol_type = other DNA
atatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaa

SEQ ID NO: 3 - DNA, 5031 bp, Homo sapiens
  source 1..5031: mol_type = other DNA
atgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaa

SEQ ID NO: 4 - protein, 1682 aa, Homo sapiens
MPGSLGREASGRAGPTGCGAFAFGLRCRYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 5 - DNA, 5482 bp, Homo sapiens
  source 1..5482: mol_type = other DNA
cccgcctcttttgcagccagcgcgctaggatgccggggagtcttgggcgcgaggcttccggccgcgcggggccgacgggatgcggtgccttcgccttcggattgcgttgcagatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaa

SEQ ID NO: 6 - DNA, 5031 bp, synthetic construct
  misc_feature 1..5031: note = Synthetic
  source 1..5031: mol_type = other DNA
atgggtctttggggaatactttgtcttttaattttcctggacaaaacttggggacaggaacaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaa

SEQ ID NO: 7 - DNA, 5031 bp, synthetic construct
  misc_feature 1..5031: note = Synthetic
  source 1..5031: mol_type = other DNA
atgggccttttgggaatactttgttttttaatcttcctgggaaaaacttggggacaggagcaaacatatgtcatttcagcaccaaaaatattccgtgttggagcatctgaaaacattgtgattcaagtttatggatacactgaagcatttgatgcaacaatctctattaaaagttatcctgataaaaaatttagttactcctcaggccatgttcatttatcctcagagaataaattccaaaactcggcagtcttaacaatacaaccaaaacaattacctggaggacaaaaccaagtttcttatgtgtatttggaagttgtatcaaagcatttttcaaaatcaaaaaaaattccaataacctatgacaatggatttctcttcattcatacagacaaacctgtttatactccagaccaatcagtaaaggttagagtttattcgttgaatgatgacttgaagccagccaaaagagaaactgtcttaactttcatagatcctgaaggatcagaaattgacatggtagaagaaattgatcatattggaattatctcttttcctgacttcaagattccgtctaatcctagatatggtatgtggacgatccaggctaaatataaagaggacttttcaacaactggaactgcattttttgaagttaaagaatatgtcttgccacatttttctgtctcagtagaaccagaaagtaatttcattggttataagaactttaagaattttgaaattactataaaagcaagatatttttataataaagtagtcactgaggctgatgtttatatcacatttggaataagagaagacttaaaagatgatcaaaaagaaatgatgcaaacagcaatgcaaaacacaatgttgataaatggaattgctgaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatattccatcaaggtgcaggttaaagatgcgcttgaccagttggtaggaggggtcccagtaacactgaatgcacaaacaattgatgtcaaccaagagacatctgacttggagccaaggaaaagtgtaacacgtgttgatgatggagtagcttcgtttgtggttaatctcccatctggagtgacggtgctggagtttaatgtcaaaactgatgctccagatcttccagacgaaaatcaggccagggaaggttaccgagcaatagcatactcatctctcagccaaagttacctttatatcgattggactgataaccacaaggctttgctagtgggagaatatttgaatattattgttacccccaaaagcccatatattgacaaaataactcactataattacttgattttatccaagggcaaaattatccactttggcacaagggagaaactttcagatgcatcttatcaaagtataaacattccagtaacgcagaacatggttccttcatcccgactcttggtctattacatcgtcacaggagagcagacagcagaattagtgtctgattcagtctggttaaatattgaagaaaaatgtggcaaccagctccaggttcatctgtctcctgatgcagatacatattctccaggccaaactgtgtctcttaatatggtaactgggatggattcctgggtggcattaacagcagtggacagcgctgtgtatggagtccaaagaagagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattatcaggccaagaagaatgctacaagagaagatagaagaaatagctgctaaatataaacatttagtagtgaagaaatgttgttacgatggagtccgtattaatcatgatgaaacctgtgagcagcgagctgcacggattagtgtagggccgagatgcgtcaaagctttcactgaatgttgtgtcgtcgcaagccagctccgtgctaataactctcataaagacttgcaattgggaaggctacacatgaagaccctgttaccagtaagcaagccagaaattcggagttattttccagaaagctggttatgggaagttcatcttgttcccagaagaaaacagttgcagtttgccctacctgattctgtaactacctgggaaattcaaggtgttggcatttcaaacagtggtatatgtgttgctgatactattaaggcaaaggtgttcaaagatgtcttcctggaaatgaatataccatattctgttgtacgaggagaacaggtccagttgaaaggaactgtttacaactataggacttctgggatgcagttctgtgttaaaatgtctgctgtggagggaatctgcacttcagaaagcccagtcattgatcatcagggcacaaagtcctccaaatgtgtgcgacagaaagtagagggctcctctaatcacttggtgacctttactgtgcttcctctggaaattggccttcagaacatcaatttctcactggagacttcgtttggaaaagaaatcttagtaaaatcgttacgagtggtgccagaaggtgtcaaaagggaaagctattctggtattactttggatcctaggggtatttatggtaccattagcagacgaaaggagttcccatacaggataccattagatttggtccccaaaacagaaatcaaaaggattttgagtgtaaaaggactgcttgtaggtgagatcttgtctgcagttctaagtcgggaaggcatcaatatcctaacccacctccccaaagggagtgcagaggcggagctgatgagcgttgtcccagtattctatgtttttcactacctggaaacaggaaatcattggaacatttttcattccgacccattaattgaaaagcggaacctggagaaaaaattaaaagaagggatggtgagcattatgtcctacagaaatgctgactattcttacagcgtgtggaagggtggcagtgctagcacttggttaacagcttttgctttaagagtacttggacaagtacataaatatgtagagcagaaccaaaattcaatatgtaattctttattgtggctggttgagaattatcagttagataatggatctttcaaggaaaattcacagtatcaaccaataaaattacagggtaccttgcctgttgaagcccgagagaacagcttatatcttacagcctttactgtgattggaattagaaaggcttttgatatatgccccctggtgaaaatcaacacagctctaattaaagctgacacctttctgcttgaaaatacactgccagcccagagcacctttacattggccatttctgcctatgctctttccctgggagataaaactcacccacagtttcgttcaattgtttcagctttgaagagagaagctttggttaaaggtaatccacccatttatcgtttttggaaagacagtcttcaacataaagacagctctgtacctaacactggtacagcacgtatggtagaaacaactgcctatgctttactcaccagtctgaacttgaaagacataaattatgttaacccaatcatcaaatggctatcagaagagcagaggtatggaggtggcttttattcaacccaggacacaatcaatgccatcgagggcctgacagaatattcactcctggttaaacagctccgcttgaatatggacatcgatgttgcttacaagcataaaggtcccttacataattataaaatgacagacaagaatttccttgggaggccagtagaggtgcttctcaatgatgacctcgttgtcagtacaggatttggcagtggcttggctacggtacatgtaacaactgtagttcacaaaaccagtacctctgaggaagtttgcagcttttatttgaaaattgatactcaggatattgaagcatcccactacagaggctacggaaactctgattacaaacgcatagtagcatgtgccagctacaagcccagcaaggaagaatcatcttctggatcctctcatgcagtgatggacatctccttgcctactggaatcaatgcaaatgaagaagacttaaaagctcttgtggaaggggtggatcagctattcactgattaccaaataaaagatggacatgttattctgcaactgaattcgatcccctccagtgatttcctttgtgtacgattccggatttttgaactctttgaagttgggtttcttagtcctgccactttcacagtgtatgaataccacagaccagataaacagtgtaccatgttttatagcacttccaatatcaaaattcagaaagtctgtgaaggagccacgtgcaagtgtatagaagctgattgtgggcaaatgcagaaagaattggatctgacaatctctgcagagactagaaaacaaacagcatgtaacccagagattgcatatgcttataaagttatcatcacatccatcactacagaaaatgtttttgtcaagtacaaggcaacccttctggatatctacaaaactggggaagctgttgctgaaaaagactctgaaatcaccttcattaaaaaggtaacctgcactaacgctgagctggtgaaaggaagacagtacttaattatggggaaagaagctctccagataaaatacaatttcactttcaggtacatctaccctttagattccttgacctggattgaatactggcctagagacacaacatgttcatcgtgtcaagcatttttagctaatttagatgaatttgctgaagacatctttttaaatggatgctaa

SEQ ID NO: 8 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 9 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 10 - DNA, 4218 bp, synthetic construct
  misc_feature 1..4218: note = Synthetic
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 11 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 12 - RNA, 4514 nt, synthetic construct
  misc_feature 1..4514: note = Synthetic
  source 1..4514: mol_type = other RNA
gggtcccgcagtcggcgtccagcggctctgcttgttcgtgtgtgtgtcgttgcaggccttattcggatccatggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatgagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 13 - protein, 7 aa, synthetic construct
  REGION 1..7: note = Synthetic
PKKKRKV

SEQ ID NO: 14 - protein, 7 aa, synthetic construct
  REGION 1..7: note = Synthetic
PKKKRRV

SEQ ID NO: 15 - protein, 16 aa, synthetic construct
  REGION 1..16: note = Synthetic
KRPAATKKAGQAKKKK

SEQ ID NO: 16 - RNA, 16 nt, synthetic construct
  misc_feature 1..16: note = Synthetic
  source 1..16: mol_type = other RNA
gttttagagctatgct

SEQ ID NO: 17 - RNA, 22 nt, synthetic construct
  misc_feature 1..22: note = Synthetic
  source 1..22: mol_type = other RNA
gttttagagctatgctgttttg

SEQ ID NO: 18 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 19 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 20 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 21 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 22 - RNA, 82 nt, synthetic construct; identical to SEQ ID NO: 20
  misc_feature 1..82: note = Synthetic
  source 1..82: mol_type = other RNA

SEQ ID NO: 23 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 24 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 25 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 26 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 27 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 28 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 29 - RNA, 100 nt, synthetic construct
  misc_feature 1..100: note = Synthetic
  misc_difference 1..20: note = n is a, c, g, or u
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 30-32 - skipped (3 entries)

SEQ ID NO: 33 - RNA, 20 nt, synthetic construct
  misc_feature 1..20: note = Synthetic
  source 1..20: mol_type = other RNA
ccttgacctggattgaatac

SEQ ID NO: 34 - RNA, 20 nt, synthetic construct
  misc_feature 1..20: note = Synthetic
  source 1..20: mol_type = other RNA
accctttagattccttgacc

SEQ ID NO: 35 - RNA, 20 nt, synthetic construct
  misc_feature 1..20: note = Synthetic
  source 1..20: mol_type = other RNA
ggaagacagtacttaattat

SEQ ID NO: 36 - RNA, 20 nt, synthetic construct
  misc_feature 1..20: note = Synthetic
  source 1..20: mol_type = other RNA
aggaagacagtacttaatta

SEQ ID NO: 37 - RNA, 20 nt, synthetic construct
  misc_feature 1..20: note = Synthetic
  source 1..20: mol_type = other RNA
agttttgtagatatccagaa

SEQ ID NO: 38 - RNA, 20 nt, synthetic construct
  misc_feature 1..20: note = Synthetic
  source 1..20: mol_type = other RNA
cagttttgtagatatccaga

SEQ ID NO: 39 - RNA, 20 nt, synthetic construct
  misc_feature 1..20: note = Synthetic
  source 1..20: mol_type = other RNA
aactcaccatatgcaatctc

SEQ ID NO: 40 - RNA, 20 nt, synthetic construct
  misc_feature 1..20: note = Synthetic
  source 1..20: mol_type = other RNA
gactttctgaattttgatat

SEQ ID NO: 41 - RNA, 20 nt, synthetic construct
  misc_feature 1..20: note = Synthetic
  source 1..20: mol_type = other RNA
tacacaaaggaaatcactgg

SEQ ID NO: 42 - RNA, 20 nt, synthetic construct
  misc_feature 1..20: note = Synthetic
  source 1..20: mol_type = other RNA
ggagttgattacctggtctg

SEQ ID NO: 43 - RNA, 20 nt, synthetic construct
  misc_feature 1..20: note = Synthetic
  source 1..20: mol_type = other RNA
ttagcatggagttgattacc

SEQ ID NO: 44 - RNA, 20 nt, synthetic construct
  misc_feature 1..20: note = Synthetic
  source 1..20: mol_type = other RNA
ctcttttcccccttagcttg

SEQ ID NO: 45 - RNA, 20 nt, synthetic construct
  misc_feature 1..20: note = Synthetic
  source 1..20: mol_type = other RNA
ttcccccttagcttgtggaa

SEQ ID NO: 46 - RNA, 20 nt, synthetic construct
  misc_feature 1..20: note = Synthetic
  source 1..20: mol_type = other RNA
atggacatctccttgcctac

SEQ ID NO: 47 - RNA, 20 nt, synthetic construct
  misc_feature 1..20: note = Synthetic
  source 1..20: mol_type = other RNA
gcatcccactacagaggcta

SEQ ID NO: 48 - RNA, 20 nt, synthetic construct
  misc_feature 1..20: note = Synthetic
  source 1..20: mol_type = other RNA
tcacaaaaccagtacctctg

SEQ ID NO: 49 - RNA, 20 nt, synthetic construct
  misc_feature 1..20: note = Synthetic
  source 1..20: mol_type = other RNA
aaagctgcaaacttcctcag

SEQ ID NO: 50 - RNA, 20 nt, synthetic construct
  misc_feature 1..20: note = Synthetic
  source 1..20: mol_type = other RNA
tacaggatttggcagtggct

SEQ ID NO: 51 - RNA, 20 nt, synthetic construct
  misc_feature 1..20: note = Synthetic
  source 1..20: mol_type = other RNA
gtcagtacaggatttggcag

SEQ ID NO: 52 - RNA, 20 nt, synthetic construct
  misc_feature 1..20: note = Synthetic
  source 1..20: mol_type = other RNA
cagacaagaatttccttggg

SEQ ID NO: 53 - RNA, 20 nt, synthetic construct
  misc_feature 1..20: note = Synthetic
  source 1..20: mol_type = other RNA
agttcttcatttacctctac

SEQ ID NO: 54 - RNA, 20 nt, synthetic construct
  misc_feature 1..20: note = Synthetic
  source 1..20: mol_type = other RNA
ctatcagaagagcagaggta

SEQ ID NO: 55 - RNA, 20 nt, synthetic construct
  misc_feature 1..20: note = Synthetic
  source 1..20: mol_type = other RNA
gacagctctgtacctaacac

SEQ ID NO: 56 - RNA, 20 nt, synthetic construct
  misc_feature 1..20: note = Synthetic
  source 1..20: mol_type = other RNA
atccacccatttatcgtttt

SEQ ID NO: 57 - RNA, 20 nt, synthetic construct
  misc_feature 1..20: note = Synthetic
  source 1..20: mol_type = other RNA
gaaacaattgaacgaaactg

SEQ ID NO: 58 - RNA, 20 nt, synthetic construct
  misc_feature 1..20: note = Synthetic
  source 1..20: mol_type = other RNA
agcccagagcacctttacat

SEQ ID NO: 59 - RNA, 20 nt, synthetic construct
  misc_feature 1..20: note = Synthetic
  source 1..20: mol_type = other RNA
ctgttctctcgggcttcaac

SEQ ID NO: 60 - RNA, 20 nt, synthetic construct
  misc_feature 1..20: note = Synthetic
  source 1..20: mol_type = other RNA
ctctcgggcttcaacaggca

SEQ ID NO: 61 - RNA, 20 nt, synthetic construct
  misc_feature 1..20: note = Synthetic
  source 1..20: mol_type = other RNA
tgcactccctttggggaggt

SEQ ID NO: 62 - RNA, 20 nt, synthetic construct
  misc_feature 1..20: note = Synthetic
  source 1..20: mol_type = other RNA
attctatgtttttcactacc

SEQ ID NO: 63 - RNA, 20 nt, synthetic construct
  misc_feature 1..20: note = Synthetic
  source 1..20: mol_type = other RNA
gacgaaaggagttcccatac

SEQ ID NO: 64 - RNA, 20 nt, synthetic construct
  misc_feature 1..20: note = Synthetic
  source 1..20: mol_type = other RNA
aggtaccattagcagacgaa

SEQ ID NO: 65 - RNA, 20 nt, synthetic construct
  misc_feature 1..20: note = Synthetic
  source 1..20: mol_type = other RNA
tgcctaccataaatacccct

SEQ ID NO: 66 - RNA, 20 nt, synthetic construct
  misc_feature 1..20: note = Synthetic
  source 1..20: mol_type = other RNA
ttagccagaaggtgtcaaaa

SEQ ID NO: 67 - RNA, 20 nt, synthetic construct
  misc_feature 1..20: note = Synthetic
  source 1..20: mol_type = other RNA
tgccctgatgatcaatgact

SEQ ID NO: 68 - RNA, 20 nt, synthetic construct
  misc_feature 1..20: note = Synthetic
  source 1..20: mol_type = other RNA
gtgccctgatgatcaatgac

SEQ ID NO: 69 - RNA, 20 nt, synthetic construct
  misc_feature 1..20: note = Synthetic
  source 1..20: mol_type = other RNA
agcccagtcattgatcatca

SEQ ID NO: 70 - RNA, 20 nt, synthetic construct
  misc_feature 1..20: note = Synthetic
  source 1..20: mol_type = other RNA
aagcccagtcattgatcatc

SEQ ID NO: 71 - RNA, 20 nt, synthetic construct
  misc_feature 1..20: note = Synthetic
  source 1..20: mol_type = other RNA
tttacaactataggacttct

SEQ ID NO: 72 - RNA, 20 nt, synthetic construct
  misc_feature 1..20: note = Synthetic
  source 1..20: mol_type = other RNA
tctcctcgtacaacagaata

SEQ ID NO: 73 - RNA, 20 nt, synthetic construct
  misc_feature 1..20: note = Synthetic
  source 1..20: mol_type = other RNA
aaggaactgtttacaactat

SEQ ID NO: 74 - RNA, 20 nt, synthetic construct
  misc_feature 1..20: note = Synthetic
  source 1..20: mol_type = other RNA
gtttacaactataggacttc

SEQ ID NO: 75 - RNA, 20 nt, synthetic construct
  misc_feature 1..20: note = Synthetic
  source 1..20: mol_type = other RNA
ataccatattctgttgtacg

SEQ ID NO: 76 - RNA, 20 nt, synthetic construct
  misc_feature 1..20: note = Synthetic
  source 1..20: mol_type = other RNA
cgaatttctggcttgcttac

SEQ ID NO: 77 - RNA, 20 nt, synthetic construct
  misc_feature 1..20: note = Synthetic
  source 1..20: mol_type = other RNA
cagtaagcaagccagaaatt

SEQ ID NO: 78 - RNA, 20 nt, synthetic construct
  misc_feature 1..20: note = Synthetic
  source 1..20: mol_type = other RNA
tggcttgcttactggtaaca

SEQ ID NO: 79 - RNA, 20 nt, synthetic construct
  misc_feature 1..20: note = Synthetic
  source 1..20: mol_type = other RNA
ctggcttgcttactggtaac

SEQ ID NO: 80 - RNA, 20 nt, synthetic construct
  misc_feature 1..20: note = Synthetic
  source 1..20: mol_type = other RNA
ccgtgcagctcgctgctcac

SEQ ID NO: 81 - RNA, 20 nt, synthetic construct
  misc_feature 1..20: note = Synthetic
  source 1..20: mol_type = other RNA
gtgaagaaatgttgttacga

SEQ ID NO: 82 - RNA, 20 nt, synthetic construct
  misc_feature 1..20: note = Synthetic
  source 1..20: mol_type = other RNA
ttcttagagaagagtgatct

SEQ ID NO: 83 - RNA, 20 nt, synthetic construct
  misc_feature 1..20: note = Synthetic
  source 1..20: mol_type = other RNA
attcttagagaagagtgatc

SEQ ID NO: 84 - RNA, 20 nt, synthetic construct
  misc_feature 1..20: note = Synthetic
  source 1..20: mol_type = other RNA
gccaatgtgttccacctagc

SEQ ID NO: 85 - RNA, 20 nt, synthetic construct
  misc_feature 1..20: note = Synthetic
  source 1..20: mol_type = other RNA
tgaggaaggtaagtccagct

SEQ ID NO: 86 - RNA, 20 nt, synthetic construct
  misc_feature 1..20: note = Synthetic
  source 1..20: mol_type = other RNA
ggaaggtaagtccagctagg

SEQ ID NO: 87 - RNA, 20 nt, synthetic construct
  misc_feature 1..20: note = Synthetic
  source 1..20: mol_type = other RNA
tccagctaggtggaacacat

SEQ ID NO: 88 - RNA, 20 nt, synthetic construct
  misc_feature 1..20: note = Synthetic
  source 1..20: mol_type = other RNA
aactctttccaagggctttt

SEQ ID NO: 89 - RNA, 20 nt, synthetic construct
  misc_feature 1..20: note = Synthetic
  source 1..20: mol_type = other RNA
cattacttaactctttccaa

SEQ ID NO: 90 - RNA, 20 nt, synthetic construct
  misc_feature 1..20: note = Synthetic
  source 1..20: mol_type = other RNA
ccatattaagagacacagtt

SEQ ID NO: 91 - RNA, 20 nt, synthetic construct
  misc_feature 1..20: note = Synthetic
  source 1..20: mol_type = other RNA
atttgtggcttacctggagc

SEQ ID NO: 92 - RNA, 20 nt, synthetic construct
  misc_feature 1..20: note = Synthetic
  source 1..20: mol_type = other RNA
aagggcaaaattatccactt

SEQ ID NO: 93 - RNA, 20 nt, synthetic construct
  misc_feature 1..20: note = Synthetic
  source 1..20: mol_type = other RNA
aattagtgtctgattcagtc

SEQ ID NO: 94 - RNA, 20 nt, synthetic construct
  misc_feature 1..20: note = Synthetic
  source 1..20: mol_type = other RNA
tttgtcaatatatgggcttt

SEQ ID NO: 95 - RNA, 20 nt, synthetic construct
  misc_feature 1..20: note = Synthetic
  source 1..20: mol_type = other RNA
gagttattttgtcaatatat

SEQ ID NO: 96 - RNA, 20 nt, synthetic construct
  misc_feature 1..20: note = Synthetic
  source 1..20: mol_type = other RNA
ttgtcaatatatgggctttt

SEQ ID NO: 97 - RNA, 20 nt, synthetic construct
  misc_feature 1..20: note = Synthetic
  source 1..20: mol_type = other RNA
gagatgagtatgctattgct

SEQ ID NO: 98 - RNA, 20 nt, synthetic construct
  misc_feature 1..20: note = Synthetic
  source 1..20: mol_type = other RNA
tttgtgcattcagtgttact

SEQ ID NO: 99 - RNA, 20 nt, synthetic construct
  misc_feature 1..20: note = Synthetic
  source 1..20: mol_type = other RNA
gtttgtgcattcagtgttac

SEQ ID NO: 100 - RNA, 20 nt, synthetic construct
  misc_feature 1..20: note = Synthetic
  source 1..20: mol_type = other RNA
agtgtaacacgtgttgatga

SEQ ID NO: 101 - RNA, 20 nt, synthetic construct
  misc_feature 1..20: note = Synthetic
  source 1..20: mol_type = other RNA
ccagcaccgtcactccagat

SEQ ID NO: 102 - RNA, 20 nt, synthetic construct
  misc_feature 1..20: note = Synthetic
  source 1..20: mol_type = other RNA
ttgttttgacaaacaggtgc

SEQ ID NO: 103 - RNA, 20 nt, synthetic construct
  misc_feature 1..20: note = Synthetic
  source 1..20: mol_type = other RNA
cccatctggagtgacggtgc

SEQ ID NO: 104 - RNA, 20 nt, synthetic construct
  misc_feature 1..20: note = Synthetic
  source 1..20: mol_type = other RNA
tccagcaccgtcactccaga

SEQ ID NO: 105 - RNA, 20 nt, synthetic construct
  misc_feature 1..20: note = Synthetic
  source 1..20: mol_type = other RNA
cttaggtggattttctgaag

SEQ ID NO: 106 - RNA, 20 nt, synthetic construct
  misc_feature 1..20: note = Synthetic
  source 1..20: mol_type = other RNA
atgcttgccatttccttagg

SEQ ID NO: 107 - RNA, 20 nt, synthetic construct
  misc_feature 1..20: note = Synthetic
  source 1..20: mol_type = other RNA
ctctccctacaaactgaatt

SEQ ID NO: 108 - RNA, 20 nt, synthetic construct
  misc_feature 1..20: note = Synthetic
  source 1..20: mol_type = other RNA
gagaggacatatttgatgcc

SEQ ID NO: 109 - RNA, 20 nt, synthetic construct
  misc_feature 1..20: note = Synthetic
  source 1..20: mol_type = other RNA
gcaaccaaattcagtttgta

SEQ ID NO: 110 - RNA, 20 nt, synthetic construct
  misc_feature 1..20: note = Synthetic
  source 1..20: mol_type = other RNA
gcttccacagatatggtatg

SEQ ID NO: 111 - RNA, 20 nt, synthetic construct
  misc_feature 1..20: note = Synthetic
  source 1..20: mol_type = other RNA
ttagacggaatcttgaagtc

SEQ ID NO: 112 - RNA, 20 nt, synthetic construct
  misc_feature 1..20: note = Synthetic
  source 1..20: mol_type = other RNA
aagacagtttctcttttggc

SEQ ID NO: 113 - RNA, 20 nt, synthetic construct
  misc_feature 1..20: note = Synthetic
  source 1..20: mol_type = other RNA
aatccattgtcataggttat

SEQ ID NO: 114 - RNA, 20 nt, synthetic construct
  misc_feature 1..20: note = Synthetic
  source 1..20: mol_type = other RNA
tctctgaggataaatgaaca

SEQ ID NO: 115 - RNA, 20 nt, synthetic construct
  misc_feature 1..20: note = Synthetic
  source 1..20: mol_type = other RNA
ggataaatgaacatggcctg

SEQ ID NO: 116 - RNA, 20 nt, synthetic construct
  misc_feature 1..20: note = Synthetic
  source 1..20: mol_type = other RNA
gcaccaaaaatattccgtgt

SEQ ID NO: 117 - RNA, 20 nt, synthetic construct
  misc_feature 1..20: note = Synthetic
  source 1..20: mol_type = other RNA
gctccaacacggaatatttt

SEQ ID NO: 118 - RNA, 20 nt, synthetic construct
  misc_feature 1..20: note = Synthetic
  source 1..20: mol_type = other RNA
tttcctgctacctccaacca

SEQ ID NO: 119 - RNA, 20 nt, synthetic construct
  misc_feature 1..20: note = Synthetic
  source 1..20: mol_type = other RNA
cctccaaccatgggcctttt

SEQ ID NO: 120 - RNA, 20 nt, synthetic construct
  misc_feature 1..20: note = Synthetic
  source 1..20: mol_type = other RNA
acctccaaccatgggccttt

SEQ ID NO: 121 - RNA, 42 nt, synthetic construct
  misc_feature 1..42: note = Synthetic
  source 1..42: mol_type = other RNA
ccttgacctggattgaatacgttttagagctatgctgttttg

SEQ ID NO: 122 - RNA, 42 nt, synthetic construct
  misc_feature 1..42: note = Synthetic
  source 1..42: mol_type = other RNA
accctttagattccttgaccgttttagagctatgctgttttg

SEQ ID NO: 123 - RNA, 42 nt, synthetic construct
  misc_feature 1..42: note = Synthetic
  source 1..42: mol_type = other RNA
ggaagacagtacttaattatgttttagagctatgctgttttg

SEQ ID NO: 124 - RNA, 42 nt, synthetic construct
  misc_feature 1..42: note = Synthetic
  source 1..42: mol_type = other RNA
aggaagacagtacttaattagttttagagctatgctgttttg

SEQ ID NO: 125 - RNA, 42 nt, synthetic construct
  misc_feature 1..42: note = Synthetic
  source 1..42: mol_type = other RNA
agttttgtagatatccagaagttttagagctatgctgttttg

SEQ ID NO: 126 - RNA, 42 nt, synthetic construct
  misc_feature 1..42: note = Synthetic
  source 1..42: mol_type = other RNA
cagttttgtagatatccagagttttagagctatgctgttttg

SEQ ID NO: 127 - RNA, 42 nt, synthetic construct
  misc_feature 1..42: note = Synthetic
  source 1..42: mol_type = other RNA
aactcaccatatgcaatctcgttttagagctatgctgttttg

SEQ ID NO: 128 - RNA, 42 nt, synthetic construct
  misc_feature 1..42: note = Synthetic
  source 1..42: mol_type = other RNA
gactttctgaattttgatatgttttagagctatgctgttttg

SEQ ID NO: 129 - RNA, 42 nt, synthetic construct
  misc_feature 1..42: note = Synthetic
  source 1..42: mol_type = other RNA
tacacaaaggaaatcactgggttttagagctatgctgttttg

SEQ ID NO: 130 - RNA, 42 nt, synthetic construct
  misc_feature 1..42: note = Synthetic
  source 1..42: mol_type = other RNA
ggagttgattacctggtctggttttagagctatgctgttttg

SEQ ID NO: 131 - RNA, 42 nt, synthetic construct
  misc_feature 1..42: note = Synthetic
  source 1..42: mol_type = other RNA
ttagcatggagttgattaccgttttagagctatgctgttttg

SEQ ID NO: 132 - RNA, 42 nt, synthetic construct
  misc_feature 1..42: note = Synthetic
  source 1..42: mol_type = other RNA
ctcttttcccccttagcttggttttagagctatgctgttttg

SEQ ID NO: 133 - RNA, 42 nt, synthetic construct
  misc_feature 1..42: note = Synthetic
  source 1..42: mol_type = other RNA
ttcccccttagcttgtggaagttttagagctatgctgttttg

SEQ ID NO: 134 - RNA, 42 nt, synthetic construct
  misc_feature 1..42: note = Synthetic
  source 1..42: mol_type = other RNA
atggacatctccttgcctacgttttagagctatgctgttttg

SEQ ID NO: 135 - RNA, 42 nt, synthetic construct
  misc_feature 1..42: note = Synthetic
  source 1..42: mol_type = other RNA
gcatcccactacagaggctagttttagagctatgctgttttg

SEQ ID NO: 136 - RNA, 42 nt, synthetic construct
  misc_feature 1..42: note = Synthetic
  source 1..42: mol_type = other RNA
tcacaaaaccagtacctctggttttagagctatgctgttttg

SEQ ID NO: 137 - RNA, 42 nt, synthetic construct
  misc_feature 1..42: note = Synthetic
  source 1..42: mol_type = other RNA
aaagctgcaaacttcctcaggttttagagctatgctgttttg

SEQ ID NO: 138 - RNA, 42 nt, synthetic construct
  misc_feature 1..42: note = Synthetic
  source 1..42: mol_type = other RNA
tacaggatttggcagtggctgttttagagctatgctgttttg

SEQ ID NO: 139 - RNA, 42 nt, synthetic construct
  misc_feature 1..42: note = Synthetic
  source 1..42: mol_type = other RNA
gtcagtacaggatttggcaggttttagagctatgctgttttg

SEQ ID NO: 140 - RNA, 42 nt, synthetic construct
  misc_feature 1..42: note = Synthetic
  source 1..42: mol_type = other RNA
cagacaagaatttccttggggttttagagctatgctgttttg

SEQ ID NO: 141 - RNA, 42 nt, synthetic construct
  misc_feature 1..42: note = Synthetic
  source 1..42: mol_type = other RNA
agttcttcatttacctctacgttttagagctatgctgttttg

SEQ ID NO: 142 - RNA, 42 nt, synthetic construct
  misc_feature 1..42: note = Synthetic
  source 1..42: mol_type = other RNA
ctatcagaagagcagaggtagttttagagctatgctgttttg

SEQ ID NO: 143 - RNA, 42 nt, synthetic construct
  misc_feature 1..42: note = Synthetic
  source 1..42: mol_type = other RNA
gacagctctgtacctaacacgttttagagctatgctgttttg

SEQ ID NO: 144 - RNA, 42 nt, synthetic construct
  misc_feature 1..42: note = Synthetic
  source 1..42: mol_type = other RNA
atccacccatttatcgttttgttttagagctatgctgttttg

SEQ ID NO: 145 - RNA, 42 nt, synthetic construct
  misc_feature 1..42: note = Synthetic
  source 1..42: mol_type = other RNA
gaaacaattgaacgaaactggttttagagctatgctgttttg

SEQ ID NO: 146 - RNA, 42 nt, synthetic construct
  misc_feature 1..42: note = Synthetic
  source 1..42: mol_type = other RNA
agcccagagcacctttacatgttttagagctatgctgttttg

SEQ ID NO: 147 - RNA, 42 nt, synthetic construct
  misc_feature 1..42: note = Synthetic
  source 1..42: mol_type = other RNA
ctgttctctcgggcttcaacgttttagagctatgctgttttg

SEQ ID NO: 148 - RNA, 42 nt, synthetic construct
  misc_feature 1..42: note = Synthetic
  source 1..42: mol_type = other RNA
ctctcgggcttcaacaggcagttttagagctatgctgttttg

SEQ ID NO: 149 - RNA, 42 nt, synthetic construct
  misc_feature 1..42: note = Synthetic
  source 1..42: mol_type = other RNA
tgcactccctttggggaggtgttttagagctatgctgttttg

SEQ ID NO: 150 - RNA, 42 nt, synthetic construct
  misc_feature 1..42: note = Synthetic
  source 1..42: mol_type = other RNA
attctatgtttttcactaccgttttagagctatgctgttttg

SEQ ID NO: 151 - RNA, 42 nt, synthetic construct
  misc_feature 1..42: note = Synthetic
  source 1..42: mol_type = other RNA
gacgaaaggagttcccatacgttttagagctatgctgttttg

SEQ ID NO: 152 - RNA, 42 nt, synthetic construct
  misc_feature 1..42: note = Synthetic
  source 1..42: mol_type = other RNA
aggtaccattagcagacgaagttttagagctatgctgttttg

SEQ ID NO: 153 - RNA, 42 nt, synthetic construct
  misc_feature 1..42: note = Synthetic
  source 1..42: mol_type = other RNA
tgcctaccataaatacccctgttttagagctatgctgttttg

SEQ ID NO: 154 - RNA, 42 nt, synthetic construct
  misc_feature 1..42: note = Synthetic
  source 1..42: mol_type = other RNA
ttagccagaaggtgtcaaaagttttagagctatgctgttttg

SEQ ID NO: 155 - RNA, 42 nt, synthetic construct
  misc_feature 1..42: note = Synthetic
  source 1..42: mol_type = other RNA
tgccctgatgatcaatgactgttttagagctatgctgttttg

SEQ ID NO: 156 - RNA, 42 nt, synthetic construct
  misc_feature 1..42: note = Synthetic
  source 1..42: mol_type = other RNA
gtgccctgatgatcaatgacgttttagagctatgctgttttg

SEQ ID NO: 157 - RNA, 42 nt, synthetic construct
  misc_feature 1..42: note = Synthetic
  source 1..42: mol_type = other RNA
agcccagtcattgatcatcagttttagagctatgctgttttg

SEQ ID NO: 158 - RNA, 42 nt, synthetic construct
  misc_feature 1..42: note = Synthetic
  source 1..42: mol_type = other RNA
aagcccagtcattgatcatcgttttagagctatgctgttttg

SEQ ID NO: 159 - RNA, 42 nt, synthetic construct
  misc_feature 1..42: note = Synthetic
  source 1..42: mol_type = other RNA
tttacaactataggacttctgttttagagctatgctgttttg

SEQ ID NO: 160 - RNA, 42 nt, synthetic construct
  misc_feature 1..42: note = Synthetic
  source 1..42: mol_type = other RNA
tctcctcgtacaacagaatagttttagagctatgctgttttg

SEQ ID NO: 161 - RNA, 42 nt, synthetic construct
  misc_feature 1..42: note = Synthetic
  source 1..42: mol_type = other RNA
aaggaactgtttacaactatgttttagagctatgctgttttg

SEQ ID NO: 162 - RNA, 42 nt, synthetic construct
  misc_feature 1..42: note = Synthetic
  source 1..42: mol_type = other RNA
gtttacaactataggacttcgttttagagctatgctgttttg

SEQ ID NO: 163 - RNA, 42 nt, synthetic construct
  misc_feature 1..42: note = Synthetic
  source 1..42: mol_type = other RNA
ataccatattctgttgtacggttttagagctatgctgttttg

SEQ ID NO: 164 - RNA, 42 nt, synthetic construct
  misc_feature 1..42: note = Synthetic
  source 1..42: mol_type = other RNA
cgaatttctggcttgcttacgttttagagctatgctgttttg

SEQ ID NO: 165 - RNA, 42 nt, synthetic construct
  misc_feature 1..42: note = Synthetic
  source 1..42: mol_type = other RNA
cagtaagcaagccagaaattgttttagagctatgctgttttg

SEQ ID NO: 166 - RNA, 42 nt, synthetic construct
  misc_feature 1..42: note = Synthetic
  source 1..42: mol_type = other RNA
tggcttgcttactggtaacagttttagagctatgctgttttg

SEQ ID NO: 167 - RNA, 42 nt, synthetic construct
  misc_feature 1..42: note = Synthetic
  source 1..42: mol_type = other RNA
ctggcttgcttactggtaacgttttagagctatgctgttttg

SEQ ID NO: 168 - RNA, 42 nt, synthetic construct
  misc_feature 1..42: note = Synthetic
  source 1..42: mol_type = other RNA
ccgtgcagctcgctgctcacgttttagagctatgctgttttg

SEQ ID NO: 169 - RNA, 42 nt, synthetic construct
  misc_feature 1..42: note = Synthetic
  source 1..42: mol_type = other RNA
gtgaagaaatgttgttacgagttttagagctatgctgttttg

SEQ ID NO: 170 - RNA, 42 nt, synthetic construct
  misc_feature 1..42: note = Synthetic
  source 1..42: mol_type = other RNA
ttcttagagaagagtgatctgttttagagctatgctgttttg

SEQ ID NO: 171 - RNA, 42 nt, synthetic construct
  misc_feature 1..42: note = Synthetic
  source 1..42: mol_type = other RNA
attcttagagaagagtgatcgttttagagctatgctgttttg

SEQ ID NO: 172 - RNA, 42 nt, synthetic construct
  misc_feature 1..42: note = Synthetic
  source 1..42: mol_type = other RNA
gccaatgtgttccacctagcgttttagagctatgctgttttg

SEQ ID NO: 173 - RNA, 42 nt, synthetic construct
  misc_feature 1..42: note = Synthetic
  source 1..42: mol_type = other RNA
tgaggaaggtaagtccagctgttttagagctatgctgttttg

SEQ ID NO: 174 - RNA, 42 nt, synthetic construct
  misc_feature 1..42: note = Synthetic
  source 1..42: mol_type = other RNA
ggaaggtaagtccagctagggttttagagctatgctgttttg

SEQ ID NO: 175 - RNA, 42 nt, synthetic construct
  misc_feature 1..42: note = Synthetic
  source 1..42: mol_type = other RNA
tccagctaggtggaacacatgttttagagctatgctgttttg

SEQ ID NO: 176 - RNA, 42 nt, synthetic construct
  misc_feature 1..42: note = Synthetic
  source 1..42: mol_type = other RNA
aactctttccaagggcttttgttttagagctatgctgttttg

SEQ ID NO: 177 - RNA, 42 nt, synthetic construct
  misc_feature 1..42: note = Synthetic
  source 1..42: mol_type = other RNA
cattacttaactctttccaagttttagagctatgctgttttg

SEQ ID NO: 178 - RNA, 42 nt, synthetic construct
  misc_feature 1..42: note = Synthetic
  source 1..42: mol_type = other RNA
ccatattaagagacacagttgttttagagctatgctgttttg

SEQ ID NO: 179 - RNA, 42 nt, synthetic construct
  misc_feature 1..42: note = Synthetic
  source 1..42: mol_type = other RNA
atttgtggcttacctggagcgttttagagctatgctgttttg

SEQ ID NO: 180 - RNA, 42 nt, synthetic construct
  misc_feature 1..42: note = Synthetic
  source 1..42: mol_type = other RNA
aagggcaaaattatccacttgttttagagctatgctgttttg

SEQ ID NO: 181 - RNA, 42 nt, synthetic construct
  misc_feature 1..42: note = Synthetic
  source 1..42: mol_type = other RNA
aattagtgtctgattcagtcgttttagagctatgctgttttg

SEQ ID NO: 182 - RNA, 42 nt, synthetic construct
  misc_feature 1..42: note = Synthetic
  source 1..42: mol_type = other RNA
tttgtcaatatatgggctttgttttagagctatgctgttttg

SEQ ID NO: 183 - RNA, 42 nt, synthetic construct
  misc_feature 1..42: note = Synthetic
  source 1..42: mol_type = other RNA
gagttattttgtcaatatatgttttagagctatgctgttttg

SEQ ID NO: 184 - RNA, 42 nt, synthetic construct
  misc_feature 1..42: note = Syntheic
  source 1..42: mol_type = other RNA
ttgtcaatatatgggcttttgttttagagctatgctgttttg

SEQ ID NO: 185 - RNA, 42 nt, synthetic construct
  misc_feature 1..42: note = Synthetic
  source 1..42: mol_type = other RNA
gagatgagtatgctattgctgttttagagctatgctgttttg

SEQ ID NO: 186 - RNA, 42 nt, synthetic construct
  misc_feature 1..42: note = Synthetic
  source 1..42: mol_type = other RNA
tttgtgcattcagtgttactgttttagagctatgctgttttg

SEQ ID NO: 187 - RNA, 42 nt, synthetic construct
  misc_feature 1..42: note = Synthetic
  source 1..42: mol_type = other RNA
gtttgtgcattcagtgttacgttttagagctatgctgttttg

SEQ ID NO: 188 - RNA, 42 nt, synthetic construct
  misc_feature 1..42: note = Synthetic
  source 1..42: mol_type = other RNA
agtgtaacacgtgttgatgagttttagagctatgctgttttg

SEQ ID NO: 189 - RNA, 42 nt, synthetic construct
  misc_feature 1..42: note = Synthetic
  source 1..42: mol_type = other RNA
ccagcaccgtcactccagatgttttagagctatgctgttttg

SEQ ID NO: 190 - RNA, 42 nt, synthetic construct
  misc_feature 1..42: note = Synthetic
  source 1..42: mol_type = other RNA
ttgttttgacaaacaggtgcgttttagagctatgctgttttg

SEQ ID NO: 191 - RNA, 42 nt, synthetic construct
  misc_feature 1..42: note = Synthetic
  source 1..42: mol_type = other RNA
cccatctggagtgacggtgcgttttagagctatgctgttttg

SEQ ID NO: 192 - RNA, 42 nt, synthetic construct
  misc_feature 1..42: note = Synthetic
  source 1..42: mol_type = other RNA
tccagcaccgtcactccagagttttagagctatgctgttttg

SEQ ID NO: 193 - RNA, 42 nt, synthetic construct
  misc_feature 1..42: note = Synthetic
  source 1..42: mol_type = other RNA
cttaggtggattttctgaaggttttagagctatgctgttttg

SEQ ID NO: 194 - RNA, 42 nt, synthetic construct
  misc_feature 1..42: note = Synthetic
  source 1..42: mol_type = other RNA
atgcttgccatttccttagggttttagagctatgctgttttg

SEQ ID NO: 195 - RNA, 42 nt, synthetic construct
  misc_feature 1..42: note = Synthetic
  source 1..42: mol_type = other RNA
ctctccctacaaactgaattgttttagagctatgctgttttg

SEQ ID NO: 196 - RNA, 42 nt, synthetic construct
  misc_feature 1..42: note = Synthetic
  source 1..42: mol_type = other RNA
gagaggacatatttgatgccgttttagagctatgctgttttg

SEQ ID NO: 197 - RNA, 42 nt, synthetic construct
  misc_feature 1..42: note = Synthetic
  source 1..42: mol_type = other RNA
gcaaccaaattcagtttgtagttttagagctatgctgttttg

SEQ ID NO: 198 - RNA, 42 nt, synthetic construct
  misc_feature 1..42: note = Synthetic
  source 1..42: mol_type = other RNA
gcttccacagatatggtatggttttagagctatgctgttttg

SEQ ID NO: 199 - RNA, 42 nt, synthetic construct
  misc_feature 1..42: note = Synthetic
  source 1..42: mol_type = other RNA
ttagacggaatcttgaagtcgttttagagctatgctgttttg

SEQ ID NO: 200 - RNA, 42 nt, synthetic construct
  misc_feature 1..42: note = Synthetic
  source 1..42: mol_type = other RNA
aagacagtttctcttttggcgttttagagctatgctgttttg

SEQ ID NO: 201 - RNA, 42 nt, synthetic construct
  misc_feature 1..42: note = Synthetic
  source 1..42: mol_type = other RNA
aatccattgtcataggttatgttttagagctatgctgttttg

SEQ ID NO: 202 - RNA, 42 nt, synthetic construct
  misc_feature 1..42: note = Synthetic
  source 1..42: mol_type = other RNA
tctctgaggataaatgaacagttttagagctatgctgttttg

SEQ ID NO: 203 - RNA, 42 nt, synthetic construct
  misc_feature 1..42: note = Synthetic
  source 1..42: mol_type = other RNA
ggataaatgaacatggcctggttttagagctatgctgttttg

SEQ ID NO: 204 - RNA, 42 nt, synthetic construct
  misc_feature 1..42: note = Synthetic
  source 1..42: mol_type = other RNA
gcaccaaaaatattccgtgtgttttagagctatgctgttttg

SEQ ID NO: 205 - RNA, 42 nt, synthetic construct
  misc_feature 1..42: note = Synthetic
  source 1..42: mol_type = other RNA
gctccaacacggaatattttgttttagagctatgctgttttg

SEQ ID NO: 206 - RNA, 42 nt, synthetic construct
  misc_feature 1..42: note = Synthetic
  source 1..42: mol_type = other RNA
tttcctgctacctccaaccagttttagagctatgctgttttg

SEQ ID NO: 207 - RNA, 42 nt, synthetic construct
  misc_feature 1..42: note = Synthetic
  source 1..42: mol_type = other RNA
cctccaaccatgggccttttgttttagagctatgctgttttg

SEQ ID NO: 208 - RNA, 42 nt, synthetic construct
  misc_feature 1..42: note = Synthetic
  source 1..42: mol_type = other RNA
acctccaaccatgggcctttgttttagagctatgctgttttg

SEQ ID NO: 209 - DNA, 20 bp, synthetic construct
  misc_feature 1..20: note = Synthetic
  source 1..20: mol_type = other DNA
ccttgacctggattgaatac

SEQ ID NO: 210 - DNA, 20 bp, synthetic construct
  misc_feature 1..20: note = Synthetic
  source 1..20: mol_type = other DNA
accctttagattccttgacc

SEQ ID NO: 211 - DNA, 20 bp, synthetic construct
  misc_feature 1..20: note = Synthetic
  source 1..20: mol_type = other DNA
ggaagacagtacttaattat

SEQ ID NO: 212 - DNA, 20 bp, synthetic construct
  misc_feature 1..20: note = Synthetic
  source 1..20: mol_type = other DNA
aggaagacagtacttaatta

SEQ ID NO: 213 - DNA, 20 bp, synthetic construct
  misc_feature 1..20: note = Synthetic
  source 1..20: mol_type = other DNA
agttttgtagatatccagaa

SEQ ID NO: 214 - DNA, 20 bp, synthetic construct
  misc_feature 1..20: note = Synthetic
  source 1..20: mol_type = other DNA
cagttttgtagatatccaga

SEQ ID NO: 215 - DNA, 20 bp, synthetic construct
  misc_feature 1..20: note = Synthetic
  source 1..20: mol_type = other DNA
aactcaccatatgcaatctc

SEQ ID NO: 216 - DNA, 20 bp, synthetic construct
  misc_feature 1..20: note = Synthetic
  source 1..20: mol_type = other DNA
gactttctgaattttgatat

SEQ ID NO: 217 - DNA, 20 bp, synthetic construct
  misc_feature 1..20: note = Synthetic
  source 1..20: mol_type = other DNA
tacacaaaggaaatcactgg

SEQ ID NO: 218 - DNA, 20 bp, synthetic construct
  misc_feature 1..20: note = Synthetic
  source 1..20: mol_type = other DNA
ggagttgattacctggtctg

SEQ ID NO: 219 - DNA, 20 bp, synthetic construct
  misc_feature 1..20: note = Synthetic
  source 1..20: mol_type = other DNA
ttagcatggagttgattacc

SEQ ID NO: 220 - DNA, 20 bp, synthetic construct
  misc_feature 1..20: note = Synthetic
  source 1..20: mol_type = other DNA
ctcttttcccccttagcttg

SEQ ID NO: 221 - DNA, 20 bp, synthetic construct
  misc_feature 1..20: note = Synthetic
  source 1..20: mol_type = other DNA
ttcccccttagcttgtggaa

SEQ ID NO: 222 - DNA, 20 bp, synthetic construct
  misc_feature 1..20: note = Synthetic
  source 1..20: mol_type = other DNA
atggacatctccttgcctac

SEQ ID NO: 223 - DNA, 20 bp, synthetic construct
  misc_feature 1..20: note = Synthetic
  source 1..20: mol_type = other DNA
gcatcccactacagaggcta

SEQ ID NO: 224 - DNA, 20 bp, synthetic construct
  misc_feature 1..20: note = Synthetic
  source 1..20: mol_type = other DNA
tcacaaaaccagtacctctg

SEQ ID NO: 225 - DNA, 20 bp, synthetic construct
  misc_feature 1..20: note = Synthetic
  source 1..20: mol_type = other DNA
aaagctgcaaacttcctcag

SEQ ID NO: 226 - DNA, 20 bp, synthetic construct
  misc_feature 1..20: note = Synthetic
  source 1..20: mol_type = other DNA
tacaggatttggcagtggct

SEQ ID NO: 227 - DNA, 20 bp, synthetic construct
  misc_feature 1..20: note = Synthetic
  source 1..20: mol_type = other DNA
gtcagtacaggatttggcag

SEQ ID NO: 228 - DNA, 20 bp, synthetic construct
  misc_feature 1..20: note = Synthetic
  source 1..20: mol_type = other DNA
cagacaagaatttccttggg

SEQ ID NO: 229 - DNA, 20 bp, synthetic construct
  misc_feature 1..20: note = Synthetic
  source 1..20: mol_type = other DNA
agttcttcatttacctctac

SEQ ID NO: 230 - DNA, 20 bp, synthetic construct
  misc_feature 1..20: note = Synthetic
  source 1..20: mol_type = other DNA
ctatcagaagagcagaggta

SEQ ID NO: 231 - DNA, 20 bp, synthetic construct
  misc_feature 1..20: note = Synthetic
  source 1..20: mol_type = other DNA
gacagctctgtacctaacac

SEQ ID NO: 232 - DNA, 20 bp, synthetic construct
  misc_feature 1..20: note = Synthetic
  source 1..20: mol_type = other DNA
atccacccatttatcgtttt

SEQ ID NO: 233 - DNA, 20 bp, synthetic construct
  misc_feature 1..20: note = Synthetic
  source 1..20: mol_type = other DNA
gaaacaattgaacgaaactg

SEQ ID NO: 234 - DNA, 20 bp, synthetic construct
  misc_feature 1..20: note = Synthetic
  source 1..20: mol_type = other DNA
agcccagagcacctttacat

SEQ ID NO: 235 - DNA, 20 bp, synthetic construct
  misc_feature 1..20: note = Synthetic
  source 1..20: mol_type = other DNA
ctgttctctcgggcttcaac

SEQ ID NO: 236 - DNA, 20 bp, synthetic construct
  misc_feature 1..20: note = Synthetic
  source 1..20: mol_type = other DNA
ctctcgggcttcaacaggca

SEQ ID NO: 237 - DNA, 20 bp, synthetic construct
  misc_feature 1..20: note = Synthetic
  source 1..20: mol_type = other DNA
tgcactccctttggggaggt

SEQ ID NO: 238 - DNA, 20 bp, synthetic construct
  misc_feature 1..20: note = Synthetic
  source 1..20: mol_type = other DNA
attctatgtttttcactacc

SEQ ID NO: 239 - DNA, 20 bp, synthetic construct
  misc_feature 1..20: note = Synthetic
  source 1..20: mol_type = other DNA
gacgaaaggagttcccatac

SEQ ID NO: 240 - DNA, 20 bp, synthetic construct
  misc_feature 1..20: note = Synthetic
  source 1..20: mol_type = other DNA
aggtaccattagcagacgaa

SEQ ID NO: 241 - DNA, 20 bp, synthetic construct
  misc_feature 1..20: note = Synthetic
  source 1..20: mol_type = other DNA
tgcctaccataaatacccct

SEQ ID NO: 242 - DNA, 20 bp, synthetic construct
  misc_feature 1..20: note = Synthetic
  source 1..20: mol_type = other DNA
ttagccagaaggtgtcaaaa

SEQ ID NO: 243 - DNA, 20 bp, synthetic construct
  misc_feature 1..20: note = Synthetic
  source 1..20: mol_type = other DNA
tgccctgatgatcaatgact

SEQ ID NO: 244 - DNA, 20 bp, synthetic construct
  misc_feature 1..20: note = Synthetic
  source 1..20: mol_type = other DNA
gtgccctgatgatcaatgac

SEQ ID NO: 245 - DNA, 20 bp, synthetic construct
  misc_feature 1..20: note = Synthetic
  source 1..20: mol_type = other DNA
agcccagtcattgatcatca

SEQ ID NO: 246 - DNA, 20 bp, synthetic construct
  misc_feature 1..20: note = Synthetic
  source 1..20: mol_type = other DNA
aagcccagtcattgatcatc

SEQ ID NO: 247 - DNA, 20 bp, synthetic construct
  misc_feature 1..20: note = Synthetic
  source 1..20: mol_type = other DNA
tttacaactataggacttct

SEQ ID NO: 248 - DNA, 20 bp, synthetic construct
  misc_feature 1..20: note = Synthetic
  source 1..20: mol_type = other DNA
tctcctcgtacaacagaata

SEQ ID NO: 249 - DNA, 20 bp, synthetic construct
  misc_feature 1..20: note = Synthetic
  source 1..20: mol_type = other DNA
aaggaactgtttacaactat

SEQ ID NO: 250 - DNA, 20 bp, synthetic construct
  misc_feature 1..20: note = Synthetic
  source 1..20: mol_type = other DNA
gtttacaactataggacttc

SEQ ID NO: 251 - DNA, 20 bp, synthetic construct
  misc_feature 1..20: note = Synthetic
  source 1..20: mol_type = other DNA
ataccatattctgttgtacg

SEQ ID NO: 252 - DNA, 20 bp, synthetic construct
  misc_feature 1..20: note = Synthetic
  source 1..20: mol_type = other DNA
cgaatttctggcttgcttac

SEQ ID NO: 253 - DNA, 20 bp, synthetic construct
  misc_feature 1..20: note = Synthetic
  source 1..20: mol_type = other DNA
cagtaagcaagccagaaatt

SEQ ID NO: 254 - DNA, 20 bp, synthetic construct
  misc_feature 1..20: note = Synthetic
  source 1..20: mol_type = other DNA
tggcttgcttactggtaaca

SEQ ID NO: 255 - DNA, 20 bp, synthetic construct
  misc_feature 1..20: note = Synthetic
  source 1..20: mol_type = other DNA
ctggcttgcttactggtaac

SEQ ID NO: 256 - DNA, 20 bp, synthetic construct
  misc_feature 1..20: note = Synthetic
  source 1..20: mol_type = other DNA
ccgtgcagctcgctgctcac

SEQ ID NO: 257 - DNA, 20 bp, synthetic construct
  misc_feature 1..20: note = Synthetic
  source 1..20: mol_type = other DNA
gtgaagaaatgttgttacga

SEQ ID NO: 258 - DNA, 20 bp, synthetic construct
  misc_feature 1..20: note = Synthetic
  source 1..20: mol_type = other DNA
ttcttagagaagagtgatct

SEQ ID NO: 259 - DNA, 20 bp, synthetic construct
  misc_feature 1..20: note = Synthetic
  source 1..20: mol_type = other DNA
attcttagagaagagtgatc

SEQ ID NO: 260 - DNA, 20 bp, synthetic construct
  misc_feature 1..20: note = Synthetic
  source 1..20: mol_type = other DNA
gccaatgtgttccacctagc

SEQ ID NO: 261 - DNA, 20 bp, synthetic construct
  misc_feature 1..20: note = Synthetic
  source 1..20: mol_type = other DNA
tgaggaaggtaagtccagct

SEQ ID NO: 262 - DNA, 20 bp, synthetic construct
  misc_feature 1..20: note = Synthetic
  source 1..20: mol_type = other DNA
ggaaggtaagtccagctagg

SEQ ID NO: 263 - DNA, 20 bp, synthetic construct
  misc_feature 1..20: note = Synthetic
  source 1..20: mol_type = other DNA
tccagctaggtggaacacat

SEQ ID NO: 264 - DNA, 20 bp, synthetic construct
  misc_feature 1..20: note = Synthetic
  source 1..20: mol_type = other DNA
aactctttccaagggctttt

SEQ ID NO: 265 - DNA, 20 bp, synthetic construct
  misc_feature 1..20: note = Synthetic
  source 1..20: mol_type = other DNA
cattacttaactctttccaa

SEQ ID NO: 266 - DNA, 20 bp, synthetic construct
  misc_feature 1..20: note = Synthetic
  source 1..20: mol_type = other DNA
ccatattaagagacacagtt

SEQ ID NO: 267 - DNA, 20 bp, synthetic construct
  misc_feature 1..20: note = Synthetic
  source 1..20: mol_type = other DNA
atttgtggcttacctggagc

SEQ ID NO: 268 - DNA, 20 bp, synthetic construct
  misc_feature 1..20: note = Synthetic
  source 1..20: mol_type = other DNA
aagggcaaaattatccactt

SEQ ID NO: 269 - DNA, 20 bp, synthetic construct
  misc_feature 1..20: note = Synthetic
  source 1..20: mol_type = other DNA
aattagtgtctgattcagtc

SEQ ID NO: 270 - DNA, 20 bp, synthetic construct
  misc_feature 1..20: note = Synthetic
  source 1..20: mol_type = other DNA
tttgtcaatatatgggcttt

SEQ ID NO: 271 - DNA, 20 bp, synthetic construct
  misc_feature 1..20: note = Synthetic
  source 1..20: mol_type = other DNA
gagttattttgtcaatatat

SEQ ID NO: 272 - DNA, 20 bp, synthetic construct
  misc_feature 1..20: note = Synthetic
  source 1..20: mol_type = other DNA
ttgtcaatatatgggctttt

SEQ ID NO: 273 - DNA, 20 bp, synthetic construct
  misc_feature 1..20: note = Synthetic
  source 1..20: mol_type = other DNA
gagatgagtatgctattgct

SEQ ID NO: 274 - DNA, 20 bp, synthetic construct
  misc_feature 1..20: note = Synthetic
  source 1..20: mol_type = other DNA
tttgtgcattcagtgttact

SEQ ID NO: 275 - DNA, 20 bp, synthetic construct
  misc_feature 1..20: note = Synthetic
  source 1..20: mol_type = other DNA
gtttgtgcattcagtgttac

SEQ ID NO: 276 - DNA, 20 bp, synthetic construct
  misc_feature 1..20: note = Synthetic
  source 1..20: mol_type = other DNA
agtgtaacacgtgttgatga

SEQ ID NO: 277 - DNA, 20 bp, synthetic construct
  misc_feature 1..20: note = Synthetic
  source 1..20: mol_type = other DNA
ccagcaccgtcactccagat

SEQ ID NO: 278 - DNA, 20 bp, synthetic construct
  misc_feature 1..20: note = Synthetic
  source 1..20: mol_type = other DNA
ttgttttgacaaacaggtgc

SEQ ID NO: 279 - DNA, 20 bp, synthetic construct
  misc_feature 1..20: note = Synthetic
  source 1..20: mol_type = other DNA
cccatctggagtgacggtgc

SEQ ID NO: 280 - DNA, 20 bp, synthetic construct
  misc_feature 1..20: note = Synthetic
  source 1..20: mol_type = other DNA
tccagcaccgtcactccaga

SEQ ID NO: 281 - DNA, 20 bp, synthetic construct
  misc_feature 1..20: note = Synthetic
  source 1..20: mol_type = other DNA
cttaggtggattttctgaag

SEQ ID NO: 282 - DNA, 20 bp, synthetic construct
  misc_feature 1..20: note = Synthetic
  source 1..20: mol_type = other DNA
atgcttgccatttccttagg

SEQ ID NO: 283 - DNA, 20 bp, synthetic construct
  misc_feature 1..20: note = Synthetic
  source 1..20: mol_type = other DNA
ctctccctacaaactgaatt

SEQ ID NO: 284 - DNA, 20 bp, synthetic construct
  misc_feature 1..20: note = Synthetic
  source 1..20: mol_type = other DNA
gagaggacatatttgatgcc

SEQ ID NO: 285 - DNA, 20 bp, synthetic construct
  misc_feature 1..20: note = Synthetic
  source 1..20: mol_type = other DNA
gcaaccaaattcagtttgta

SEQ ID NO: 286 - DNA, 20 bp, synthetic construct
  misc_feature 1..20: note = Synthetic
  source 1..20: mol_type = other DNA
gcttccacagatatggtatg

SEQ ID NO: 287 - DNA, 20 bp, synthetic construct
  misc_feature 1..20: note = Synthetic
  source 1..20: mol_type = other DNA
ttagacggaatcttgaagtc

SEQ ID NO: 288 - DNA, 20 bp, synthetic construct
  misc_feature 1..20: note = Synthetic
  source 1..20: mol_type = other DNA
aagacagtttctcttttggc

SEQ ID NO: 289 - DNA, 20 bp, synthetic construct
  misc_feature 1..20: note = Synthetic
  source 1..20: mol_type = other DNA
aatccattgtcataggttat

SEQ ID NO: 290 - DNA, 20 bp, synthetic construct
  misc_feature 1..20: note = Synthetic
  source 1..20: mol_type = other DNA
tctctgaggataaatgaaca

SEQ ID NO: 291 - DNA, 20 bp, synthetic construct
  misc_feature 1..20: note = Synthetic
  source 1..20: mol_type = other DNA
ggataaatgaacatggcctg

SEQ ID NO: 292 - DNA, 20 bp, synthetic construct
  misc_feature 1..20: note = Synthetic
  source 1..20: mol_type = other DNA
gcaccaaaaatattccgtgt

SEQ ID NO: 293 - DNA, 20 bp, synthetic construct
  misc_feature 1..20: note = Synthetic
  source 1..20: mol_type = other DNA
gctccaacacggaatatttt

SEQ ID NO: 294 - DNA, 20 bp, synthetic construct
  misc_feature 1..20: note = Synthetic
  source 1..20: mol_type = other DNA
tttcctgctacctccaacca

SEQ ID NO: 295 - DNA, 20 bp, synthetic construct
  misc_feature 1..20: note = Synthetic
  source 1..20: mol_type = other DNA
cctccaaccatgggcctttt

SEQ ID NO: 296 - DNA, 20 bp, synthetic construct
  misc_feature 1..20: note = Synthetic
  source 1..20: mol_type = other DNA
acctccaaccatgggccttt

SEQ ID NO: 297 - RNA, 100 nt, synthetic construct
  misc_feature 1..100: note = Synthetic
  source 1..100: mol_type = other RNA
tgcctaccataaatacccctgttttagagctagaaatagcaagttaaaataaggctagtccgttatcaacttgaaaaagtggcaccgagtcggtgctttt

SEQ ID NO: 298 - RNA, 100 nt, synthetic construct
  misc_feature 1..100: note = Synthetic
  source 1..100: mol_type = other RNA
ctctcgggcttcaacaggcagttttagagctagaaatagcaagttaaaataaggctagtccgttatcaacttgaaaaagtggcaccgagtcggtgctttt

SEQ ID NO: 299 - RNA, 100 nt, synthetic construct
  misc_feature 1..100: note = Synthetic
  source 1..100: mol_type = other RNA
gagatgagtatgctattgctgttttagagctagaaatagcaagttaaaataaggctagtccgttatcaacttgaaaaagtggcaccgagtcggtgctttt

SEQ ID NO: 300 - RNA, 100 nt, synthetic construct
  misc_feature 1..100: note = Synthetic
  source 1..100: mol_type = other RNA
tccagctaggtggaacacatgttttagagctagaaatagcaagttaaaataaggctagtccgttatcaacttgaaaaagtggcaccgagtcggtgctttt

SEQ ID NO: 301 - RNA, 100 nt, synthetic construct
  misc_feature 1..100: note = Synthetic
  source 1..100: mol_type = other RNA
tgaggaaggtaagtccagctgttttagagctagaaatagcaagttaaaataaggctagtccgttatcaacttgaaaaagtggcaccgagtcggtgctttt

SEQ ID NO: 302 - RNA, 100 nt, synthetic construct
  misc_feature 1..100: note = Synthetic
  source 1..100: mol_type = other RNA
tgccctgatgatcaatgactgttttagagctagaaatagcaagttaaaataaggctagtccgttatcaacttgaaaaagtggcaccgagtcggtgctttt

SEQ ID NO: 303 - RNA, 100 nt, synthetic construct
  misc_feature 1..100: note = Synthetic
  source 1..100: mol_type = other RNA
cctccaaccatgggccttttgttttagagctagaaatagcaagttaaaataaggctagtccgttatcaacttgaaaaagtggcaccgagtcggtgctttt

SEQ ID NO: 304 - RNA, 100 nt, synthetic construct
  misc_feature 1..100: note = Synthetic
  source 1..100: mol_type = other RNA
ttcttagagaagagtgatctgttttagagctagaaatagcaagttaaaataaggctagtccgttatcaacttgaaaaagtggcaccgagtcggtgctttt

SEQ ID NO: 305 - RNA, 100 nt, synthetic construct
  misc_feature 1..100: note = Synthetic
  source 1..100: mol_type = other RNA
gactttctgaattttgatatgttttagagctagaaatagcaagttaaaataaggctagtccgttatcaacttgaaaaagtggcaccgagtcggtgctttt

SEQ ID NO: 306 - RNA, 100 nt, synthetic construct
  misc_feature 1..100: note = Synthetic
  source 1..100: mol_type = other RNA
gacagctctgtacctaacacgttttagagctagaaatagcaagttaaaataaggctagtccgttatcaacttgaaaaagtggcaccgagtcggtgctttt

SEQ ID NO: 307 - RNA, 100 nt, synthetic construct
  misc_feature 1..100: note = Synthetic
  source 1..100: mol_type = other RNA
tgcactccctttggggaggtgttttagagctagaaatagcaagttaaaataaggctagtccgttatcaacttgaaaaagtggcaccgagtcggtgctttt

SEQ ID NO: 308 - RNA, 100 nt, synthetic construct
  misc_feature 1..100: note = Synthetic
  source 1..100: mol_type = other RNA
cagtaagcaagccagaaattgttttagagctagaaatagcaagttaaaataaggctagtccgttatcaacttgaaaaagtggcaccgagtcggtgctttt

SEQ ID NO: 309 - RNA, 100 nt, synthetic construct
  misc_feature 1..100: note = Synthetic
  source 1..100: mol_type = other RNA
gccaatgtgttccacctagcgttttagagctagaaatagcaagttaaaataaggctagtccgttatcaacttgaaaaagtggcaccgagtcggtgctttt

SEQ ID NO: 310 - RNA, 100 nt, synthetic construct
  misc_feature 1..100: note = Synthetic
  source 1..100: mol_type = other RNA
tttgtcaatatatgggctttgttttagagctagaaatagcaagttaaaataaggctagtccgttatcaacttgaaaaagtggcaccgagtcggtgctttt

SEQ ID NO: 311 - RNA, 100 nt, synthetic construct
  misc_feature 1..100: note = Synthetic
  source 1..100: mol_type = other RNA
atgcttgccatttccttagggttttagagctagaaatagcaagttaaaataaggctagtccgttatcaacttgaaaaagtggcaccgagtcggtgctttt

SEQ ID NO: 312 - RNA, 100 nt, synthetic construct
  misc_feature 1..100: note = Synthetic
  source 1..100: mol_type = other RNA
atccacccatttatcgttttgttttagagctagaaatagcaagttaaaataaggctagtccgttatcaacttgaaaaagtggcaccgagtcggtgctttt

SEQ ID NO: 313 - RNA, 95 nt, synthetic construct
  misc_feature 1..95: note = Synthetic
  source 1..95: mol_type = other RNA
gcaatggtgtagcgggttttagagctagaaatagcaagttaaaataaggctagtccgttatcaacttgaaaaagtggcaccgagtcggtgctttt

SEQ ID NO: 314 - RNA, 100 nt, synthetic construct
  misc_feature 1..100: note = Synthetic
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 315 - RNA, 100 nt, synthetic construct
  misc_feature 1..100: note = Synthetic
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 316 - RNA, 100 nt, synthetic construct; identical to SEQ ID NO: 297
  misc_feature 1..100: note = Synthetic
  source 1..100: mol_type = other RNA

SEQ ID NO: 317 - RNA, 100 nt, synthetic construct; identical to SEQ ID NO: 298
  misc_feature 1..100: note = Synthetic
  source 1..100: mol_type = other RNA

SEQ ID NO: 318 - RNA, 100 nt, synthetic construct; identical to SEQ ID NO: 299
  misc_feature 1..100: note = Synthetic
  source 1..100: mol_type = other RNA

SEQ ID NO: 319 - RNA, 100 nt, synthetic construct; identical to SEQ ID NO: 300
  misc_feature 1..100: note = Synthetic
  source 1..100: mol_type = other RNA

SEQ ID NO: 320 - RNA, 100 nt, synthetic construct; identical to SEQ ID NO: 301
  misc_feature 1..100: note = Synthetic
  source 1..100: mol_type = other RNA

SEQ ID NO: 321 - RNA, 100 nt, synthetic construct; identical to SEQ ID NO: 302
  misc_feature 1..100: note = Synthetic
  source 1..100: mol_type = other RNA

SEQ ID NO: 322 - RNA, 100 nt, synthetic construct; identical to SEQ ID NO: 303
  misc_feature 1..100: note = Synthetic
  source 1..100: mol_type = other RNA

SEQ ID NO: 323 - RNA, 100 nt, synthetic construct; identical to SEQ ID NO: 304
  misc_feature 1..100: note = Synthetic
  source 1..100: mol_type = other RNA

SEQ ID NO: 324 - RNA, 100 nt, synthetic construct; identical to SEQ ID NO: 305
  misc_feature 1..100: note = Synthetic
  source 1..100: mol_type = other RNA

SEQ ID NO: 325 - RNA, 100 nt, synthetic construct; identical to SEQ ID NO: 306
  misc_feature 1..100: note = Synthetic
  source 1..100: mol_type = other RNA

SEQ ID NO: 326 - RNA, 100 nt, synthetic construct; identical to SEQ ID NO: 307
  misc_feature 1..100: note = Synthetic
  source 1..100: mol_type = other RNA

SEQ ID NO: 327 - RNA, 100 nt, synthetic construct; identical to SEQ ID NO: 308
  misc_feature 1..100: note = Synthetic
  source 1..100: mol_type = other RNA

SEQ ID NO: 328 - RNA, 100 nt, synthetic construct; identical to SEQ ID NO: 309
  misc_feature 1..100: note = Synthetic
  source 1..100: mol_type = other RNA

SEQ ID NO: 329 - RNA, 100 nt, synthetic construct; identical to SEQ ID NO: 310
  misc_feature 1..100: note = Synthetic
  source 1..100: mol_type = other RNA

SEQ ID NO: 330 - RNA, 100 nt, synthetic construct; identical to SEQ ID NO: 311
  misc_feature 1..100: note = Synthetic
  source 1..100: mol_type = other RNA

SEQ ID NO: 331 - RNA, 100 nt, synthetic construct; identical to SEQ ID NO: 312
  misc_feature 1..100: note = Synthetic
  source 1..100: mol_type = other RNA

SEQ ID NO: 332 - RNA, 95 nt, synthetic construct; identical to SEQ ID NO: 313
  misc_feature 1..95: note = Synthetic
  source 1..95: mol_type = other RNA

SEQ ID NO: 333 - RNA, 100 nt, synthetic construct; identical to SEQ ID NO: 314
  misc_feature 1..100: note = Synthetic
  source 1..100: mol_type = other RNA

SEQ ID NO: 334 - RNA, 100 nt, synthetic construct; identical to SEQ ID NO: 315
  misc_feature 1..100: note = Synthetic
  source 1..100: mol_type = other RNA

SEQ ID NO: 335 - DNA, 4140 bp, synthetic construct
  misc_feature 1..4140: note = Synthetic
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 336 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 337 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 338 - RNA, 4423 nt, synthetic construct
  misc_feature 1..4423: note = Synthetic
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 339 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 340 - DNA, 357 bp, synthetic construct
  misc_feature 1..357: note = Synthetic
  source 1..357: mol_type = other DNA
caggtgcagctggtggagtctgggggaggcgtggtccagcctggaaggtccctgagactctcctgtgtagcgtctggattcaccttcagtagttatggcattcactgggtccgccaggctccaggcaaggggctggagtgggtggcagttatatgggatgatggaaataatataaactattcagactccgtgaagggccgattcatcatctccagagacaattccaggaagacagtgtatctgcaaatgaacagcctgagaggcgaggacacggctgtttattactgtgcgagagatgcccccatagcaccagtccctgactattggggccagggaaccctggtcaccgtctcctca

SEQ ID NO: 341 - protein, 119 aa, synthetic construct
  REGION 1..119: note = Synthetic
QVQLVESGGGVVQPGRSLRLSCVASGFTFSSYGIHWVRQAPGKGLEWVAVIWDDGNNINYSDSVKGRFIISRDNSRKTVYLQMNSLRGEDTAVYYCARDAPIAPVPDYWGQGTLVTVSS

SEQ ID NO: 342 - DNA, 24 bp, synthetic construct
  misc_feature 1..24: note = Synthetic
  source 1..24: mol_type = other DNA
ggattcaccttcagtagttatggc

SEQ ID NO: 343 - protein, 8 aa, synthetic construct
  REGION 1..8: note = Synthetic
GFTFSSYG

SEQ ID NO: 344 - DNA, 24 bp, synthetic construct
  misc_feature 1..24: note = Synthetic
  source 1..24: mol_type = other DNA
atatgggatgatggaaataatata

SEQ ID NO: 345 - protein, 8 aa, synthetic construct
  REGION 1..8: note = Synthetic
IWDDGNNI

SEQ ID NO: 346 - DNA, 36 bp, synthetic construct
  misc_feature 1..36: note = Synthetic
  source 1..36: mol_type = other DNA
gcgagagatgcccccatagcaccagtccctgactat

SEQ ID NO: 347 - protein, 12 aa, synthetic construct
  REGION 1..12: note = Synthetic
ARDAPIAPVPDY

SEQ ID NO: 348 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagacactggggtcccatcaaggttcagcggcagtggatctgggacagagttcactctcaccatcagcagcctgcagcctgatgattttgcaacttattactgccaacagtataatacttattcgtacacttttggcctggggaccaaactggagatcaaa

SEQ ID NO: 349 - protein, 107 aa, synthetic construct
  REGION 1..107: note = Synthetic
DIQMTQSPSTLSASVGDRVTITCRASQSISSWLAWYQQKPGKAPKLLIYKASSLDTGVPSRFSGSGSGTEFTLTISSLQPDDFATYYCQQYNTYSYTFGLGTKLEIK

SEQ ID NO: 350 - DNA, 18 bp, synthetic construct
  misc_feature 1..18: note = Synthetic
  source 1..18: mol_type = other DNA
cagagtattagtagttgg

SEQ ID NO: 351 - protein, 6 aa, synthetic construct
  REGION 1..6: note = Synthetic
QSISSW

SEQ ID NO: 352-353 - skipped (2 entries)

SEQ ID NO: 354 - DNA, 27 bp, synthetic construct
  misc_feature 1..27: note = Synthetic
  source 1..27: mol_type = other DNA
caacagtataatacttattcgtacact

SEQ ID NO: 355 - protein, 9 aa, synthetic construct
  REGION 1..9: note = Synthetic
QQYNTYSYT

SEQ ID NO: 356 - DNA, 357 bp, synthetic construct
  misc_feature 1..357: note = Synthetic
  source 1..357: mol_type = other DNA
caggtgcaactggtggagtctgggggaggcttggtcaagcctggagggtccctgagactctcctgtgcagcttctggattcaccttcagtgactactacatgagctggatccgccaggctccagggaaggggctggagtgggtttcatatattagcagtagtggtaataccataaaatatgcagactctatgaagggccgattcaccatctccagggacaacgccaagaaatcactgtttgtggaaatgaacagcctgagagccgaggacacggccgtgtattactgtgcgaggtataaaagttcgtccgactactttgaccactggggccagggaaccctggtcaccgtctcctca

SEQ ID NO: 357 - protein, 119 aa, synthetic construct
  REGION 1..119: note = Synthetic
QVQLVESGGGLVKPGGSLRLSCAASGFTFSDYYMSWIRQAPGKGLEWVSYISSSGNTIKYADSMKGRFTISRDNAKKSLFVEMNSLRAEDTAVYYCARYKSSSDYFDHWGQGTLVTVSS

SEQ ID NO: 358 - DNA, 24 bp, synthetic construct
  misc_feature 1..24: note = Synthetic
  source 1..24: mol_type = other DNA
ggattcaccttcagtgactactac

SEQ ID NO: 359 - protein, 8 aa, synthetic construct
  REGION 1..8: note = Synthetic
GFTFSDYY

SEQ ID NO: 360 - DNA, 24 bp, synthetic construct
  misc_feature 1..24: note = Synthetic
  source 1..24: mol_type = other DNA
attagcagtagtggtaataccata

SEQ ID NO: 361 - protein, 8 aa, synthetic construct
  REGION 1..8: note = Synthetic
ISSSGNTI

SEQ ID NO: 362 - DNA, 36 bp, synthetic construct
  misc_feature 1..36: note = Synthetic
  source 1..36: mol_type = other DNA
gcgaggtataaaagttcgtccgactactttgaccac

SEQ ID NO: 363 - protein, 12 aa, synthetic construct
  REGION 1..12: note = Synthetic
ARYKSSSDYFDH

SEQ ID NO: 364 - DNA, 321 bp, synthetic construct
  misc_feature 1..321: note = Synthetic
  source 1..321: mol_type = other DNA
gaaattgtgttgacacagtctccagccaccctgtctttgtctccaggggaaagagccaccctctcctgcagggccagtcagagtgttaggagttacttagcctggtaccaacagaaacctggccaggctcccaggctcctcatctatgatgcatccaacagggccactgccatcccagccaggttcagtggcagtgggtctgggacagacttcactctcaccatcagcagcctagagcctgaagatttagcagtttattactgtcagcagtctggcaactggccgctcactttcggcggagggaccaaggtggagatcaaa

SEQ ID NO: 365 - protein, 107 aa, synthetic construct
  REGION 1..107: note = Synthetic
EIVLTQSPATLSLSPGERATLSCRASQSVRSYLAWYQQKPGQAPRLLIYDASNRATAIPARFSGSGSGTDFTLTISSLEPEDLAVYYCQQSGNWPLTFGGGTKVEIK

SEQ ID NO: 366 - DNA, 18 bp, synthetic construct
  misc_feature 1..18: note = Synthetic
  source 1..18: mol_type = other DNA
cagagtgttaggagttac

SEQ ID NO: 367 - protein, 6 aa, synthetic construct
  REGION 1..6: note = Synthetic
QSVRSY

SEQ ID NO: 368-369 - skipped (2 entries)

SEQ ID NO: 370 - DNA, 27 bp, synthetic construct
  misc_feature 1..27: note = Synthetic
  source 1..27: mol_type = other DNA
cagcagtctggcaactggccgctcact

SEQ ID NO: 371 - protein, 9 aa, synthetic construct
  REGION 1..9: note = Synthetic
QQSGNWPLT

SEQ ID NO: 372 - DNA, 357 bp, synthetic construct
  misc_feature 1..357: note = Synthetic
  source 1..357: mol_type = other DNA
caggtgcagctggtggagtctgggggaggcgtggtccagcctgggaggtccttgagactctcctgtggagcgtctggattcaccttcagtacttatggcatgcactgggtccgccaggctccaggcaaggggctggagtgggtggcagttatctgggatgatggaaataataaatattatgcagactccgtgaagggccgattcaccatctccagagacaattcgaagaacacgctgtatctgcagatgaacagcctgagagccgaggacacggctgtttattactgtgcgagagattcagaggtcgccccagttggggactactggggccagggcaccctggtcaccgtctcctca

SEQ ID NO: 373 - protein, 119 aa, synthetic construct
  REGION 1..119: note = Synthetic
QVQLVESGGGVVQPGRSLRLSCGASGFTFSTYGMHWVRQAPGKGLEWVAVIWDDGNNKYYADSVKGRFTISRDNSKNTLYLQMNSLRAEDTAVYYCARDSEVAPVGDYWGQGTLVTVSS

SEQ ID NO: 374 - DNA, 24 bp, synthetic construct
  misc_feature 1..24: note = Synthetic
  source 1..24: mol_type = other DNA
ggattcaccttcagtacttatggc

SEQ ID NO: 375 - protein, 8 aa, synthetic construct
  REGION 1..8: note = Synthetic
GFTFSTYG

SEQ ID NO: 376 - DNA, 24 bp, synthetic construct
  misc_feature 1..24: note = Synthetic
  source 1..24: mol_type = other DNA
atctgggatgatggaaataataaa

SEQ ID NO: 377 - protein, 8 aa, synthetic construct
  REGION 1..8: note = Synthetic
IWDDGNNK

SEQ ID NO: 378 - DNA, 36 bp, synthetic construct
  misc_feature 1..36: note = Synthetic
  source 1..36: mol_type = other DNA
gcgagagattcagaggtcgccccagttggggactac

SEQ ID NO: 379 - protein, 12 aa, synthetic construct
  REGION 1..12: note = Synthetic
ARDSEVAPVGDY

SEQ ID NO: 380 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcactatcatttgccgggccagtcagagtattaacaggtggttggcctggtatcagcagaaaccagggaaggcccctaaactcctgatctataaggcgtctagtttagaaagtggggtcccatcaaggttcagcggcagtggatctgggacagaattcactctcaccatcagcagcctgcagcctgatgattttgcagcttattactgccaacagtataatgattattcgtacacttttggccaggggaccaagctggagatcaaa

SEQ ID NO: 381 - protein, 107 aa, synthetic construct
  REGION 1..107: note = Synthetic
DIQMTQSPSTLSASVGDRVTIICRASQSINRWLAWYQQKPGKAPKLLIYKASSLESGVPSRFSGSGSGTEFTLTISSLQPDDFAAYYCQQYNDYSYTFGQGTKLEIK

SEQ ID NO: 382 - DNA, 18 bp, synthetic construct
  misc_feature 1..18: note = Synthetic
  source 1..18: mol_type = other DNA
cagagtattaacaggtgg

SEQ ID NO: 383 - protein, 6 aa, synthetic construct
  REGION 1..6: note = Synthetic
QSINRW

SEQ ID NO: 384-385 - skipped (2 entries)

SEQ ID NO: 386 - DNA, 27 bp, synthetic construct
  misc_feature 1..27: note = Synthetic
  source 1..27: mol_type = other DNA
caacagtataatgattattcgtacact

SEQ ID NO: 387 - protein, 9 aa, synthetic construct
  REGION 1..9: note = Synthetic
QQYNDYSYT

SEQ ID NO: 388 - DNA, 366 bp, synthetic construct
  misc_feature 1..366: note = Synthetic
  source 1..366: mol_type = other DNA
gaggtgcagctggtggagtctgggggagacttggtccagcctggagggtccctgagactctcctgtgcagcctctggattcaccttcagtgaccactatatggactgggtccgccaggctccagggaaggggctggactggattggccgtattagaaacaaagctaacgcttataacacagaatacgccgcgtctgtgagaggcagattcaccatctcaagagatgattcacagaatttactgtatctgcaaatgaacagcctgaaaaccgatgacacggccgtatattattgtgttagagtctggaactacgcctacttcgctatggacgtctggggccaagggaccacggtcaccgtctcctca

SEQ ID NO: 389 - protein, 122 aa, synthetic construct
  REGION 1..122: note = Synthetic
EVQLVESGGDLVQPGGSLRLSCAASGFTFSDHYMDWVRQAPGKGLDWIGRIRNKANAYNTEYAASVRGRFTISRDDSQNLLYLQMNSLKTDDTAVYYCVRVWNYAYFAMDVWGQGTTVTVSS

SEQ ID NO: 390 - DNA, 24 bp, synthetic construct
  misc_feature 1..24: note = Synthetic
  source 1..24: mol_type = other DNA
ggattcaccttcagtgaccactat

SEQ ID NO: 391 - protein, 8 aa, synthetic construct
  REGION 1..8: note = Synthetic
GFTFSDHY

SEQ ID NO: 392 - DNA, 30 bp, synthetic construct
  misc_feature 1..30: note = Synthetic
  source 1..30: mol_type = other DNA
attagaaacaaagctaacgcttataacaca

SEQ ID NO: 393 - protein, 10 aa, synthetic construct
  REGION 1..10: note = Synthetic
IRNKANAYNT

SEQ ID NO: 394 - DNA, 39 bp, synthetic construct
  misc_feature 1..39: note = Synthetic
  source 1..39: mol_type = other DNA
gttagagtctggaactacgcctacttcgctatggacgtc

SEQ ID NO: 395 - protein, 13 aa, synthetic construct
  REGION 1..13: note = Synthetic
VRVWNYAYFAMDV

SEQ ID NO: 396 - DNA, 321 bp, synthetic construct
  misc_feature 1..321: note = Synthetic
  source 1..321: mol_type = other DNA
gacatccagatgacccagtctccatcctccctatctgcatctgtgggagacagagtcaccatcacttgccggtcaagtcagaacattggaatctttttaaactggtatcaacaaaaaccaggggaagcccctaacctcctgatctccgctgcatccagtttacacagtggggtcccttcaaggttcagtggcagtgggtctgggacagatttcactctcaccatcggcagtctgcagcctgaagattttgcgacttactactgtcaacagacgtacaataccatattcactttcggccctgggaccaaagtggatatcaaa

SEQ ID NO: 397 - protein, 107 aa, synthetic construct
  REGION 1..107: note = Synthetic
DIQMTQSPSSLSASVGDRVTITCRSSQNIGIFLNWYQQKPGEAPNLLISAASSLHSGVPSRFSGSGSGTDFTLTIGSLQPEDFATYYCQQTYNTIFTFGPGTKVDIK

SEQ ID NO: 398 - DNA, 18 bp, synthetic construct
  misc_feature 1..18: note = Synthetic
  source 1..18: mol_type = other DNA
cagaacattggaatcttt

SEQ ID NO: 399 - protein, 6 aa, synthetic construct
  REGION 1..6: note = Synthetic
QNIGIF

SEQ ID NO: 400-401 - skipped (2 entries)

SEQ ID NO: 402 - DNA, 27 bp, synthetic construct
  misc_feature 1..27: note = Synthetic
  source 1..27: mol_type = other DNA
caacagacgtacaataccatattcact

SEQ ID NO: 403 - protein, 9 aa, synthetic construct
  REGION 1..9: note = Synthetic
QQTYNTIFT

SEQ ID NO: 404 - DNA, 363 bp, synthetic construct
  misc_feature 1..363: note = Synthetic
  source 1..363: mol_type = other DNA
gaggtgcagctggtggagtctgggggagacttggtacagcctggggggtccctgagactctcctgtgcagcctctggattcacctttagcagctatgccatgaactgggtccgccagggtccagggaagggactggagtgggtctcagctattagtggtcgtggtgatagtacatactacgcagactccgtgaagggccggctcaccatctccagagacaattccaagaacacgctgtatctgcaaatgaacagcctgagagccgaggacacggccgtatattactgtgtgaaagagggggagcaactcgtctactggtacttcgatctctggggccgtggcaccctggtcaccgtctcctca

SEQ ID NO: 405 - protein, 121 aa, synthetic construct
  REGION 1..121: note = Synthetic
EVQLVESGGDLVQPGGSLRLSCAASGFTFSSYAMNWVRQGPGKGLEWVSAISGRGDSTYYADSVKGRLTISRDNSKNTLYLQMNSLRAEDTAVYYCVKEGEQLVYWYFDLWGRGTLVTVSS

SEQ ID NO: 406 - DNA, 24 bp, synthetic construct
  misc_feature 1..24: note = Synthetic
  source 1..24: mol_type = other DNA
ggattcacctttagcagctatgcc

SEQ ID NO: 407 - protein, 8 aa, synthetic construct
  REGION 1..8: note = Synthetic
GFTFSSYA

SEQ ID NO: 408 - DNA, 24 bp, synthetic construct
  misc_feature 1..24: note = Synthetic
  source 1..24: mol_type = other DNA
attagtggtcgtggtgatagtaca

SEQ ID NO: 409 - protein, 8 aa, synthetic construct
  REGION 1..8: note = Synthetic
ISGRGDST

SEQ ID NO: 410 - DNA, 42 bp, synthetic construct
  misc_feature 1..42: note = Synthetic
  source 1..42: mol_type = other DNA
gtgaaagagggggagcaactcgtctactggtacttcgatctc

SEQ ID NO: 411 - protein, 14 aa, synthetic construct
  REGION 1..14: note = Synthetic
VKEGEQLVYWYFDL

SEQ ID NO: 412 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagaccattagcaactttttacattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatttttcaacttacttctgtcaacagagttacactaccccgctcactttcggcggagggaccaaggtggagatcaaa

SEQ ID NO: 413 - protein, 107 aa, synthetic construct
  REGION 1..107: note = Synthetic
DIQMTQSPSSLSASVGDRVTITCRASQTISNFLHWYQQKPGKAPKLLIYAASSLQSGVPSRFSGSGSGTDFTLTISSLQPEDFSTYFCQQSYTTPLTFGGGTKVEIK

SEQ ID NO: 414 - DNA, 18 bp, synthetic construct
  misc_feature 1..18: note = Synthetic
  source 1..18: mol_type = other DNA
cagaccattagcaacttt

SEQ ID NO: 415 - protein, 6 aa, synthetic construct
  REGION 1..6: note = Synthetic
QTISNF

SEQ ID NO: 416-417 - skipped (2 entries)

SEQ ID NO: 418 - DNA, 27 bp, synthetic construct
  misc_feature 1..27: note = Synthetic
  source 1..27: mol_type = other DNA
caacagagttacactaccccgctcact

SEQ ID NO: 419 - protein, 9 aa, synthetic construct
  REGION 1..9: note = Synthetic
QQSYTTPLT

SEQ ID NO: 420 - DNA, 363 bp, synthetic construct
  misc_feature 1..363: note = Synthetic
  source 1..363: mol_type = other DNA
gaggtgcagctggtggagtctgggggaggcttggtgaggtcgggggggtccctgagactctcctgtgcagcctctggattcacctttaacagatatgccatgacctgggtccgccaggctccagggaaggggctggagtgggtctcagctataagtggtagtggtagcagcacatactacacagactccgtgaaaggccggttcaccatctccagagacaattccaagaattcggtggatctgcaaatgcacagcctgagagtcgaagacacggccatatattattgtgcgagagggactacagtcactacggggtacggtatggacgtctggggccaagggaccacggtcaccgtctcctca

SEQ ID NO: 421 - protein, 121 aa, synthetic construct
  REGION 1..121: note = Synthetic
EVQLVESGGGLVRSGGSLRLSCAASGFTFNRYAMTWVRQAPGKGLEWVSAISGSGSSTYYTDSVKGRFTISRDNSKNSVDLQMHSLRVEDTAIYYCARGTTVTTGYGMDVWGQGTTVTVSS

SEQ ID NO: 422 - DNA, 24 bp, synthetic construct
  misc_feature 1..24: note = Synthetic
  source 1..24: mol_type = other DNA
ggattcacctttaacagatatgcc

SEQ ID NO: 423 - protein, 8 aa, synthetic construct
  REGION 1..8: note = Synthetic
GFTFNRYA

SEQ ID NO: 424 - DNA, 24 bp, synthetic construct
  misc_feature 1..24: note = Synthetic
  source 1..24: mol_type = other DNA
ataagtggtagtggtagcagcaca

SEQ ID NO: 425 - protein, 8 aa, synthetic construct
  REGION 1..8: note = Synthetic
ISGSGSST

SEQ ID NO: 426 - DNA, 42 bp, synthetic construct
  misc_feature 1..42: note = Synthetic
  source 1..42: mol_type = other DNA
gcgagagggactacagtcactacggggtacggtatggacgtc

SEQ ID NO: 427 - protein, 14 aa, synthetic construct
  REGION 1..14: note = Synthetic
ARGTTVTTGYGMDV

SEQ ID NO: 428 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccttcacttgccaggcgagtcaggacattaccaattctttaaattggtatcaacagaaacctgggagagcccctaagctcctgatctacgatgcatcgtatttgaaggcaggggtcccatcaagattcagtggaagtggatctgggacagattttactttcaccatcagcagcctgcagcctgaagatattgcaacatattactgtcaacaatatgatgatctcccatacacttttggccaggggaccaagctggagatcaaa

SEQ ID NO: 429 - protein, 107 aa, synthetic construct
  REGION 1..107: note = Synthetic
DIQMTQSPSSLSASVGDRVTFTCQASQDITNSLNWYQQKPGRAPKLLIYDASYLKAGVPSRFSGSGSGTDFTFTISSLQPEDIATYYCQQYDDLPYTFGQGTKLEIK

SEQ ID NO: 430 - DNA, 18 bp, synthetic construct
  misc_feature 1..18: note = Synthetic
  source 1..18: mol_type = other DNA
caggacattaccaattct

SEQ ID NO: 431 - protein, 6 aa, synthetic construct
  REGION 1..6: note = Synthetic
QDITNS

SEQ ID NO: 432-433 - skipped (2 entries)

SEQ ID NO: 434 - DNA, 27 bp, synthetic construct
  misc_feature 1..27: note = Synthetic
  source 1..27: mol_type = other DNA
caacaatatgatgatctcccatacact

SEQ ID NO: 435 - protein, 9 aa, synthetic construct
  REGION 1..9: note = Synthetic
QQYDDLPYT

SEQ ID NO: 436 - DNA, 360 bp, synthetic construct
  misc_feature 1..360: note = Synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatatttgactactggggccagggaaccctggtcaccgtctcctca

SEQ ID NO: 437 - protein, 120 aa, synthetic construct
  REGION 1..120: note = Synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 438 - DNA, 24 bp, synthetic construct
  misc_feature 1..24: note = Synthetic
  source 1..24: mol_type = other DNA
ggtgactccgtcagtagttcctac

SEQ ID NO: 439 - protein, 8 aa, synthetic construct
  REGION 1..8: note = Synthetic
GDSVSSSY

SEQ ID NO: 440 - DNA, 21 bp, synthetic construct
  misc_feature 1..21: note = Synthetic
  source 1..21: mol_type = other DNA
atctattacagtgggagttcc

SEQ ID NO: 441 - protein, 7 aa, synthetic construct
  REGION 1..7: note = Synthetic
IYYSGSS

SEQ ID NO: 442 - DNA, 42 bp, synthetic construct
  misc_feature 1..42: note = Synthetic
  source 1..42: mol_type = other DNA
gcgagagaagggaacgtggatacaactatgatatttgactac

SEQ ID NO: 443 - protein, 14 aa, synthetic construct
  REGION 1..14: note = Synthetic
AREGNVDTTMIFDY

SEQ ID NO: 444 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggacgttcggccaagggaccaaggtggaaatcaaa

SEQ ID NO: 445 - protein, 107 aa, synthetic construct
  REGION 1..107: note = Synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 446 - DNA, 18 bp, synthetic construct
  misc_feature 1..18: note = Synthetic
  source 1..18: mol_type = other DNA
cagggcattagaaatgat

SEQ ID NO: 447 - protein, 6 aa, synthetic construct
  REGION 1..6: note = Synthetic
QGIRND

SEQ ID NO: 448-449 - skipped (2 entries)

SEQ ID NO: 450 - DNA, 27 bp, synthetic construct
  misc_feature 1..27: note = Synthetic
  source 1..27: mol_type = other DNA
ctacaagatttcaattacccgtggacg

SEQ ID NO: 451 - protein, 9 aa, synthetic construct
  REGION 1..9: note = Synthetic
LQDFNYPWT

SEQ ID NO: 452 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtcatcaagatttcaattacccgtggacgttcggccaagggaccaaggtggaaatcaaa

SEQ ID NO: 453 - protein, 107 aa, synthetic construct
  REGION 1..107: note = Synthetic
AIQMTQSPSSLSASVGDRVTITCRASQGIRNDLGWYQQKPGKAPKLLIYAASSLQSGVPSRFAGRGSGTDFTLTISSLQPEDFATYYCHQDFNYPWTFGQGTKVEIK

SEQ ID NO: 454 - DNA, 18 bp, synthetic construct
  misc_feature 1..18: note = Synthetic
  source 1..18: mol_type = other DNA
cagggcattagaaatgat

SEQ ID NO: 455 - protein, 6 aa, synthetic construct
  REGION 1..6: note = Synthetic
QGIRND

SEQ ID NO: 456-457 - skipped (2 entries)

SEQ ID NO: 458 - DNA, 27 bp, synthetic construct
  misc_feature 1..27: note = Synthetic
  source 1..27: mol_type = other DNA
catcaagatttcaattacccgtggacg

SEQ ID NO: 459 - protein, 9 aa, synthetic construct
  REGION 1..9: note = Synthetic
HQDFNYPWT

SEQ ID NO: 460 - DNA, 360 bp, synthetic construct
  misc_feature 1..360: note = Synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaacataacgtggatacaactatgatatttgactactggggccagggaaccctggtcaccgtctcctca

SEQ ID NO: 461 - protein, 120 aa, synthetic construct
  REGION 1..120: note = Synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREHNVDTTMIFDYWGQGTLVTVSS

SEQ ID NO: 462 - DNA, 24 bp, synthetic construct
  misc_feature 1..24: note = Synthetic
  source 1..24: mol_type = other DNA
ggtgactccgtcagtagttcctac

SEQ ID NO: 463 - protein, 8 aa, synthetic construct
  REGION 1..8: note = Synthetic
GDSVSSSY

SEQ ID NO: 464 - DNA, 21 bp, synthetic construct
  misc_feature 1..21: note = Synthetic
  source 1..21: mol_type = other DNA
atctattacagtgggagttcc

SEQ ID NO: 465 - protein, 7 aa, synthetic construct
  REGION 1..7: note = Synthetic
IYYSGSS

SEQ ID NO: 466 - DNA, 42 bp, synthetic construct
  misc_feature 1..42: note = Synthetic
  source 1..42: mol_type = other DNA
gcgagagaacataacgtggatacaactatgatatttgactac

SEQ ID NO: 467 - protein, 14 aa, synthetic construct
  REGION 1..14: note = Synthetic
AREHNVDTTMIFDY

SEQ ID NO: 468 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggcacttcggccaagggaccaaggtggaaatcaaa

SEQ ID NO: 469 - protein, 107 aa, synthetic construct
  REGION 1..107: note = Synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWHFGQGTKVEIK

SEQ ID NO: 470 - DNA, 18 bp, synthetic construct
  misc_feature 1..18: note = Synthetic
  source 1..18: mol_type = other DNA
cagggcattagaaatgat

SEQ ID NO: 471 - protein, 6 aa, synthetic construct
  REGION 1..6: note = Synthetic
QGIRND

SEQ ID NO: 472-473 - skipped (2 entries)

SEQ ID NO: 474 - DNA, 27 bp, synthetic construct
  misc_feature 1..27: note = Synthetic
  source 1..27: mol_type = other DNA
ctacaagatttcaattacccgtggcac

SEQ ID NO: 475 - protein, 9 aa, synthetic construct
  REGION 1..9: note = Synthetic
LQDFNYPWH

SEQ ID NO: 476 - DNA, 360 bp, synthetic construct
  misc_feature 1..360: note = Synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatacatgactactggggccagggaaccctggtcaccgtctcctca

SEQ ID NO: 477 - protein, 120 aa, synthetic construct
  REGION 1..120: note = Synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIHDYWGQGTLVTVSS

SEQ ID NO: 478 - DNA, 24 bp, synthetic construct
  misc_feature 1..24: note = Synthetic
  source 1..24: mol_type = other DNA
ggtgactccgtcagtagttcctac

SEQ ID NO: 479 - protein, 8 aa, synthetic construct
  REGION 1..8: note = Synthetic
GDSVSSSY

SEQ ID NO: 480 - DNA, 21 bp, synthetic construct
  misc_feature 1..21: note = Synthetic
  source 1..21: mol_type = other DNA
atctattacagtgggagttcc

SEQ ID NO: 481 - protein, 7 aa, synthetic construct
  REGION 1..7: note = Synthetic
IYYSGSS

SEQ ID NO: 482 - DNA, 42 bp, synthetic construct
  misc_feature 1..42: note = Synthetic
  source 1..42: mol_type = other DNA
gcgagagaagggaacgtggatacaactatgatacatgactac

SEQ ID NO: 483 - protein, 14 aa, synthetic construct
  REGION 1..14: note = Synthetic
AREGNVDTTMIHDY

SEQ ID NO: 484 - DNA, 360 bp, synthetic construct
  misc_feature 1..360: note = Synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatcacactatgatatttgactactggggccagggaaccctggtcaccgtctcctca

SEQ ID NO: 485 - protein, 120 aa, synthetic construct
  REGION 1..120: note = Synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDHTMIFDYWGQGTLVTVSS

SEQ ID NO: 486 - DNA, 24 bp, synthetic construct
  misc_feature 1..24: note = Synthetic
  source 1..24: mol_type = other DNA
ggtgactccgtcagtagttcctac

SEQ ID NO: 487 - protein, 8 aa, synthetic construct
  REGION 1..8: note = Synthetic
GDSVSSSY

SEQ ID NO: 488 - DNA, 21 bp, synthetic construct
  misc_feature 1..21: note = Synthetic
  source 1..21: mol_type = other DNA
atctattacagtgggagttcc

SEQ ID NO: 489 - protein, 7 aa, synthetic construct
  REGION 1..7: note = Synthetic
IYYSGSS

SEQ ID NO: 490 - DNA, 42 bp, synthetic construct
  misc_feature 1..42: note = Synthetic
  source 1..42: mol_type = other DNA
gcgagagaagggaacgtggatcacactatgatatttgactac

SEQ ID NO: 491 - protein, 14 aa, synthetic construct
  REGION 1..14: note = Synthetic
AREGNVDHTMIFDY

SEQ ID NO: 492 - DNA, 360 bp, synthetic construct
  misc_feature 1..360: note = Synthetic
  source 1..360: mol_type = other DNA
caggtgcagctggtggagtctgggggaggcttggtcaagcctggagggtccctgagactctcctgtgcagcctctggattcaccttcagtgactcctacatgtcctggatccgtcaggctccagggaagggactagagtggatttcatacattggtagtagtggtaataccttttactacgcagactctgtgaagggccggttcaccatttccagagacaacgccaacaatttactgtatctgcaaatgaccagcctgagagccgaggacacggccgtgtattactgtgcgagagaagaaggcgatttttggagtgccgttgactcctggggccagggaaccctggtcaccgtctcctca

SEQ ID NO: 493 - protein, 120 aa, synthetic construct
  REGION 1..120: note = Synthetic
QVQLVESGGGLVKPGGSLRLSCAASGFTFSDSYMSWIRQAPGKGLEWISYIGSSGNTFYYADSVKGRFTISRDNANNLLYLQMTSLRAEDTAVYYCAREEGDFWSAVDSWGQGTLVTVSS

SEQ ID NO: 494 - DNA, 24 bp, synthetic construct
  misc_feature 1..24: note = Synthetic
  source 1..24: mol_type = other DNA
ggattcaccttcagtgactcctac

SEQ ID NO: 495 - protein, 8 aa, synthetic construct
  REGION 1..8: note = Synthetic
GFTFSDSY

SEQ ID NO: 496 - DNA, 24 bp, synthetic construct
  misc_feature 1..24: note = Synthetic
  source 1..24: mol_type = other DNA
attggtagtagtggtaataccttt

SEQ ID NO: 497 - protein, 8 aa, synthetic construct
  REGION 1..8: note = Synthetic
IGSSGNTF

SEQ ID NO: 498 - DNA, 39 bp, synthetic construct
  misc_feature 1..39: note = Synthetic
  source 1..39: mol_type = other DNA
gcgagagaagaaggcgatttttggagtgccgttgactcc

SEQ ID NO: 499 - protein, 13 aa, synthetic construct
  REGION 1..13: note = Synthetic
AREEGDFWSAVDS

SEQ ID NO: 500 - DNA, 321 bp, synthetic construct
  misc_feature 1..321: note = Synthetic
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccaggtaaagcccctaaactcctgatccatactgcatccactttgcaaagtggggtcccatcaaggttcagcggcagtggatctgggacagaattcactctcacaatcagcaacctgcagcctgaagattttgcaacttattactgtcaacagcttaatagttacccattcactttcggccctgggaccaaagtggatatcaaa

SEQ ID NO: 501 - protein, 107 aa, synthetic construct
  REGION 1..107: note = Synthetic
DIQLTQSPSFLSASVGDRVTITCWASQGISSYLAWYQQKPGKAPKLLIHTASTLQSGVPSRFSGSGSGTEFTLTISNLQPEDFATYYCQQLNSYPFTFGPGTKVDIK

SEQ ID NO: 502 - DNA, 18 bp, synthetic construct
  misc_feature 1..18: note = Synthetic
  source 1..18: mol_type = other DNA
cagggcattagcagttat

SEQ ID NO: 503 - protein, 6 aa, synthetic construct
  REGION 1..6: note = Synthetic
QGISSY

SEQ ID NO: 504-505 - skipped (2 entries)

SEQ ID NO: 506 - DNA, 27 bp, synthetic construct
  misc_feature 1..27: note = Synthetic
  source 1..27: mol_type = other DNA
caacagcttaatagttacccattcact

SEQ ID NO: 507 - protein, 9 aa, synthetic construct
  REGION 1..9: note = Synthetic
QQLNSYPFT

SEQ ID NO: 508 - DNA, 366 bp, synthetic construct
  misc_feature 1..366: note = Synthetic
  source 1..366: mol_type = other DNA
caggtgcagctggtggagtctgggggaggcgtggtccagcctggggggtccctgagactctcctgtgcagcctctggattcaccttcggtggccatgccatgcactgggtccgccaggctccaggcaaggggctggagtggctggcagttatatcatctgatggcagtaataaacagtatgcagattctgtgaagggccgattcaccatctccagggacaatcccaagaacacgctgtatctgcaaatgaacagtctgagagttggggacacggctatttattactgtgcgaaagaggtggcacctcgttattattattacggtctggacgtctggggccaagggaccacggtcaccgtctcctca

SEQ ID NO: 509 - protein, 122 aa, synthetic construct
  REGION 1..122: note = Synthetic
QVQLVESGGGVVQPGGSLRLSCAASGFTFGGHAMHWVRQAPGKGLEWLAVISSDGSNKQYADSVKGRFTISRDNPKNTLYLQMNSLRVGDTAIYYCAKEVAPRYYYYGLDVWGQGTTVTVSS

SEQ ID NO: 510 - DNA, 24 bp, synthetic construct
  misc_feature 1..24: note = Synthetic
  source 1..24: mol_type = other DNA
ggattcaccttcggtggccatgcc

SEQ ID NO: 511 - protein, 8 aa, synthetic construct
  REGION 1..8: note = Synthetic
GFTFGGHA

SEQ ID NO: 512 - DNA, 24 bp, synthetic construct
  misc_feature 1..24: note = Synthetic
  source 1..24: mol_type = other DNA
atatcatctgatggcagtaataaa

SEQ ID NO: 513 - protein, 8 aa, synthetic construct
  REGION 1..8: note = Synthetic
ISSDGSNK

SEQ ID NO: 514 - DNA, 45 bp, synthetic construct
  misc_feature 1..45: note = Synthetic
  source 1..45: mol_type = other DNA
gcgaaagaggtggcacctcgttattattattacggtctggacgtc

SEQ ID NO: 515 - protein, 15 aa, synthetic construct
  REGION 1..15: note = Synthetic
AKEVAPRYYYYGLDV

SEQ ID NO: 516 - DNA, 318 bp, synthetic construct
  misc_feature 1..318: note = Synthetic
  source 1..318: mol_type = other DNA
gacatccagatgacccagtctccatcctccctgtctgcatctgtgggagacagagtcaccatcacttgccgggcgagtcaggacattagcaattttttagcctggtatcagcagaaaccagggaaggttcctaaactcctgatctatactgcatccactttacaatcaggggtcccatctcggttcagtggcagtggatctgggacagatttcactctcaccgtcagcagcctacagcctgaagatgttgcaacttattactgtcaaaagtatgccggcgccctcactttcggccctgggaccaaagtggatatcaaa

SEQ ID NO: 517 - protein, 106 aa, synthetic construct
  REGION 1..106: note = Synthetic
DIQMTQSPSSLSASVGDRVTITCRASQDISNFLAWYQQKPGKVPKLLIYTASTLQSGVPSRFSGSGSGTDFTLTVSSLQPEDVATYYCQKYAGALTFGPGTKVDIK

SEQ ID NO: 518 - DNA, 18 bp, synthetic construct
  misc_feature 1..18: note = Synthetic
  source 1..18: mol_type = other DNA
caggacattagcaatttt

SEQ ID NO: 519 - protein, 6 aa, synthetic construct
  REGION 1..6: note = Synthetic
QDISNF

SEQ ID NO: 520-521 - skipped (2 entries)

SEQ ID NO: 522 - DNA, 24 bp, synthetic construct
  misc_feature 1..24: note = Synthetic
  source 1..24: mol_type = other DNA
caaaagtatgccggcgccctcact

SEQ ID NO: 523 - protein, 8 aa, synthetic construct
  REGION 1..8: note = Synthetic
QKYAGALT

SEQ ID NO: 524 - DNA, 357 bp, synthetic construct
  misc_feature 1..357: note = Synthetic
  source 1..357: mol_type = other DNA
gaggtgcagctggtggagtctgggggaggcttggcacagcctggggggtccctgagactctcctgtgcagcctctggattcacgtttagaagctatgccatgagctgggtccgccaggctccagggaaggggccggagtgggtctcaggtataggtggtaatggtgttaccacatactacgcagactccgtgaagggccggttcaccatctccagagacaattccaagaacacgctgtttctgcaaatgaatagcctgagagccgaggacacggccgtatattattgtgtgcaggggggtttaggtggttattttacaggctactggggccagggaaccctggtcaccgtctcctca

SEQ ID NO: 525 - protein, 119 aa, synthetic construct
  REGION 1..119: note = Synthetic
EVQLVESGGGLAQPGGSLRLSCAASGFTFRSYAMSWVRQAPGKGPEWVSGIGGNGVTTYYADSVKGRFTISRDNSKNTLFLQMNSLRAEDTAVYYCVQGGLGGYFTGYWGQGTLVTVSS

SEQ ID NO: 526 - DNA, 24 bp, synthetic construct
  misc_feature 1..24: note = Synthetic
  source 1..24: mol_type = other DNA
ggattcacgtttagaagctatgcc

SEQ ID NO: 527 - protein, 8 aa, synthetic construct
  REGION 1..8: note = Synthetic
GFTFRSYA

SEQ ID NO: 528 - DNA, 24 bp, synthetic construct
  misc_feature 1..24: note = Synthetic
  source 1..24: mol_type = other DNA
ataggtggtaatggtgttaccaca

SEQ ID NO: 529 - protein, 8 aa, synthetic construct
  REGION 1..8: note = Synthetic
IGGNGVTT

SEQ ID NO: 530 - DNA, 36 bp, synthetic construct
  misc_feature 1..36: note = Synthetic
  source 1..36: mol_type = other DNA
gtgcaggggggtttaggtggttattttacaggctac

SEQ ID NO: 531 - protein, 12 aa, synthetic construct
  REGION 1..12: note = Synthetic
VQGGLGGYFTGY

SEQ ID NO: 532 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagagtattagtacctatttaaattggtatcagcagaatccagggaaagcccctaaactcctgatctttgatgcatccagtttgcaaagtggggtcccatcaaggttcagtggcagtggatctgggacagatttcactctcaccatcagaggtctgcaacctgaagattttgcaacttactactgtcaacagagttacagtgccccgctcactttcggcggagggaccaaggtggagatcaaa

SEQ ID NO: 533 - protein, 107 aa, synthetic construct
  REGION 1..107: note = Synthetic
DIQMTQSPSSLSASVGDRVTITCRASQSISTYLNWYQQNPGKAPKLLIFDASSLQSGVPSRFSGSGSGTDFTLTIRGLQPEDFATYYCQQSYSAPLTFGGGTKVEIK

SEQ ID NO: 534 - DNA, 18 bp, synthetic construct
  misc_feature 1..18: note = Synthetic
  source 1..18: mol_type = other DNA
cagagtattagtacctat

SEQ ID NO: 535 - protein, 6 aa, synthetic construct
  REGION 1..6: note = Synthetic
QSISTY

SEQ ID NO: 536-537 - skipped (2 entries)

SEQ ID NO: 538 - DNA, 27 bp, synthetic construct
  misc_feature 1..27: note = Synthetic
  source 1..27: mol_type = other DNA
caacagagttacagtgccccgctcact

SEQ ID NO: 539 - protein, 9 aa, synthetic construct
  REGION 1..9: note = Synthetic
QQSYSAPLT

SEQ ID NO: 540 - DNA, 357 bp, synthetic construct
  misc_feature 1..357: note = Synthetic
  source 1..357: mol_type = other DNA
caggtgcagctggtggagtctgggggaggcgtggtccagcctgggaggtccctgagactctcctgtgcagcgtctggattcaccttcagtggttatggcatgcactgggtccgccaggctccaggcaaggggctggagtgggtggcacttatatggcttgatggaagtaatgactactatgcagactccgtgaagggccgattcaccatctccagagacaattccaagaacacgttatatctgcaaatgaacagactgagagccgaggacacggctgtgtattactgtgcgagagatggcccggttgctgctatacccgactactggggccagggaaccctggtcaccgtctcctca

SEQ ID NO: 541 - protein, 119 aa, synthetic construct
  REGION 1..119: note = Synthetic
QVQLVESGGGVVQPGRSLRLSCAASGFTFSGYGMHWVRQAPGKGLEWVALIWLDGSNDYYADSVKGRFTISRDNSKNTLYLQMNRLRAEDTAVYYCARDGPVAAIPDYWGQGTLVTVSS

SEQ ID NO: 542 - DNA, 24 bp, synthetic construct
  misc_feature 1..24: note = Synthetic
  source 1..24: mol_type = other DNA
ggattcaccttcagtggttatggc

SEQ ID NO: 543 - protein, 8 aa, synthetic construct
  REGION 1..8: note = Synthetic
GFTFSGYG

SEQ ID NO: 544 - DNA, 24 bp, synthetic construct
  misc_feature 1..24: note = Synthetic
  source 1..24: mol_type = other DNA
atatggcttgatggaagtaatgac

SEQ ID NO: 545 - protein, 8 aa, synthetic construct
  REGION 1..8: note = Synthetic
IWLDGSND

SEQ ID NO: 546 - DNA, 36 bp, synthetic construct
  misc_feature 1..36: note = Synthetic
  source 1..36: mol_type = other DNA
gcgagagatggcccggttgctgctatacccgactac

SEQ ID NO: 547 - protein, 12 aa, synthetic construct
  REGION 1..12: note = Synthetic
ARDGPVAAIPDY

SEQ ID NO: 548 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtaggtggttggcctggtatcagctgaaaccagggaaagcccctaagctcctgatctataaggcgtctagtttagaaagtggggtcccatcaaggttcagcggcagtggatctgggacagacttcactctcaccatcagcagcctgcaacctgatgattttgcaacttattactgccaacagtataatacttattcgtacacttttggccaggggaccaagctggagatcaaa

SEQ ID NO: 549 - protein, 107 aa, synthetic construct
  REGION 1..107: note = Synthetic
DIQMTQSPSTLSASVGDRVTITCRASQSISRWLAWYQLKPGKAPKLLIYKASSLESGVPSRFSGSGSGTDFTLTISSLQPDDFATYYCQQYNTYSYTFGQGTKLEIK

SEQ ID NO: 550 - DNA, 18 bp, synthetic construct
  misc_feature 1..18: note = Synthetic
  source 1..18: mol_type = other DNA
cagagtattagtaggtgg

SEQ ID NO: 551 - protein, 6 aa, synthetic construct
  REGION 1..6: note = Synthetic
QSISRW

SEQ ID NO: 552-553 - skipped (2 entries)

SEQ ID NO: 554 - DNA, 27 bp, synthetic construct
  misc_feature 1..27: note = Synthetic
  source 1..27: mol_type = other DNA
caacagtataatacttattcgtacact

SEQ ID NO: 555 - protein, 9 aa, synthetic construct
  REGION 1..9: note = Synthetic
QQYNTYSYT

SEQ ID NO: 556 - DNA, 363 bp, synthetic construct
  misc_feature 1..363: note = Synthetic
  source 1..363: mol_type = other DNA
gaggtgcagctggtggagtctgggggaggtgtggtacggcctggggggtccctgagactctcctgtgcagcctctggattcacctttgatgaatatggcatgacttgggtccgccaagttccagggaaggggctggagtgggtctctggtattacttggaatggtggtttcacagattatacagactctgtgaagggccgattcaccagctccagagacaacgccaagaactccctgtatctgcaaatgaacagtctgagagccgaggacacggccttgtattactgtgcgagagatggatatagcagctcgtggggggcttatgatatatggggccaagggacaatggtcaccgtctcttca

SEQ ID NO: 557 - protein, 121 aa, synthetic construct
  REGION 1..121: note = Synthetic
EVQLVESGGGVVRPGGSLRLSCAASGFTFDEYGMTWVRQVPGKGLEWVSGITWNGGFTDYTDSVKGRFTSSRDNAKNSLYLQMNSLRAEDTALYYCARDGYSSSWGAYDIWGQGTMVTVSS

SEQ ID NO: 558 - DNA, 24 bp, synthetic construct
  misc_feature 1..24: note = Synthetic
  source 1..24: mol_type = other DNA
ggattcacctttgatgaatatggc

SEQ ID NO: 559 - protein, 8 aa, synthetic construct
  REGION 1..8: note = Synthetic
GFTFDEYG

SEQ ID NO: 560 - DNA, 24 bp, synthetic construct
  misc_feature 1..24: note = Synthetic
  source 1..24: mol_type = other DNA
attacttggaatggtggtttcaca

SEQ ID NO: 561 - protein, 8 aa, synthetic construct
  REGION 1..8: note = Synthetic
ITWNGGFT

SEQ ID NO: 562 - DNA, 42 bp, synthetic construct
  misc_feature 1..42: note = Synthetic
  source 1..42: mol_type = other DNA
gcgagagatggatatagcagctcgtggggggcttatgatata

SEQ ID NO: 563 - protein, 14 aa, synthetic construct
  REGION 1..14: note = Synthetic
ARDGYSSSWGAYDI

SEQ ID NO: 564 - DNA, 321 bp, synthetic construct
  misc_feature 1..321: note = Synthetic
  source 1..321: mol_type = other DNA
gacatccagatgacccagtctccatcatccctgtctgcatctgtgggagacagagtcaccatcacttgccgggcaagtcagagcattagcacctatttaaattggtatcagcagaaaccagggaaagcccctaagctcctgatctatgctgcatccagtttgcaaagtggggtcccattaaggttcagtggcagtggatctgggactgatttcactctcaccatcagcagtctgcaacctgaagattttgcaagttatttctgtcaacagagttacagtaccccgtacacttttggccaggggaccaagctggagatcaaa

SEQ ID NO: 565 - protein, 107 aa, synthetic construct
  REGION 1..107: note = Synthetic
DIQMTQSPSSLSASVGDRVTITCRASQSISTYLNWYQQKPGKAPKLLIYAASSLQSGVPLRFSGSGSGTDFTLTISSLQPEDFASYFCQQSYSTPYTFGQGTKLEIK

SEQ ID NO: 566 - DNA, 18 bp, synthetic construct
  misc_feature 1..18: note = Synthetic
  source 1..18: mol_type = other DNA
cagagcattagcacctat

SEQ ID NO: 567 - protein, 6 aa, synthetic construct
  REGION 1..6: note = Synthetic
QSISTY

SEQ ID NO: 568-569 - skipped (2 entries)

SEQ ID NO: 570 - DNA, 27 bp, synthetic construct
  misc_feature 1..27: note = Synthetic
  source 1..27: mol_type = other DNA
caacagagttacagtaccccgtacact

SEQ ID NO: 571 - protein, 9 aa, synthetic construct
  REGION 1..9: note = Synthetic
QQSYSTPYT

SEQ ID NO: 572 - DNA, 360 bp, synthetic construct
  misc_feature 1..360: note = Synthetic
  source 1..360: mol_type = other DNA
gaagtgcagctggtggagtctgggggaggcgtggtacagcctggggggtccctgagactctcctgtgcagcctctggattcacctttaatgattatgccatgcactgggtccgtcaagctccagggaagggtctggagtgggtctctcttattagtggagatggtggtaacacatactatgcagactctgtgaagggccgactcaccatctccagagacaacagcaaaaactccctgtatctgcaaatgaacagtctgagaacagaggacaccgccttatattactgtgcaaaagataagggctggaacttcggttacttcgatctctggggccgtggcaccctggtcactgtctcctca

SEQ ID NO: 573 - protein, 120 aa, synthetic construct
  REGION 1..120: note = Synthetic
EVQLVESGGGVVQPGGSLRLSCAASGFTFNDYAMHWVRQAPGKGLEWVSLISGDGGNTYYADSVKGRLTISRDNSKNSLYLQMNSLRTEDTALYYCAKDKGWNFGYFDLWGRGTLVTVSS

SEQ ID NO: 574 - DNA, 24 bp, synthetic construct
  misc_feature 1..24: note = Synthetic
  source 1..24: mol_type = other DNA
ggattcacctttaatgattatgcc

SEQ ID NO: 575 - protein, 8 aa, synthetic construct
  REGION 1..8: note = Synthetic
GFTFNDYA

SEQ ID NO: 576 - DNA, 24 bp, synthetic construct
  misc_feature 1..24: note = Synthetic
  source 1..24: mol_type = other DNA
attagtggagatggtggtaacaca

SEQ ID NO: 577 - protein, 8 aa, synthetic construct
  REGION 1..8: note = Synthetic
ISGDGGNT

SEQ ID NO: 578 - DNA, 39 bp, synthetic construct
  misc_feature 1..39: note = Synthetic
  source 1..39: mol_type = other DNA
gcaaaagataagggctggaacttcggttacttcgatctc

SEQ ID NO: 579 - protein, 13 aa, synthetic construct
  REGION 1..13: note = Synthetic
AKDKGWNFGYFDL

SEQ ID NO: 580 - DNA, 327 bp, synthetic construct
  misc_feature 1..327: note = Synthetic
  source 1..327: mol_type = other DNA
gacatccagatgacccagtctccatcctccctgtctacatctgtgggagacagagtcaccatcacttgccgggcaagtcagaacattgacacctatttaaattggtatcagcagaaaccagggaaagcccctaaactcctgatctatgatgcatccagtttacaaagtggggtcccatcacggttcagtggcagcggatctgggacagatttcactctcaccatcaccagtctgcaacctgaagattttgccacttactactgtcaacagaatgacaatattcttcaccctctcactttcggcggagggaccaaggtggagatcaaa

SEQ ID NO: 581 - protein, 109 aa, synthetic construct
  REGION 1..109: note = Synthetic
DIQMTQSPSSLSTSVGDRVTITCRASQNIDTYLNWYQQKPGKAPKLLIYDASSLQSGVPSRFSGSGSGTDFTLTITSLQPEDFATYYCQQNDNILHPLTFGGGTKVEIK

SEQ ID NO: 582 - DNA, 18 bp, synthetic construct
  misc_feature 1..18: note = Synthetic
  source 1..18: mol_type = other DNA
cagaacattgacacctat

SEQ ID NO: 583 - protein, 6 aa, synthetic construct
  REGION 1..6: note = Synthetic
QNIDTY

SEQ ID NO: 584-585 - skipped (2 entries)

SEQ ID NO: 586 - DNA, 33 bp, synthetic construct
  misc_feature 1..33: note = Synthetic
  source 1..33: mol_type = other DNA
caacagaatgacaatattcttcaccctctcact

SEQ ID NO: 587 - protein, 11 aa, synthetic construct
  REGION 1..11: note = Synthetic
QQNDNILHPLT

SEQ ID NO: 588 - DNA, 369 bp, synthetic construct
  misc_feature 1..369: note = Synthetic
  source 1..369: mol_type = other DNA
gaggtgcagctggtggagtctgggggaggcttggtccaaccgggggggtccctgagactctcctgtgcagcctctggattccactctaatagatattggatggactgggtccgccaggctccagggaaggggctggagtgggtggccaacataaagcaagatggaagtgaggaaaactatgtggactctgtgaagggccgattcaccatctccagagacaacgccaagaactcactttatctgcaaatgaacagcctgagagccgaggacacggctgtgtattactgtgcgagagatcgaagcacctcgtgggtcccttactggttcttcgatctctggggccgtggcaccctggtcactgtctcctca

SEQ ID NO: 589 - protein, 123 aa, synthetic construct
  REGION 1..123: note = Synthetic
EVQLVESGGGLVQPGGSLRLSCAASGFHSNRYWMDWVRQAPGKGLEWVANIKQDGSEENYVDSVKGRFTISRDNAKNSLYLQMNSLRAEDTAVYYCARDRSTSWVPYWFFDLWGRGTLVTVSS

SEQ ID NO: 590 - DNA, 24 bp, synthetic construct
  misc_feature 1..24: note = Synthetic
  source 1..24: mol_type = other DNA
ggattccactctaatagatattgg

SEQ ID NO: 591 - protein, 8 aa, synthetic construct
  REGION 1..8: note = Synthetic
GFHSNRYW

SEQ ID NO: 592 - DNA, 24 bp, synthetic construct
  misc_feature 1..24: note = Synthetic
  source 1..24: mol_type = other DNA
ataaagcaagatggaagtgaggaa

SEQ ID NO: 593 - protein, 8 aa, synthetic construct
  REGION 1..8: note = Synthetic
IKQDGSEE

SEQ ID NO: 594 - DNA, 48 bp, synthetic construct
  misc_feature 1..48: note = Synthetic
  source 1..48: mol_type = other DNA
gcgagagatcgaagcacctcgtgggtcccttactggttcttcgatctc

SEQ ID NO: 595 - protein, 16 aa, synthetic construct
  REGION 1..16: note = Synthetic
ARDRSTSWVPYWFFDL

SEQ ID NO: 596 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 597 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 598 - DNA, 18 bp, synthetic construct
  misc_feature 1..18: note = Synthetic
  source 1..18: mol_type = other DNA
cagagcattagcagctat

SEQ ID NO: 599 - protein, 6 aa, synthetic construct
  REGION 1..6: note = Synthetic
QSISSY

SEQ ID NO: 600-601 - skipped (2 entries)

SEQ ID NO: 602 - DNA, 30 bp, synthetic construct
  misc_feature 1..30: note = Synthetic
  source 1..30: mol_type = other DNA
caacagagttacagtacccctccgatcacc

SEQ ID NO: 603 - protein, 10 aa, synthetic construct
  REGION 1..10: note = Synthetic
QQSYSTPPIT

SEQ ID NO: 604 - DNA, 360 bp, synthetic construct
  misc_feature 1..360: note = Synthetic
  source 1..360: mol_type = other DNA
gaagtgcagctggtggagtctgggggaggcgtggtacagcggggggagtccctgagactctcctgttcagcctctgacttcatctttaaagattatgccatgtactgggtccgtcaaattccagggaagggtctagagtggatctctcttattagtggtgatggtgacactacatggtatggagactctgtgaagggccgattcaccatctccagagacaacaacgaaaactccctctttctgcaaatgaacgatctgagaactgaggacaccgccatgtactactgtgcaagagatatggggtggaacttctttcagttgcaatactggggccagggaaccctggtcaccgtctcctca

SEQ ID NO: 605 - protein, 120 aa, synthetic construct
  REGION 1..120: note = Synthetic
EVQLVESGGGVVQRGESLRLSCSASDFIFKDYAMYWVRQIPGKGLEWISLISGDGDTTWYGDSVKGRFTISRDNNENSLFLQMNDLRTEDTAMYYCARDMGWNFFQLQYWGQGTLVTVSS

SEQ ID NO: 606 - DNA, 24 bp, synthetic construct
  misc_feature 1..24: note = Synthetic
  source 1..24: mol_type = other DNA
gacttcatctttaaagattatgcc

SEQ ID NO: 607 - protein, 8 aa, synthetic construct
  REGION 1..8: note = Synthetic
DFIFKDYA

SEQ ID NO: 608 - DNA, 24 bp, synthetic construct
  misc_feature 1..24: note = Synthetic
  source 1..24: mol_type = other DNA
attagtggtgatggtgacactaca

SEQ ID NO: 609 - protein, 8 aa, synthetic construct
  REGION 1..8: note = Synthetic
ISGDGDTT

SEQ ID NO: 610 - DNA, 39 bp, synthetic construct
  misc_feature 1..39: note = Synthetic
  source 1..39: mol_type = other DNA
gcaagagatatggggtggaacttctttcagttgcaatac

SEQ ID NO: 611 - protein, 13 aa, synthetic construct
  REGION 1..13: note = Synthetic
ARDMGWNFFQLQY

SEQ ID NO: 612 - DNA, 360 bp, synthetic construct
  misc_feature 1..360: note = Synthetic
  source 1..360: mol_type = other DNA
caggtgcagctgcaggagtcgggccccgcactggtgaagccttcacagaccctgtccctcacctgcactgtctctggtggctccatcatcagaggtagtacctactggagttgggtccgccaattcccagggaagggcctggagtggattggatacagttattacagtgggaccgcctactataatccgtccctcgagagtcgagctaccatttctgtagacacgtctaagaaccagttctccctgaacctgaagtctgtgacggccgcggacacggccgtgtattattgtacaagagaaataggagtggctggtctctttgacatctggggccagggaaccctggtcaccgtctcctca

SEQ ID NO: 613 - protein, 120 aa, synthetic construct
  REGION 1..120: note = Synthetic
QVQLQESGPALVKPSQTLSLTCTVSGGSIIRGSTYWSWVRQFPGKGLEWIGYSYYSGTAYYNPSLESRATISVDTSKNQFSLNLKSVTAADTAVYYCTREIGVAGLFDIWGQGTLVTVSS

SEQ ID NO: 614 - DNA, 30 bp, synthetic construct
  misc_feature 1..30: note = Synthetic
  source 1..30: mol_type = other DNA
ggtggctccatcatcagaggtagtacctac

SEQ ID NO: 615 - protein, 10 aa, synthetic construct
  REGION 1..10: note = Synthetic
GGSIIRGSTY

SEQ ID NO: 616 - DNA, 21 bp, synthetic construct
  misc_feature 1..21: note = Synthetic
  source 1..21: mol_type = other DNA
agttattacagtgggaccgcc

SEQ ID NO: 617 - protein, 7 aa, synthetic construct
  REGION 1..7: note = Synthetic
SYYSGTA

SEQ ID NO: 618 - DNA, 36 bp, synthetic construct
  misc_feature 1..36: note = Synthetic
  source 1..36: mol_type = other DNA
acaagagaaataggagtggctggtctctttgacatc

SEQ ID NO: 619 - protein, 12 aa, synthetic construct
  REGION 1..12: note = Synthetic
TREIGVAGLFDI

SEQ ID NO: 620 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 621 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 622 - DNA, 36 bp, synthetic construct
  misc_feature 1..36: note = Synthetic
  source 1..36: mol_type = other DNA
agagcaagtcagtcagtctctagctcttatctcgcc

SEQ ID NO: 623 - protein, 12 aa, synthetic construct
  REGION 1..12: note = Synthetic
RASQSVSSSYLA

SEQ ID NO: 624 - DNA, 21 bp, synthetic construct
  misc_feature 1..21: note = Synthetic
  source 1..21: mol_type = other DNA
ggggcaagttccagggccacc

SEQ ID NO: 625 - protein, 7 aa, synthetic construct
  REGION 1..7: note = Synthetic
GASSRAT

SEQ ID NO: 626 - DNA, 27 bp, synthetic construct
  misc_feature 1..27: note = Synthetic
  source 1..27: mol_type = other DNA
caacagtacggaagcagcccgtggacg

SEQ ID NO: 627 - protein, 9 aa, synthetic construct
  REGION 1..9: note = Synthetic
QQYGSSPWT

SEQ ID NO: 628 - DNA, 357 bp, synthetic construct
  misc_feature 1..357: note = Synthetic
  source 1..357: mol_type = other DNA
caggagcagttggtgcagtctggggctgaggtgaagaagcctggggcctcagtgaaggtctcctgtaaggcttctggatacaccttcaccggctactatatacattgggtgcgacaggcccctggactagggcttgaatggatgggatggatcaaccctaacagtggtggcacaaaatatgcacagaagtttcagggcagggtcaccatgaccagggacacgtccatcaatacagcctacatggagctgaaaagactgaaatctgacgactcggccgtatattactgtgcgagagacgcccctccccatgatgtttttgatatctggggccaagggacattggtcaccgtctcttca

SEQ ID NO: 629 - protein, 119 aa, synthetic construct
  REGION 1..119: note = Synthetic
QEQLVQSGAEVKKPGASVKVSCKASGYTFTGYYIHWVRQAPGLGLEWMGWINPNSGGTKYAQKFQGRVTMTRDTSINTAYMELKRLKSDDSAVYYCARDAPPHDVFDIWGQGTLVTVSS

SEQ ID NO: 630 - DNA, 24 bp, synthetic construct
  misc_feature 1..24: note = Synthetic
  source 1..24: mol_type = other DNA
ggatacaccttcaccggctactat

SEQ ID NO: 631 - protein, 8 aa, synthetic construct
  REGION 1..8: note = Synthetic
GYTFTGYY

SEQ ID NO: 632 - DNA, 24 bp, synthetic construct
  misc_feature 1..24: note = Synthetic
  source 1..24: mol_type = other DNA
atcaaccctaacagtggtggcaca

SEQ ID NO: 633 - protein, 8 aa, synthetic construct
  REGION 1..8: note = Synthetic
INPNSGGT

SEQ ID NO: 634 - DNA, 36 bp, synthetic construct
  misc_feature 1..36: note = Synthetic
  source 1..36: mol_type = other DNA
gcgagagacgcccctccccatgatgtttttgatatc

SEQ ID NO: 635 - protein, 12 aa, synthetic construct
  REGION 1..12: note = Synthetic
ARDAPPHDVFDI

SEQ ID NO: 636 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaattggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 637 - protein, 107 aa, synthetic construct
  REGION 1..107: note = Synthetic
DIQMTQSPSSLSASVGDRVTITCRASQGIRNDLGWYQQKPGKAPKRLIYAASSLQIGVPSRFSGSGSGTEFTLTISSLQPEDFATYYCLQHNSYPLTFGGGTKVEIK

SEQ ID NO: 638 - DNA, 18 bp, synthetic construct
  misc_feature 1..18: note = Synthetic
  source 1..18: mol_type = other DNA
cagggcattagaaatgat

SEQ ID NO: 639 - protein, 6 aa, synthetic construct
  REGION 1..6: note = Synthetic
QGIRND

SEQ ID NO: 640-641 - skipped (2 entries)

SEQ ID NO: 642 - DNA, 27 bp, synthetic construct
  misc_feature 1..27: note = Synthetic
  source 1..27: mol_type = other DNA
ctacagcataatagttacccgctcact

SEQ ID NO: 643 - protein, 9 aa, synthetic construct
  REGION 1..9: note = Synthetic
LQHNSYPLT

SEQ ID NO: 644 - DNA, 357 bp, synthetic construct
  misc_feature 1..357: note = Synthetic
  source 1..357: mol_type = other DNA
caggtgcagctgcaagagtcgggcccaggactggtgaagccttcacagaccctgtccctcacctgcactgtctctggtggctccatcagtagtggtgcttaccactggagctggatccgccagcacccagggaagggcctagagtggattggatacatctattacaatggggacacctactataatccgtccctcaagagtcgcgttaccatttcagtggacacgtctaagaaccaattcttcctgaaggtgacctctgtgactgccgcggacacggccatgtattactgtgcgggagaaaagcagctgactgcttttgatatctggggccaagggacattggtcaccgtctcttca

SEQ ID NO: 645 - protein, 119 aa, synthetic construct
  REGION 1..119: note = Synthetic
QVQLQESGPGLVKPSQTLSLTCTVSGGSISSGAYHWSWIRQHPGKGLEWIGYIYYNGDTYYNPSLKSRVTISVDTSKNQFFLKVTSVTAADTAMYYCAGEKQLTAFDIWGQGTLVTVSS

SEQ ID NO: 646 - DNA, 30 bp, synthetic construct
  misc_feature 1..30: note = Synthetic
  source 1..30: mol_type = other DNA
ggtggctccatcagtagtggtgcttaccac

SEQ ID NO: 647 - protein, 10 aa, synthetic construct
  REGION 1..10: note = Synthetic
GGSISSGAYH

SEQ ID NO: 648 - DNA, 21 bp, synthetic construct
  misc_feature 1..21: note = Synthetic
  source 1..21: mol_type = other DNA
atctattacaatggggacacc

SEQ ID NO: 649 - protein, 7 aa, synthetic construct
  REGION 1..7: note = Synthetic
IYYNGDT

SEQ ID NO: 650 - DNA, 33 bp, synthetic construct
  misc_feature 1..33: note = Synthetic
  source 1..33: mol_type = other DNA
gcgggagaaaagcagctgactgcttttgatatc

SEQ ID NO: 651 - protein, 11 aa, synthetic construct
  REGION 1..11: note = Synthetic
AGEKQLTAFDI

SEQ ID NO: 652 - DNA, 321 bp, synthetic construct
  misc_feature 1..321: note = Synthetic
  source 1..321: mol_type = other DNA
gtcatccagatgacccagtctccatcctccctgtctgcatctgttggagacagagtcaccataacttgccgggcgagtcaggacattaataattttttaaattggtatcaacagaaattagggaaagcccctaaactcctgatctccgatgcatccaatttgcagacaggagtcccgtcaaggttcagtggaagtggatctgggacagattttactttcaccatcagcagcctgcagcctgaagatattgctgcatattactgtcaacaatatgatcatttcccgtatacttttggccaggggaccagactggagaacaat

SEQ ID NO: 653 - protein, 107 aa, synthetic construct
  REGION 1..107: note = Synthetic
VIQMTQSPSSLSASVGDRVTITCRASQDINNFLNWYQQKLGKAPKLLISDASNLQTGVPSRFSGSGSGTDFTFTISSLQPEDIAAYYCQQYDHFPYTFGQGTRLENN

SEQ ID NO: 654 - DNA, 18 bp, synthetic construct
  misc_feature 1..18: note = Synthetic
  source 1..18: mol_type = other DNA
caggacattaataatttt

SEQ ID NO: 655 - protein, 6 aa, synthetic construct
  REGION 1..6: note = Synthetic
QDINNF

SEQ ID NO: 656-657 - skipped (2 entries)

SEQ ID NO: 658 - DNA, 27 bp, synthetic construct
  misc_feature 1..27: note = Synthetic
  source 1..27: mol_type = other DNA
caacaatatgatcatttcccgtatact

SEQ ID NO: 659 - protein, 9 aa, synthetic construct
  REGION 1..9: note = Synthetic
QQYDHFPYT

SEQ ID NO: 660 - DNA, 357 bp, synthetic construct
  misc_feature 1..357: note = Synthetic
  source 1..357: mol_type = other DNA
gaggtgcagttggtggagtctgggggaggtgtggttcggcctggggggtccctgagactctcctgtgcagcctctggattcacctttgatgattatggcatgacctgggtccgccaagctccagggaaggggctggagtgggtctctggtattaattggaatggcgatagcacagagtattcagactctgtgaagggccgattcaccatctccagagacaacgccaagaactccctgtatctgcaaatgaacagtctgagagccgaggacacggccttctatcactgtgcgagagagaataactggaacttctactttgactactggggccagggaaccctggtcaccgtctcctca

SEQ ID NO: 661 - protein, 119 aa, synthetic construct
  REGION 1..119: note = Synthetic
EVQLVESGGGVVRPGGSLRLSCAASGFTFDDYGMTWVRQAPGKGLEWVSGINWNGDSTEYSDSVKGRFTISRDNAKNSLYLQMNSLRAEDTAFYHCARENNWNFYFDYWGQGTLVTVSS

SEQ ID NO: 662 - DNA, 24 bp, synthetic construct
  misc_feature 1..24: note = Synthetic
  source 1..24: mol_type = other DNA
ggattcacctttgatgattatggc

SEQ ID NO: 663 - protein, 8 aa, synthetic construct
  REGION 1..8: note = Synthetic
GFTFDDYG

SEQ ID NO: 664 - DNA, 24 bp, synthetic construct
  misc_feature 1..24: note = Synthetic
  source 1..24: mol_type = other DNA
attaattggaatggcgatagcaca

SEQ ID NO: 665 - protein, 8 aa, synthetic construct
  REGION 1..8: note = Synthetic
INWNGDST

SEQ ID NO: 666 - DNA, 36 bp, synthetic construct
  misc_feature 1..36: note = Synthetic
  source 1..36: mol_type = other DNA
gcgagagagaataactggaacttctactttgactac

SEQ ID NO: 667 - protein, 12 aa, synthetic construct
  REGION 1..12: note = Synthetic
ARENNWNFYFDY

SEQ ID NO: 668 - DNA, 321 bp, synthetic construct
  misc_feature 1..321: note = Synthetic
  source 1..321: mol_type = other DNA
gaaatagtgatgacgcagtctccagccaccctgtctgtgtctcgaggggaaagagccaccctctcctgtagggccagtcagagtgttagcagcaacttagcctggtaccagcagaaacttggccaggctcccaggctcctcatctatggtgcatccaccagggccactggtatcccagccaggttcagtggcagtgggtctgggacagagttcactctcaccatcagcagcctgcagtctgaagattttgcagtttattattgtcagcagtataataactggccgtggacgttcggccaagggaccaaggtggaaatcaaa

SEQ ID NO: 669 - protein, 107 aa, synthetic construct
  REGION 1..107: note = Synthetic
EIVMTQSPATLSVSRGERATLSCRASQSVSSNLAWYQQKLGQAPRLLIYGASTRATGIPARFSGSGSGTEFTLTISSLQSEDFAVYYCQQYNNWPWTFGQGTKVEIK

SEQ ID NO: 670 - DNA, 18 bp, synthetic construct
  misc_feature 1..18: note = Synthetic
  source 1..18: mol_type = other DNA
cagagtgttagcagcaac

SEQ ID NO: 671 - protein, 6 aa, synthetic construct
  REGION 1..6: note = Synthetic
QSVSSN

SEQ ID NO: 672-673 - skipped (2 entries)

SEQ ID NO: 674 - DNA, 27 bp, synthetic construct
  misc_feature 1..27: note = Synthetic
  source 1..27: mol_type = other DNA
cagcagtataataactggccgtggacg

SEQ ID NO: 675 - protein, 9 aa, synthetic construct
  REGION 1..9: note = Synthetic
QQYNNWPWT

SEQ ID NO: 676 - DNA, 351 bp, synthetic construct
  misc_feature 1..351: note = Synthetic
  source 1..351: mol_type = other DNA
caggtccacctggtacagtctggggctgaggtgaagaagcctggggcctcagtgaaggtctcctgcaaggtttccggaaacaccctcactgaattatccatgcactgggtgcgacaggctcctggaaaagggcttgagtggatgggaggttttgatcctgaagatggtgacacaatctactcacagaagttccagggcagagtcaccttgaccgaggacacatctacagacacagcctacatggagctgagcagcctgagatctgaggacacggccgtgtattactgttcaacagtggggggacctacctctgactgctggggccagggaaccctggtcaccgtctcctca

SEQ ID NO: 677 - protein, 117 aa, synthetic construct
  REGION 1..117: note = Synthetic
QVHLVQSGAEVKKPGASVKVSCKVSGNTLTELSMHWVRQAPGKGLEWMGGFDPEDGDTIYSQKFQGRVTLTEDTSTDTAYMELSSLRSEDTAVYYCSTVGGPTSDCWGQGTLVTVSS

SEQ ID NO: 678 - DNA, 24 bp, synthetic construct
  misc_feature 1..24: note = Synthetic
  source 1..24: mol_type = other DNA
ggaaacaccctcactgaattatcc

SEQ ID NO: 679 - protein, 8 aa, synthetic construct
  REGION 1..8: note = Synthetic
GNTLTELS

SEQ ID NO: 680 - DNA, 24 bp, synthetic construct
  misc_feature 1..24: note = Synthetic
  source 1..24: mol_type = other DNA
tttgatcctgaagatggtgacaca

SEQ ID NO: 681 - protein, 8 aa, synthetic construct
  REGION 1..8: note = Synthetic
FDPEDGDT

SEQ ID NO: 682 - DNA, 30 bp, synthetic construct
  misc_feature 1..30: note = Synthetic
  source 1..30: mol_type = other DNA
tcaacagtggggggacctacctctgactgc

SEQ ID NO: 683 - protein, 10 aa, synthetic construct
  REGION 1..10: note = Synthetic
STVGGPTSDC

SEQ ID NO: 684 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaaggtcctgatcttcgatgcatccaatttagaaccaggggtcccatcaaggttcagtggaagtggatctgggacagattttactttcaccatcatcagcctgcagcctgaagatattgcaacatattactgtcaacaatatgataatctcccgatcaccttcggccaggggacacgactggacattaaa

SEQ ID NO: 685 - protein, 107 aa, synthetic construct
  REGION 1..107: note = Synthetic
DIQMTQSPSSLSASVGDRVTITCQASQDISNYLNWYQQKPGKAPKVLIFDASNLEPGVPSRFSGSGSGTDFTFTIISLQPEDIATYYCQQYDNLPITFGQGTRLDIK

SEQ ID NO: 686 - DNA, 18 bp, synthetic construct
  misc_feature 1..18: note = Synthetic
  source 1..18: mol_type = other DNA
caggacattagcaactat

SEQ ID NO: 687 - protein, 6 aa, synthetic construct
  REGION 1..6: note = Synthetic
QDISNY

SEQ ID NO: 688-689 - skipped (2 entries)

SEQ ID NO: 690 - DNA, 27 bp, synthetic construct
  misc_feature 1..27: note = Synthetic
  source 1..27: mol_type = other DNA
caacaatatgataatctcccgatcacc

SEQ ID NO: 691 - protein, 9 aa, synthetic construct
  REGION 1..9: note = Synthetic
QQYDNLPIT

SEQ ID NO: 692 - protein, 9 aa, synthetic construct
  REGION 1..9: note = Synthetic
NMATGMDSW

SEQ ID NO: 693 - protein, 20 aa, synthetic construct
  REGION 1..20: note = Synthetic
WEVHLVPRRKQLQFALPDSL

SEQ ID NO: 694 - protein, 7 aa, synthetic construct
  REGION 1..7: note = Synthetic
ATGMDSW

SEQ ID NO: 695 - protein, 13 aa, synthetic construct
  REGION 1..13: note = Synthetic
WEVHLVPRRKQLQ

SEQ ID NO: 696 - protein, 9 aa, synthetic construct
  REGION 1..9: note = Synthetic
LVPRRKQLQ

SEQ ID NO: 697 - protein, 447 aa, synthetic construct
  REGION 1..447: note = Synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 698 - protein, 214 aa, synthetic construct
  REGION 1..214: note = Synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 699 - protein, 18 aa, synthetic construct
  REGION 1..18: note = Synthetic
KDMQLGRLHMKTLLPVSK

SEQ ID NO: 700 - protein, 9 aa, synthetic construct
  REGION 1..9: note = Synthetic
NDETCEQRA

SEQ ID NO: 701 - protein, 7 aa, synthetic construct
  REGION 1..7: note = Synthetic
SHKDMQL

SEQ ID NO: 702 - protein, 451 aa, synthetic construct
  REGION 1..451: note = Synthetic
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 703 - protein, 217 aa, synthetic construct
  REGION 1..217: note = Synthetic
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 704 - protein, 1676 aa, Homo sapiens; identical to SEQ ID NO: 1

SEQ ID NO: 705 - protein, 1658 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVHQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 706 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 707 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 708 - DNA, 130 bp, synthetic construct
  misc_feature 1..130: note = Synthetic
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct